(19) United States
(12) Patent Application Publication
(10) Pub. No.: US 2015/0087673 A1
(43) Pub. Date: Mar. 26, 2015

(54) METHODS FOR USING AND BIOMARKERS FOR AMPK-ACTIVATING COMPOUNDS

(71) Applicants: Yasumichi Hitoshi, Brisbane, CA (US); Yonchu Jenkins, Oakland, CA (US); Vadim Markovtsov, San Mateo, CA (US); Todd Kinsella, Redwood City, CA (US); Tian-Qiang Sun, San Francisco, CA (US)

(72) Inventors: Yasumichi Hitoshi, Brisbane, CA (US); Yonchu Jenkins, Oakland, CA (US); Vadim Markovtsov, San Mateo, CA (US); Todd Kinsella, Redwood City, CA (US); Tian-Qiang Sun, San Francisco, CA (US)

(73) Assignee: RIGEL PHARMACEUTICALS, INC., South San Francisco, CA (US)

(21) Appl. No.: 14/498,921

(22) Filed: Sep. 26, 2014

Related U.S. Application Data
(60) Provisional application No. 61/883,126, filed on Sep. 26, 2013.

(51) Int. Cl. A61K 31/4545; C07D 401/14; A61K 45/06; G01N 33/573

(52) U.S. Cl. CPC A61K 31/4545; G01N 33/573; C07D 401/14; A61K 45/06; G01N 2500/04
USPC 514/316

(57) ABSTRACT

Disclosed are methods of using AMPK-activating compounds, for example, in the treatment of cancer and disorders of vascular flow. Also disclosed are biomarkers for AMPK and uses thereof, for example, in the diagnosis and treatment of AMPK-linked disorders. In certain embodiments, the AMPK-activating compounds have the structural formula

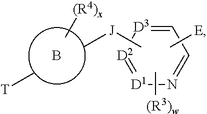

wherein E, J, T, D¹, D², D³, the ring system denoted by “B”, T, R³, R⁴, w and x are as described herein.

### CROSS-REFERENCE TO RELATED APPLICATIONS

This application claims the benefit of priority of U.S. Provisional Patent Application Ser. No. 61/883,126, filed Sep. 26, 2013, which is hereby incorporated herein by reference in its entirety.

### BACKGROUND

1. Field

This disclosure relates generally to methods of use of certain compounds and compositions containing them, as well as to certain biomarkers of the effects of the compounds and methods for using them. This disclosure relates more particularly to methods of use of certain substituted pyridine compounds and pharmaceutical compositions thereof.

2. Technical Background

The kinase 5″-AMP-activated protein kinase (AMPK) is well established as an important sensor and regulator of cellular energy homeostasis. Being a multi-substrate enzyme, AMPK regulates a variety of metabolic processes, such as glucose transport, glycolysis and lipid metabolism. It acts as a sensor of cellular energy homeostasis and is activated in response to certain hormones and muscle contraction as well as to intracellular metabolic stress signals such as exercise, ischemia, hypoxia and nutrient deprivation. Once activated, AMPK switches on catabolic pathways (such as fatty acid oxidation and glycolysis) and switches off ATP-consuming pathways (such as lipogenesis). Activation of the AMPK pathway improves insulin sensitivity by directly stimulating glucose uptake in adipocytes and muscle and by increasing fatty acid oxidation in liver and muscle, resulting in reduced circulating fatty acid levels and reduced intracellular triglyceride contents. Moreover, activation of the AMPK pathway decreases glycogen concentration by reducing the activity of glycogen synthase. Activation of the AMPK pathway also plays a protective role against inflammation and atherosclerosis. It suppresses the expression of adhesion molecules in vascular endothelial cells and cytokine production from macrophages, thus inhibiting the inflammatory processes that occur during the early phases of atherosclerosis.

### SUMMARY

Disclosed herein are certain methods of using AMPK-activating compounds, for example, AMPK-activating compounds having structural formula (I)

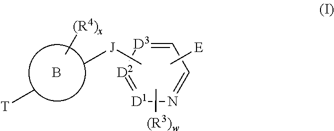

and pharmaceutically acceptable salts, prodrugs and N-oxides thereof (and solvates and hydrates thereof), in which the variables are as described herein.

In certain aspects, the disclosure provides methods for sensitizing a cancer cell to apoptosis; upregulating p53 activity in a cancer cell; or inducing a cytotoxic effect in a cancer cell, the methods including contacting the cancer cell with an effective amount of an AMPK-activating compound or a pharmaceutically acceptable salt, prodrug or N-oxide thereof, or a solvate or hydrate thereof.

In other aspects, the disclosure provides methods for treating cancer in a subject in need thereof, the methods including administering to the subject a therapeutically-effective amount of an AMPK-activating compound or a pharmaceutically acceptable salt, prodrug or N-oxide thereof, or a solvate or hydrate thereof, optionally in combination with other anticancer therapy.

In other aspects, the disclosure provides methods for increasing vascular flow and treating disorders of vascular flow in a subject in need thereof, the methods including administering to the subject a therapeutically-effective amount of an AMPK-activating compound or a pharmaceutically acceptable salt, prodrug or N-oxide thereof, or a solvate or hydrate thereof, optionally in combination with other anticancer therapy.

In other aspects, the disclosure provides a method for treating cancer in a subject in need thereof, the cancer being selected from the group consisting of melanoma, myeloma, endometrial carcinosarcoma, soft tissue sarcoma, hepatocellular carcinoma, lung adenocarcinoma, large lung cell carcinoma and colorectal carcinoma, the method including administering to the subject a therapeutically-effective amount of an AMPK-activating compound or a pharmaceutically acceptable salt, prodrug or N-oxide thereof (or a solvate or hydrate thereof).

In other aspects, the disclosure provides a method for treating pulmonary arterial hypertension in a subject in need thereof, the method including administering to the subject a therapeutically-effective amount of an AMPK-activating compound or a pharmaceutically acceptable salt, prodrug or N-oxide thereof (or a solvate or hydrate thereof).

In other aspects, the disclosure provides a method for treating vasculitis or venous ulcers in a subject in need thereof, the method including administering to the subject a therapeutically-effective amount of an AMPK-activating compound or a pharmaceutically acceptable salt, prodrug or N-oxide thereof (or a solvate or hydrate thereof).

In other aspects, the disclosure provides a method for down-regulating UHRF1 (Np95) in a cell, the method comprising contacting the cell with an AMPK-activating compound (e.g., a compound as disclosed herein) or a pharmaceutically acceptable salt, prodrug or N-oxide thereof (or a solvate or hydrate thereof).

Other aspects of the disclosure relate to biomarkers of AMPK activation, such as branched chain amino acids, tyrosine, phenylalanine, acylcarnitine intermediates, insulin-like growth factor-binding protein-1, ketone bodies, citric acid cycle intermediates and fatty acids.

In certain aspects of the disclosure, a method of determining the degree of AMPK activation in a subject includes:
administering to the subject an AMPK-activating compound; then
obtaining a sample from the subject; and
measuring the concentration of a biomarker of AMPK activation in the sample from the subject.

In other aspects of the disclosure, a method of determining the degree of AMPK activation in a subject includes:
obtaining a first sample from the subject;
measuring the initial concentration of a biomarker of AMPK activation in the first sample from the subject;
after obtaining the first sample from the subject, administering to the subject an AMPK-activating compound;
after administration, obtaining a second sample from the subject; and
measuring the concentration of the biomarker in the second sample from the subject.

In other aspects of the disclosure, a method of activating the AMPK pathway in a subject in need thereof includes:
obtaining a first sample from the subject;
measuring the concentration of a biomarker of AMPK activation in the first sample from the subject;
after obtaining the first sample, administering to the subject an AMPK-activating compound at a test dosage;
after administration, obtaining a second sample from the subject;
measuring the concentration of the biomarker of AMPK activation in the second sample from the subject;
selecting a therapeutic dosage of the AMPK-activating compound based on the concentration of the biomarker of AMPK activation in the second sample, optionally together with the concentration of the biomarker of AMPK activation in the first sample; and
administering to the subject the AMPK-activating compound at at least about the therapeutic dosage.

In other aspects, the disclosure provides
administering to the subject an AMPK-activating compound at a test dosage;
after administration, obtaining a sample from the subject;
measuring the concentration of the biomarker of AMPK activation in the sample from the subject;
selecting a therapeutic dosage of the AMPK-activating compound based on the concentration of the biomarker of AMPK activation in the sample; and
administering to the subject the AMPK-activating compound at at least about the therapeutic dosage.

In certain embodiments, the therapeutic dosage is selected to be effective in treating an AMPK-linked disorder.

Various other aspects of the disclosure are described in the sections below.

### BRIEF DESCRIPTION OF THE DRAWINGS

FIG. 1 is a set of graphs ¹³CO₂ versus ¹²CO₂ enrichment following [U-¹³C]-palmitate tracer administration for skeletal muscle, liver and adipose tissue.

FIG. 2 is a set of graphs ¹³CO₂ versus ¹²CO₂ enrichment following [U-¹³C]-D-glucose tracer administration for skeletal muscle, liver and adipose tissue.

### DETAILED DESCRIPTION

Tissue homeostasis is maintained by a balance between the rate of cell proliferation and the rate of cell death. Apoptosis, or programmed cell death, is one mechanism by which cell proliferation is balanced. Apoptosis is also necessary for the sustenance of tissue viability, as the constant renewal of tissue provides a physiologic scaffold for regenerative metabolism. When cellular renewal is homeostatically balanced, the integrity of proliferative, immunomodulatory and angiogenic components of tissue metabolism are maintained. However, loss of regulation of any one of, or a combination of these processes may result in a lack of apoptic control. A perturbation of the link between cell growth and cell death can result in the development of cancer through aberrant cell proliferation, including the growth of tumor cells.

The tumor suppressor protein p53 is a short lived, latent transcription factor that is activated and stabilized in response to a wide range of cellular stresses, including DNA damage and activated oncogenes. Under healthy conditions, p53 can recognize when the integrity of a cell is compromised, and commits it to apoptosis via employment of the Bcl-2 protein family in the mitochondria, leading to nuclear fragmentation. Due to its role in conserving stability by preventing genome mutation, p53 has been called “the guardian of the genome.” p53 has been shown to participate in the regulation of several processes, which might inhibit tumor growth, including differentiation, senescence and angiogenesis. Loss of the ability to induce p53 or other loss of p53 activity can lead to uncontrolled cell proliferation and tumor growth. In many human cancers, a wild-type p53 gene is retained. In such cancers, a frequent defect is a failure to stabilize and activate p53 to prevent uncontrolled cell growth and tumor development. In other cancers, p53 itself is mutated so as to be inactive, or even absent.

One aspect of the disclosure is a method of sensitizing a cancer cell to apoptosis, the method including contacting the cancer cell with an effective amount of an AMPK-activating compound or a pharmaceutically acceptable salt, prodrug or N-oxide thereof (or a solvate or hydrate thereof). Another aspect of the disclosure is a method of upregulating p53 activity in a cancer cell, the method comprising contacting the cancer cell with an effective amount of an AMPK-activating compound or a pharmaceutically acceptable salt, prodrug or N-oxide thereof (or a solvate or hydrate thereof). Another aspect of the disclosure is a method of inducing a cytotoxic effect in a cancer cell, the method comprising contacting the cancer cell with an effective amount of an AMPK-activating compound or a pharmaceutically acceptable salt, prodrug or N-oxide thereof (or a solvate or hydrate thereof). Myc has been shown to activate the AMPK pathway, which induces mitochondrial accumulation of p53, which in turn induces apoptosis. See A. I. Nieminen et al., “Myc-induced AMPK-phospho p53 pathway activates Bak to sensitize mitochondrial apoptosis,” PNAS110(20):E1839-48 (2013), which is hereby incorporated herein by reference in its entirety. Accordingly, direct activation of AMPK will also induce mitochondrial accumulation of p53, and thus apoptosis.

In rapidly proliferating cells, Myc deregulates the cell cycle independent of nutrient availability. Dividing cells require constant anabolic metabolism, at the expense of ATP production, for macromolecule synthesis and production of biomass. Cells undergoing Myc-transformed metabolism will maintain low levels of ATP, and thus will be especially sensitized to apoptosis. In contrast, in normal cells ATP is rapidly replenished, making the accumulation of p53 a transient and relatively harmless event.

Because induction of apoptosis can arrest undesirable cell proliferation such as tumor growth, AMPK activation can be effective in treating cancer and other cell proliferation disorders. Accordingly, another aspect of the disclosure is a method of treating cancer in a subject in need thereof, the method including administering to the subject a therapeutically-effective amount of an AMPK-activating compound or a pharmaceutically acceptable salt, prodrug or N-oxide thereof (or a solvate or hydrate thereof). The person of ordinary skill in the art will determine a therapeutically-effective amount for a particular patient and a particular cancer using conventional methods.

In certain embodiments, the cancer or cancer cell is selected from the group consisting of breast cancer, pancreas cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteosarcoma, chondrosarcoma, Ewing's sarcoma, malignant fibrous histiocytoma, fibrosarcoma, multiple myeloma, reticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, glioblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas. In certain embodiments of the methods of the disclosure, the cancer or cancer cell is selected from cancers of the breast, pancreas, skin, prostate, liver, lung, lymphoid system, bladder, kidney, brain, colon and bone. In certain embodiments of the methods of the disclosure, the cancer is selected from the group consisting of melanoma, prostate adenocarcinoma, lymphoma, pancreatic ductal carcinoma, renal carcinoma, hepatocellular carcinoma, small cell lung carcinoma, non-small cell lung carcinoma, urothelial cell carcinoma, colon carcinoma, glioblastoma, breast lobular or ductal carcinoma, osteosarcoma, chondrosarcoma, and multiple myeloma.

In certain particular embodiments, the cancer or cancer cell is selected from the group consisting of melanoma, myeloma, endometrial carcinosarcoma, soft tissue sarcoma, hepatocellular carcinoma, lung adenocarcinoma, large lung cell carcinoma and colorectal carcinoma. For example, in one embodiment, the cancer of cancer cell is melanoma. In another embodiment, the cancer or cancer cell is myeloma. In another embodiment, the cancer or cancer cell is endometrial carcinosarcoma. In another embodiment, the cancer or cancer cell is soft tissue sarcoma. In another embodiment, the cancer or cancer cell is hepatocellular carcinoma. In another embodiment, the cancer or cancer cell is lung adenocarcinoma. In another embodiment, the cancer or cancer cell is large lung cell carcinoma. In another embodiment, the cancer or cancer cell is colorectal carcinoma. Without being limited to any particular theory, it is currently is believed that the compounds exert their antiproliferative effects in these cancers by down regulating UHRF1 (Np95), which is a ubiquitin ligase. Accordingly, another aspect of the disclosure is a method for down-regulating UHRF1 (Np95) in a cell, the method comprising contacting the cell with an AMPK-activating compound (e.g., a compound as disclosed herein) or a pharmaceutically acceptable salt, prodrug or N-oxide thereof (or a solvate or hydrate thereof).

In certain embodiments as described above, the cancer or cancer cell is one in which wild-type p53 is expressed. In other embodiments as described above, the cancer or cancer cell is one in which p53 is mutated but remains functional.

In certain embodiments as described herein, an AMPK-activating compound or the pharmaceutically acceptable salt, prodrug or N-oxide thereof (or the solvate or hydrate thereof) is used in combination with other anticancer therapy in the treatment of cancer. For example, in one embodiment, a method of treating cancer in a subject in need thereof includes administering to the subject a therapeutically-effective amount of an AMPK-activating compound or a pharmaceutically acceptable salt, prodrug or N-oxide thereof (or a solvate or hydrate thereof) in combination with ionizing radiation therapy. In another embodiment, a method of treating cancer in a subject in need thereof includes administering to the subject a therapeutically-effective amount of an AMPK-activating compound or a pharmaceutically acceptable salt, prodrug or N-oxide thereof (or a solvate or hydrate thereof) in combination with a chemotherapeutic agent. The AMPK-activating compound or the pharmaceutically acceptable salt, prodrug or N-oxide thereof (or the solvate or hydrate thereof) can be, for example, administered substantially simultaneously with the other cancer therapy. Of course, in other embodiments, the AMPK-activating compound or the pharmaceutically acceptable salt, prodrug or N-oxide thereof (or the solvate or hydrate thereof) is not administered substantially simultaneously with the other cancer therapy. In certain such embodiments, the AMPK-activating compound or the pharmaceutically acceptable salt, prodrug or N-oxide thereof (or the solvate or hydrate thereof) is administered such that an effective amount (e.g., at least about 5% of Cₘₐₓ, at least about 10% of Cₘₐₓ, at least about 20% of Cₘₐₓ, or even at least about 50% of Cₘₐₓ) of an AMPK-activating compound remains in the subject at a time that during which the other anticancer therapy is active.

A wide variety of chemotherapeutic agents can be used in combination with the AMPK-activating compound. For example, the chemotherapeutic agent can be an alkylating agent (e.g., cyclophosphamide, mechlorethamine, chlorambucil, melphalan; ifosfamide; streptozocin, carmustine, lomustine, busulfan, dacarbazine, temozolomide, thiotepa, altretamine); an anthracycline (e.g., daunorubicin, doxorubicin, epirubicin, idarubicin, mitoxantrone, valrubicin); a taxane (e.g., paclitaxel, docetaxel); an epothilone (e.g., ixabepalone); a histone deacetylase inhibitor (e.g., vorinostat, romidepsin), a topoisomerase inhibitor (e.g., etoposide, irinotecan, tafluposide, teniposide, toptecan); a kinase inhibitor (e.g., bortezomib, erlotinib, gefitinib, imatinib, vemerafenib, vismodegib); a monoclonal antibody (e.g., bevacizumab, cetuximab, ipilmumab, ofatumumab, ocrelizumab, panitumab, rituximab); a nucleoside/nucleotide analog (e.g., azacitidine, azathioprine, capecitabine, clofarabine, cytarabine, doxifluridine, floxuridine, fludarabine, 5-fluorouracil, gemcitabine, hydroxyurea, 6-mercaptopurine, methotrexate, pemetrexed, pentostatin, tioguanine); an anti-tumor antibiotic (e.g., bleomycin, actinomycin-D, mitomycin-C, mitoxantrone); a plantinum-based agent (e.g., carboplatin, cisplatin, oxaliplatin); a corticosteroid (e.g. prednisone, methylprednisone, dexamethasone); a retinoid (e.g., tretinoin, alitretinoin, bexarotene); a vinca alkaloid/derivative (e.g., vinblastine, vincristine, vindesine, vinorelbine); a CTLA 4 antibody (e.g., ipilimumab, marketed under the trade name YERVOY® by Bristol-Myers Squibb Co.); a checkpoint pathway inhibitor (e.g., a PD-1 inhibitors, such as nivolumab or lambrolizumab; a PD-Ll inhibitor, such as pembrolizumab, MEDI-4736 or MPDL3280A/RG744; or an nti-LAG-3 agents, such as BMS-986016 (MDX-1408)); an anti-SLAMF7 agent (e.g., the humanized monoclonal antibody elotuzumab (BMS-901608)); an anti-KIR agents (e.g., anti-KIR monoclonal antibody lirilumab (BMS-986015)); or an anti-CD 137 agent (e.g., the fully human monoclonal antibody urelumab (BMS-663513)). Of course, other chemotherapeutic agents can be used in combination with the AMPK-activating compound. Moreover, as is common in cancer chemotherapy, combinations of chemotherapeutic agents can be used, either simultaneously or sequentially.

In another embodiment, a method of treating a hyperproliferative disorder (e.g., cancer) in a subject in need thereof includes administering to the subject a therapeutically-effective amount of an AMPK-activating compound or a pharmaceutically acceptable salt, prodrug or N-oxide thereof (or a solvate or hydrate thereof) in combination with p53 gene therapy. There are a number of techniques known in the art that augment the wild-type p53 expression in cancer cells (especially cancer cells that lack p53, or have mutated p53) through gene therapy techniques. Such techniques increase the levels of wild-type p53, or of some other functional p53 in the cell. The use of AMPK-activating compounds as described herein can be used, for example, to augment gene therapy, helping to stabilize the p53. In other embodiments, gene therapy is used in combination with chemotherapy or radiation therapy, supplemented by the AMPK-activating compound.

The person of ordinary skill in the art will appreciate that a variety of art-recognized p53 activity assays can be used in determining the p53 upregulation caused by the AMPK-activating compounds. For example, p53 activity assays are commercially available from SABiosciences, Cayman Chemical, Pierce, and Perkin-Elmer. Conventional methods can be used to determine cytotoxicity and apoptosis; assays for each are commercially available, e.g., from Abcam, Cayman Chemical and Promega.

The presently disclosed AMPK-activating compounds act on particular aspects of metabolism; for example, the present compounds negatively regulate glycogen synthase and positively regulate glycogen phosphorylase. Thus, the present compounds are useful in treating disorders of glycogen storage, such as Pompe disease. The present compounds also increase autophagy, which is decreased in Pompe disease. The present compounds can be used to treat Pompe disease either alone or in adjunctively with enzyme replacement therapy, such as alglucosidase alfa (sold under the trade name MYOZYME) or the targeted enzyme therapy BMN-701 (IFG2-GAA). The compounds are useful in treating other rare metabolic disorders, including Fabry disease.

Another aspect of the disclosure is a method of increasing vascular flow in a subject in need thereof, the method including administering to the subject a therapeutically-effective amount of an AMPK-activating compound or a pharmaceutically acceptable salt, prodrug or N-oxide thereof (or a solvate or hydrate thereof). Accordingly, one embodiment of the disclosure is a method of treating a disorder of vascular flow in a subject in need thereof, the method including administering to the subject a therapeutically-effective amount of an AMPK-activating compound or a pharmaceutically acceptable salt, prodrug or N-oxide thereof (or a solvate or hydrate thereof). In certain embodiments, the disorder of vascular flow is selected from erectile dysfunction, primary or secondary Reynaud's disease, peripheral vascular disease, diabetic angiopathy and peripheral artery disease. In other embodiments, the disorder of vascular flow is selected from arteriosclerosis obliterans and Buerger's disease, and progressive systemic sclerosis, systemic erythematosus, vibration syndrome, aneurysm, and vasculitis. The person of ordinary skill in the art will determine a therapeutically-effective amount for a particular patient and a particular cancer using standard methods in the art.

Another aspect of the disclosure is a method of treating pulmonary arterial hypertension in a subject in need thereof, the method including administering to the subject a therapeutically-effective amount of an AMPK-activating compound or a pharmaceutically acceptable salt, prodrug or N-oxide thereof (or a solvate or hydrate thereof). Pulmonary arterial hypertension is a life-threatening disease involving endothelial dysfunction, vasoconstriction in small pulmonary arteries, dysregulated proliferation of certain vascular cells, and dysregulated inflammatory signaling leading to vascular remodeling, pulmonary fibrosis, and right ventricular hypertrophy. The presently disclosed compounds have antioxidative and anti-inflammatory properties, and exert beneficial effects on endothelial dysfunction, as well as inhibiting excessive proliferation of certain cells. Pulmonary arterial hypertension is described in S. L. Archer et al., Circulation, vol. 121, 2045-66 (2010), which is hereby incorporated herein by reference in its entirety. The person of ordinary skill in the art will determine a therapeutically-effective amount for a particular patient and a particular pulmonary arterial hypertensive state using standard methods in the art.

Another aspect of the disclosure is a method of treating vasculitis or venous ulcers in a subject in need thereof, the method including administering to the subject a therapeutically-effective amount of an AMPK-activating compound or a pharmaceutically acceptable salt, prodrug or N-oxide thereof (or a solvate or hydrate thereof). Accordingly, one embodiment of the disclosure is a method of treating a vasculitis in a subject in need thereof, the method including administering to the subject a therapeutically-effective amount of an AMPK-activating compound or a pharmaceutically acceptable salt, prodrug or N-oxide thereof (or a solvate or hydrate thereof). Another embodiment of the disclosure is a method of treating a venous ulcers in a subject in need thereof, the method including administering to the subject a therapeutically-effective amount of an AMPK-activating compound or a pharmaceutically acceptable salt, prodrug or N-oxide thereof (or a solvate or hydrate thereof). The person of ordinary skill in the art will determine a therapeutically-effective amount for a particular patient and a particular disorder to be treated using standard methods in the art.

The methods described herein can be useful with a wide variety of subjects. For example, in certain embodiments, the subject suffers from oxidative stress. In other embodiments, the subject does not suffer from oxidative stress. Similarly, in certain embodiments, the subject suffers from diabetes or hyperglycemia. In other embodiments, the subject does not suffer from diabetes or hyperglycemia.

Other aspects of the disclosure relate to biomarkers of AMPK activation by an AMPK-activating compound. These biomarkers have a wide variety of potential applications, as described in further detail below.

In certain embodiments, a biomarker of AMPK activation is a branched chain amino acid, such as, for example, valine, leucine and isoleucine. In other embodiments, a biomarker of AMPK activation is tyrosine or phenylalanine. Branched chain amino acids and related metabolites are strongly associated with metabolic disease. See, e.g., C. B. Newgard, “Interplay between Lipids and Branched-Chain Amino Acids in Development of Insulin Resistance,” Cell Metabolism, 15, 606 (2012), which is hereby incorporated herein by reference in its entirety. Thus, decreasing levels of branched chain amino acids, tyrosine and phenylalanine correlate with increasing AMPK activation, for example, by the AMPK-activating compounds described herein. Assay kits for branched chain amino acids and phenylalanine are commercially available, for example, from vendors such as Abcam and Biovision. Analysis of tyrosine may also be performed as described in A. Kumar & G. D. Christian, “Assay of L-Tyrosine in Serum by Amperometric Measurement of Tyrosinase-catalyzed Oxygen Consumption,” Clin. Chem. 21/3, 325-29 (1975), which is hereby incorporated herein by reference in its entirety, or by using a commercially-available assay kit. Moreover, diagnostic testing services, such as Quest Diagnostics, can provide assays of biological materials for branched chain amino acids, tyrosine and phenylalanine.

In other embodiments, a biomarker of AMPK activation is an acylcarnitine intermediate. Acylcarnitine are metabolites of branched chain amino acids, as described above. For example, in one embodiment, the acylcarnitine intermediate is isobutyrlcarnitine (a metabolite of valine), 2-methylbutyrylcarnitine (a metabolite isoleucine) or isovalerylcarnitine (a metabolite of leucine). Decreasing levels of acylcarnitine intermediates correlate with increasing AMPK activation, for example, by the AMPK-activating compounds described herein. Diagnostic testing services, such as Mayo Medical Laboratories and ARUP Laboratories, can provide assays of biological materials for acylcarnitines. Acylcarnitines may also be assayed using the procedures described in D. S. Millington et al., “3. Acylcarnitines: Analysis in Plasma and Whole Blood Using Tandem Mass Spectrometry,” Methods in Molecular Biology, 708, 55-72 (2011), which is hereby incorporated herein by reference in its entirety.

In other embodiments, a biomarker of AMPK activation is insulin-like growth factor-binding protein-1 (IGFBP1). AMPK stimulates secretion of IGFBP1. See, e.g., M. S. Lewitt, “Stimulation of IGF-Binding Protein-1 Secretion by AMP-Activated Protein Kinase,” Biochem. & Biophys. Res. Comms., 282, 1126-31 (2001), which is hereby incorporated herein by reference. Thus, increasing levels of IGFBP1 correlate with increasing AMPK activation, for example, by the AMPK-activating compounds described herein. Assay kits for IGFBP1 are commercially available, for example, from Abcam and Alpha Diagnostics International.

In other embodiments, a biomarker of AMPK activation is a ketone body. For example, in one embodiment, the ketone body is 3-hydroxybutyrate. In other embodiments, the ketone body is acetone or acetoacetate. Increasing levels of ketone bodies correlate with increasing AMPK activation, for example, by the AMPK-activating compounds described herein. Assay kits for ketone bodies are commercially available, for example, from Abnova and Wako Chemicals GmbH. Moreover, acetoacetate can be measured as described in S. K. Kundu & A. M. Judilla, “Novel sold-phase assay of ketone bodies in urine,” Clin. Chem., 37(9), 1565-69 (1991), which is hereby incorporated herein by reference in its entirety.

In other embodiments, a biomarker of AMPK activation is a citric acid cycle intermediate. For example, in one embodiment, the citric acid cycle intermediate is citrate, fumarate or malate. Increasing levels of citric acid cycle intermediates correlate with increasing AMPK activation, for example, by the AMPK-activating compounds described herein. Citric acid cycle intermediate assay kits are commercially available, for example, from BioVision, Abcam, Abnova and Sigma-Aldrich.

In other embodiments, a biomarker of AMPK activation is citrulline. Increasing levels of citrulline correlate with increasing AMPK activation, for example, by the AMPK-activating compounds described herein. The formation of citrulline from arginine via inducible nitric oxide synthase (iNOS) can impose pro-inflammatory signaling through the generation of nitric oxide. Citrulline assay kits are commercially available from, for example, CUSABIO and MyBioSource. In general, small molecule analytes (including citrulline) were determined according to the methods of Evans et al. (Evans, A. M., et al., “Integrated, nontargeted ultrahigh performance liquid chromatography/electrospray ionization tandem mass spectrometry platform for the identification and relative quantification of the small-molecule complement of biological systems,” Anal. Chem., 2009. 81(16): p. 6656-67, which is hereby incorporated herein by reference in its entirety) In other embodiments, a biomarker of AMPK activation is a fatty acid. For example, in one embodiment, the fatty acid is palmitate or myristate (e.g., as found in skeletal muscle). Decreasing levels of fatty acid correlate with increasing AMPK activation, for example, by the AMPK-activating compounds described herein. For example, treatment with the compounds described herein markedly reduces absolute levels of skeletal palmitate and myristate. Fatty acid assay kits are commercially available, for example, from MyBioSource, SigmaAldrich, and Abcam. Moreover, fatty acids can be measured using techniques described in K. Kishiro and H. Yasuda, “A reliable analysis of tissue free fatty acids by gas-liquid chromatography,” Anal. Biochem., 175(2), 516-520 (1988), which is hereby incorporated herein by reference in its entirety.

The biomarkers of AMPK activation disclosed herein can be used in a variety of ways. For example, in one aspect of the disclosure, a method for determining the degree of AMPK activation in a subject includes administering to the subject an AMPK-activating compound; then obtaining a sample from the subject; and measuring the concentration of the biomarker of AMPK activation in the sample from the subject. The concentration of the biomarker of AMPK activation can be correlated with AMPK activation as described above. In one embodiment, the method can be used to determine a therapeutic dosage of the AMPK-activating compound for the subject. For example, in one embodiment, the concentration of the biomarker of AMPK activation is correlated with a therapeutic dosage. The method can further include administering to the subject (e.g., on a continuing basis) the AMPK-activating compound at at least about the therapeutic dosage, for example, in order to activate the AMPK pathway in the subject. Moreover, in certain embodiments, methods according to this aspect can be used to monitor the progress of treatment using the AMPK-activating compound. For example, as measured biomarker concentration (and thus the degree of AMPK activation) deviates from a desired level, the dosage of the AMPK-activating compound can be increased or decreased accordingly.

In another aspect of the disclosure, a method for determining the degree of AMPK activation caused by administration of in a subject includes obtaining a first sample from the subject; measuring the initial concentration of a biomarker of AMPK activation in the first sample from the subject; after obtaining the first sample from the subject, administering to the subject an AMPK-activating compound; and after administration, obtaining a second sample from the subject; and measuring the concentration of the biomarker in the second sample from the subject. The concentration of the biomarker of AMPK activation in the second sample can be correlated with AMPK activation as described above. In certain embodiments, the concentration of the biomarker of AMPK activation in the first sample and the concentration of the biomarker of AMPK activation in the second sample are together correlated with AMPK activation. For example, the difference (or the ratio, or some other mathematical comparison) in the concentrations of the biomarker of AMPK activation as measured before and after administration (i.e., as measured in the first and second samples) can be correlated to the degree of AMPK activation. As the person of ordinary skill in the art will appreciate, the cycle of administering another test dosage, obtaining another sample and measuring the concentration of the biomarker of AMPK activation in the sample can be repeated one or more times to provide additional information for use in determining the degree of AMPK activation. In certain embodiments, the method can be used to determine a therapeutic dosage of the AMPK-activating compound for the subject. For example, in one embodiment, the concentration of the biomarker of AMPK activation in the second sample, optionally together with the concentration of the biomarker of AMPK activation in the first sample (e.g., as described above with respect to correlation with the AMPK activation), is correlated with a therapeutic dosage. The method can further include administering to the subject (e.g., on a continuing basis) the AMPK-activating compound at at least about the therapeutic dosage, for example, in order to activate the AMPK pathway in the subject.

Determination of the degree of AMPK activation can be used diagnostically. For example, in certain embodiments, in the methods of determining the degree of AMPK activation described herein, the methods can be used to diagnose an AMPK-related disorder, for example, by correlating the concentration(s) of the biomarker of AMPK activation in the sample(s) with the presence, absence, or degree of progression of an AMPK-related disorder. In one aspect, the biomarker correlation with AMPK activation may be performed by directly measuring AMPK activation in a subject as described in patent application publications US2012-0178098A1 and WO2012/094173A1, which are hereby incorporated herein by reference in their entireties.

In another aspect of the disclosure, a method of activating the AMPK pathway in a subject in need thereof includes obtaining a first sample from the subject; measuring the concentration of a biomarker of AMPK activation in the first sample from the subject; after obtaining the first sample, administering to the subject an AMPK-activating compound at a test dosage; after administration, obtaining a second sample from the subject; measuring the concentration of the biomarker of AMPK activation in the second sample from the subject; selecting a therapeutic dosage of the AMPK-activating compound based on the concentration of the biomarker of AMPK activation in the second sample, optionally together with the concentration of the biomarker of AMPK activation in the first sample (e.g., as described above with respect to correlation with the AMPK activation); and administering to the subject the AMPK-activating compound at at least about the therapeutic dosage. As the person of ordinary skill in the art will appreciate, the cycle of administering another test dosage, obtaining another sample and measuring the concentration of the biomarker of AMPK activation in the sample can be repeated one or more times to provide additional information for use in determining the therapeutic dosage.

In another aspect of the disclosure, a method of activating the AMPK pathway in a subject in need thereof includes administering to the subject an AMPK-activating compound at a test dosage; after administration, obtaining a sample from the subject; measuring the concentration of the biomarker of AMPK activation in the sample from the subject; selecting a therapeutic dosage of the AMPK-activating compound based on the concentration of the biomarker of AMPK activation in the sample; and administering to the subject the AMPK-activating compound at at least about the therapeutic dosage. As the person of ordinary skill in the art will appreciate, the cycle of administering another test dosage, obtaining another sample and measuring the concentration of the biomarker of AMPK activation in the sample can be repeated one or more times to provide additional information for use in determining the therapeutic dosage.

The biomarkers of AMPK activation can also be used in the diagnosis, prognosis and treatment of particular disorders linked to inadequate AMPK activation. Accordingly, in the methods for AMPK activation described above, the therapeutic dosage can be selected to be effective in treating an AMPK-linked disorder, and thus the methods can be used to treat the AMPK-linked disorder.

For example, in one embodiment, a method of treating an AMPK-linked disorder in a subject in need thereof includes obtaining a first sample from the subject; measuring the concentration of a biomarker of AMPK activation in the first sample from the subject; after obtaining the first sample, administering to the subject an AMPK-activating compound at a test dosage; after administration, obtaining a second sample from the subject; measuring the concentration of the biomarker of AMPK activation in the second sample from the subject; selecting a therapeutic dosage of the AMPK-activating compound based on the difference in the concentration of the biomarker in the first sample and the second sample; and administering to the subject the AMPK-activating compound at at least about the therapeutic dosage. As the person of ordinary skill in the art will appreciate, the cycle of administering another test dosage, obtaining another sample and measuring the concentration of the biomarker in the sample can be repeated one or more times to provide additional information for use in determining the therapeutic dosage.

In another aspect of the disclosure, a method of treating an AMPK-linked disorder in a subject in need thereof includes administering to the subject an AMPK-activating compound at a test dosage; after administration, obtaining a sample from the subject; measuring the concentration of the biomarker of AMPK activation in the sample from the subject; selecting a therapeutic dosage of the AMPK-activating compound based on the concentration of the biomarker of AMPK activation in the sample; and administering to the subject the AMPK-activating compound at at least about the therapeutic dosage. As the person of ordinary skill in the art will appreciate, the cycle of administering another test dosage, obtaining another sample and measuring the concentration of the biomarker of AMPK activation in the sample can be repeated one or more times to provide additional information for use in determining the therapeutic dosage.

In one aspect, certain conditions can be diagnosed using the presently disclosed biomarkers and treated using the disclosed compounds and methods. For example, elevated levels of branched chain amino acids can be correlated with insulin resistance, type 2 diabetes and cardiovascular disease. Accordingly, in one aspect, a subject for treatment with the present compounds can be identified by testing branch chain amino acid levels (Newgard, Cell Metabolism 15, p. 606, 2012, which is hereby incorporated herein by reference in its entirety). Thus, in certain embodiments, the disclosure provides a method activating the AMPK pathway in a subject in need thereof. The method includes obtaining a first sample from the subject; measuring the concentration of a biomarker of AMPK activation in the first sample from the subject; and selecting a therapeutic dosage of the AMPK-activating compound based on the concentration of the biomarker of AMPK activation in the first sample. The method can further include administering to the subject the AMPK-activating compound at at least about the therapeutic dosage. As described above, the therapeutic dosage can be selected to be effective in treating an AMPK-linked disorder, and thus the methods can be used to treat the AMPK-linked disorder.

There are a variety of AMPK-linked disorders that can be diagnosed or treated as described above. For example, in certain embodiments, the AMPK-linked disorder is a hyperproliferative disorder such as cancer, as described above. In other embodiments, the AMPK-linked disorder is a disorder of vascular flow, as described above. In other embodiments, the AMPK-linked disorder is a disorder of glycogen storage, as described above.

In other embodiments, the AMPK-linked disorder is selected from increased triglyceride levels, decreased insulin sensitivity, metabolic disorders such as diabetes (e.g., type I diabetes, type II diabetes), hyperglycemia, hyperinsulinemia and hypertriglyceridemia), atherosclerosis and cardiovascular disease.

Activation of the AMPK pathway has the effect of increasing glucose uptake, decreasing glycogen synthesis and increasing fatty acid oxidation, thereby reducing glycogen, intracellular triglyceride and fatty acid concentration and causing an increase in insulin sensitivity. AMPK activating compounds should also inhibit the inflammatory processes which occur during the early phases of atherosclerosis. Accordingly, AMPK-activating compounds can be useful in the treatment of type II diabetes and in the treatment and prevention of atherosclerosis, cardiovascular disease, obesity and non-alcoholic fatty liver disease; in certain embodiments of the methods described herein, the AMPK-linked compound is one of these.

In one aspect and without limitation to theory, the present compounds exert AMPK activating activity by binding to an adiponectin receptor, acting as effective adiponectin mimetics. Adiponectin is a protein hormone exclusively expressed in and secreted from adipose tissue and is the most abundant adipose-specific protein. Adiponectin has been implicated in the modulation of glucose and lipid metabolism in insulin-sensitive tissues. Decreased circulating adiponectin levels have been demonstrated in some insulin-resistant states, such as obesity and type 2 diabetes mellitus and also in patients with coronary artery disease, atherosclerosis and hypertension. Adiponectin levels are positively correlated with insulin sensitivity, HDL (high density lipoprotein) levels and insulin stimulated glucose disposal and inversely correlated with adiposity and glucose, insulin and triglyceride levels. Thiazolidinedione drugs, which enhance insulin sensitivity through activation of the peroxisome proliferator-activated receptor-γ, increase endogenous adiponectin production in humans. Adiponectin binds its receptors in liver and skeletal muscle and thereby activates the AMPK pathway. Similarly, in one aspect, the present compounds act as adiponectin receptor agonists. Adiponectin receptors 1 and 2 are membrane-bound proteins found in skeletal muscle and liver tissue.

In other aspects, the AMPK-linked disorder is a disorder of decreased or insufficient metabolic efficiency. The presently disclosed AMPK-activating compounds are useful for increasing metabolic efficiency, for example by increasing muscle fiber oxidative capacity, endurance and aerobic workload. In certain embodiments, the AMPK-linked disorder is a disorder of mitochondrial function, including, without limitation, exercise intolerance, chronic fatigue syndrome, muscle weakness, myoclonus, myoclonus epilepsy, such as associated with ragged-red fibers syndrome, Kearns-Sayre syndrome, Leigh's syndrome, mitochondrial myopathy encephalopathy lactacidosis stroke (MELAS) syndrome and stroke like episodes. In other embodiments, the AMPK-linked disorder is insufficient muscle fiber oxidative capacity. In other embodiments, the AMPK-linked disorder is a muscular dystrophic state, such as Duchenne's and Becker's muscular dystrophies and Friedreich's ataxia.

The presently disclosed AMPK-activating compounds and methods also function to reduce oxidative stress and secondary effects of such stress. Many diseases, including several of those listed above, have secondary effects caused by damage due to excessive oxidative stress which can be treated using the compounds and methods disclosed herein. Accordingly, in one embodiment, the AMPK-linked disorder is increased oxidative stress. For example, free radical damage has been implicated in neurological disorders, such as Parkinson's disease, amyotrophic lateral sclerosis (Lou Gehrig's disease) and Alzheimers disease; in certain embodiments, the AMPK-linked disorder is one of these disorders. Additional disorders in which excessive free radical damage occurs generally include hypoxic conditions and a variety of other disorders. More specifically, in certain embodiments, the AMPK-linked disorder is selected from the group consisting of ischemia, ischemic reperfusion injury (such as coronary or cerebral reperfusion injury), myocardial ischemia or infarction, cerebrovascular accidents (such as a thromboembolic or hemorrhagic stroke) that can lead to ischemia in the brain, operative ischemia, traumatic hemorrhage (for example, a hypovolemic stroke that can lead to CNS hypoxia or anoxia), resuscitation injury, spinal cord trauma, inflammatory diseases, autoimmune disorders (such as rheumatoid arthritis or systemic lupus erythematosis), Down's syndrome, Hallervorden-Spatz disease, Huntingtons chorea, Wilson's disease, diabetic angiopathy (such as peripheral vascular disease or retinal degeneration), uveitis, chronic obstructive pulmonary disease (COPD), including chronic bronchitis and emphysema, asthma, neoplasia, Crohn's disease, inflammatory bowel disease and pancreatitis. Free radical damage is also implicated in a variety of age-related disorders, particularly ophthalmic conditions such as cataracts and age-related macular degeneration; in certain embodiments of the methods described herein, the AMPK-linked disorder is one of these disorders. In other embodiments, the AMPK-linked disorder is free radical damage.

In particular embodiments, the presently disclosed compounds and methods are useful for treating neurological disorders associated with reduced mitochondrial function, oxidative stress, or both. For example, in certain embodiments of the methods described herein, the AMPK-linked disorder is selected from Alzheimer's disease, dementia and Parkinson's disease. The present AMPK-activating compounds also may be useful in increasing neurogenesis, particularly hippocampal neurogenesis, and thus may be useful in treating neurological disorders, including Alzheimer's disease, amyotrophic lateral sclerosis, Parkinson's disease, cognitive deficiency and the like, for this additional reason.

Metabolic efficiency is enhanced by the disclosed AMPK-activating compounds and methods. Thus, in certain embodiments, the methods disclosed herein can be used improve exercise efficiency, exercise endurance and athletic performance of the subject. Moreover, in certain embodiments of the methods described herein, the AMPK-linked condition is selected from hypoxic states, angina pectoris, coronary ischemia and organ damage secondary to coronary vessel occlusion, intermittent claudication, multi-infarct dementia, myocardial infarction, stroke, high altitude sickness and heart failure, including congestive heart failure.

In certain embodiments, the AMPK-linked disorder is an inflammatory disorder. For example, in one aspect, the present compounds are particularly useful for treating lung inflammation, such as is involved in asthma, COPD and transplant rejection; in one embodiment, the AMPK-linked disorder is selected from these. Similarly, in other embodiments, the AMPK-linked disorder is organ inflammation, particularly macrophage-associated inflammation, such as inflammation of the kidney, liver and other organs. The anti-inflammatory activity of the presently disclosed compounds can be assessed as is known to those of skill in the art, for example, by using the mixed lymphocyte response in vitro.

As the person of ordinary skill in the art will appreciate, the sample obtained from the subject may be in a variety of forms. For example, in certain embodiments, the sample is a blood sample (including fractions thereof, e.g., plasma or serum); a tissue sample or a urine sample. The selection of the type of sample may be performed by the person of ordinary skill in the art based on the particular biomarker to be measured and the particular assay methodology to be used. Methods of obtaining biological samples including tissue resections, biopsies and body fluids, e.g., blood samples, are well known in the art. In certain embodiments, the sample is obtained and examined in situ, e.g., through direct detection methods.

As the person of ordinary skill in the art will appreciate, a variety of methods can be used to correlate biomarker concentrations with AMPK activation and/or a therapeutic dosage for the subject. For example, the person of ordinary skill in the art can compare the biomarker concentration (or a difference between pre- and post-administration concentrations) to a standard level that indicates a desired level of AMPK activation. Through routine experimentation, the person of ordinary skill in the art can determine desired levels of the biomarkers that indicate an adequate or desirable degree of AMPK activation, the presence, absence or progression of an AMPK-related disorder, or a desired degree of treatment of an AMPK-related disorder.

In certain embodiments of the therapeutic methods described herein, the AMPK-activating compound is administered at a level sufficient to cause the measured concentration of the biomarker post-administration to be within about 40%, within about 20%, or even within about 10% of a control concentration. A control concentration can be, for example, a biomarker concentration that indicates a desired degree of AMPK activation, e.g., sufficient to treat or ameliorate an AMPK-linked disorder. The control concentration can be determined, for example, by measuring concentrations of the biomarker in healthy individuals to provide a control concentration value. In other embodiments, a control concentration is determined for the subject by determining the concentration of the biomarker when the subject is in a state in which AMPK activation is at a desired level (e.g., the subject is not suffering from an AMPK-related disorder).

In other embodiments of the therapeutic methods described herein, the AMPK-activating compound is administered at a level sufficient to cause the measured concentration of the biomarker post-administration to be, for a biomarker whose concentration is positively correlated with AMPK activation, at least about 60%, at least about 80%, at least about 90%, or even at least about 100% of a control concentration, and for a biomarker whose concentration is negatively correlated with AMPK activation, no greater than about 140%, no greater than about 120%, no greater than about 110%, or even no greater than about 100% of a control concentration. A control concentration can be, for example, a biomarker concentration that indicates a desired degree of AMPK activation, e.g., sufficient to treat or ameliorate an AMPK-linked disorder. The control concentration can be determined, for example, by measuring concentrations of the biomarker in healthy individuals to provide a control concentration value. In other embodiments, a control concentration is determined for the subject by determining the concentration of the biomarker when the subject is in a state in which AMPK activation is at a desired level (e.g., the subject is not suffering from an AMPK-related disorder).

In other embodiments of the therapeutic methods described herein, the AMPK-activating compound is administered at a level sufficient to cause the measured concentration of the biomarker post-administration to change by at least about 10%, at least about 20%, or even at least about 40% as compared to the measured concentration pre-administration. That is, for biomarkers whose concentration is positively correlated with AMPK activation, the measured concentration of the biomarker post-administration is at least about 10%, at least about 20%, or even at least about 40% greater than the measured concentration pre-administration; and for biomarkers whose concentration is negatively correlated with AMPK activation, the measured concentration of the biomarker post-administration is at least about 10%, at least about 20%, or even at least about 40% less than the measured concentration pre-administration.

In another embodiment, a method comprises modulating the AMPK pathway in a subject by administering to the subject an AMPK-activating compound or a pharmaceutically acceptable salt, prodrug or N-oxide thereof (or solvate or hydrate thereof), in an amount sufficient to modulate the AMPK activity; obtaining a sample from the subject; and measuring the concentration of a biomarker in the subject. Such methods are useful for studying the AMPK pathway and its role in biological mechanisms and disease states.

Compounds can be assayed for binding to a membrane-bound adiponectin receptor by performing a competitive binding assay with adiponectin. In one such procedure, HEK 293 cellular membrane is coated onto a COSTAR 384 plate, which is then blocked with 1% casein. Polyhistidine-tagged globular adiponectin and a candidate compound are incubated with the membrane in HEPES buffer. Unbound ligands are washed away and the degree of binding of the adiponectin is determined using horseradish peroxidase-conjugated anti-polyhistidine. Compounds that compete with adiponectin binding to the membrane (i.e., give a reduced signal compared to a control performed without a candidate compound) can be chosen as hits and further screened using the below-described functional assays to identify adiponectin receptor agonists.

An in-cell western assay can be performed to demonstrate the activation of the AMPK pathway in human liver cells by globular adiponectin using glutathione S-transferase (GST). AMPK activity can be measured by the relative concentration of phosphorylated acetyl Co-A carboxylase, which is one of the products of AMPK. An increase in pACC correlates with an increase in the rate of fatty acid oxidation.

In certain embodiments of the methods described herein, the AMPK-activating compound is an AMPK-activating compound having an EC₅₀ for AMPK activation of less than about 10 μM, less than about 5 μM, less than about 1 μM, less than about 500 nM, less than about 100 nM, or even less than about 50 nM. Exemplary compounds exhibited an EC₅₀ for AMPK activation of less than 1 nM or of from about 1 nM to about 75 nM, such as from about 5 nM to about 50 nM or to about 25 nM. The AMPK-activating compounds described herein have are compounds having structural formula (I):

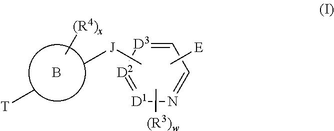

or a pharmaceutically acceptable salt, prodrug or N-oxide thereof (or a solvate or hydrate thereof), in which
0 or 1 of D¹, D² and D³ is N, with the others independently being CH or C substituted by one of the w R³;
E is —R², —C(O)NR¹R², —NR¹R² or —NR¹C(O)R², in which R¹ and R² together with the nitrogen to which they are bound form Hca, or R¹ is H, —(C₁-C₄ alkyl), —C(O)—(C₁-C₄ alkyl) or —C(O)O—(C₁-C₄ alkyl), and R² is —C(O)Hca, —(C₀-C₃ alkyl)-Ar, —(C₁-C₃ alkyl)-O—Ar, —(C₁-C₃ alkyl)-O-Het, —(C₀-C₃ alkyl)-Het, —(C₀-C₃ alkyl)-Cak or —(C₀-C₃ alkyl)-Hca;
each R³ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
each R⁴ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo, and two R⁴ on different carbons optionally combine to form a —(C₀-C₄ alkylene)- bridge;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene,

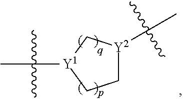

wherein each of Y¹ and Y² is N, C or CH, provided that at least one of Y¹ and Y² is N;
p is 0, 1, 2, 3 or 4, q is 1, 2, 3 or 4, and the sum of p and q is 1, 2, 3, 4, 5 or 6, or

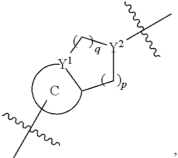

wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6;
T is H, —(C₁-C₆ alkyl), —(C₁-C₆ alkyl)-R²³ in which R²³ is Het or Ar and in which one or more non-adjacent carbons of the alkyl is optionally replaced by —O— or —S—, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰ or

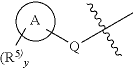

wherein
Q is —O—(C₀-C₃ alkyl)-, —S(O)₂—, -L- or (C₀-C₃ alkyl)-, in which each carbon of the —(C₀-C₃ alkyl)- is optionally and independently substituted with one or two R¹⁶;
the ring system denoted by “A” is heteroaryl, aryl, cycloalkyl or heterocycloalkyl;
each R⁵ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —N₃, —SF₅, —NO₂ and —CN; and
y is 0, 1, 2, 3 or 4;
in which
   each L is independently selected
      from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR⁹)NR⁹—, —NR⁹SO₂—, —SO₂NR⁹— and —NR⁹SO₂NR⁹—,
   each R⁶, R⁷, R⁸ and R¹⁰ is independently selected from H, —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-(C₀-C₆ alkyl), —(C₀-C₆ alkyl)-NR⁹—(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl),
   each R⁹ is independently selected from —H, —(C₁-C₄ alkyl), —C(O)—(C₁-C₄ alkyl) and —C(O)O—(C₁-C₄ alkyl),
   each Ar is an optionally substituted aryl,
   each Het is an optionally substituted heteroaryl,
   each Cak is an optionally substituted cycloalkyl,
   each Hca is an optionally substituted heterocycloalkyl, and
   each alkyl is optionally substituted.

In certain embodiments as described above, the AMPK-activating compound is a compound of structural formula (II):

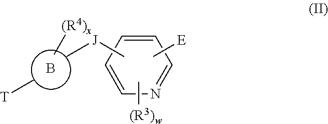

or a pharmaceutically acceptable salt, prodrug or N-oxide thereof (or a solvate or hydrate thereof), in which
E is —R², —C(O)NR¹R², —NR¹R², —NR¹C(O)R², in which R¹ and R² together with the nitrogen to which they are bound form Hca, or R¹ is H, —(C₁-C₄ alkyl), —C(O)—(C₁-C₄ alkyl) or —C(O)O—(C₁-C₄ alkyl), and R² is —C(O)Hca, —(C₀-C₃ alkyl)-Ar, —(C₀-C₃ alkyl)-Het, —(C₀-C₃ alkyl)-Cak or —(C₀-C₃ alkyl)-Hca;
each R³ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
each R⁴ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene, or

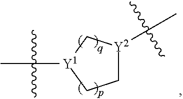

wherein each of Y¹ and Y² is N, C or CH, provided that at least one of Y¹ and Y² is N;
p is 0, 1, 2, 3 or 4, q is 1, 2, 3 or 4, and the sum of p and q is 2, 3, 4, 5 or 6;
T is H, —(C₁-C₆ alkyl), —(C₁-C₆ alkyl)-R²³ in which R²³ is Het or Ar and in which one or more non-adjacent carbons of the alkyl is optionally replaced by —O— or —S—, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰ or

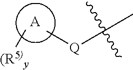

wherein
Q is —O—(C₀-C₃ alkyl)-, —S(O)₂—, -L- or (C₀-C₃ alkyl)-, in which each carbon of the —(C₀-C₃ alkyl)- is optionally and independently substituted with one or two R¹⁶;
the ring system denoted by “A” is heteroaryl, aryl, cycloalkyl or heterocycloalkyl;
each R⁵ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN; and
y is 0, 1, 2, 3 or 4;
in which
   each L is independently selected
      from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR⁹)NR⁹—, —NR⁹SO₂—, —SO₂NR⁹— and —NR⁹SO₂NR⁹—,
   each R⁶, R⁷, R⁸ and R¹⁰ is independently selected from H, —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-(C₀-C₆ alkyl), —(C₀-C₆ alkyl)-NR⁹—(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl),
   each R⁹ is independently selected from —H, —(C₁-C₄ alkyl), —C(O)—(C₁-C₄ alkyl) and —C(O)O—(C₁-C₄ alkyl),
   each Ar is an optionally substituted aryl,
   each Het is an optionally substituted heteroaryl,
   each Cak is an optionally substituted cycloalkyl,
   each Hca is an optionally substituted heterocycloalkyl, and
   each alkyl is optionally substituted.

In certain embodiments as described above, the compound is not
5-(4-(4-cyanobenzyl)piperazine-1-carbonyl)-N-(1-(4-cyanobenzyl)piperidin-4-yl)picolinamide;
N-(1-(4-cyanobenzyl)piperidin-4-yl)-5-(4-(4-fluorobenzyl)piperazine-1-carbonyl)picolinamide;
N-(1-(4-cyanobenzyl)piperidin-4-yl)-5-(4-(4-(trifluoromethyl)benzyl)piperazine-1-carbonyl)picolinamide
(S)-5-(4-(4-chlorophenyl)piperazine-1-carbonyl)-N-(1-(4-fluorobenzyl)pyrrolidin-3-yl)picolinamide;
(S)-5-(4-(4-chlorophenyl)piperazine-1-carbonyl)-N-(1-(pyridin-4-ylmethyl)pyrrolidin-3-yl)picolinamide;
(S)-5-(4-(4-chlorophenyl)piperazine-1-carbonyl)-N-(1-(4-cyanobenzyl)pyrrolidin-3-yl)picolinamide;
N-(1-(4-chlorobenzyl)pyrrolidin-3-yl)-5-(4-(4-chlorophenyl)piperazine-1-carbonyl)picolinamide; or
5-(4-(4-chlorophenyl)piperazine-1-carbonyl)-N-(1-(4-(trifluoromethyl)benzyl)pyrrolidin-3-yl)picolinamide.
   or a pharmaceutically acceptable salt, prodrug or N-oxide thereof (or a solvate or hydrate thereof).

In one embodiment, the presently disclosed compounds are not compounds disclosed in Darwish et al., International Patent Application no. PCT/US10/22411, filed Jan. 28, 2010, which is hereby incorporated by reference in its entirety.

In certain embodiments as described above, in the AMPK-activating compounds of structural formula (I) and (II), D¹, D² and D³ are independently CH or C substituted by one of the w R³. In other embodiments, D¹ is N and D² and D³ are independently CH or C substituted by one of the w R³. In other embodiments, D² is N and D¹ and D³ are independently CH or C substituted by one of the w R³. In other embodiments, D³ is N and D¹ and D² are independently CH or C substituted by one of the w R³.

In certain embodiments as described above, in the AMPK-activating compounds of structural formula (I) and (II), J is —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl). In certain embodiments of the compounds of structural formula (I) and (II) as described above, R¹³ is H. In other embodiments, R¹³ is unsubstituted (C₁-C₄ alkyl). In certain embodiments of the compounds of structural formula (I) and (II) as described above, J is —C(O)—. In other embodiments, J is —NR¹³— (for example, —NH—). In still other embodiments, J is —NR¹³C(O)— (for example, —NHC(O)—). In other embodiments, J is —C(O)NR¹³— (for example, —C(O)NH—). In still other embodiments, J is absent.

In certain embodiments as described above, in the AMPK-activating compounds of structural formula (I) and (II), the ring system denoted by “B” is absent, arylene, heteroarylene,

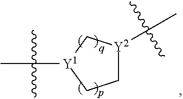

in which each of Y¹ and Y² is N, C or CH, provided that at least one of Y¹ and Y² is N; p is 0, 1, 2, 3 or 4, q is 1, 2, 3 or 4, and the sum of p and q is 1, 2, 3, 4, 5 or 6,

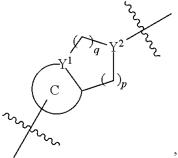

wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6.

For example, in certain embodiments as described above, in the AMPK-activating compounds of structural formula (I) and (II), (for example, those described below with respect to structural formula (IV)), the ring system denoted by “B” is arylene or heteroarylene. In certain embodiments, the ring system denoted by “B” is arylene (for example, phenylene such as 1,4-phenylene). In other embodiments, the ring system denoted by “B” is heteroarylene (for example, 1H-pyrazolylene, 1H-1,2,3-triazolylene, pyridylene, furanylene or thienylene). In certain embodiments as described above, in the AMPK-activating compounds of structural formula (I) and (II), the ring system denoted by “B” is monocyclic arylene or heteroarylene.

In certain embodiments as described above, in the AMPK-activating compounds of structural formula (I) and (II), the ring system denoted by “B” is absent.

In certain embodiments as described above, in the AMPK-activating compounds of structural formula (I) and (II), the ring system denoted by “B” is

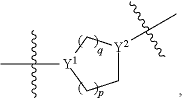

wherein each of Y¹ and Y² is N, C or CH, provided that at least one of Y¹ and Y² is N; p is 0, 1, 2, 3 or 4, q is 1, 2, 3 or 4, and the sum of p and q is 2, 3, 4, 5 or 6. For example, in certain embodiments, Y¹ is N and Y² is C or CH. (When Y¹ or Y² is C, it is substituted by one of the x R⁴.) In other embodiments, Y¹ is C or CH and Y² is N. In other embodiments, Y¹ is CF and Y² is N. In other embodiments, Y¹ and Y² are each N. In certain embodiments as described above, in the AMPK-activating compounds of structural formula (I) and (II), p is 1 and q is 2. For example, in one embodiment, the ring system denoted by “B” is a piperidine linked to the T moiety through its nitrogen atom. In another embodiment, the ring system denoted by “B” is a piperidine linked to the J moiety through its piperidine nitrogen. In another embodiment, the ring system denoted by “B” is a piperazine. In other embodiments as described above, in the AMPK-activating compounds of structural formula (I) and (II), p is 1 and q is 1. For example, in certain embodiments, the ring system denoted by “B” is a pyrrolidine, for example, linked to the J moiety through its pyrrolidine nitrogen. In still other embodiments as described above, in the AMPK-activating compounds of structural formula (I) and (II), p is 0 and q is 1. For example, in certain embodiments, the ring system denoted by “B” is an azetidine, for example, linked to the J moiety through its azetidine nitrogen.

In certain embodiments as described above, in the AMPK-activating compounds of structural formula (I) and (II), the ring system denoted by “B” is

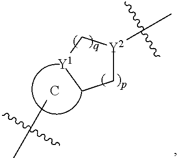

wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6. For example, in certain embodiments, Y¹ is N and Y² is C or CH. (When Y² is C, it can be substituted by one of the x R⁴.) In other embodiments, Y¹ is C and Y² is N. In other embodiments, Y¹ and Y² are each N. In certain embodiments as described above, in the AMPK-activating compounds of structural formula (I) and (II), p is 1 and q is 2. In other embodiments of the presently disclosed compounds of structural formula (I) as described above, p is 1 and q is 1. The heteroarylene can be, for example, a pyridine, a pyrazine, a pyrimidine, a triazine, a pyrrole, a pyrazole, an imidazole, or a triazole. In one example, the ring system denoted by “B” is

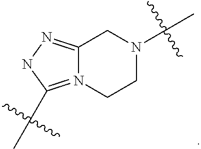

In the various aspects of the disclosure presently disclosed, in the AMPK-activating compounds of structural formula (I) and (II), x, the number of substituents on the ring system denoted by “B”, is 0, 1, 2, 3 or 4. In one embodiment, x is 0, 1, 2 or 3. For example, in certain embodiments, x is 0. In other embodiments, x can be 1 or 2.

In certain embodiments as described above, in the AMPK-activating compounds of structural formula (I) and (II) (for example, when the ring system denoted by “B” is

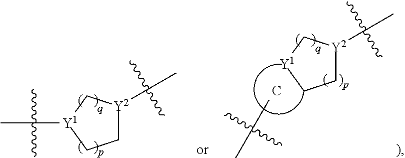

two R⁴ groups combine to form an oxo. The oxo can be bound, for example, at the position alpha to a nitrogen atom of the ring system. In other embodiments, no two R⁴ groups combine to form an oxo.

In certain embodiments as described above, in the AMPK-activating compounds of structural formula (I) and (II) (for example, when the ring system denoted by “B” is

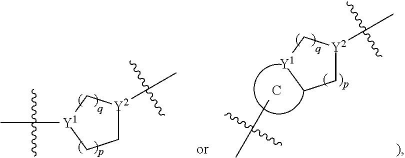

two R⁴ groups on different carbons combine to form a —(C₀-C₄ alkylene)- bridge. The alkylene bridge can form bicyclic system, for example, a [3.2.1] system, a [3.2.0] system, a [3.1.0] system, [2.2.2] system, a [2.2.1] system, a [2.1.1] system, a [2.2.0] system or a [2.1.0] system. For example, in one embodiment, ring system denoted by “B” is substituted with R⁴ groups to form

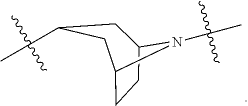 [SMILES: *C1CC2CCC(C1)N2*]

In certain embodiments the —(C₀-C₄ alkylene)- bridge is unsubstituted. In other embodiments, it is substituted only with one or more halogens.

In certain embodiments as described above, in the AMPK-activating compounds of structural formula (I) and (II) (for example, when the ring system denoted by “B” is

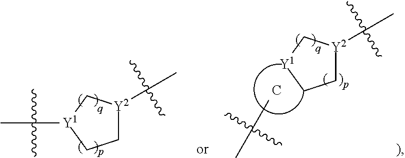

two R⁴ moieties (for example, on the same carbon) are (C₁-C₄ alkyl) (for example, methyl), as described below.

In certain embodiments as described above, in the AMPK-activating compounds of structural formula (I) and (II), when x is 4, not all four R⁴ groups are (C₁-C₆ alkyl).

In certain embodiments as described above, in the AMPK-activating compounds of structural formula (I) and (II), each R⁴ is independently selected from —(C₁-C₆ alkyl), —(C₁-C₆ haloalkyl) (for example, difluoromethyl, trifluoromethyl and the like), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, in which each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl), and in which no alkyl or haloalkyl is substituted with an aryl-, heteroaryl-, cycloalkyl- or heterocycloalkyl-containing group. For example, in one embodiment, each R⁴ is —(C₁-C₃ alkyl), —(C₁-C₃ haloalkyl), —(C₀-C₃ alkyl)-L-R⁷, —(C₀-C₃alkyl)-NR⁸R⁹, —(C₀-C₃ alkyl)-OR¹⁰, —(C₀-C₃ alkyl)-C(O)R¹⁰, —(C₀-C₃ alkyl)-S(O)₀₋₂R¹⁰, -halogen, —NO₂ and —CN, in which each R⁷, R⁸ and R¹⁰ is independently selected from H, —(C₁-C₂ alkyl), —(C₁-C₂ haloalkyl), —(C₀-C₂ alkyl)-L-(C₀-C₂ alkyl), —(C₀-C₂ alkyl)-NR⁹(C₀-C₂ alkyl), —(C₀-C₂ alkyl)-O—(C₀-C₂ alkyl), —(C₀-C₂ alkyl)-C(O)—(C₀-C₂ alkyl) and —(C₀-C₂ alkyl)-S(O)₀₋₂—(C₀-C₂ alkyl), and in which no alkyl or haloalkyl is substituted with an aryl-, heteroaryl-, cycloalkyl- or heterocycloalkyl-containing group. In certain embodiments, each R⁴ is independently halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl), and two R₄ optionally come together to form oxo. In certain embodiments, each R⁴ is independently methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂ or —CN, and two R₄ optionally come together to form oxo.

In the various aspects of the disclosure presently disclosed, in the AMPK-activating compounds of structural formula (I) and (II), E is —R², —C(O)NR¹R², —NR¹R² or —NR¹C(O)R², in which R¹ and R² together with the nitrogen to which they are bound form Hca, or R¹ is H, —(C₁-C₄ alkyl), —C(O)—(C₁-C₄ alkyl) or —C(O)O—(C₁-C₄ alkyl); and R² is —C(O)Hca, —(C₀-C₃ alkyl)-Ar, —(C₀-C₃ alkyl)-Het, —(C₀-C₃ alkyl)-Cak or —(C₀-C₃ alkyl)-Hca. In certain embodiments, E is —C(O)NR¹R². In other embodiments, E is —NR¹R². In other embodiments, E is —R². In still other embodiments, E is —NR¹C(O)R².

In certain embodiments as described above, in the AMPK-activating compounds of structural formula (I) and (II), R¹ is H, —(C₁-C₄ alkyl), —C(O)—(C₁-C₄ alkyl) or —C(O)O—(C₁-C₄ alkyl); and R² is —C(O)Hca, —(C₀-C₃ alkyl)-Ar, —(C₀-C₃ alkyl)-Het, —(C₀-C₃ alkyl)-Cak or —(C₀-C₃ alkyl)-Hca. In certain embodiments as described above, in the AMPK-activating compounds of structural formula (I) and (II), R¹ is H. In other embodiments, R¹ is (C₁-C₄ alkyl), for example methyl, ethyl, n-propyl or isopropyl. In still other embodiments, R¹ is —C(O)—O—(C₁-C₄ alkyl), for example —C(O)OCH₃ or —C(O)—O-t-butyl. In certain embodiments, no alkyl of R¹ is substituted with an aryl-, heteroaryl-, cycloalkyl- or heterocycloalkyl-containing group. In certain embodiments, any alkyl of R¹ is unsubstituted.

In certain embodiments as described above, in the AMPK-activating compounds of structural formula (I) and (II), R² is -Hca. In certain embodiments, R² is an optionally-substituted monocyclic heterocycloalkyl. By way of example, such optionally substituted R² moieties include, without limitation, -(optionally-substituted azetidinyl), -(optionally-substituted pyrrolidinyl), -(optionally-substituted piperidinyl), -(optionally-substituted piperazinyl) or -(optionally-substituted azepanyl). For example, in one embodiment, R² can be -(optionally substituted piperidinyl) or -(optionally substituted pyrrolidinyl). In one embodiment, R² is -(optionally substituted piperidinyl). In another embodiment, R² is -(optionally substituted pyrrolidinyl). In another embodiment, R² is -(optionally substituted piperazinyl).

In certain particular embodiments as described above, in the AMPK-activating compounds of structural formula (I) and (II), R² is -(optionally-substituted azetidin-3-yl), -(optionally substituted piperidin-4-yl), -(optionally substituted piperazin-4-yl), -(optionally substituted pyrrolidin-3-yl) or -(optionally-substituted azepan-4-yl). For example, in one embodiment, R² is -(optionally substituted piperidin-4-yl). In another embodiment, R² is -(optionally substituted pyrrolidin-3-yl). In another embodiment, R² is -(optionally substituted piperazin-4-yl).

In certain particular embodiments, when R² is -(optionally substituted piperidin-4-yl), it is unsubstituted at its 2- and 3-positions.

In other embodiments, when R² is -(optionally substituted piperidin-4-yl), it is substituted with F at a 3-position. For example, R² can be

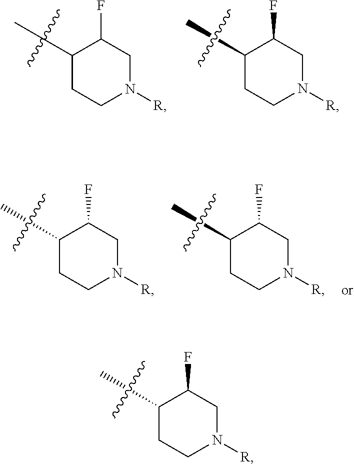

in which the R group is a further substituent, for example, as described below (e.g., a -G-R¹⁷ substituent). Such compounds can be provided as mixtures of diastereomers or enantiomers, or in diastereomerically and/or enantiomerically enriched form. In certain embodiments, the compound is provided in substantially diastereomerically pure form, for example, as substantially diastereomerically pure cis compound, or diastereomerically pure trans compound. In certain embodiments, a compound is provided in substantially enantiomerically pure form.

In certain embodiments as described above, in the AMPK-activating compounds of structural formula (I) and (II), the azetidinyl, pyrrolidinyl, piperidinyl, piperazinyl and azepanyl R² moieties described above are substituted, for example, at their 1-positions. In certain alternative embodiments, they can be substituted at their 4-positions (e.g., when a piperidin-1-yl) or 3 positions (e.g., when a pyrrolidin-5-yl). For example, in one embodiment, R² is substituted (e.g., at its 1-position) with —(C₀-C₃ alkyl)-Ar or —(C₀-C₃ alkyl)-Het, for example -(unsubstituted C₀-C₃ alkyl)-Ar or -(unsubstituted C₀-C₃ alkyl)-Het. For example, in one particular embodiment, the azetidinyl, pyrrolidinyl, piperidinyl, piperazinyl or azepanyl R² moiety is substituted (e.g., at its 1-position) with an optionally substituted benzyl or an optionally substituted phenyl. In another embodiment, the azetidinyl, pyrrolidinyl, piperidinyl, piperazinyl or azepanyl R² moiety is substituted (e.g., at its 1-position) with a benzyl substituted with an electron withdrawing group; or a phenyl substituted with an electron withdrawing group. For example, the benzyl or phenyl can be substituted with an electron withdrawing group selected from the group consisting of halo, cyano, —(C₁-C₄ fluoroalkyl), —O—(C₁-C₄ fluoroalkyl), —C(O)—(C₀-C₄ alkyl), —C(O)O—(C₀-C₄ alkyl), —C(O)N(C₀-C₄ alkyl)(C₀-C₄ alkyl), —S(O)₂O—(C₀-C₄ alkyl), SF₅, NO₂ and —C(O)—Hca in which the Hca includes a nitrogen atom to which the —C(O)— is bound, in which no alkyl, fluoroalkyl or heterocycloalkyl is substituted with an aryl, heteroaryl, cycloalkyl or heterocycloalkyl-containing group. In other embodiments, the azetidinyl, pyrrolidinyl, piperidinyl, piperazinyl or azepanyl R² moiety is substituted (e.g., at its 1-position) with an unsubstituted benzyl or an unsubstituted phenyl. In other embodiments, the azetidinyl, pyrrolidinyl, piperidinyl, piperazinyl or azepanyl R² moiety is substituted (e.g., at its 1-position) with —CH(CH₃)Ar, CH(C(O)OCH₃)Ar or —C(CH₃)₂Ar.

In other embodiments as described above, in the AMPK-activating compounds of structural formula (I) and (II), the azetidinyl, pyrrolidinyl, piperidinyl, piperazinyl or azepanyl R² moiety is substituted (e.g., at its 1-position) with an optionally substituted pyridinylmethyl, an optionally substituted furanylmethyl, an optionally substituted thienylmethyl, an optionally substituted oxazolylmethyl, or an optionally substituted imidazolylmethyl. For example, the azetidinyl, pyrrolidinyl, piperidinyl, piperazinyl or azepanyl R² moiety can be substituted with an unsubstituted pyridinylmethyl, an unsubstituted furanylmethyl, an unsubstituted thienylmethyl, an unsubstituted oxazolylmethyl, or an unsubstituted imidazolylmethyl. In other embodiments, the azetidinyl, pyrrolidinyl, piperidinyl or azepanyl R² moiety can be substituted with an pyridinylmethyl, furanylmethyl, thienylmethyl, oxazolylmethyl or imidazolylmethyl substituted with an electron withdrawing group as described above.

In certain embodiments as described above, in the AMPK-activating compounds of structural formula (I) and (II), the azetidinyl, pyrrolidinyl, piperidinyl, piperazinyl or azepanyl R² moiety is substituted (e.g., at its 1-position) with -L-Ar or -L-Het, in which Ar and Het can be, for example, as described above with reference to —(C₀-C₃ alkyl)-Ar or —(C₀-C₃ alkyl)-Het. In one such embodiment, L is —C(O)—NR⁹—, such as —C(O)—NH—. In other embodiments of the presently disclosed compounds of structural formula (I) as described above, the azetidinyl, pyrrolidinyl, piperidinyl, piperazinyl or azepanyl R² moiety is substituted (e.g., at its 1-position) with —C(O)—O(C₀-C₆ alkyl), —C(O)—Het, —C(O)—Ar, —S(O)₂-Het, —S(O)₂—Ar or —S(O)₂—O(C₀-C₆ alkyl), in which Ar and Het can be, for example, as described above with reference to —(C₀-C₃ alkyl)-Ar or —(C₀-C₃ alkyl)-Het. In one embodiment, the azetidinyl, pyrrolidinyl, piperidinyl, piperazinyl or azepanyl R² moiety is substituted (e.g., at its 1-position) with —C(O)—Het or —C(O)—Ar; in another embodiment, it is substituted (e.g., at its 1-position) with —S(O)₂-Het or —S(O)₂—Ar. For example, in certain embodiments, the azetidinyl, pyrrolidinyl, piperidinyl, piperazinyl or azepanyl R² moiety is substituted (e.g., at its 1-position) with an optionally-substituted benzoyl (for example, substituted with an electron withdrawing group as described above); or with an optionally-substituted nicotinyl, isonicotinyl or picolinyl (for example, optionally substituted with an electron withdrawing group as described above). In other embodiments, the azetidinyl, pyrrolidinyl, piperidinyl, piperazinyl or azepanyl R² moiety is substituted (e.g., at its 1-position) with an unsubstituted benzoyl; or an unsubstituted nicotinoyl, isonicotinoyl or picolinoyl.

In other embodiments as described above, in the AMPK-activating compounds of structural formula (I) and (II), the azetidinyl, pyrrolidinyl, piperidinyl, piperazinyl or azepanyl R² moiety is substituted (e.g., at its 1-position) with —(C₀-C₃ alkyl)-Cak, for example -(unsubstituted C₀-C₃ alkyl)-Cak (e.g., —CH₂-Cak) or —C(O)—Cak.

In one embodiment, R² is not an oxo-substituted heterocycloalkyl. In another embodiment, R² is not a tetramethyl-substituted heterocycloalkyl.

In certain embodiments as described above, in the AMPK-activating compounds of structural formula (I) and (II)(for example, those in which E is —C(O)NR¹R²), R¹ and R² together with the nitrogen to which they are bound form Hca. In such embodiments, Hca can be, for example, an optionally-substituted piperidinyl, an optionally-substituted pyrrolidinyl, or an optionally-substituted piperazinyl. When R¹ and R² together to form Hca, it can be defined and substituted as described above for R² wherein it is Hca.

In certain embodiments as described above, in the AMPK-activating compounds of structural formula (I) and (II)(for example, those in which E is —R², or —NR¹R²), R² is —C(O)Hca. In certain such embodiments, the Hca is linked to the —C(O)— through a nitrogen. In other such embodiments, the Hca can be linked to the —C(O)— through a carbon atom. The Hca can be defined and substituted, for example, as described above with respect to R² when it is Hca.

In certain embodiments as described above, in the AMPK-activating compounds of structural formula (I) and (II) (for example, those in which E is —R² or —C(O)NR¹R²), R² is —(C₀-C₃ alkyl)-Ar or —(C₀-C₃ alkyl)-Het. For example, in certain embodiments, R² is Ar, in which the Ar can be, for example, monocyclic, such as optionally-substituted phenyl. In other embodiments, R² is —(C₁-C₃ alkyl)-(optionally-substituted phenyl), for example optionally-substituted benzyl. In other embodiments, R² is Het, in which the Het can be, for example, monocyclic, such as optionally-substituted pyridinyl or optionally-substituted 1H-pyrazolyl. In other embodiments of the compounds of structural formula (I) as described above (for example, those in which E is —C(O)NR¹R²), R² is —(C₀-C₃ alkyl)-Cak, in which the Cak can be, for example, monocyclic, such as optionally-substituted cyclohexyl. The aryl, heteroaryl or cycloalkyl of R² can be substituted, for example, as described above with reference to R² when it is Hca. For example, in certain embodiments, the aryl, heteroaryl or cycloalkyl of R² is substituted with —(C₀-C₃ alkyl)-Ar or —(C₀-C₃ alkyl)-Het, substituted as described above. In other embodiments, the aryl, heteroaryl or cycloalkyl of R² is substituted with —O—(C₀-C₃ alkyl)-Ar or —O—(C₀-C₃ alkyl)-Het. In other embodiments, the aryl, heteroaryl or cycloalkyl of R² is substituted with an optionally-substituted heterocycloalkyl, such as a morpholin-1-yl, a 4-methylpiperazin-1-yl, or a pyrrolidin-1-yl. The ring system of the R² moiety can be substituted at any position. For example, in certain embodiments, the ring of a monocyclic R² moiety is substituted at the 4-position, as counted from the attachment to the central pyridine, pyrazine, pyridazine or pyrimidine, or the nitrogen or carbonyl of the E moiety. In other embodiments, the ring of a monocyclic R² moiety is substituted at the 3-position, as counted from the attachment to the central pyridine, pyrazine, pyridazine or pyrimidine, or the nitrogen or carbonyl of the E moiety.

In certain embodiments as described above, in the AMPK-activating compounds of structural formula (I) and (II), the compound has structural formula (III)

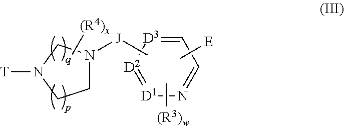

in which E is —R², —C(O)NR¹R², —NR¹R² or —NR¹C(O)R², in which R¹ and R² together with the nitrogen to which they are bound form Hca, or R¹ is H, —(C₁-C₄ alkyl), —C(O)—(C₁-C₄ alkyl) or —C(O)O—(C₁-C₄ alkyl); and R² is —C(O)Hca, —(C₀-C₃ alkyl)-Ar, —(C₀-C₃ alkyl)-Het, —(C₀-C₃ alkyl)-Cak or —(C₀-C₃ alkyl)-Hca. All other variables are as described above with reference to structural formulae (I) and (II). In certain such embodiments, E is R², —NR¹R² or —NR¹C(O)R². In certain embodiments of the compounds of structural formula (III), J is —C(O)—.

In certain embodiments as described above, in the AMPK-activating compounds of any of structural formulae (I)-(III) (for example, those in which E is —C(O)NR¹R²), when R² is Hca (for example, pyrrolidine or piperidine), it is substituted with at least one fluorine, and further optionally substituted, for example, as described below. In certain embodiments of compounds of structural formula (III) (for example, those in which E is —C(O)NR¹R²), when R² is Hca (for example, pyrollidine or piperazine), it is substituted (for example, at the nitrogen) with —C(O)—R²², —S(O)₂—R²², —C(O)—Cak, —CH₂-Cak, —CH(CH₃)—R²², —C(CH₃)₂—R²², —CH(C(O)—O(C₁-C₄ alkyl))Het, in which R²² is Ar or Het, and further optionally substituted, for example, as described below.

In certain embodiments as described above, in the AMPK-activating compounds of any of structural formulae (I)-(III)(for example, those in which E is —C(O)NR¹R²), R¹ and R² together with the nitrogen to which they are bound form Hca, as described below. For example, R¹ and R² can together to form an optionally substituted piperazine or an optionally-substituted pyrrolidine, as described below. In other embodiments, R¹ and R² together with the nitrogen to which they are bound form an optionally-substituted spirocyclic heterocycloalkyl (for example, 2,8-diazaspiro[4.5]decanyl), as described below.

In certain embodiments as described above, in the AMPK-activating compounds of any of structural formulae (I)-(III)(for example, those in which E is —C(O)NR¹Hca), T is H, —C(O)—(C₁-C₆ alkyl) or (C₁-C₆ alkyl), for example, as described below. In other embodiments as described above, in the AMPK-activating compounds of any of structural formulae (I)-(III)(for example, those in which E is —C(O)NR¹Hca), T is —C(CH₃)₂Ar, —CH₂-Het, -Het, —CH₂-Cak or Hca, for example, as described below. In other embodiments as described above, in the AMPK-activating compounds of any of structural formulae (I)-(III)(for example, those in which E is —C(O)NR¹Hca), T is

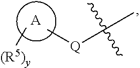

in which Q is —C(O)— or —S(O)₂—, for example, as described below.

In certain embodiments as described above, in the AMPK-activating compounds of any of structural formulae (I) or (II), the compound has structural formula (IV)

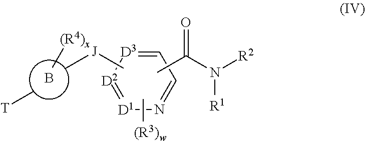

in which J is absent, —NR¹³—, —NR¹³C(O)— or —C(O)NR¹³—; and the ring system denoted by “B” is arylene, heteroarylene, or absent, and all other variables are as described with respect to structural formulae (I)-(III). For example, in certain embodiments as described above, in the AMPK-activating compounds of structural formula (IV), J is absent. In other embodiments, J is —NR¹³—, such as —NH—. In other embodiments, J is —NR¹³C(O)—, such as —NHC(O)—. In certain embodiments, the ring system denoted by “B” is arylene, such as phenylene); or heteroarylene, such as 1H-pyrazolylene, 1H-1,2,3-triazolylene), with particular examples being described below. In other embodiments, the ring system denoted by “B” is absent, with particular examples being described below. In certain embodiments as described above, in the AMPK-activating compounds of structural formula (IV), (for example, those in which E is —C(O)NR¹R²), R² is Hca, such as piperidinyl, with particular examples being described below.

In certain embodiments as described above, in the AMPK-activating compounds of any of structural formulae (I) or (II), the compound has structural formula (V)

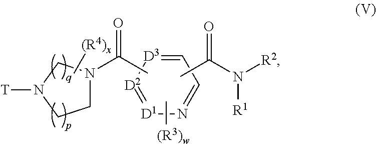

in which the variables are as described above with reference to structural formulae (I)-(III). In certain embodiments as described above, in the compounds of structural formula (V), R² is Hca (for example, pyrrolidine or piperidine), for example, described below. In other embodiments as described above, in the compounds of structural formula (V), R² is Cak, such as cyclohexyl, for example, described below.

In certain embodiments as described above, in the AMPK-activating compounds of any of structural formulae (I) or (II), the compound has structural formula (VI)

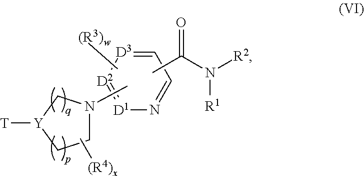

in which Y is N, C, CF or CH, and all other variables are as described above with reference to structural formulae (I)-(III). For example, in certain embodiments, Y is N. In other embodiments, Y is CF or CH. In certain embodiments as described above, in the compounds of structural formula (VI), p is 1 and q is 2. In other embodiments (for example, when Y is C, CF or CH), q is 1 and p is 1. In certain embodiments as described above, in the compounds of structural formula (VI), R² is Hca, such as pyrrolidine or piperidine.

In certain embodiments as described above, in the AMPK-activating compounds of any of structural formulae (I) or (II), the compound has structural formula (VII)

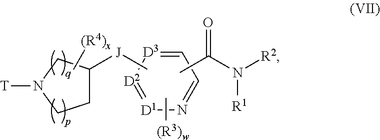

in which J is absent, —NR¹³—, —NR¹³C(O)— or —C(O)NR¹³—, and all other variables are as described above with reference to structural formulae (I)-(III). For example, in one embodiment, J is —NR¹³—C(O)—. In other embodiments, J is —NR¹³—. In certain embodiments as described above, in the compounds of structural formula (VII), p is 1 and q is 2. In other embodiments, q is 1 and p is 1. In other embodiments (for example, when Y is C, CF or CH), q is 1 and p is 0. In certain embodiments as described above, in the compounds of structural formula (VII), R² is Hca, such as pyrrolidine or piperidine, particular examples of which are further described below.

In certain embodiments as described above, in the AMPK-activating compounds of any of structural formulae (I) or (II), the compound has structural formula (VIII)

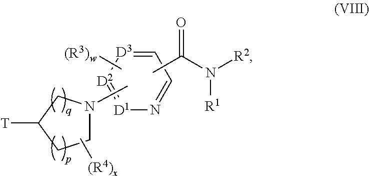

in which the variables are as described above with reference to structural formulae (I)-(III). In certain embodiments as described above, in the compounds of structural formula (VIII), p is 1 and q is 2. In other embodiments, q is 1 and p is 1. In other embodiments (for example, when Y is C, CF or CH), q is 1 and p is 0. In certain embodiments as described above, in the compounds of structural formula (VIII), R² is Hca.

In certain embodiments as described above, in the AMPK-activating compounds of any of structural formulae (I)-(VIII), T is

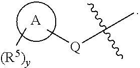

In such embodiments, Q is —O—(C₀-C₃ alkyl)-, —S(O)₂—, L or —(C₀-C₃ alkyl)- in which each carbon of the (C₀-C₃ alkyl) is optionally and independently substituted with one or two R¹⁶, in which each R¹⁶ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and optionally two of R¹⁶ on the same carbon combine to form oxo. In certain embodiments, each R¹⁶ is independently selected from —(C₁-C₆ alkyl), —(C₁-C₆ haloalkyl) (for example, difluoromethyl, trifluoromethyl and the like), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R¹⁶ on the same carbon optionally combine to form an oxo, in which each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl), and —(C₀-C₆ alkyl)-S(O)₀₋₂—(C₀-C₆ alkyl), and in which no alkyl or haloalkyl is substituted with an aryl-, heteroaryl-, cycloalkyl- or heterocycloalkyl-containing group. For example, in particular compounds, each R¹⁶ is —(C₁-C₃ alkyl), —(C₁-C₃ haloalkyl), —(C₀-C₃ alkyl)-L-R⁷, —(C₀-C₃ alkyl)-NR⁸R⁹, —(C₀-C₃ alkyl)-OR¹⁰, —(C₀-C₃ alkyl)-C(O)R¹⁰, —(C₀-C₃ alkyl)-S(O)₀₋₂R¹⁰, -halogen, —NO₂ and —CN, and two R¹⁶ on the same carbon optionally combine to form an oxo, in which each R⁷, R⁸ and R¹⁰ is independently selected from H, —(C₁-C₂ alkyl), —(C₁-C₂ haloalkyl), —(C₀-C₂ alkyl)-L-(C₀-C₂ alkyl), —(C₀-C₂ alkyl)-NR⁹(C₀-C₂ alkyl), —(C₀-C₂ alkyl)-O—(C₀-C₂ alkyl), —(C₀-C₂ alkyl)-C(O)—(C₀-C₂ alkyl) and —(C₀-C₂ alkyl)-S(O)₀₋₂—(C₀-C₂ alkyl), and in which no alkyl or haloalkyl is substituted with an aryl-, heteroaryl-, cycloalkyl- or heterocycloalkyl-containing group. In certain embodiments, each R¹⁶ is independently methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂, N₃, —SF₅, or —CN, and two R¹⁶ optionally come together to form oxo. In certain embodiments, Q has at most one R¹⁶ or an oxo substituted thereon. Q can be, for example, an unsubstituted —(C₀-C₃ alkyl)- (for example, a single bond, —CH₂— or —CH₂—CH₂—). In other embodiments, Q is a (C₁-C₃ alkyl) having as its only substitution a single oxo group. For example, in certain embodiments of the compounds of structural formulae (I)-(VII) as described above, Q is —CH—; —CH₂CH₂—; —OCH₂CH₂—; O; a single bond; —S(O)₂—; —C(O)—; —CHF—; —CH(OH)—, —C(CH₃)₂—, or —CH(CH₃)—.

In certain embodiments as described above, in the AMPK-activating compounds of any of structural formulae (I)-(VIII), T is

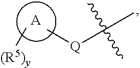

in which Q is —C(O)— or —S(O)₂—. In other embodiments, T is

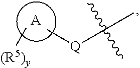

in which Q is —C(CH₃)₂—, —CH₂CH₂—, —CH(CH₃)—, —CH(OH)— or —CHF—.

In certain embodiments as described above, in the AMPK-activating compounds of any of structural formulae (I)-(VIII) (for example, those in which T is not bound to a nitrogen), T is

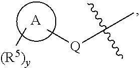

in which Q is O.

In certain embodiments as described above, in the AMPK-activating compounds of any of structural formulae (I)-(VIII) (for example, those in which the ring system denoted by “B” is absent), T is

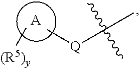

in which Q is —O—(C₁-C₃ alkyl)-, for example, —OCH₂— or —OCH₂CH₂—.

The number of substituents, y, on the ring system denoted by “A”, is 0, 1, 2, 3 or 4. For example, in some embodiments as described above, in the AMPK-activating compounds of any of structural formulae (I)-(VIII), y is 0, 1, 2 or 3, such as 1. In one embodiment, y is not zero and at least one R⁵ is halo, cyano, —(C₁-C₄ haloalkyl), —O—(C₁-C₄ haloalkyl), —(C₁-C₄ alkyl), —O—(C₁-C₄ alkyl), —C(O)—(C₀-C₄ alkyl), —C(O)O—(C₀-C₄ alkyl), —C(O)N(C₀-C₄ alkyl)(C₀-C₄ alkyl), —N₃, —SF₅, NO₂ or —C(O)—Hca wherein the Hca contains a ring nitrogen atom through which it is bound to the —C(O)—, and wherein no alkyl, haloalkyl or heterocycloalkyl is substituted by an aryl, heteroaryl, cycloalkyl or heterocycloalkyl-containing group.

In certain embodiments as described above, in the AMPK-activating compounds of any of structural formulae (I)-(VIII), each R⁵ is independently selected from —(C₁-C₆ alkyl), —(C₁-C₆ haloalkyl) (for example, difluoromethyl, trifluoromethyl and the like), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —N₃, —SF₅, —NO₂ and —CN, in which each R⁷, R⁸ and R¹⁰ is independently selected from H, —(C₁-C₆ alkyl), —(C₁-C₆ haloalkyl) (for example, difluoromethyl, trifluoromethyl and the like), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl), and in which no alkyl or haloalkyl is substituted with an aryl-, heteroaryl-, cycloalkyl- or heterocycloalkyl-containing group. For example, in one embodiment, each R⁵ is —(C₁-C₃ alkyl), —(C₁-C₃ haloalkyl), —(C₀-C₃ alkyl)-L-R⁷, —(C₀-C₃ alkyl)-NR⁸R⁹, —(C₀-C₃ alkyl)-OR¹⁰, —(C₀-C₃ alkyl)-C(O)R¹⁰, —(C₀-C₃ alkyl)-S(O)₀₋₂R¹⁰, -halogen, —N₃, —SF₅, —NO₂ and —CN, in which each R⁷, R⁸ and R¹⁰ is independently selected from H, —(C₁-C₂ alkyl), —(C₁-C₂ haloalkyl), —(C₀-C₂ alkyl)-L-(C₀-C₂ alkyl), —(C₀-C₂ alkyl)-NR⁹(C₀-C₂ alkyl), —(C₀-C₂ alkyl)-O—(C₀-C₂ alkyl), —(C₀-C₂ alkyl)-C(O)—(C₀-C₂ alkyl) and —(C₀-C₂ alkyl)-S(O)₀₋₂—(C₀-C₂ alkyl), and in which no alkyl or haloalkyl is substituted with an aryl-, heteroaryl-, cycloalkyl- or heterocycloalkyl-containing group. In certain embodiments, each R⁵ is independently halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, each R⁵ is independently methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂, N₃, —SF₅, or —CN.

In one embodiment as described above, in the AMPK-activating compounds of any of structural formulae (I)-(VIII), y is 0. In another embodiment, y is 1. In another embodiment, y is 2.

In various aspects as described above, in the AMPK-activating compounds of structural formulae (I)-(VIII), the ring system denoted by “A” is heteroaryl, aryl, cycloalkyl or heterocycloalkyl. For example, in one embodiment, the ring system denoted by “A” is an aryl or a heteroaryl. The ring system denoted by “A” can be, for example, a monocyclic aryl or heteroaryl. In one embodiment, when the “A” ring system is aryl, Q is a —(C₀-C₃ alkyl)- optionally substituted with oxo, and optionally substituted with one or more R¹⁶. For example, Q can be a —(C₁-C₃ alkyl)- having its only substitution a single oxo, or an unsubstituted —(C₀-C₃ alkyl)-. In certain embodiments, the ring system denoted by “A” is an aryl or a heteroaryl and Q is —CH₂—; —CH₂CH₂—; a single bond; —S(O)₂—; —C(O)—; or —CH(CH₃)—. In other embodiments, the ring system denoted by “A” is an aryl or a heteroaryl and Q is —CF—, —CH(OH)— or —C(CH₃)₂—. In other embodiments, the ring system denoted by “A” is an aryl or a heteroaryl and Q is —O—, —OCH₂— or —OCH₂CH₂—.

For example, in certain embodiments as described above, in the AMPK-activating compounds of any of structural formulae (I)-(VIII), the ring system denoted by “A” is monocyclic aryl, such as phenyl. In one embodiment, y is 1 and R⁵ is attached to the phenyl in the para position relative to Q. In one embodiment, y is 1 and R⁵ is attached to the phenyl in the meta position relative to Q. In certain embodiments, y is 1 and R⁵ is selected from the group consisting of halo, cyano, —(C₁-C₄ haloalkyl), —O—(C₁-C₄ haloalkyl), —(C₁-C₄ alkyl), —O—(C₁-C₄ alkyl), —C(O)—(C₀-C₄ alkyl), —C(O)O—(C₀-C₄ alkyl), —C(O)N(C₀-C₄ alkyl)(C₀-C₄ alkyl), NO₂ and —C(O)—Hca in which the Hca contains a ring nitrogen atom through which it is bound to the —C(O)—, and in which no (C₀-C₄ alkyl) or (C₁-C₄ alkyl) is substituted by an aryl, heteroaryl, cycloalkyl or heterocycloalkyl-containing group. R⁵ can be, for example, —Cl, —F, cyano, —N₃, SF₅, —C(O)CH₃, —C(O)OH, —C(O)NH₂, methoxy, trifluoromethyl, difluoromethyl, difluoromethoxy or trifluoromethoxy. In another embodiment, the

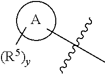

moiety is a 3,4-dihalophenyl, a 3,5-dihalophenyl, a 3-cyano-5-methoxyphenyl, a 4-cyano-3-halophenyl, or a 3-halo-4-methoxyphenyl.

In another embodiment as described above, in the AMPK-activating compounds of any of structural formulae (I)-(VIII), the ring system denoted by “A” is a heteroaryl. For example, in certain embodiments, the ring system denoted by “A” is a pyridyl, a thienyl, or a furanyl. In another embodiment, the ring system denoted by “A” is an isoxazolyl. In one embodiment, when the “A” ring system is heteroaryl, Q is a —(C₀-C₃ alkyl)- optionally substituted with oxo, and optionally substituted with one or more R¹⁶. For example, Q can be a —(C₁-C₃ alkyl)- having its only substitution a single oxo, or an unsubstituted —(C₀-C₃ alkyl)-. In certain embodiments, the ring system denoted by “A” is an aryl or a heteroaryl and Q is —CH₂—; a single bond; —S(O)₂—; —C(O)—; or —CH(CH₃)—. In other embodiments, the ring system denoted by “A” is an aryl or a heteroaryl and Q is —O—, —CF—, —CH(OH)— or —C(CH₃)₂. In other embodiments, the ring system denoted by “A” is an aryl or a heteroaryl and Q is —O—, —OCH₂— or —OCH₂CH₂—.

In another embodiment as described above, in the AMPK-activating compounds of any of structural formulae (I)-(VIII), the ring system denoted by “A” is a heterocycloalkyl. For example, in certain embodiments, the ring system denoted by “A” is a tetrahydro-2H-pyranyl or a morpholino. In one such embodiment, when the “A” ring system is a heterocycloalkyl, Q is a single bond. In another such embodiment, Q is —CH₂— or —C(O)—. In another such embodiment, Q is —O—, —OCH₂— or —OCH₂CH₂—.

In another embodiment as described above, in the AMPK-activating compounds of any of structural formulae (I)-(VIII), the ring system denoted by “A” is a cycloalkyl. For example, in certain embodiments, the ring system denoted by “A” is a cyclohexyl. In one such embodiment, when the “A” ring system is a cycloalkyl, Q is —CH₂— or —C(O)—. In another such embodiment, Q is a single bond. In another such embodiment, Q is —O—, —OCH₂— or —OCH₂CH₂—.

In certain embodiments as described above, in the AMPK-activating compounds of any of structural formulae (I)-(VIII), T is H, —(C₁-C₆ alkyl) or —C(O)(C₁-C₆ alkyl). In certain such embodiments, the alkyl moieties of T are unsubstituted. In other such embodiments, the alkyl moieties of T are optionally substituted as described below. For example, in certain embodiments, T is H, ispropropyl, or —C(O)-t-butyl.

In certain embodiments as described above, in the AMPK-activating compounds of any of structural formulae (I)-(VIII), T is —C(CH₃)₂Ar, —CH₂-Het, -Het, —CH₂-Cak or -Hca. The —Ar, -Het, -Cak and -Hca moieties can, for example, be substituted with y R⁵ moieties, as described above with reference to the ring system denoted by “A”.

In certain embodiments as described above, in the AMPK-activating compounds of any of structural formulae (I)-(VIII), the T moiety is selected from the group consisting of

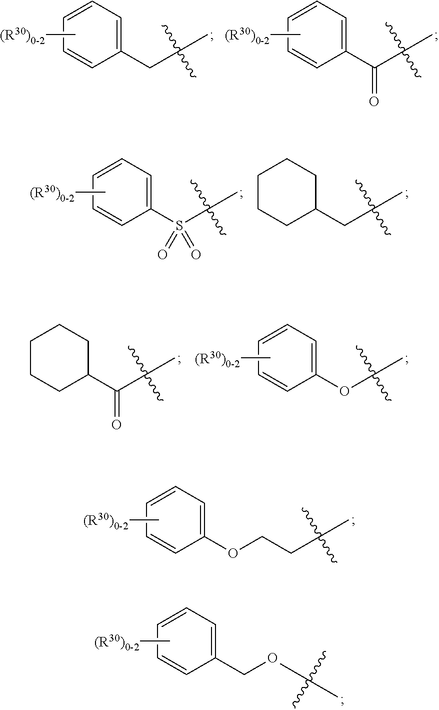

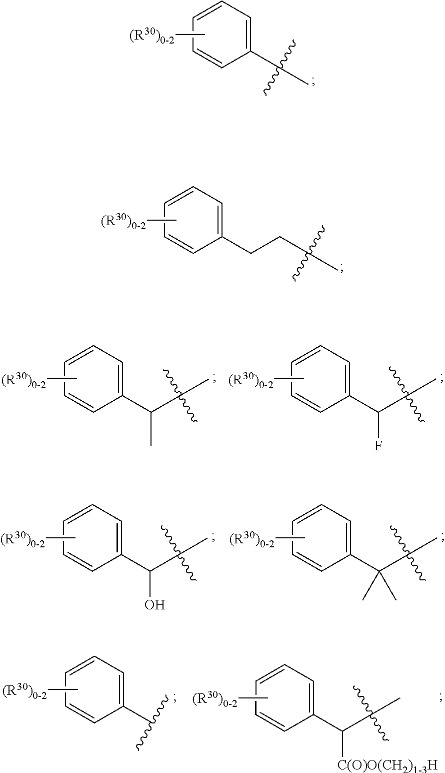

monocyclic heterocycloalkyl (for example, tetrahydropyranyl, morpholinyl, piperidinyl, piperazinyl) substituted with 0, 1 or 2 R³⁰, monocyclic heteroaryl (for example, pyridyl, isoxazolyl, oxazolyl, pyrrolyl, thienyl) substituted with 0, 1 or 2 R³⁰; monocyclic heteroarylmethyl- (for example, pyridylmethyl, isoxazolylmethyl, oxazolylmethyl, pyrrolylmethyl, thienylmethyl), in which the heteroaryl is substituted with 0, 1 or 2 R³⁰; or monocyclic heteroaryloxy- (for example, pyridyloxy, isoxazolyloxy, oxazolyloxy, pyrrolyloxy, thienyloxy), in which the heteroaryl is substituted with 0, 1 or 2 R³⁰; in which each R³⁰ is independently selected from halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, no R³⁰ is substituted on the ring of the T moiety. In other embodiments, one R³⁰ is substituted on the ring of the T moiety, for example, at a para-position of a phenyl, a meta-position of a phenyl, or at a 3- or 4- position of a heteroaryl or heterocycloalkyl (as counted from the attachment point of the ring system denoted by “B”). Certain particular identities of the T moiety will be found by the person of skill in the art in the compounds described below with respect to Table 1. Those of skill in the art will understand that combinations of such T moieties with other subcombinations of features disclosed herein is specifically contemplated.

For example, in certain embodiments as described above, in the AMPK-activating compounds of any of structural formulae (I)-(VIII), T moiety is selected from

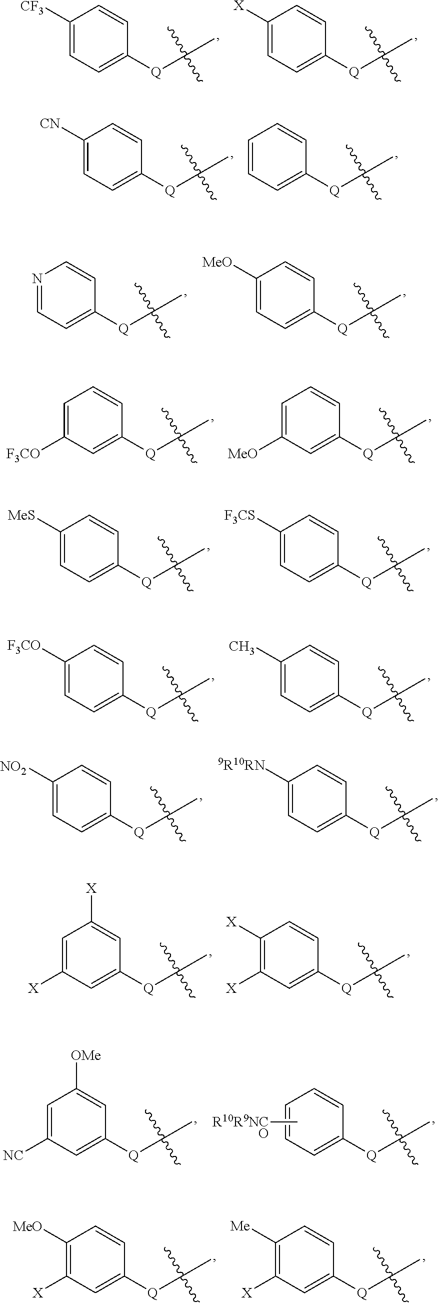

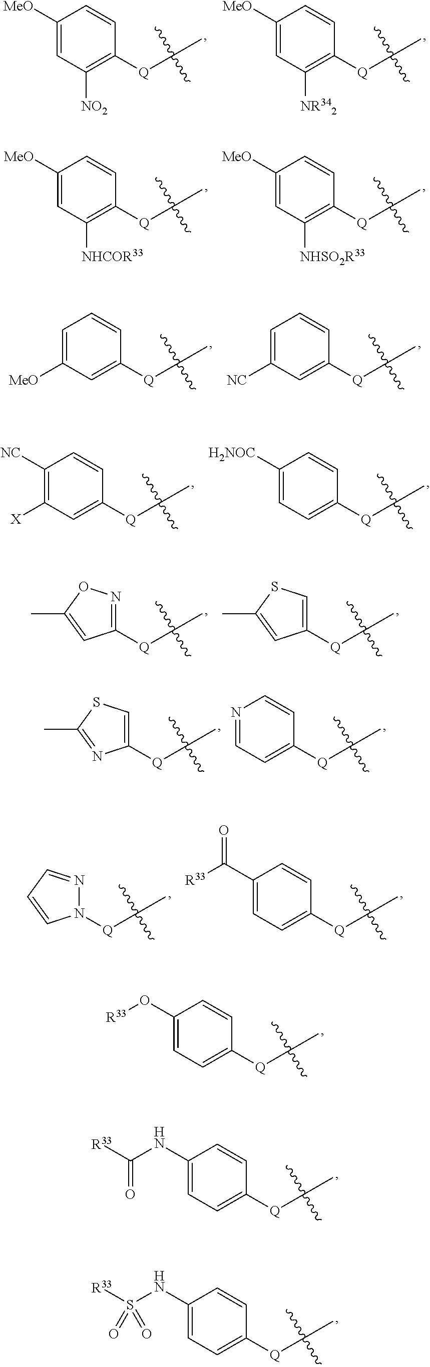

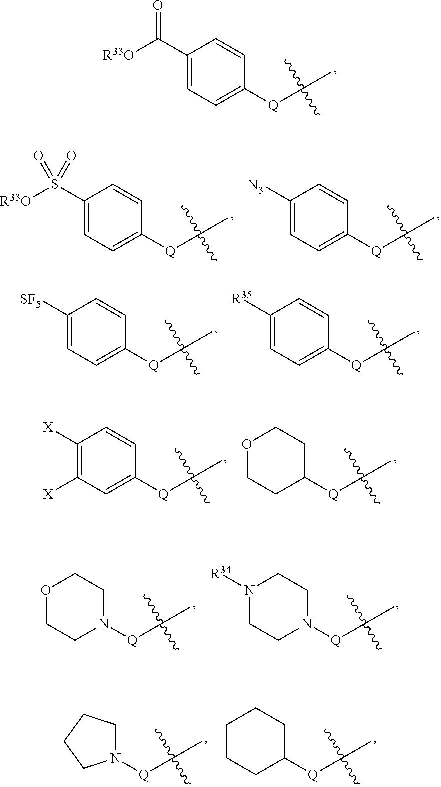

heterocycloalkyl optionally substituted by alkyl and/or halogen, -Q-heteroaryl optionally substituted by unsubstituted (C₁-C₄alkyl) and/or halogen, H, C(O)tBu and isopropyl, in which each X is independently F, Cl or Br (preferably F or Cl), each R³³ is unsubstituted (C₁-C₄ alkyl), unsubstituted (C₁-C₄ haloalkyl) or cycloalkyl optionally substituted with unsubstituted alkyl, unsubstituted (C₁-C₄ alkyl), unsubstituted (C₁-C₄ haloalkyl) or cycloalkyl optionally substituted with unsubstituted alkyl, and each R³⁵ is heterocycloalkyl, optionally substituted with unsubstituted alkyl. In certain such embodiments, Q is a single bond, —CH₂—, —CH₂O—, —OCH₂CH₂—, —CH₂CH₂—, —O—, —CHF—, —CH(CH₃)—, —C(CH₃)₂—, —CH(OH)—, —CH(COOMe)-, —CH(COOEt)-, —C(O)— or —S(O)₂—.

In one embodiment as described above, in the AMPK-activating compounds of any of structural formulae (I)-(VIII), the compound has structural formula (IX):

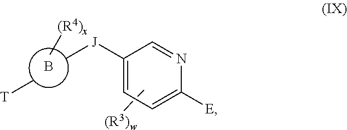

in which the variables are defined as described above with reference to any of structural formulae (I)-(VIII).

In another embodiment as described above, in the AMPK-activating compounds of any of structural formulae (I)-(VIII), the compound has structural formula (X):

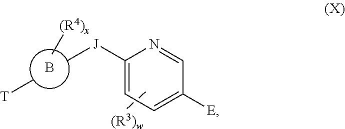

in which the variables are defined as described above with reference to any of structural formulae (I)-(VIII). For example, in certain embodiments, R² can be

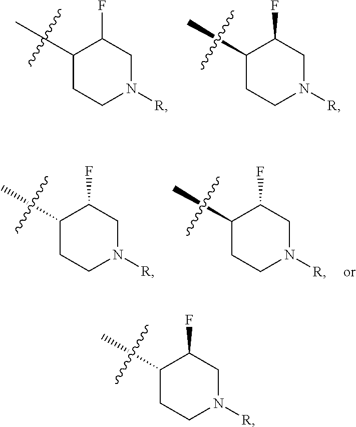

in which the R group is a further substituent, for example, as described herein.

In another embodiment as described above, in the AMPK-activating compounds of any of structural formulae (I)-(VIII), the compound has structural formula (XI):

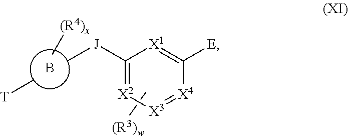

in which one of X¹, X², X³ and X⁴ are N, and the others are carbons (for example, independently CH or C substituted with one of the w R³ groups), and all other variables are defined as described above with reference to any of structural formulae (I)-(VIII). For example, in one embodiment, X¹ is N and X², X³ and X⁴ are carbons. In another embodiment, X² is N and X¹, X³ and X⁴ are carbons. In another embodiment, X³ is N and X¹, X² and X⁴ are carbons. In another embodiment, X⁴ is N and X¹, X² and X³ are carbons.

In another embodiment as described above, in the AMPK-activating compounds of any of structural formulae (I)-(VIII), the compound has structural formula (XII):

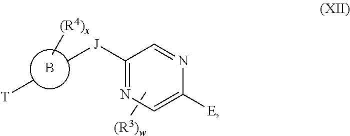

in which the variables are defined as described above with reference to any of structural formulae (I)-(VIII).

In another embodiment as described above, in the AMPK-activating compounds of any of structural formulae (I)-(VIII), the compound has structural formula (XIII):

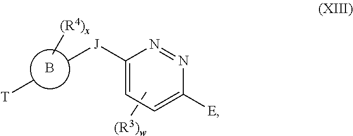

in which the variables are defined as described above with reference to any of structural formulae (I)-(VIII).

In the various aspects of the disclosure presently disclosed, in the AMPK-activating compounds of structural formulae (I)-(XIII) as described above, w, the number of substituents on the central pyridine, pyridazine, pyrazine or pyrimidine, is 0, 1, 2 or 3. For example, in one embodiment, w is 0, 1 or 2. In another embodiment, w is 0. In other embodiments, w is at least 1, and at least one R³ is selected from the group consisting of halo, cyano, —(C₁-C₄ fluoroalkyl), —O—(C₁-C₄ fluoroalkyl), —C(O)—(C₀-C₄ alkyl), —C(O)O—(C₀-C₄ alkyl), —C(O)N(C₀-C₄ alkyl)(C₀-C₄ alkyl), —S(O)₂O—(C₀-C₄ alkyl), NO₂ and —C(O)—Hca in which the Hca includes a nitrogen atom to which the —C(O)— is bound, in which no alkyl, fluoroalkyl or heterocycloalkyl is substituted with an aryl, heteroaryl, cycloalkyl or heterocycloalkyl-containing group. For example, in certain embodiments, at least one R³ is halo (for example, chloro) or —(C₁-C₄ alkyl) (for example, methyl, ethyl or propyl). In certain embodiments, an R³ is substituted on the central pyridine, pyrazine, pyridazine or pyrimidine in the meta position relative to the J moiety.

In certain embodiments as described above, in the AMPK-activating compounds of any of structural formulae (I)-(XIII), each R³ is independently selected from —(C₁-C₆ alkyl), —(C₁-C₆ haloalkyl) (for example, difluoromethyl, trifluoromethyl and the like), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, in which each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl), and —(C₀-C₆ alkyl)-S(O)₀₋₂—(C₀-C₆ alkyl), and in which no alkyl or haloalkyl is substituted with an aryl-, heteroaryl-, cycloalkyl- or heterocycloalkyl-containing group. For example, in one embodiment, each R³ is —(C₁-C₃ alkyl), —(C₁-C₃ haloalkyl), —(C₀-C₃ alkyl)-L-R⁷, —(C₀-C₃ alkyl)-NR⁸R⁹, —(C₀-C₃ alkyl)-OR¹⁰, —(C₀-C₃ alkyl)-C(O)R¹⁰, —(C₀-C₃ alkyl)-S(O)₀₋₂R¹⁰, -halogen, —NO₂ and —CN, in which each R⁷, R⁸ and R¹⁰ is independently selected from H, —(C₁-C₂ alkyl), —(C₁-C₂ haloalkyl), —(C₀-C₂ alkyl)-L-(C₀-C₂ alkyl), —(C₀-C₂ alkyl)-NR⁹(C₀-C₂ alkyl), —(C₀-C₂ alkyl)-O—(C₀-C₂ alkyl), —(C₀-C₂ alkyl)-C(O)—(C₀-C₂ alkyl) and —(C₀-C₂ alkyl)-S(O)₀₋₂—(C₀-C₂ alkyl), and in which no alkyl or haloalkyl is substituted with an aryl-, heteroaryl-, cycloalkyl- or heterocycloalkyl-containing group. For example, in certain embodiments, each R³ is halo (for example, chloro) or —(C₁-C₄ alkyl) (for example, methyl, ethyl or propyl). In certain embodiments, each R³ is independently halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, each R³ is independently methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂ or —CN.

In certain embodiments as described above, in the AMPK-activating compounds of any of structural formulae (I)-(XIII), w is at least one, and at least one R³ is —NR⁸R⁹. For example, in one embodiment, w is 1. In certain such embodiments, an R³ is substituted on the central pyridine, pyrazine, pyridazine or pyrimidine in the meta position relative to the J moiety.

In other embodiments as described above, in the AMPK-activating compounds of any of structural formulae (I)-(XIII), w is at least one, and at least one R³ is —(C₀-C₃ alkyl)-Y¹—(C₁-C₃ alkyl)-Y²—(C₀-C₃ alkyl), in which each of Y¹ and Y² is independently L, —O—, —S— or —NR⁹—. For example, in one embodiment, w is 1. In certain such embodiments, R³ is substituted on the central pyridine, pyrazine, pyridazine or pyrimidine in the meta position relative to the J moiety. In one particular embodiment, R³ is —CH₂—N(CH₃)—CH₂—C(O)—OCH₃.

In certain embodiments as described above, in the AMPK-activating compounds of any of structural formulae (I)-(XIII), the compound has the structural formula (XIV):

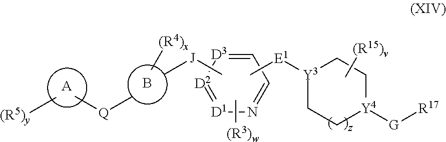

in which E¹ is absent, —C(O)—, —C(O)NR¹— or —NR¹C(O)—; z is 0 or 1; Y³ is N, C or CH and Y⁴ is N, C or CH; Q and G are each independently a single bond, —CH₂—, —C(H)(R¹⁶)—, —C(R¹⁶)₂—, —CH₂CH₂—, L (for example, —C(O)—NR⁹— or —NR⁹—C(O)—), -L-C(R¹⁶)₂—, —O—(C₀-C₃ alkyl)- in which the (C₀-C₃ alkyl) is bound to the R¹⁷ moiety or the ring system denoted by “A”, or —S(O)₂—; v is 0, 1, 2, 3 or 4; each R¹⁵ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R¹⁵ on the same carbon optionally combine to form oxo; and R¹⁷ is Het or Ar, and all other variables are defined as described above with reference to any of structural formula (I)-(XIII).

In certain embodiments as described above, in the presently disclosed compounds of structural formula (XIV) (for example, those in which E¹ is —C(O)— or absent, Y³ is N and Y⁴ is N. In other embodiments, (for example, those in which E¹ is —C(O)—NR¹—), Y³ is C or CH and Y⁴ is N. In other embodiments, Y³ is N and Y⁴ is C or CH. In other embodiments, Y³ is C or CH and Y⁴ is C or CH; in such embodiments, the E¹ and G moieties can be disposed, for example, cis to one another on the cycloalkyl ring. In certain embodiments as described above, in the presently disclosed compounds of structural formula (XIV), z is 1. In other embodiments, z is 0.

In certain embodiments as described above, in the AMPK-activating compounds of any of structural formulae (I)-(XIV), D¹, D² and D³ are all CH or C substituted by one of the w R³, and the R² moiety is an optionally-substituted piperidine. For example, in one embodiment, a compound has structural formula (XV):

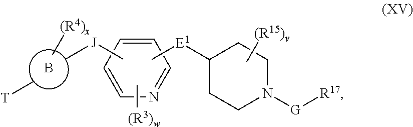

in which all variables are and all as described above with respect to any of structural formulae (I)-(XIV). In one such embodiment, v is 0.

In other embodiments as described above, in the AMPK-activating compounds of structural formula (XV), one of the R¹⁵ is F. For example, the F can be substituted at the carbon alpha to the E¹ moiety. Accordingly, in certain embodiments, a compound has structural formula (XVI):

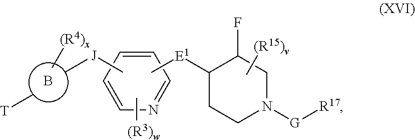

in which v is 0, 1, 2 or 3 and all other variables are as described above with respect to any of structural formulae (I)-(XIV). In certain such embodiments, v is 0. In one embodiment, the E¹ moiety and the F are disposed in a cis relationship to one another. In other embodiment, the E¹ moeity and the F are disposed in a trans relationship to one another. For example, the compound of structural formula (XVI) can be provided as any of the four diastereomers of structural formulae (XVII)-(XX):

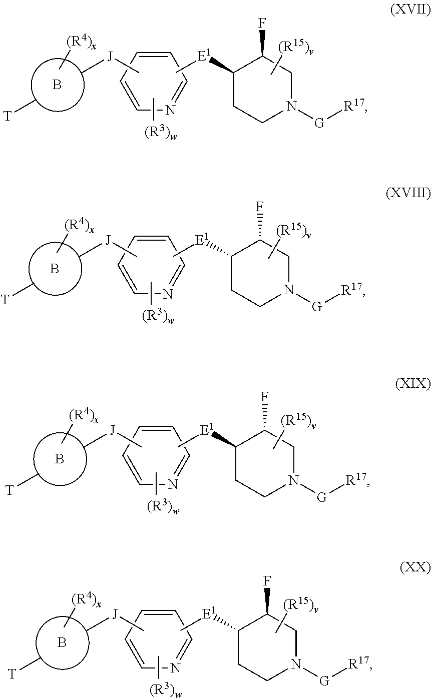

in which v is 0, 1, 2 or 3 (e.g., 0), and all other variables are and all as described above with respect to any of structural formulae (I)-(XVI). Compounds can be provided as mixtures of diastereomers or enantiomers, or in diastereomerically and/or enantiomerically enriched form. In certain embodiments, the compound is provided in substantially diastereomerically pure form, for example, as substantially diastereomerically pure cis compound, or diastereomerically pure trans compound. In certain embodiments, a compound is provided in substantially enantiomerically pure form, for example, as one of the compounds of structural formulae (XVII)-(XX).

In certain embodiments as described above, in the AMPK-activating compounds of any of structural formulae (XV)-(XX), the compound has structural formula (XXI):

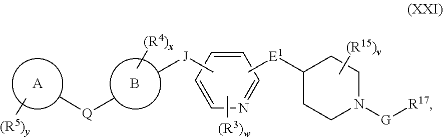

in which all variables are as described above with respect to any of structural formulae (I)-(XX). For example, the

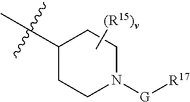

moiety can be selected from

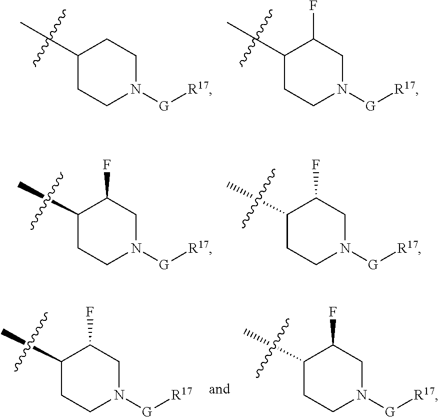

in which the G-R¹⁷ group is as described herein. Such compounds can be provided as mixtures of diastereomers or enantiomers, or in diastereomerically and/or enantiomerically enriched form. In certain embodiments, the compound is provided in substantially diastereomerically pure form, for example, as substantially diastereomerically pure cis compound, or diastereomerically pure trans compound. In certain embodiments, a compound is provided in substantially enantiomerically pure form.

In the compounds of structural formulae (XV)-(XXI), the regiochemistry around the central pyridine can be as described with respect to any of claims (IX)-(XI). Moreover, the E¹ moiety of any such compounds can be absent, —C(O)—, —C(O)NR¹— or —NR¹C(O)—. In one such embodiment, a compound of any of structural formula (XV)-(XXI) is of structural formula (XXII):

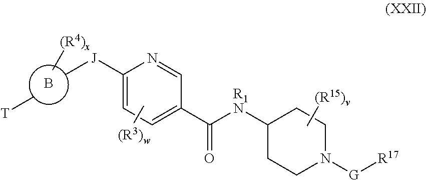

in which all variables are as described above with respect to any of structural formulae (I)-(XXI). For example, the

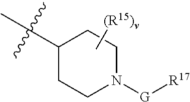

moiety can be selected from

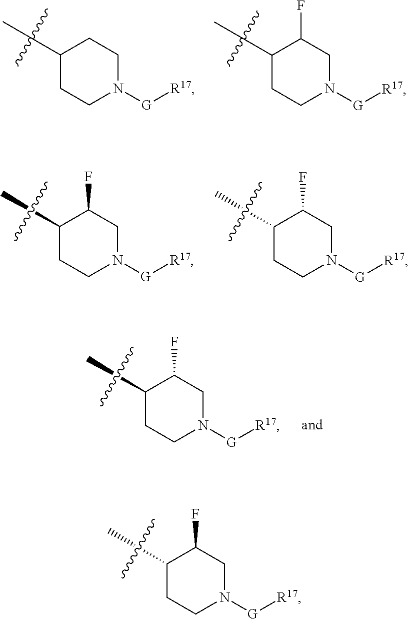

in which the G-R¹⁷ group is as described herein.

In certain embodiments as described above, in the AMPK-activating compounds of any of structural formulae (XV)-(XXII), the ring denoted by “B” is

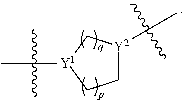

In certain such embodiments, Y² is N and Y¹ is CH or C substituted by one of the x R⁴. In other such embodiments, both Y¹ and Y² are N. For example, in certain embodiments, compounds according to structural formulae (XV)-(XXII) have structural formula (XXIII):

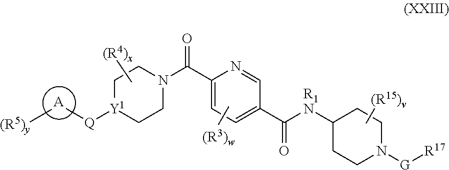

in which in which all variables are as described above with respect to any of structural formulae (I)-(XXII). In one embodiment, Y¹ is N. In another embodiment, Y¹ is CH, or is C substituted by one of the x R⁴. For example, in certain embodiments as described above, in the AMPK-activating compounds of any of structural formulae (XXIV)-(XXIX):

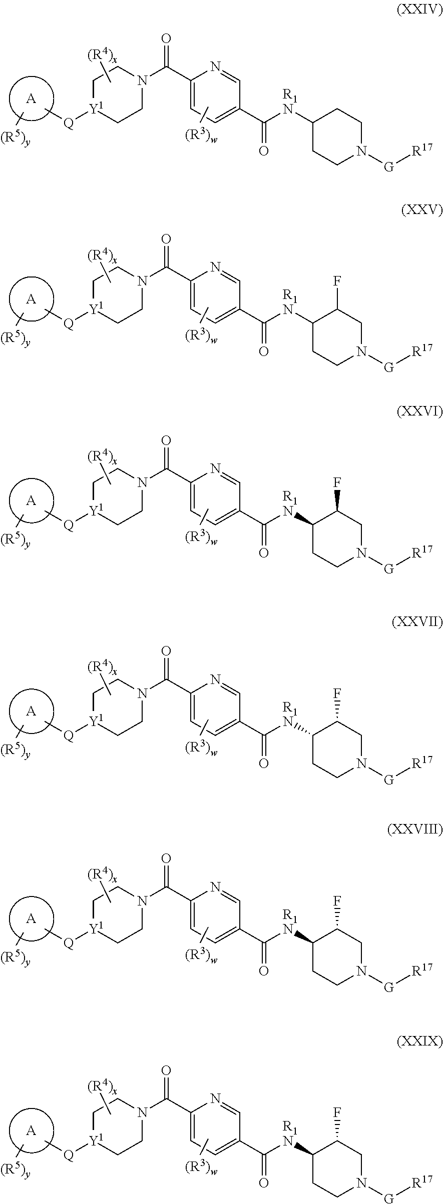

in which in which all variables are as described above with respect to any of structural formulae (I)-(XXII). In certain embodiments as described above, in the AMPK-activating compounds of any of structural formulae (XXIV)-(XXIX), Y¹ is CH or C substituted by one of the x R⁴. In certain embodiments as described above, in the AMPK-activating compounds of any of structural formulae (XXIV)-(XXIX), w is 0. In other such embodiments, x is 0. In still other such embodiments, both w and x are 0. In any such embodiments, R¹ can be, for example, H, or unsubstituted (C₁-C₄ alkyl) such as methyl. Compounds according to structural formulae (XXVI)-(XXIX) can be provided as mixtures of diastereomers or enantiomers, or in diastereomerically and/or enantiomerically enriched form. In certain embodiments, the compound is provided in substantially diastereomerically pure form, for example, as substantially diastereomerically pure cis compound, or diastereomerically pure trans compound. In certain embodiments, a compound is provided in substantially enantiomerically pure form.

In the compounds of structural formulae (XV)-(XXIX) as described above, G and Q can be as described above with reference to structural formulae (I)-(XIV). For example, in certain embodiments, G is CH₂, CO, or SO₂. In certain embodiments, Q is CH₂, CO, SO₂ or O.

In the compounds of structural formulae (XV)-(XXIX) as described above, R¹⁷ and T can be as described above with reference to structural formulae (I)-(XIV). For example, in certain embodiments, R¹⁷ is an optionally substituted phenyl, for example, substituted with 0-2 R³⁰ groups as described above. In other embodiments, R¹⁷ is an optionally substituted heteroaryl, for example, substituted with 0-2 R³⁰ groups as described above. In certain embodiments, T is

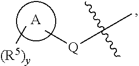

in which Q is as described above. The ring system denoted by A and its optional R⁵ substituents can be, for example, phenyl substituted by 0-2 R³⁰ groups as described above. In other embodiments, ring system denoted by A and its optional R⁵ substituents are heteroaryl, for example, substituted with 0-2 R³⁰ groups as described above.

As examples, in certain embodiments, the AMPK-activating compound has one of structural formulae (XXX)-(XXXV):

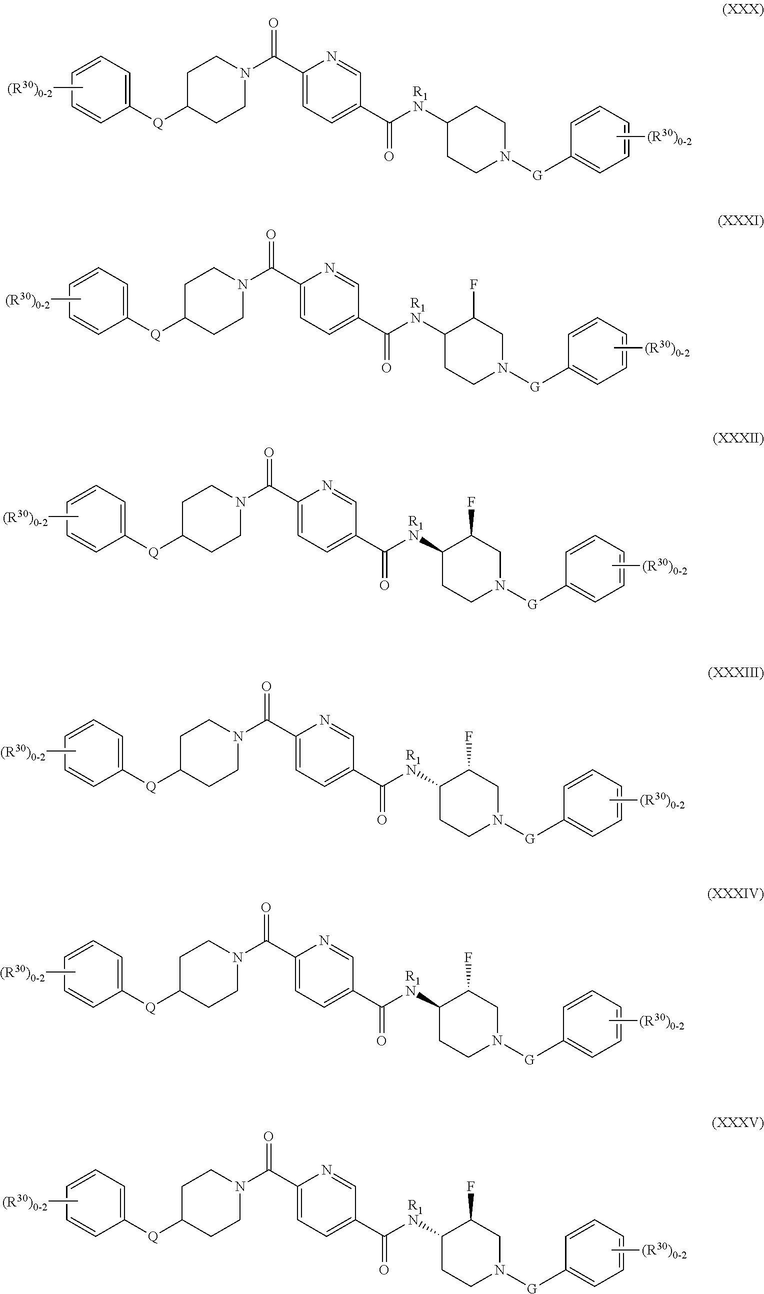

in which Q, G, R¹ and R³⁰ are as described above with reference to structural formulae (I)-(XXIX). In certain such embodiments, R¹ is H. In certain embodiments, G is CH₂, CO, or SO₂. In certain embodiments, Q is CH₂, CO, SO₂ or O. Compounds according to structural formulae (XXX)-(XXXV) can be provided as mixtures of diastereomers or enantiomers, or in diastereomerically and/or enantiomerically enriched form. In certain embodiments, the compound is provided in substantially diastereomerically pure form, for example, as substantially diastereomerically pure cis compound, or diastereomerically pure trans compound. In certain embodiments, a compound is provided in substantially enantiomerically pure form.

In other embodiments as described above, in the AMPK-activating compounds of any of structural formulae (I)-(XIII) as described above, the compound has the structural formula (XXXVI):

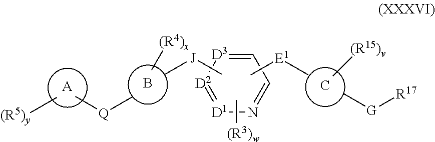

in which the ring system denoted by “C” is a monocyclic arylene or heteroarylene, or a monocyclic arylene fused to a heterocycloalkyl, and all other variables are as defined above with respect to any of structural formulae (I)-(XIV). For example, in certain embodiments, the ring system denoted by “C” is a phenylene, for example, a 1,4-phenylene. In other embodiments, the ring system denoted by “C” is a monocyclic heteroarylene, such as a pyridylene (for example, a 2,5-pyridylene); a 1,3-pyrazolylene (for example, a 1,3-pyrazolylene); a furanylene (for example, a 2,4-furanylene); or a thienylene (for example, a 2,4-thienylene). In other embodiments, the ring system denoted by “C” is a 1,2,3,4-tetrahydroisoquinolinylene (for example, a 1,2,3,4-tetrahydroisoquinolin-2,6-ylene).

In other embodiments as described above, in the AMPK-activating compounds of any of structural formulae (I)-(XIII) as described above, the compound has the structural formula (XVI):

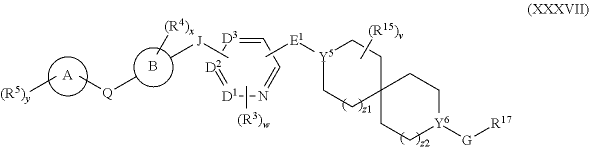

in which z1 is 0 or 1; z2 is 0 or 1; Y⁵ is N, C or CH; Y⁶ is N, C or CH; each of the v R¹⁵ can be disposed either spiro-fused ring; and all other variables are as defined above with respect to any of structural formulae (I)-(XIV).

In certain embodiments as described above, in the AMPK-activating compounds of structural formula (XXXVII) (for example, those in which E¹ is —C(O)— or absent), Y⁵ is N and Y⁶ is N. In other embodiments, (for example, those in which E¹ is —C(O)—NR¹—), Y⁵ is C or CH and Y⁶ is N. In other embodiments, Y⁵ is N and Y⁶ is C or CH. In other embodiments, Y⁵ is C or CH and Y⁶ is C or CH. In certain embodiments as described above, in the AMPK-activating compounds of structural formula (XXXVII) as described above, z1 is 1 and z2 is 0. In other embodiments, z1 is 0 and z2 is 1.

In one embodiment as described above, in the AMPK-activating compounds of any of structural formulae (XIV)-(XXXVII), Q is a single bond. In another embodiment, Q is —CH₂—. In other embodiments, Q is —C(O)— or —S(O)₂—. In other embodiments, Q is —NH—C(O)— or —CH₂—NH—C(O)—. In other embodiments, Q is —C(CH₃)₂—, —CH₂CH₂—, —CH(CH₃)—, —CH(OH)— or —CHF—. In other embodiments, Q is —O—. In other embodiments, Q is —CH₂O— or —OCH₂CH₂—. In other embodiments, Q is —CH(COOMe)- or —CH(COOEt)-.

In one embodiment as described above, in the AMPK-activating compounds of any of structural formulae (XIV)-(XXXVII), G is —CH₂—. In other embodiments, G is —C(O)— or —S(O)₂—. In other embodiments, G is —CH(CH₃)— or —C(CH₃)₂—. In other embodiments, G is —O—. In other embodiments, G is —C(O)—NH— or —C(O)—NH—CH₂—. In other embodiments, G is —CH₂CH₂—. In other embodiments, G is a single bond. In other embodiments, G is —O—. In other embodiments, G is —OCH₂— or —CH₂CH₂O—. In other embodiments, G is —CH(COOMe)- or —CH(COOEt)-.

In various embodiments disclosed with respect to structural formulae (XIV)-(XXXVII), the above-described Q and G moieties can be combined in any possible combination. For example, in one embodiment, Q is a single bond and G is —CH₂— or —C(O)—. In another embodiment, Q is —CH₂— or —C(O)— and G is a single bond. In yet another embodiment, Q is —CH₂— or —C(O)— and G is —CH₂— or —C(O)—.

In certain embodiments as described above, in the AMPK-activating compounds of any of structural formulae (XIV)-(XXXVII), the ring system denoted by “A” is aryl or heteroaryl, as described above. In one embodiment, the ring system denoted by “A” is substituted with one or more electron-withdrawing groups as described above. In another embodiment, R¹⁷ is substituted with one or more electron-withdrawing groups as described above. In certain embodiments, the ring system denoted by “A”, R¹⁷ or both are not substituted with an aryl, heteroaryl, cycloalkyl or heterocycloalkyl-containing group. In certain embodiments, the azacycloalkyl to which -G-R¹⁷ is bound is a piperidinyl; in other embodiments, it is a pyrrolidinyl.

In various aspects of the disclosure described above with respect to structural formulae (XIV)-(XXXVII), v is 0, 1, 2, 3 or 4. In one embodiment, v is 0, 1, 2 or 3. For example, v can be 0, or can be 1 or 2.

In certain embodiments as described above, in the AMPK-activating compounds of any of structural formulae (XIV)-(XXXVII), two R¹⁵ groups combine to form an oxo. The oxo can be bound, for example, at the position alpha relative to the nitrogen of an azacycloalkyl ring. In other embodiments, no two R¹⁵ groups combine to form an oxo.

In certain embodiments as described above, in the AMPK-activating compounds of any of structural formulae (XIV)-(XXXVII), v is at least 1 (for example, 1) and at least one R¹⁵ is F. In certain embodiments, the F can be, for example, disposed at a position alpha to the E¹ moiety. When the F and E¹ are both disposed on saturated carbons, they can be disposed in a cis relationship with respect to one another. For example, in certain embodiments, a compound has structural formula (XXXVIII)

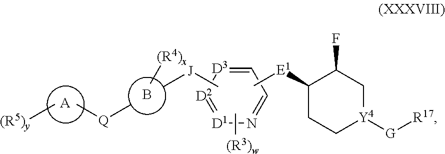

in which Y⁴ is N or CH and all variables are defined as described above with respect to structural formulae (I)-(XIV). In other embodiments, a compound has structural formula (XXXIX)

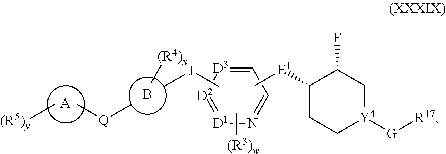

in which Y⁴ is N or CH and all variables are defined as described above with respect to structural formulae (I)-(XIV). In other embodiments, when the F and E¹ are both disposed on saturated carbons, they can be disposed in a trans relationship with respect to one another. For example, in one embodiment as described above, the AMPK-activating compound has structural formula (XL)

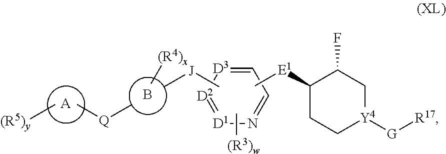

in which Y⁴ is N or CH and all variables are defined as described above with respect to structural formulae (I)-(XIV). In another embodiment, a compound has structural formula (XLI)

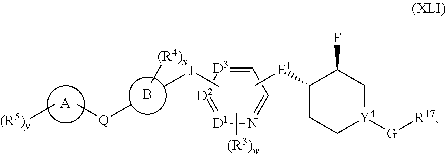

in which Y⁴ is N or CH and all variables are defined as described above with respect to structural formulae (I)-(XIV). Compounds according to structural formulae (XXXVIII)-(XLI) can be provided as mixtures of diastereomers or enantiomers, or in diastereomerically and/or enantiomerically enriched form. In certain embodiments, the compound is provided in substantially diastereomerically pure form, for example, as substantially diastereomerically pure cis compound, or diastereomerically pure trans compound. In certain embodiments, a compound is provided in substantially enantiomerically pure form.

In certain embodiments as described above, in the AMPK-activating compounds of any of structural formulae (XIV)-(XLI), when v is 4, not all four R¹⁵ moieties are (C₁-C₆ alkyl).

In certain embodiments as described above, in the AMPK-activating compounds of any of structural formulae (XIV)-(XLI), each R¹⁵ is independently selected from —(C₁-C₆ alkyl), —(C₁-C₆ haloalkyl) (for example, difluoromethyl, trifluoromethyl and the like), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN and two R¹⁵ on the same carbon optionally combine to form oxo, in which each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl), and in which no alkyl or haloalkyl is substituted with an aryl-, heteroaryl-, cycloalkyl- or heterocycloalkyl-containing group. For example, in one embodiment, each R¹⁵ is —(C₁-C₃ alkyl), —(C₁-C₃ haloalkyl), —(C₀-C₃ alkyl)-L-R⁷, —(C₀-C₃ alkyl)-NR⁸R⁹, —(C₀-C₃ alkyl)-OR¹⁰, —(C₀-C₃ alkyl)-C(O)R¹⁰, —(C₀-C₃ alkyl)-S(O)₀₋₂R¹⁰, -halogen, —NO₂ and —CN and two R¹⁵ on the same carbon optionally combine to form oxo, in which each R⁷, R⁸ and R¹⁰ is independently selected from H, —(C₁-C₂ alkyl), —(C₁-C₂ haloalkyl), —(C₀-C₂ alkyl)-L-(C₀-C₂ alkyl), —(C₀-C₂ alkyl)-NR⁹(C₀-C₂ alkyl), —(C₀-C₂ alkyl)-O—(C₀-C₂ alkyl), —(C₀-C₂ alkyl)-C(O)—(C₀-C₂ alkyl) and —(C₀-C₂ alkyl)-S(O)₀₋₂—(C₀-C₂ alkyl), and in which no alkyl or haloalkyl is substituted with an aryl-, heteroaryl-, cycloalkyl- or heterocycloalkyl-containing group. In certain embodiments, each R¹⁵ is independently halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl), and two R₄ optionally come together to form oxo. In certain embodiments, each R¹⁵ is independently methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂, N₃, —SF₅, or —CN, and two R¹⁵ on the same carbon optionally combine to form oxo. In some embodiments, one R¹⁵ is —C(O)NR⁹R⁷, which can be bound, for example, at a position alpha relative to the piperidine nitrogen, or at the position linked to the E¹ moiety.

In certain embodiments as described above, in the AMPK-activating compounds of any of structural formulae (XIV)-(XLI), R¹⁷ is an unsubstituted aryl or heteroaryl. In other embodiments, the R¹⁷ Ar or Het is substituted with 1, 2 or 3 substituents independently selected from —(C₁-C₆ alkyl), —(C₁-C₆ haloalkyl) (for example, difluoromethyl, trifluoromethyl and the like), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, in which each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl), and in which no alkyl or haloalkyl is substituted with an aryl-, heteroaryl-, cycloalkyl- or heterocycloalkyl-containing group. For example, in one embodiment, the R¹⁷ Ar or Het is substituted with 1, 2 or 3 substituents independently selected from —(C₁-C₃ alkyl), —(C₁-C₃ haloalkyl), —(C₀-C₃ alkyl)-L-R⁷, —(C₀-C₃ alkyl)-NR⁸R⁹, —(C₀-C₃ alkyl)-OR¹⁰, —(C₀-C₃ alkyl)-C(O)R¹⁰, —(C₀-C₃ alkyl)-S(O)₀₋₂R¹⁰, -halogen, —NO₂ and —CN, in which each R⁷, R⁸ and R¹⁰ is independently selected from H, —(C₁-C₂ alkyl), —(C₁-C₂ haloalkyl), —(C₀-C₂ alkyl)-L-(C₀-C₂ alkyl), —(C₀-C₂ alkyl)-NR⁹(C₀-C₂ alkyl), —(C₀-C₂ alkyl)-O—(C₀-C₂ alkyl), —(C₀-C₂ alkyl)-C(O)—(C₀-C₂ alkyl) and —(C₀-C₂ alkyl)-S(O)₀₋₂—(C₀-C₂ alkyl), and in which no alkyl or haloalkyl is substituted with an aryl-, heteroaryl-, cycloalkyl- or heterocycloalkyl-containing group. In certain embodiments, R¹⁷ is substituted with 1, 2 or 3 substituents selected from halo, cyano, —(C₁-C₄ haloalkyl), —O—(C₁-C₄ haloalkyl), —(C₁-C₄ alkyl), —O—(C₁-C₄ alkyl), —C(O)—(C₀-C₄ alkyl), —C(O)O—(C₀-C₄ alkyl), —C(O)N(C₀-C₄ alkyl)(C₀-C₄ alkyl), NO₂ and —C(O)—Hca in which no alkyl or haloalkyl is substituted with an aryl-, heteroaryl-, cycloalkyl- or heterocycloalkyl-containing group. In certain embodiments, R¹⁷ is substituted with 1, 2 or 3 substituents selected from halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl), and two R₄ optionally come together to form oxo. In certain embodiments, each R¹⁷ is substituted with 1, 2 or 3 substituents selected from methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂, N₃, —SF₅, or —CN. R¹⁷ can be substituted with, for example, one such substituent, or two such substituents.

In certain embodiments as described above, in the AMPK-activating compounds of any of structural formulae (XIV)-(XLI), at least one of R¹⁷ and the ring system denoted by “A” is substituted with —C(O)NR²⁷R²⁹, in which R²⁷ is selected from H, —(C₁-C₆ alkyl), —(C₁-C₆ haloalkyl) (for example, difluoromethyl, trifluoromethyl and the like), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl)-(C₀-C₆ alkyl)-S(O)₀₋₂—(C₀-C₆ alkyl), in which no heterocycloalkyl, alkyl or haloalkyl is substituted with an aryl-, heteroaryl-, cycloalkyl- or heterocycloalkyl-containing group, and R²⁹ is —H, —(C₁-C₄ alkyl), —C(O)—(C₁-C₄ alkyl) or —C(O)—O—(C₁-C₄ alkyl) in which no (C₁-C₄ alkyl) is substituted by an aryl, heteroaryl, cycloalkyl or heterocycloalkyl-containing group, or R²⁷ and R²⁹ together with the nitrogen to which they are bound form Hca (for example, morpholino, piperazinyl, pyrrolidinyl or piperidinyl). In certain embodiments, heterocycloalkyl, alkyl or haloalkyl groups of R²⁷ and R²⁹ are substituted with 1, 2 or 3 substituents selected from halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl), and two R₄ optionally come together to form oxo. In certain embodiments, the heterocycloalkyl, alkyl or haloalkyl groups of R²⁷ and R²⁹ are optionally substituted with acetyl, —NH₂, —OH, methoxy, ethoxy, trifluoromethoxy, —SO₂Me, -halogen, —NO₂, N₃, —SF₅, or —CN. In one embodiment, R²⁷ and R²⁹ are both H. In another embodiment, R²⁷ is CH₃ and R²⁹ is H.

In certain embodiments as described above, in the AMPK-activating compounds of any of structural formulae (XIV)-(XLI), the -G-R¹⁷ moiety is selected from the group consisting of

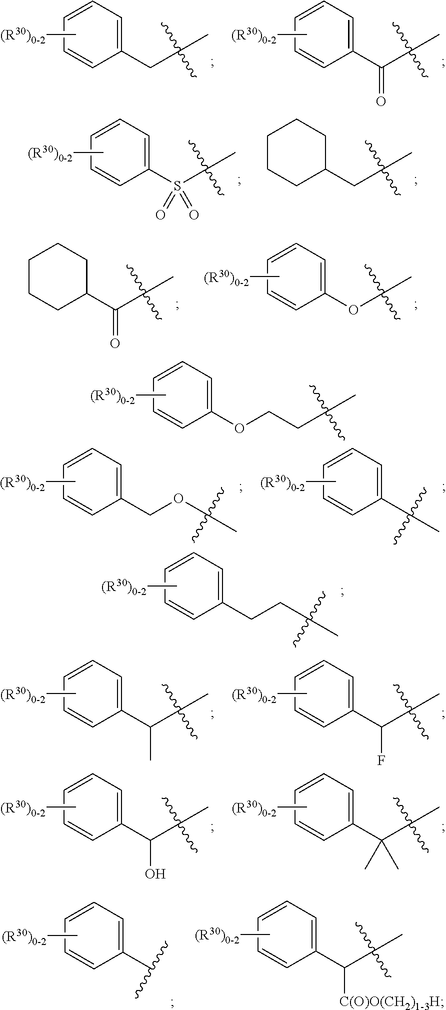

monocyclic heterocycloalkyl (for example, tetrahydropyranyl, morpholinyl, piperidinyl, piperazinyl) substituted with 0, 1 or 2 R³⁰, monocyclic heteroaryl (for example, pyridyl, isoxazolyl, oxazolyl, pyrrolyl, thienyl) substituted with 0, 1 or 2 R³⁰; monocyclic heteroarylmethyl- (for example, pyridylmethyl, isoxazolylmethyl, oxazolylmethyl, pyrrolylmethyl, thienylmethyl), in which the heteroaryl is substituted with 0, 1 or 2 R³⁰; or monocyclic heteroaryloxy- (for example, pyridyloxy, isoxazolyloxy, oxazolyloxy, pyrrolyloxy, thienyloxy), in which the heteroaryl is substituted with 0, 1 or 2 R³⁰; in which each R³⁰ is independently selected from halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, no R³⁰ is substituted on the ring of R¹⁷. In other embodiments, one R³⁰ is substituted on the ring, for example, at a para-position of a phenyl, a meta-position of a phenyl, or at a 3- or 4- position of a heteroaryl or heterocycloalkyl (as counted from the attachment point of the Y⁴, Y⁶ or the ring system denoted by “C”). Certain particular identities of the -G-R¹⁷ moiety will be found by the person of skill in the art in the compounds described below with respect to Table 1. Those of skill in the art will understand that combinations of such -G-R¹⁷ moieties with other subcombinations of features disclosed herein is specifically contemplated.

For example, in certain embodiments as described above, in the AMPK-activating compounds of any of structural formulae (XIV)-(XLI), the -G-R¹⁷ moiety is selected from

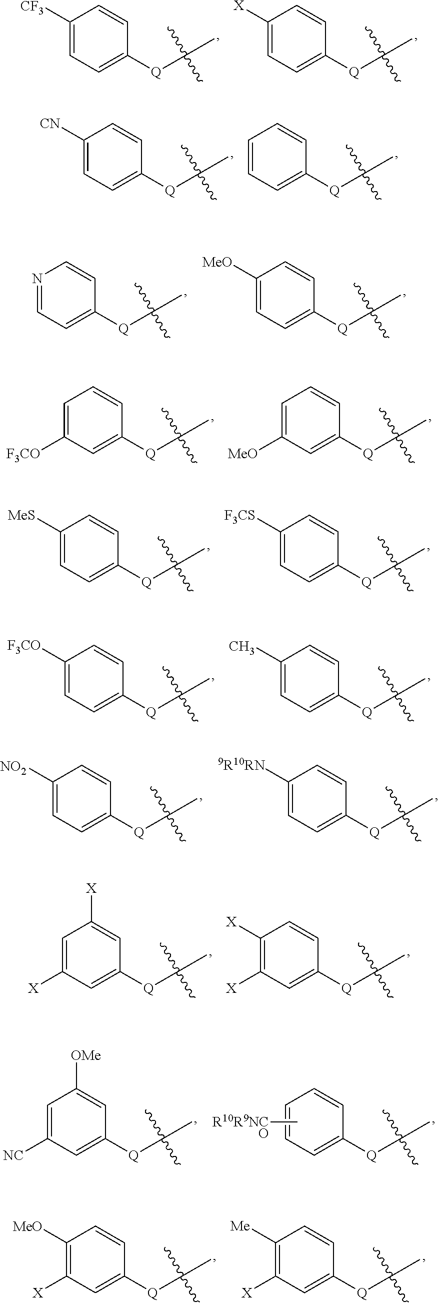

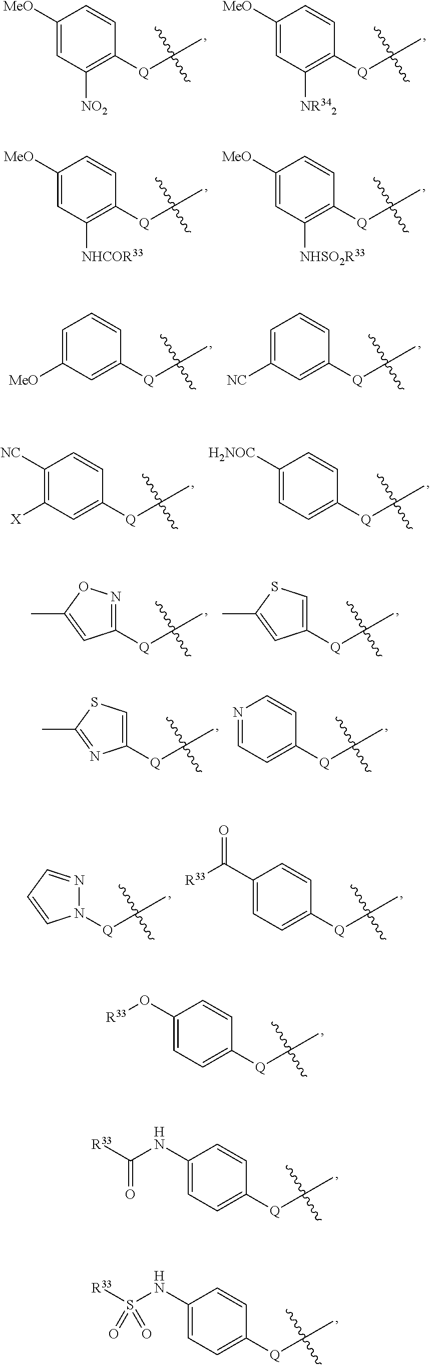

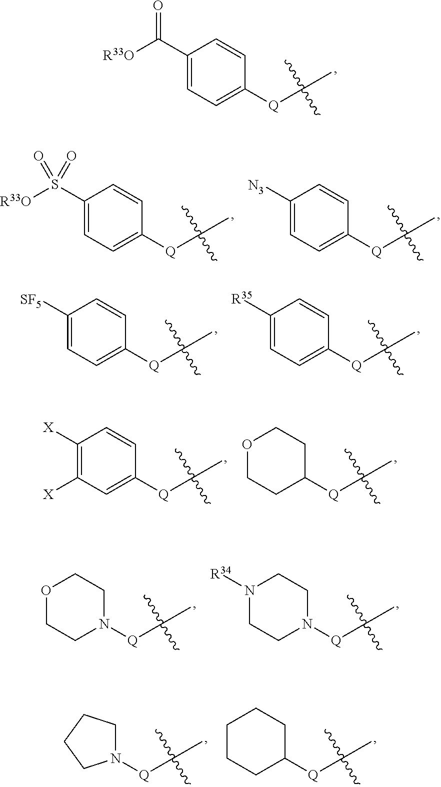

heterocycloalkyl optionally substituted by alkyl and/or halogen, -Q-heteroaryl optionally substituted by unsubstituted (C₁-C₄ alkyl) and/or halogen, H, C(O)tBu and isopropyl, in which each X is independently F, Cl or Br (preferably F or Cl), each R³³ is unsubstituted (C₁-C₄ alkyl), unsubstituted (C₁-C₄ haloalkyl) or cycloalkyl optionally substituted with unsubstituted alkyl, unsubstituted (C₁-C₄ alkyl), unsubstituted (C₁-C₄ haloalkyl) or cycloalkyl optionally substituted with unsubstituted alkyl, and each R³⁵ is heterocycloalkyl, optionally substituted with unsubstituted alkyl. In certain such embodiments, Q is a single bond, —CH₂—, —CH₂O—, —OCH₂CH₂—, —CH₂CH₂—, —O—, —CHF—, —CH(CH₃)—, —C(CH₃)₂—, —CH(OH)—, —CH(COOMe)-, —CH(COOEt)-, —C(O)— or —S(O)₂—. As the person of skill in the art will appreciate, the

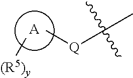

moiety and G-R¹⁷ moieties described above can be combined in virtually any combination, and such combinations are specifically contemplated by this disclosure. For example, in certain embodiments as described above, in the AMPK-activating compounds of any of structural formulae (XIV)-(XX), both the

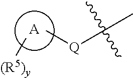

moiety and the -G-R¹⁷ moiety are

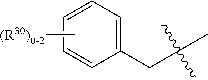

(for example, 4-fluorobenzyl or 4-cyanobenzyl). In other embodiments, the

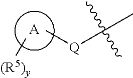

moiety is

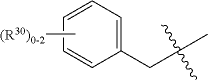

(for example, 4-fluorobenzyl or 4-cyanobenzyl), and the -G-R¹⁷ moiety is

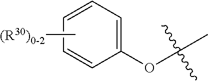

(for example, 4-methylphenoxy, 4-methoxyphenoxy, 4-chlorophenoxy, 4-cyanophenoxy, 4-cyano-2-methoxyphenoxy, 3-methylphenoxy, 3-methoxyphenoxy, 3-fluorophenoxy or 3-cyanophenoxy). Of course, the person of skill in the art will recognize that other combinations of

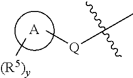

and -G-R¹⁷ can be used. Such combinations of

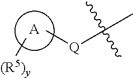

and -G-R¹⁷ in combination with other combinations of features described herein is specifically contemplated by this disclosure.

In certain embodiments as described above, the AMPK-activating compound has the structural formula (XLII):

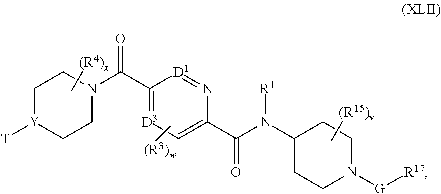

in which the variables are independently defined as described above with respect to structural formulae (I)-(XLI). In certain embodiments of the compounds of structural formula (XXI), T is H. In certain embodiments of the compounds of structural formula (XLII), T is

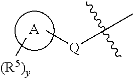

as described above with respect to structural formulae (I)-(XLI), and -G-R¹⁷ is benzoyl, benzenesulfonyl, phenyl, 1-phenylethyl, 1-methyl-1-phenylethyl, —CH(CO(O)(CH₂)₁₋₃H)-phenyl substituted with 0, 1 or 2 R³⁰ as described above, or 4-methoxybenzyl, —C(O)—Cak or —CH₂-Cak. In certain embodiments, G-R¹⁷ is as described above with respect to structural formulae (I)-(XLI), and T is benzoyl, benzenesulfonyl, 1-methyl-1-phenylethyl, heterocycloalkyl, heteroarylmethyl or heteroaryl substituted with 0, 1 or 2 R³⁰ as described above, or 3,5-difluorobenzyl, —C(O)—Cak, (C₁-C₆ alkyl)C(O)— or (C₁-C₆ alkyl). In certain embodiments, Y is N. In other embodiments, Y is CH or C substituted by one of the x R⁴.

In certain embodiments as described above, the AMPK-activating compound has the structural formula (XLIII):

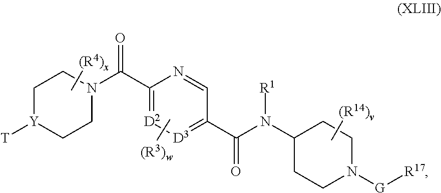

in which the variables are independently defined as described above with respect to structural formulae (I)-(XLII). In certain embodiments of the compounds of structural formula (XLIII), T is H. In certain embodiments as described above, in the compounds of structural formula (XLIII), T is

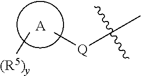

as described above with respect to structural formulae (I)-(XLII), and -G-R¹⁷ is benzoyl, benzenesulfonyl, phenyl, 1-phenylethyl, 1-methyl-1-phenylethyl, —CH(CO(O)(CH₂)₁₋₃H)-phenyl substituted with 0, 1 or 2 R³⁰ as described above, or 4-methoxybenzyl, —C(O)—Cak or —CH₂-Cak. In certain embodiments, G-R¹⁷ is as described above with respect to structural formulae (I)-(XLII), and T is benzoyl, benzenesulfonyl, 1-methyl-1-phenylethyl, heterocycloalkyl, heteroarylmethyl or heteroaryl substituted with 0, 1 or 2 R³⁰ as described above, or 3,5-difluorobenzyl, —C(O)—Cak, (C₁-C₆ alkyl)C(O)— or (C₁-C₆ alkyl). In certain embodiments, Y is N. In other embodiments, Y is CH or C substituted by one of the x R⁴.

In certain embodiments as described above, the AMPK-activating compound has the structural formula (XLIV):

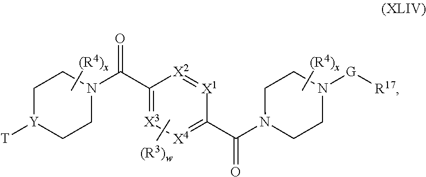

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, T is (C₁-C₆ alkyl). In other embodiments,

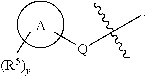

In certain embodiments, the T moiety and the G-R¹⁷ moiety are independently benzyl, 2-phenylethyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (XLV):

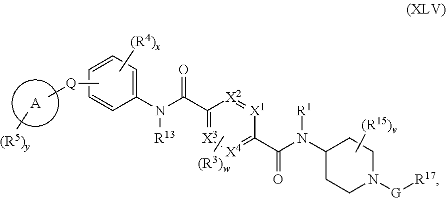

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

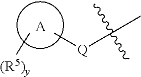

moiety and the G-R¹⁷ moiety are independently benzyl or phenyl substituted with 0, 1 or 2 R³⁰ as described above. In certain embodiments, the Q and the NR¹³ are substituted para from one another on the phenylene. In other embodiments, the Q and the NR¹³ are substituted meta from one another on the phenylene.

In certain embodiments as described above, the AMPK-activating compound has the structural formula (XLVI):

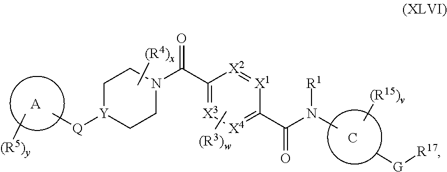

in which the ring system denoted by “C” is heteroarylene (for example, monocyclic heteroarylene), one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

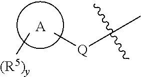

moiety and the G-R¹⁷ moiety are independently benzyl or phenyl substituted with 0, 1 or 2 R³⁰ as described above. In certain embodiments, the ring system denoted by “C” is a pyrazolylene (for example, a 1,3-pyrazolylene), a pyridylene (for example, a 2,5-pyridylene). In certain embodiments, Y is N. In other embodiments, Y is CH or C substituted by one of the x R⁴.

In certain embodiments as described above, the AMPK-activating compound has the structural formula (XLVII):

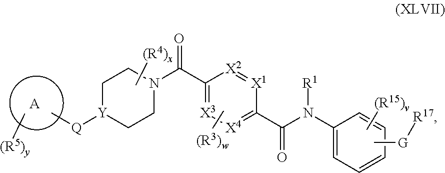

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

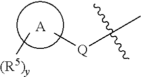

moiety and the G-R¹⁷ moiety are independently benzyl, phenylmethoxy, —C(O)NHCH₂-phenyl, heteroaryl, or phenyl substituted with 0, 1 or 2 R³⁰ as described above. In certain embodiments, the G and the NR¹ are substituted parawith respect to one another on the phenylene. In other embodiments, the G and the NR¹ are substituted metawith respect to one another on the phenylene. In other embodiments, the G and the NR¹ are substituted orthowith respect to one another on the phenylene. In certain embodiments, Y is N. In other embodiments, Y is CH or C substituted by one of the x R⁴.

In certain embodiments as described above, the AMPK-activating compound has the structural formula (XLVIII):

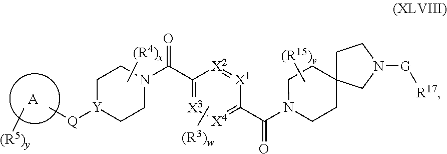

in which one or two of X¹, X², X³ and X⁴ are N, and the others are CH or C substituted by one of the w R³; each of the v R¹⁵ can be disposed either spiro-fused ring; and all other variables are independently defined as described above with respect to structural formulae (I)-(XLIII). In one embodiment, X¹ is N and X², X³ and X⁴ are CH or C substituted by one of the w R³. For example, in certain embodiments, the

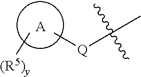

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (XLIX):

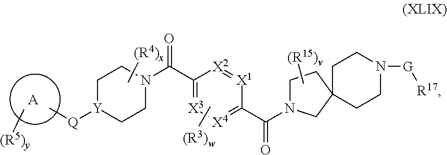

in which one or two of X¹, X², X³ and X⁴ are N, and the others are CH or C substituted by one of the w R³; each of the v R¹⁵ can be disposed either spiro-fused ring; and all other variables are independently defined as described above with respect to structural formulae (I)-(XLIII). In one embodiment, X¹ is N and X², X³ and X⁴ are CH or C substituted by one of the w R³. For example, in certain embodiments, the

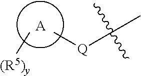

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (L):

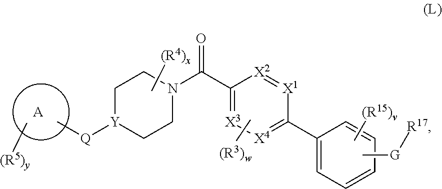

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

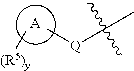

moiety and the G-R¹⁷ moiety are independently benzyl, phenylmethoxy, —C(O)NHCH₂-phenyl or phenyl substituted with 0, 1 or 2 R³⁰ as described above. In certain embodiments, the G and the NR¹ are substituted parawith respect to one another on the phenylene. In other embodiments, the G and the NR¹ are substituted metawith respect to one another on the phenylene. In other embodiments, the G and the NR¹ are substituted orthowith respect to one another on the phenylene. In certain embodiments, Y is N. In other embodiments, Y is CH or C substituted by one of the x R⁴.

In certain embodiments as described above, the AMPK-activating compound has the structural formula (LI):

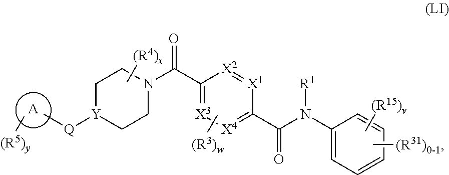

in which one or two of X¹, X², X³ and X⁴ are N, and the others are CH or C substituted by one of the w R³, R³¹ is defined as described above for R³⁰ with respect to the

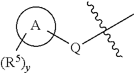

moiety and all other variables are independently defined as described above with respect to structural formulae (I)-(XLIII). In one embodiment, X¹ is N and X², X³ and X⁴ are CH or C substituted by one of the w R³. In certain embodiments, R³¹ is Br. In certain embodiments, the

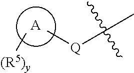

moiety is benzyl with 0, 1 or 2 R³⁰ as described above. In certain embodiments, the G and the NR¹ are substituted para with respect to one another on the phenylene. In other embodiments, the G and the NR¹ are substituted meta with respect to one another on the phenylene. In other embodiments, the G and the NR¹ are substituted ortho with respect to one another on the phenylene. In certain embodiments, Y is N. In other embodiments, Y is CH or C substituted by one of the x R⁴.

In certain embodiments as described above, the AMPK-activating compound has the structural formula (LII):

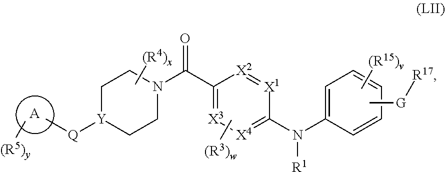

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

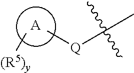

moiety and the G-R¹⁷ moiety are independently benzyl, phenoxy, phenylmethoxy, —C(O)NHCH₂-phenyl or phenyl substituted with 0, 1 or 2 R³⁰ as described above. In certain embodiments, the G and the NR¹ are substituted para with respect to one another on the phenylene. In other embodiments, the G and the NR¹ are substituted meta with respect to one another on the phenylene. In other embodiments, the G and the NR¹ are substituted ortho with respect to one another on the phenylene. In certain embodiments, Y is N. In other embodiments, Y is CH or C substituted by one of the x R⁴.

In certain embodiments as described above, the AMPK-activating compound has the structural formula (LIII):

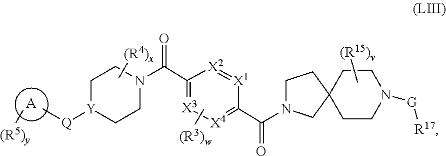

in which one or two of X¹, X², X³ and X⁴ are N; each of the v R¹⁵ can be disposed either spiro-fused ring; and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

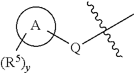

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LIV):

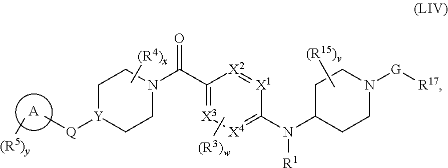

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

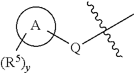

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LV):

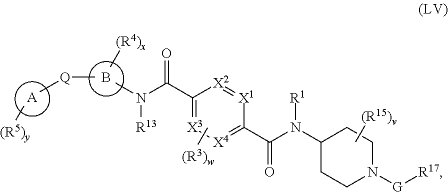

in which the ring system denoted by “B” is a heteroarylene, one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

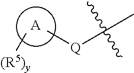

moiety and the G-R¹⁷ moiety are independently benzyl or phenyl substituted with 0, 1 or 2 R³⁰ as described above. In certain embodiments, the ring system denoted by“B” is a pyrazolylene (for example, a 1,3-pyrazolylene).

In certain embodiments as described above, the AMPK-activating compound has the structural formula (LVI):

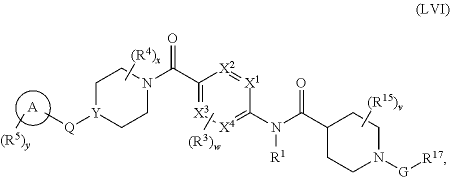

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

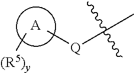

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LVII):

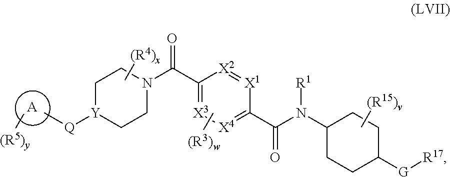

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

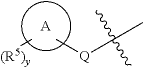

moiety and the G-R¹⁷ moiety are independently benzyl or phenyl substituted with 0, 1 or 2 R³⁰ as described above. The NR¹ and G-R¹⁷ moieties can, for example, be substituted cis with respect to one another on the cyclohexane ring. In other embodiments, the NR¹ and G-R¹⁷ moieties are substituted trans with respect to one another on the cyclohexane ring. In certain embodiments, Y is N. In other embodiments, Y is CH or C substituted by one of the x R⁴.

In certain embodiments as described above, the AMPK-activating compound has the structural formula (LVIII):

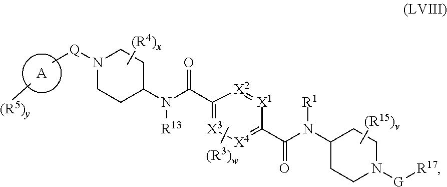

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

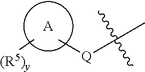

moiety and the G-R¹⁷ moiety are independently benzyl, phenoxy or phenyl substituted with 0, 1 or 2 R³⁰ as described above.

In certain embodiments as described above, the AMPK-activating compound has the structural formula (LIX):

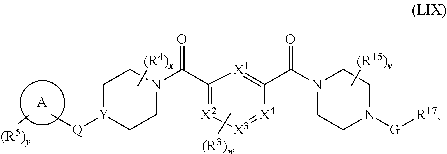

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

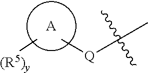

moiety and the G-R¹⁷ moiety are independently benzyl, 2-phenylethyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LX):

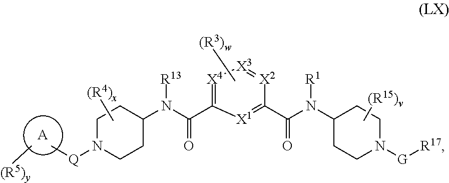

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

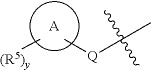

moiety and the G-R¹⁷ moiety are independently benzyl or phenyl substituted with 0, 1 or 2 R³⁰ as described above.

In certain embodiments as described above, the AMPK-activating compound has the structural formula (LXI):

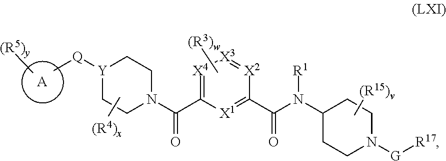

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

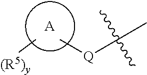

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LXII):

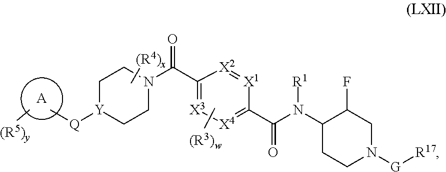

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. In certain embodiments, the fluorine atom and the —NR¹— are disposed ciswith respect to one another on the piperidine. In certain embodiments, the

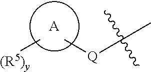

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LXIII):

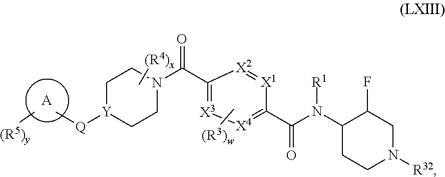

in which R³² is —H, —(C₁-C₄ alkyl), —C(O)—(C₁-C₄ alkyl) or —C(O)O—(C₁-C₄ alkyl), one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. and the other variables are independently defined as described above with respect to structural formulae (I)-(XIV). In certain embodiments, R³² is H or methyl. In certain embodiments, the fluorine atom and the —NR¹— are disposed cis with respect to one another on the piperidine. In certain embodiments, the

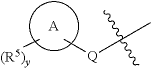

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LXIV):

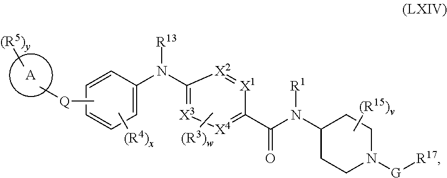

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

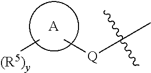

moiety and the G-R¹⁷ moiety are independently benzyl, phenoxy or phenyl substituted with 0, 1 or 2 R³⁰ as described above. In certain embodiments, the Q and the NR¹³ are substituted para from one another on the phenylene. In other embodiments, the Q and the NR¹³ are substituted meta from one another on the phenylene.

In certain embodiments as described above, the AMPK-activating compound has the structural formula (LXV):

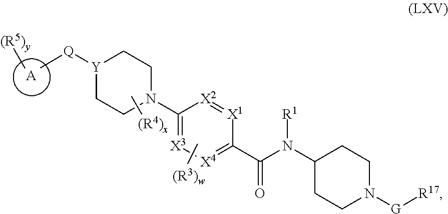

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. In certain embodiments, the

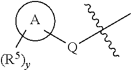

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LXVI):

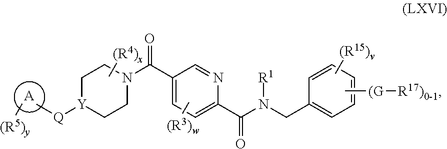

in which one or two of X¹, X², X³ and X⁴ are N, and the others are CH or C substituted by one of the w R³, and all other variables are independently defined as described above with respect to structural formulae (I)-(XLIII), and the G-R¹⁷ moiety is optional. In one embodiment, X¹ is N and X², X³ and X⁴ are CH or C substituted by one of the w R³. For example, in certain embodiments, the G-R¹⁷ moiety is absent. In certain embodiments, the

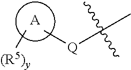

moiety and the G-R¹⁷ moiety (if present) are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LXVII):

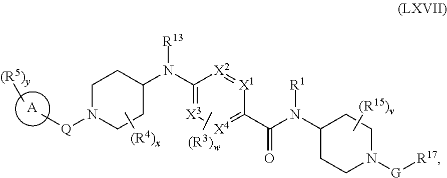

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

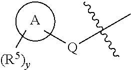

moiety and the G-R¹⁷ moiety are independently benzyl or phenyl substituted with 0, 1 or 2 R³⁰ as described above.

In certain embodiments as described above, the AMPK-activating compound has the structural formula (LXVIII):

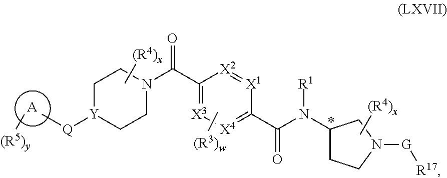

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

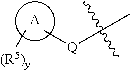

moiety and the G-R¹⁷ moiety are independently benzyl or phenyl substituted with 0, 1 or 2 R³⁰ as described above. In certain embodiments, the stereogenic center indicated by “*” is racemic. In other embodiments, it is enantiomerically enriched, for example, in the (R)-configuration (i.e., the carbon-NR¹ bond disposed above the plane of the page). In other embodiments, it is enantiomerically enriched, for example, in the (S)-configuration (i.e., the carbon-NR¹ bond disposed below the plane of the page). In certain embodiments, Y is N. In other embodiments, Y is CH or C substituted by one of the x R⁴.

In certain embodiments as described above, the AMPK-activating compound has the structural formula (LXVIII):

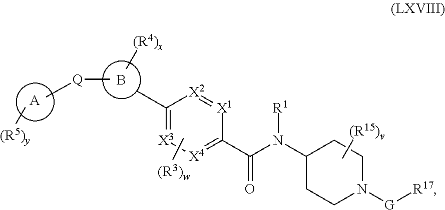

in which the ring system denoted by “B” is a heteroarylene, one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

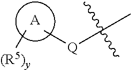

moiety and the G-R¹⁷ moiety are independently benzyl or phenyl substituted with 0, 1 or 2 R³⁰ as described above. In certain embodiments, the ring system denoted by “B” is a triazolylene (for example, a 1,2,3-triazol-1,4-ylene).

In certain embodiments as described above, the AMPK-activating compound has the structural formula (LXIX):

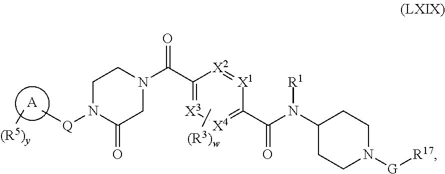

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

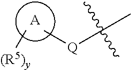

moiety and the G-R¹⁷ moiety are independently benzyl or phenyl substituted with 0, 1 or 2 R³⁰ as described above.

In certain embodiments as described above, the AMPK-activating compound has the structural formula (LXX):

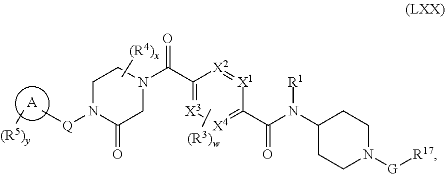

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

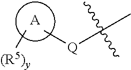

moiety and the G-R¹⁷ moiety are independently benzyl, benzoyl, 1-fluoro-1-phenylmethyl, phenoxy or phenyl substituted with 0, 1 or 2 R³⁰ as described above. In certain embodiments, the

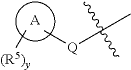

moiety is bound at the 4-position of the piperidine. In other embodiments, it is bound at the 3-position of the piperidine. In other embodiments, it is bound at the 2-position of the piperidine.

In certain embodiments as described above, the AMPK-activating compound has the structural formula (LXXI):

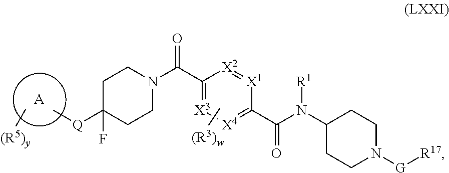

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

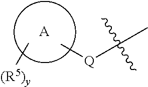

moiety and the G-R¹⁷ moiety are independently benzyl, benzoyl, 1-fluoro-1-phenylmethyl, phenoxy or phenyl substituted with 0, 1 or 2 R³⁰ as described above.

In certain embodiments as described above, the AMPK-activating compound has the structural formula (LXXII):

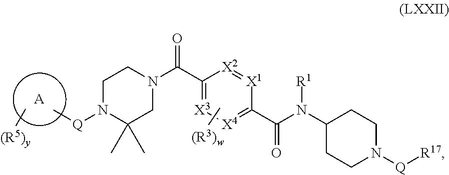

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

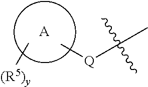

moiety and the G-R¹⁷ moiety are independently benzyl or phenyl substituted with 0, 1 or 2 R³⁰ as described above.

In certain embodiments as described above, the AMPK-activating compound has the structural formula (LXXIII):

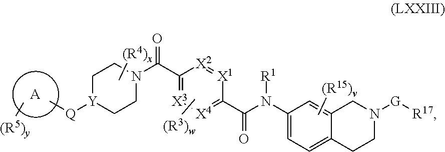

in which one or two of X¹, X², X³ and X⁴ are N and the others are CH or C substituted by one of the w R³; each of the R¹⁵ is substituted on either ring of the 1,2,3,4-tetrahydroisoquinoline; and all other variables are independently defined as described above with respect to structural formulae (I)-(XLIII). In one embodiment, X¹ is N and X², X³ and X⁴ are CH or C substituted by one of the w R³. For example, in certain embodiments, the

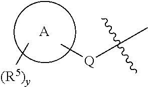

moiety and the G-R¹⁷ moiety are independently benzyl or phenyl substituted with 0, 1 or 2 R³⁰ as described above. In certain embodiments, Y is N. In other embodiments, Y is CH or C substituted by one of the x R⁴.

In certain embodiments as described above, the AMPK-activating compound has the structural formula (LXXIV):

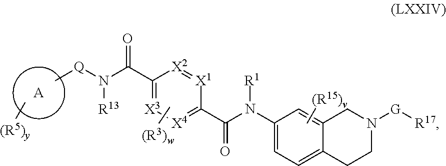

in which one or two of X¹, X², X³ and X⁴ are N, and the others are CH or C substituted by one of the w R³; each of the R¹⁵ is substituted on either ring of the 1,2,3,4-tetrahydroisoquinoline; and all other variables are independently defined as described above with respect to structural formulae (I)-(XLIII). In one embodiment, X¹ is N and X², X³ and X⁴ are CH or C substituted by one of the w R³. For example, in certain embodiments, the

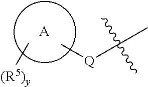

moiety and the G-R¹⁷ moiety are independently benzyl or phenyl substituted with 0, 1 or 2 R³⁰ as described above.

In certain embodiments as described above, the AMPK-activating compound has the structural formula (LXXV):

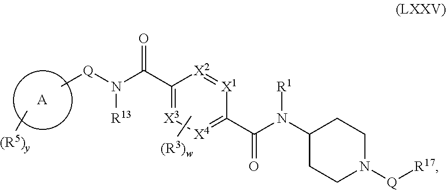

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

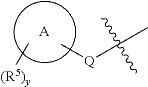

moiety and the G-R¹⁷ moiety are independently benzyl or phenyl substituted with 0, 1 or 2 R³⁰ as described above. In other embodiments, the Q moiety is —O—CH₂—CH₂—.

In certain embodiments as described above, the AMPK-activating compound has the structural formula (LXXVI):

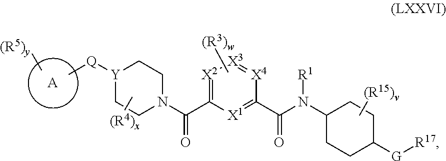

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

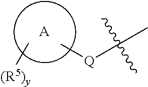

moiety and the G-R¹⁷ moiety are independently benzyl or phenyl substituted with 0, 1 or 2 R³⁰ as described above. In certain embodiments, the NR¹ and the -G-R¹⁷ are disposed cis with respect to one another on the cyclohexane ring. In other embodiments, the NR¹ and the -G-R¹⁷ are disposed trans with respect to one another on the cyclohexane ring. In certain embodiments, Y is N. In other embodiments, Y is CH or C substituted by one of the x R⁴.

In certain embodiments as described above, the AMPK-activating compound has the structural formula (LXXVII):

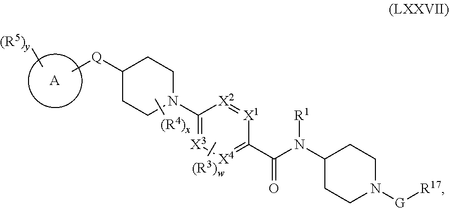

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. For example, in certain embodiments, the

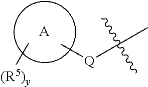

moiety and the G-R¹⁷ moiety are independently benzyl, phenoxy or phenyl substituted with 0, 1 or 2 R³⁰ as described above.

In certain embodiments as described above, the AMPK-activating compound has the structural formula (LXXVIII):

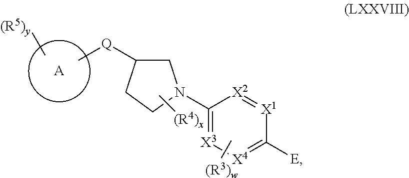

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. The E moiety can be, for example, as described with reference to any of structural formulae (XIII)-(LXXVIII). For example, in certain embodiments, the

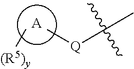

moiety and the E moiety are independently benzyl, phenoxy or phenyl substituted with 0, 1 or 2 R³⁰ as described above.

In certain embodiments as described above, the AMPK-activating compound has the structural formula (LXXIX):

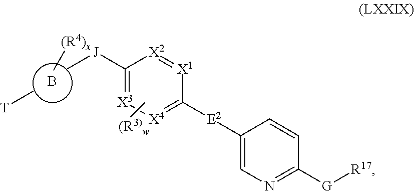

in which one or two of X¹, X², X³ and X⁴ are N, and the others are CH or C substituted by one of the w R³, E² is —CONR¹— (for example, —CONH—) or —NR¹CO— (for example, —NHCO—), and all other variables are independently defined as described above with respect to structural formulae (I)-(XLIII). In one embodiment, X¹ is N and X², X³ and X⁴ are CH or C substituted by one of the w R³. The -G-R¹⁷ moiety can be, for example, as described with reference to any of structural formulae (XIII)-(LXXVIII). Independently, the

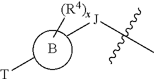

moiety can be, for example, as described with reference to any of structural formulae (XIII)-(LXXVIII). For example, in certain embodiments, the T moiety and the G-R¹⁷ moiety are independently benzyl, phenoxy or phenyl substituted with 0, 1 or 2 R³⁰ as described above. In other embodiments, G is O, CH₂, or SO₂.

In certain embodiments as described above, the AMPK-activating compound has the structural formula (LXXX):

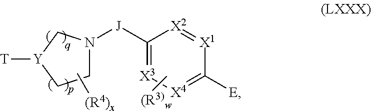

in which two R⁴ on different carbons combine to form a (C₁-C₄ alkylene) bridge, one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. The E moiety can be, for example, as described with reference to any of structural formulae (XIII)-(LXXVIII). Independently, the T moiety can be, for example, as described with reference to any of structural formulae (XIII)-(LVII). For example, in certain embodiments, the T moiety is independently benzyl, phenoxy or phenyl substituted with 0, 1 or 2 R³⁰ as described above. In certain embodiments, Y is N. In other embodiments, Y is CH or C substituted by one of the x R⁴. In certain embodiments, the

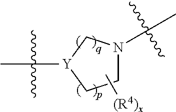

moiety is

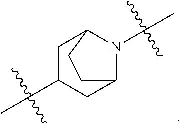 [SMILES: *C1CC2CCC(C1)N2*]

In certain embodiments as described above, the AMPK-activating compound has the structural formula (LXXXI):

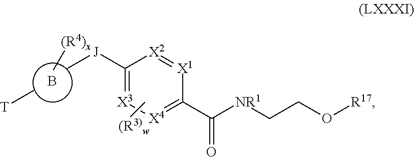

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. In one embodiment, R¹ is H. The —R¹⁷ moiety can be, for example, as described with reference to any of structural formulae (XIII)-(LXXVIII). Independently, the

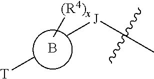

moiety can be, for example, as described with reference to any of structural formulae (XIII)-(LXXVIII). For example, in certain embodiments, the T moiety is benzyl, phenoxy or phenyl substituted with 0, 1 or 2 R³⁰ as described above; and the R¹⁷ moiety is phenyl substituted with 0, 1 or 2 R³⁰ as described above.

In certain embodiments as described above, the AMPK-activating compound has the structural formula (LXXXII):

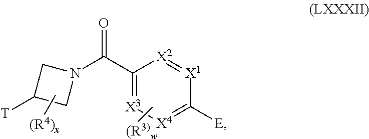

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. The E moiety can be, for example, as described with reference to any of structural formulae (XIII)-(LXXVIII). Independently, the T moiety can be, for example, as described with reference to any of structural formulae (XIII)-(LXXVIII). For example, in certain embodiments, the T moiety is benzyl, phenoxy or phenyl substituted with 0, 1 or 2 R³⁰ as described above.

In certain embodiments as described above, the AMPK-activating compound has the structural formula (LXXXIII):

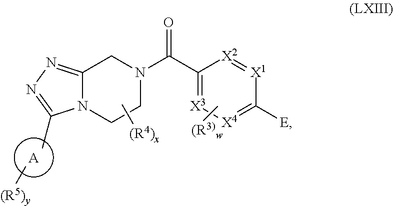

in which one or two of X¹, X², X³ and X⁴ are N, and the others are CH or C substituted by one of the w R³, and all other variables are independently defined as described above with respect to structural formulae (I)-(XLIII). In one embodiment, X¹ is N and X², X³ and X⁴ are CH or C substituted by one of the w R³. The E moiety can be, for example, as described with reference to any of structural formulae (XIII)-(LXXVIII). The A-(R⁵)_{y} moiety independently be, for example, described reference to any of structural formulae (XIII)-(LXXVIII). For example, in certain embodiments, the T moiety is benzyl, phenoxy or phenyl substituted with 0, 1 or 2 R³⁰ as described above.

In certain embodiments as described above, the AMPK-activating compound has the structural formula (LXXXIV):

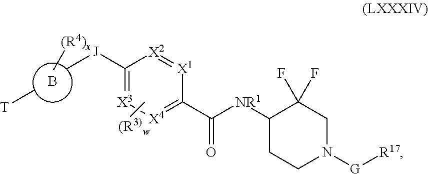

in which one or two of X¹, X², X³ and X⁴ are N, and the others are CH or C substituted by one of the w R³, and all other variables are independently defined as described above with respect to structural formulae (I)-(XXII). In one embodiment, X¹ is N and X², X³ and X⁴ are CH or C substituted by one of the w R³. In one embodiment, R¹ is H. The -G-R¹⁷ moiety can be, for example, as described with reference to any of structural formulae (XIII)-(LXXVIII). Independently, the

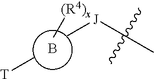

moiety can be, for example, as described with reference to any of structural formulae (XIII)-(LXXVIII). For example, in certain embodiments, the T moiety is benzyl, phenoxy or phenyl substituted with 0, 1 or 2 R³⁰ as described above; and the R¹⁷ moiety is phenyl substituted with 0, 1 or 2 R³⁰ as described above.

In certain embodiments as described above, in the AMPK-activating compounds of structural formulae (XIII)-(LXXVIII), the

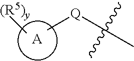

moiety is p-(trifluoromethyl)phenyl, p-fluorophenoxy, m-chloro-p-cyanophenoxy, p-trifluoromethylphenoxy, m, p-difluorophenoxy, m-cyanophenoxy, p-chlorobenzoyl, 2-(p-fluorophenoxy)ethyl, m-methoxyphenyl, m-fluoro-p-methoxybenzyl, p-methylbenzyl, α,p -difluorobenzyl, p-fluoro-α-hydroxybenzyl, 1-methyl- 1-phenylethyl, p-chlorophenyl, p-cyanophenoxy, benzenesulfonyl, tetrahydro-2H-pyran-4-yl, 5-methylisoxazol-3-yl, p-fluorobenzenesulfonyl, p-methoxybenzenesulfonyl, benzyl, p-cyano-o-methoxyphenoxy, p-methoxybenzoyl, p-methoxyphenoxy, benzoyl, p-fluorobenzoyl, cyclohexanecarbonyl, p-methoxybenzoyl, cyclohexylmethyl, pyrid-4-yl, pyrid-4-ylmethyl, phenoxy, phenyl, phenethyl, p-methoxyphenyl, p-fluorophenyl, p-cyanophenyl, p-(trifluoromethyl)benzyl, p-methoxybenzyl, p-fluorobenzyl, m,m-difluorobenzyl, p-carbamoylbenzyl, p-(pentafluorosulfanyl)benzyl, p-(pentafluorosulfanyl)phenoxy, p-(cyclopropylsulfonyl)phenoxy, p-(cyclopropylsulfonyl)benzyl, p-(methylsulfonyl)benzyl, p-(methylsulfonyl)phenoxy, p-(trifluoromethylsulfonyl)phenoxy, p-(trifluoromethylsulfonyl)phenyl, p-(methylsulfonyl)phenyl, p-(dimethylcarbamoyl)benzyl, p-(isopropylsulfonyl)phenyl, p-(cyclopropylsulfonyl)phenyl, p-azidobenzoyl, o,p-difluorobenzoyl, o,p-difluorobenzoxy, pyridin-3-yloxy, pyridin-4-yloxy, m,p-difluorobenzoyl, p-fluorobenzyloxy, p-(1-pyrrolidinyl)benzoyl, p-(trifluoromethylthio)phenoxy, m-(cyclopropanecarboxamido)phenoxy, p-acetamidophenoxy, m-acetamidophenoxy, p-cyclopropancarboxamidphenoxy, p-morpholinobenzoyl, p-(4-methylpiperazine-1-yl)benzoyl, p-methoxy-o-nitrophenoxy, p-(methylsulfinyl)benzoyl, p-(methylsulfonamido)benzoxy, p-nitrophenoxy, p-aminophenoxy or p-cyanobenzyl.

In other embodiments as described above, the AMPK-activating compound has the structural formula (LXXXV):

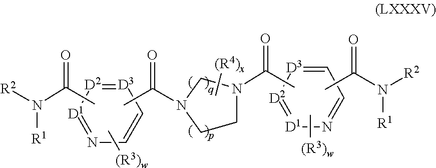

in which each of the variables is independently defined as described above with respect to structural formulae (I)-(LXXXIV). For example, in certain embodiments as described above, an AMPK-activating compound has structural formula (LXXXVI):

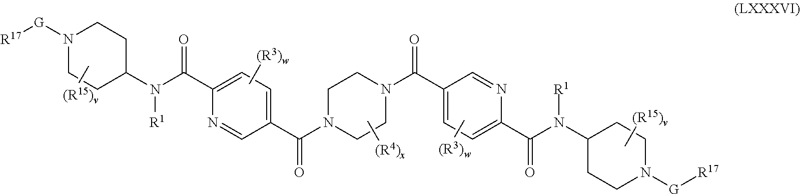

in which each of the variables is independently defined as described above with respect to structural formulae (I)-(LXXVIII).

In certain embodiments as described above, in the AMPK-activating compounds of any of structural formulae (XIII)-(LXXVI), the -G-R¹⁷ moiety is p-chlorobenzyl, p-fluorobenzyl, p-cyanobenzyl, p-cyano-m-fluorobenzyl, p-cyanobenzoyl, p-cyanobenzenesulfonyl, cyclohexanecarbonyl, benzoyl, benzyl, phenyl, cyclohexylmethyl, phenoxy, phenylmethoxy, 1-phenylethyl, p-nitrophenyl, cyanophenyl, p-(trifluoromethyl)phenyl, p-bromophenyl, 1H-pyrrol-3-yl, 4-morpholinyl, 4-methylpiperazin-1-yl, p-cyanobenzyl carbamoyl, m,m-difluorobenzyl, p-fluoro-m-methylbenzyl, p-methoxybenzyl, p-chlorobenzyl, p-methylbenzoxy, m-fluorophenoxy, p-fluorophenoxy, m-cyanophenoxy, m-methoxyphenoxy, m-methylphenoxy, p-cyanophenoxy, p-fluorophenoxy, pyrid-3-yl, thien-3-yl, phenethyl, α-carboethoxybenzyl, pyrid-4-ylmethyl, 1-(p-cyanophenyl)-1-methylethyl, p-(trifluoromethyl)benzenesulfonyl, p-(trifluoromethyl)phenoxy, p-(trifluoromethyl)benzyl, m-(trifluoromethyl)benzyl, p-methylsulfonylbenxyl, p-methylsulfonylphenoxy, p-acetylphenoxy, p-pyrrolidinylbenzyl, or p-methoxybenzyl,

As the person of skill in the art will recognize, the various embodiments and features described above can be combined to form other embodiments contemplated by the disclosure. For example, in one embodiment of the methods described herein, in the compounds of certain of structural formulae (I)-(LXXV) as described above, Q is —CH₂—, as described above, and G is —CH₂—, as described above. In another embodiment of the methods described herein, in the compounds of certain of structural formulae (I)-(LXXV) as described above, x is 0 and each w is 0. In another embodiment of the methods described herein, in the compounds of certain of structural formulae (I)-(LXXVI), x is 0, each w is 0 and each v is 0.

Moreover, the various -E moieties and T-(“B” ring system)-J- moieties described above with respect to any of structural formulae (I)-(LXXVI) can be combined around the central pyridine, pyrazine, pyridazine or pyrimidine (for example, in any of the ways described with respect to structural formulae (IX)-(XIII)) to form additional embodiments of compounds specifically contemplated by this disclosure.

In certain aspects of the methods described herein, the compound is provided as the compound itself. In other aspects, the compound is provided as the compound itself, or as pharmaceutically-acceptable salt thereof. In other aspects, the compound is provided as the compound itself, or as pharmaceutically-acceptable salt or N-oxide thereof.

Examples of compounds according to structural formula (I) include those listed in Table 1. These compounds can be made according to the general schemes described below, for example using procedures analogous to those described below in the Examples.

TABLE 1

| No. | Name | Structure |
|---|---|---|
| 1 | N-(4-(4-cyanobenzyl)piperadin-4-yl)-6-(4- (4-fluorobenzyl)piperizine-1- carbonyl)picolinamide | 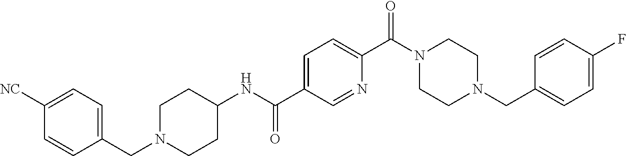 [SMILES: N#Cc1ccc(CN2CCC(NC(=O)c3ccc(C(=O)N4CCN(Cc5ccc(F)cc5)CC4)nc3)CC2)cc1] |
| 2 | N-(1-(4-cyanobenzyl)piperidin-4-yl)-5- (piperazine-1-carbonyl)picolinamide | 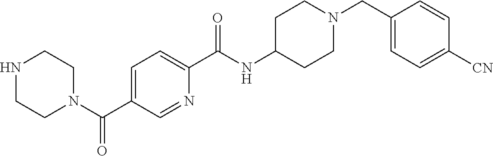 [SMILES: N#Cc1ccc(CN2CCC(NC(=O)c3ccc(C(=O)N4CCNCC4)cn3)CC2)cc1] |
| 3 | pyridine-2,5-diylbis((4-(4- fluorobenzyl)piperazin-1-yl)methanone) | 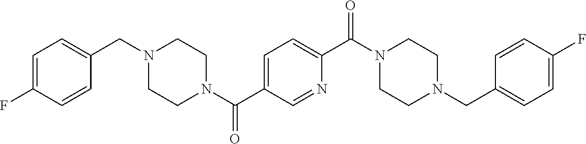 [SMILES: O=C(c1ccc(C(=O)N2CCN(Cc3ccc(F)cc3)CC2)nc1)N1CCN(Cc2ccc(F)cc2)CC1] |
| 4 | N-(1-(4-cyanobenzoyl)piperidin-4-yl)-5-(4- (4-fluorobenzyl)piperazine-1- carbonyl)picolinamide | 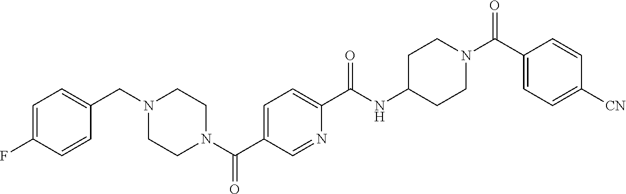 [SMILES: N#Cc1ccc(C(=O)N2CCC(NC(=O)c3ccc(C(=O)N4CCN(Cc5ccc(F)cc5)CC4)cn3)CC2)cc1] |
| 5 | N²-(1-(4-cyanobenzyl)piperidin-4-yl)-N⁵-(3- benzylphenyl)pyridine-2,5-dicarboxamide | 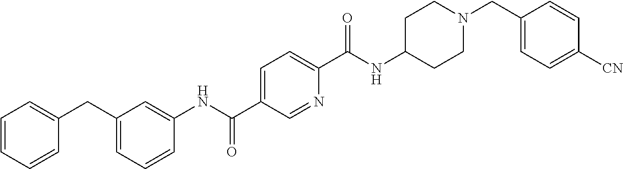 [SMILES: N#Cc1ccc(CN2CCC(NC(=O)c3ccc(C(=O)Nc4cccc(Cc5ccccc5)c4)cn3)CC2)cc1] |
| 6 | N-(4-((4-cyanophenyl)sulfonyl)piperidin-4- yl)-5-(4-(4-fluorobenzyl)piperazine-1- carbonyl)picolinamide | 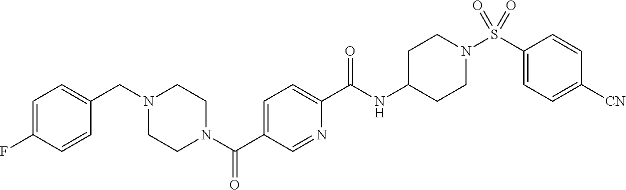 [SMILES: N#Cc1ccc(S(=O)(=O)N2CCC(NC(=O)c3ccc(C(=O)N4CCN(Cc5ccc(F)cc5)CC4)cn3)CC2)cc1] |
| 7 | N-(1-(cyclohexanecarbonyl)piperidin-4-yl)- 5-(4-(4-fluorobenzyl)piperazine-1- carbonyl)picolinamide | 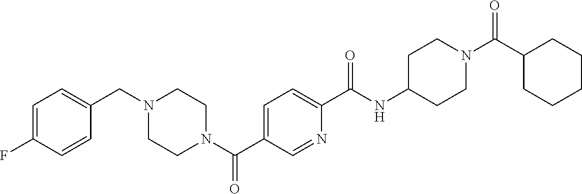 [SMILES: O=C(NC1CCN(C(=O)C2CCCCC2)CC1)c1ccc(C(=O)N2CCN(Cc3ccc(F)cc3)CC2)cn1] |
| 8 | N-(1-(benzoyl)piperidin-4-yl)-5-(4-(4- fluorobenzyl)piperazine-1- carbonyl)picolinamide | 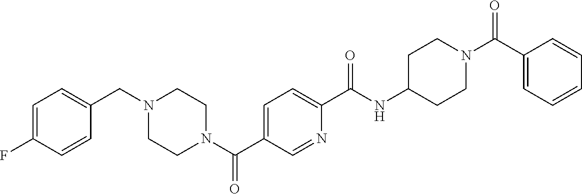 [SMILES: O=C(NC1CCN(C(=O)c2ccccc2)CC1)c1ccc(C(=O)N2CCN(Cc3ccc(F)cc3)CC2)cn1] |
| 9 | N-(1-(4-cyanobenzyl)-1H-pyrazol-3-yl)-5- (4-(4-fluorobenzyl)piperazine-1- carbonyl)picolinamide | 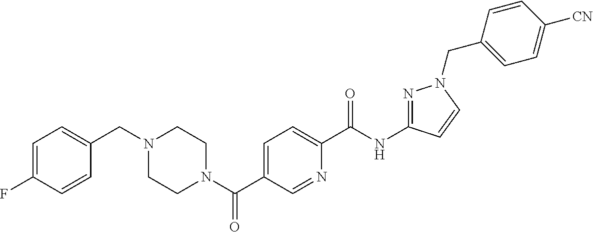 [SMILES: N#Cc1ccc(Cn2ccc(NC(=O)c3ccc(C(=O)N4CCN(Cc5ccc(F)cc5)CC4)cn3)n2)cc1] |
| 10 | N-(4-benzylphenyl)-5-(4-(4- fluorobenzyl)piperazine-1- carbonyl)picolinamide | 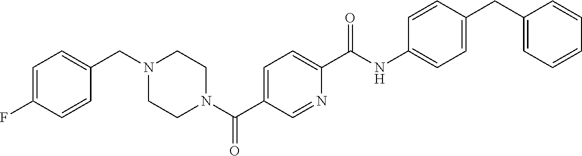 [SMILES: O=C(Nc1ccc(Cc2ccccc2)cc1)c1ccc(C(=O)N2CCN(Cc3ccc(F)cc3)CC2)cn1] |
| 11 | 5-(4-(4-fluorobenzyl)piperazine-1-carbonyl- N-(4-phenylphenyl)picolinamide | 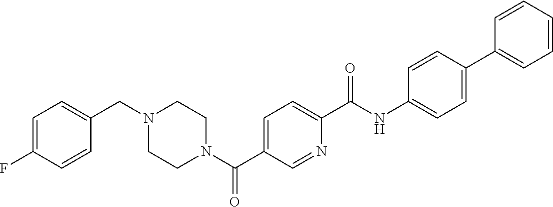 [SMILES: O=C(Nc1ccc(-c2ccccc2)cc1)c1ccc(C(=O)N2CCN(Cc3ccc(F)cc3)CC2)cn1] |
| 12 | 5-(4-(4-fluorobenzyl)piperazine-1-carbonyl- N-(3-phenylphenyl)picolinamide | 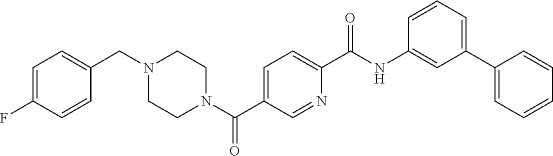 [SMILES: O=C(Nc1cccc(-c2ccccc2)c1)c1ccc(C(=O)N2CCN(Cc3ccc(F)cc3)CC2)cn1] |
| 13 | N-(1-(cyclohexylmethyl)piperidin-4-yl)-5- (4-(4-fluorobenzyl)piperazine-1- carbonyl)picolinamide | 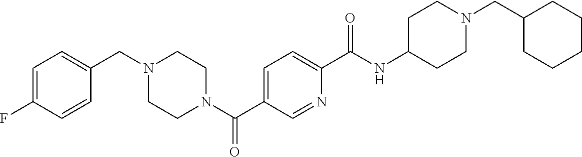 [SMILES: O=C(NC1CCN(CC2CCCCC2)CC1)c1ccc(C(=O)N2CCN(Cc3ccc(F)cc3)CC2)cn1] |
| 14 | 5-(4-(4-fluorobenzyl)piperazine-1- carbonyl)-N-(1-(phenyl)piperidin-4- yl)picolinamide | 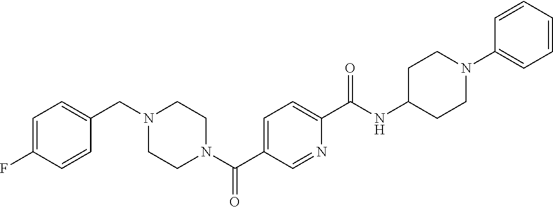 [SMILES: O=C(NC1CCN(c2ccccc2)CC1)c1ccc(C(=O)N2CCN(Cc3ccc(F)cc3)CC2)cn1] |
| 15 | 4-((8-(5-(4-(4-fluorobenzyl)piperazine-1- carbonyl)picolinoyl)-2,8- diazaspiro[4.5]decan-2- yl)methyl)benzonitrile | 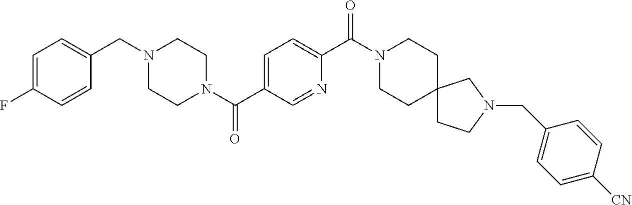 [SMILES: N#Cc1ccc(CN2CCC3(CCN(C(=O)c4ccc(C(=O)N5CCN(Cc6ccc(F)cc6)CC5)cn4)CC3)C2)cc1] |
| 16 | 5-(4-(4-fluorobenzyl)piperazine-1- carbonyl)-N-(4- phenoxyphenyl)picolinamide | 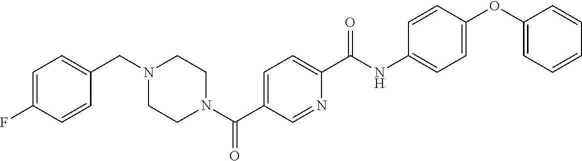 [SMILES: O=C(Nc1ccc(Oc2ccccc2)cc1)c1ccc(C(=O)N2CCN(Cc3ccc(F)cc3)CC2)cn1] |
| 17 | (4-(4-fluorobenzyl)piperazin-1-yl)(6-(4- (benzyloxy)phenyl)pyridin-3-yl)methanone | 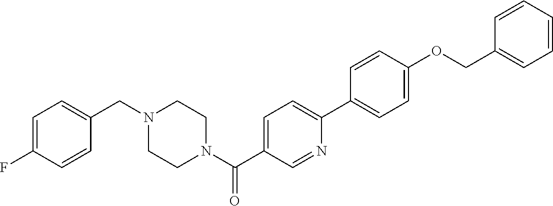 [SMILES: O=C(c1ccc(-c2ccc(OCc3ccccc3)cc2)nc1)N1CCN(Cc2ccc(F)cc2)CC1] |
| 18 | 5-(4-(4-fluorobenzyl)piperazine-1- carbonyl)-N-(1-(1-phenylethyl)piperidin-4- yl)picolinamide | 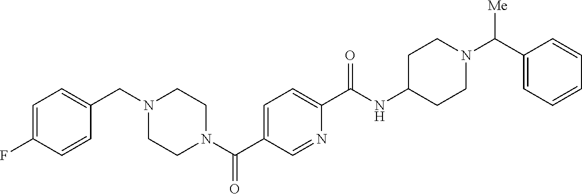 [SMILES: CC(c1ccccc1)N1CCC(NC(=O)c2ccc(C(=O)N3CCN(Cc4ccc(F)cc4)CC3)cn2)CC1] |
| 19 | 5-(4-(4-fluorobenzyl)piperazine-1- carbonyl)-N-(2-phenylphenyl)picolinamide | 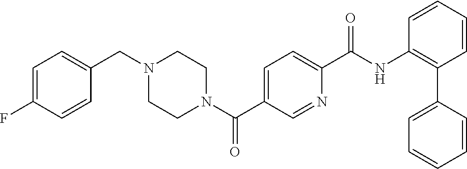 [SMILES: O=C(Nc1ccccc1-c1ccccc1)c1ccc(C(=O)N2CCN(Cc3ccc(F)cc3)CC2)cn1] |
| 20 | 5-(4-(4-fluorobenzyl)piperazine-1- carbonyl)-N-(4-(4-nitrophenyl)phenyl) picolinamide | 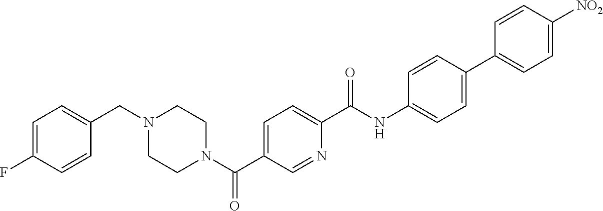 [SMILES: O=C(Nc1ccc(-c2ccc([N+](=O)[O-])cc2)cc1)c1ccc(C(=O)N2CCN(Cc3ccc(F)cc3)CC2)cn1] |
| 21 | 5-(4-(4-fluorobenzyl)piperazine-1- carbonyl)-N-(3- phenoxyphenyl)picolinamide | 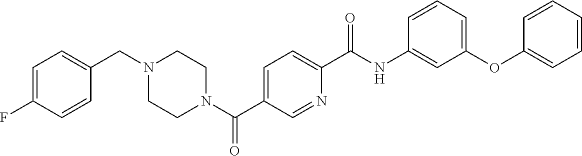 [SMILES: O=C(Nc1cccc(Oc2ccccc2)c1)c1ccc(C(=O)N2CCN(Cc3ccc(F)cc3)CC2)cn1] |
| 22 | (6-(3-(benzyloxy)phenyl)pyridin-3-yl)(4-(4- fluorobenzyl)piperazin-1-yl)methanone | 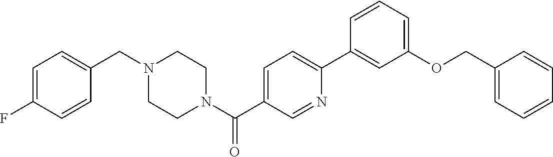 [SMILES: O=C(c1ccc(-c2cccc(OCc3ccccc3)c2)nc1)N1CCN(Cc2ccc(F)cc2)CC1] |
| 23 | N-(1-(4-cyanobenzyl)-1H-pyrazol-4-yl)-5- (4-(4-fluorobenzyl)piperazine-1- carbonyl)picolinamide | 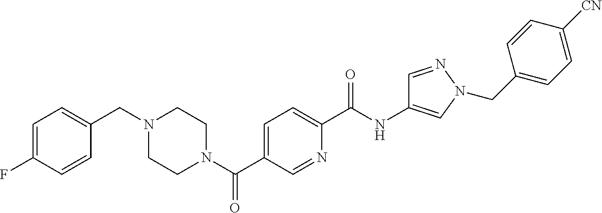 [SMILES: N#Cc1ccc(Cn2cc(NC(=O)c3ccc(C(=O)N4CCN(Cc5ccc(F)cc5)CC4)cn3)cn2)cc1] |
| 24 | N-(4-(4-cyanophenyl)phenyl)-5-(4-(4- fluorobenzyl)piperazine-1- carbonyl)picolinamide | 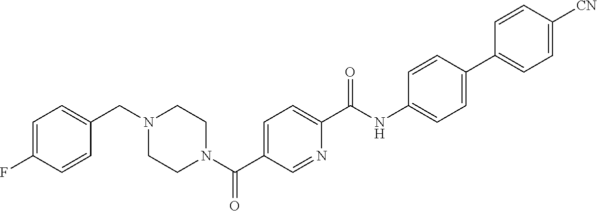 [SMILES: N#Cc1ccc(-c2ccc(NC(=O)c3ccc(C(=O)N4CCN(Cc5ccc(F)cc5)CC4)cn3)cc2)cc1] |
| 25 | 5-(4-(4-fluorobenzyl)piperazine-1- carbonyl)-N-(4-(4- trifluoromethylphenyl)phenyl)picolinamide | 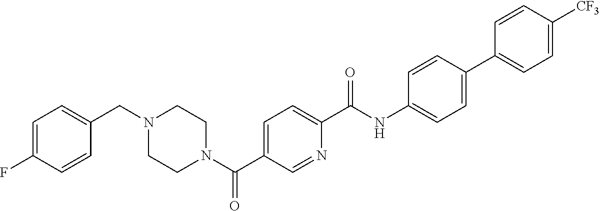 [SMILES: O=C(Nc1ccc(-c2ccc(C(F)(F)F)cc2)cc1)c1ccc(C(=O)N2CCN(Cc3ccc(F)cc3)CC2)cn1] |
| 26 | N-(4-benzoylphenyl)-5-(4-(4- fluorobenzyl)piperazine-1- carbonyl)picolinamide | 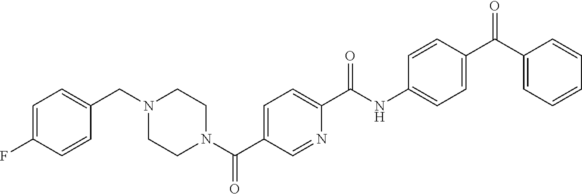 [SMILES: O=C(c1ccccc1)c1ccc(NC(=O)c2ccc(C(=O)N3CCN(Cc4ccc(F)cc4)CC3)cn2)cc1] |
| 27 | N-(4-benzyloxyphenyl)-5-(4-(4- fluorobenzyl)piperazine-1- carbonyl)picolinamide | 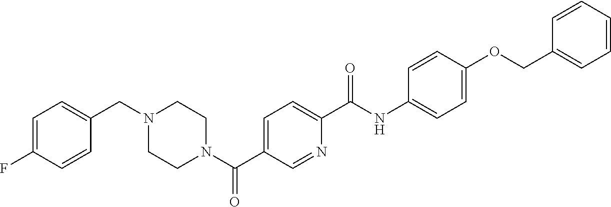 [SMILES: O=C(Nc1ccc(OCc2ccccc2)cc1)c1ccc(C(=O)N2CCN(Cc3ccc(F)cc3)CC2)cn1] |
| 28 | N-(4-bromophenyl)-5-(4-(4- fluorobenzyl)piperazine-1- carbonyl)picolinamide | 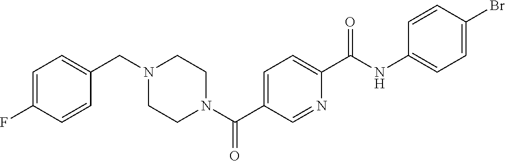 [SMILES: O=C(Nc1ccc(Br)cc1)c1ccc(C(=O)N2CCN(Cc3ccc(F)cc3)CC2)cn1] |
| 29 | N-(4-(4-methoxyphenyl)phenyl)-5-(4-(4- fluorobenzyl)piperazine-1- carbonyl)picolinamide | 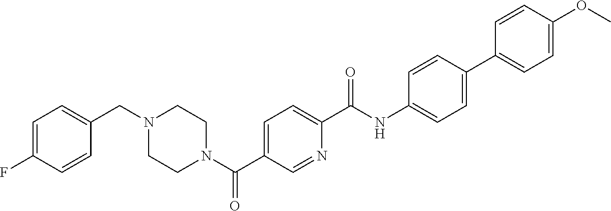 [SMILES: COc1ccc(-c2ccc(NC(=O)c3ccc(C(=O)N4CCN(Cc5ccc(F)cc5)CC4)cn3)cc2)cc1] |
| 30 | (6-(4-benzylphenylamino)pyridin-3-yl)(4- (4-fluorobenzyppiperazin-1-yl)methanone | 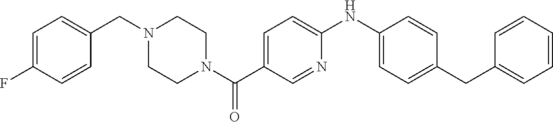 [SMILES: O=C(c1ccc(Nc2ccc(Cc3ccccc3)cc2)nc1)N1CCN(Cc2ccc(F)cc2)CC1] |
| 31 | 4-((2-(5-(4-(4-fluorobenzyl)piperazine-1- carbonyl)pyridin-2-yl)-2,8- diazaspiro[4.5]decan-8- yl)methyl)benzonitrile | 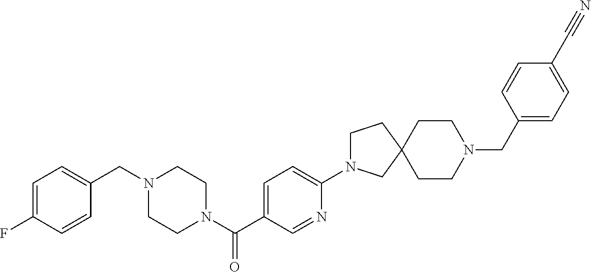 [SMILES: N#Cc1ccc(CN2CCC3(CC2)CCN(c2ccc(C(=O)N4CCN(Cc5ccc(F)cc5)CC4)cn2)C3)cc1] |
| 32 | N-(4-(3-cyanophenyl)phenyl)-5-(4-(4- fluorobenzyl)piperazine-1- carbonyl)picolinamide | 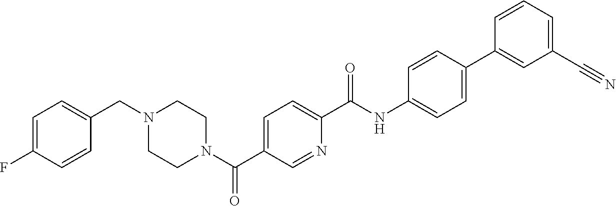 [SMILES: N#Cc1cccc(-c2ccc(NC(=O)c3ccc(C(=O)N4CCN(Cc5ccc(F)cc5)CC4)cn3)cc2)c1] |
| 33 | (6-(3-phenylphenylamino)pyridin-3-yl)(4- (4-fluorobenzyl)piperazin-1-yl)methanone | 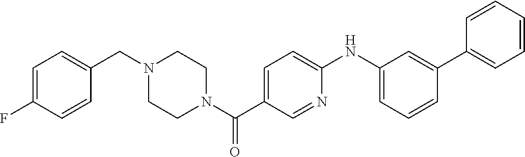 [SMILES: O=C(c1ccc(Nc2cccc(-c3ccccc3)c2)nc1)N1CCN(Cc2ccc(F)cc2)CC1] |
| 34 | (4-(4-fluorobenzyl)piperazin-1-yl)(6-(4- phenoxyphenylamino)pyridin-3- yl)methanone | 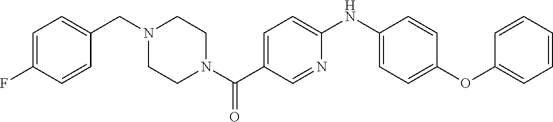 [SMILES: O=C(c1ccc(Nc2ccc(Oc3ccccc3)cc2)nc1)N1CCN(Cc2ccc(F)cc2)CC1] |
| 35 | (6-(4-(4- cyanobenzylcarbamoyl)phenyl)pyridin-3- yl)(4-(4-fluorobenzyl)piperazin-1- yl)methanone | 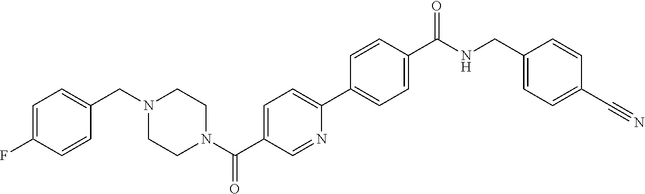 [SMILES: N#Cc1ccc(CNC(=O)c2ccc(-c3ccc(C(=O)N4CCN(Cc5ccc(F)cc5)CC4)cn3)cc2)cc1] |
| 36 | (6-(4-(cyanobenzyl)piperidin-4- ylamino)pyridin-3-yl)(4-(4- fluorobenzyl)piperazin-1-yl)methanone | 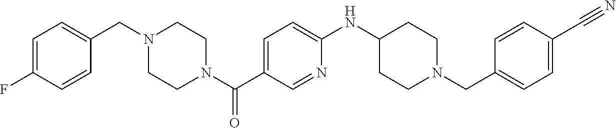 [SMILES: N#Cc1ccc(CN2CCC(Nc3ccc(C(=O)N4CCN(Cc5ccc(F)cc5)CC4)cn3)CC2)cc1] |
| 37 | (6-(4-phenylphenylamino)pyridin-3-yl)(4- (4-fluorobenzyl)piperazin-1-yl)methanone | 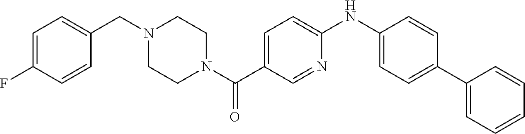 [SMILES: O=C(c1ccc(Nc2ccc(-c3ccccc3)cc2)nc1)N1CCN(Cc2ccc(F)cc2)CC1] |
| 38 | N⁵-(1-(4-cyanobenzyl)-1H-pyrazol-3-yl)-N²- (1-(4-cyanobenzyl)piperidin-4-yl)pyridine- 2,5-dicarboxamide | 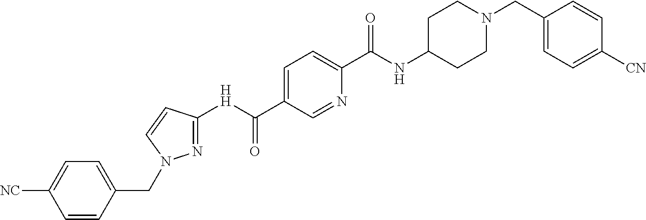 |
| 39 | 5-(4-(4-fluorobenzyl)piperazine-1- carbonyl)-N-(4-(1H-pyrrol-3- yl)phenyl)picolinamide | 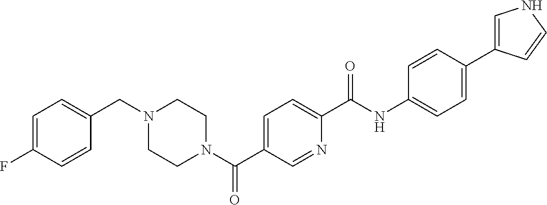 [SMILES: O=C(Nc1ccc(-c2cc[nH]c2)cc1)c1ccc(C(=O)N2CCN(Cc3ccc(F)cc3)CC2)cn1] |
| 40 | 5-(4-(4-fluorobenzyl)piperazine-1- carbonyl)-N-(4- morpholinophenyl)picolinamide | 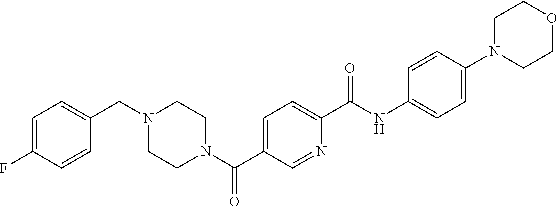 [SMILES: O=C(Nc1ccc(N2CCOCC2)cc1)c1ccc(C(=O)N2CCN(Cc3ccc(F)cc3)CC2)cn1] |
| 41 | 5-(4-(4-fluorobenzyl)piperazine-1- carbonyl)-N-(4-(4-methylpiperazin-1- yl)phenyl)picolinamide | 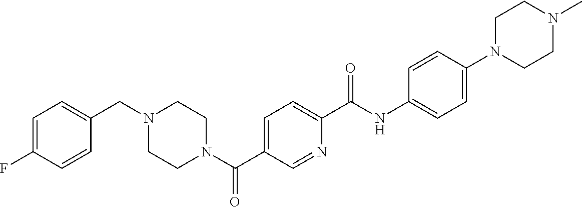 [SMILES: CN1CCN(c2ccc(NC(=O)c3ccc(C(=O)N4CCN(Cc5ccc(F)cc5)CC4)cn3)cc2)CC1] |
| 42 | (6-(3-(4- cyanobenzylcarbamoyl)phenyl)pyridin- 3yl)(4-(4-fluorobenzyl)piperazin-1- yl)methanone | 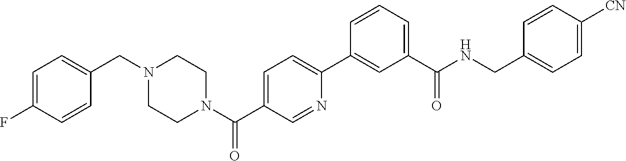 [SMILES: N#Cc1ccc(CNC(=O)c2cccc(-c3ccc(C(=O)N4CCN(Cc5ccc(F)cc5)CC4)cn3)c2)cc1] |
| 43 | N⁵-(1-(4-cyanobenzyl)-1H-pyrazol-4-yl)-N²- (1-(4-cyanobenzyl)piperidin-4-yl)pyridine- 2,5-dicarboxamide | 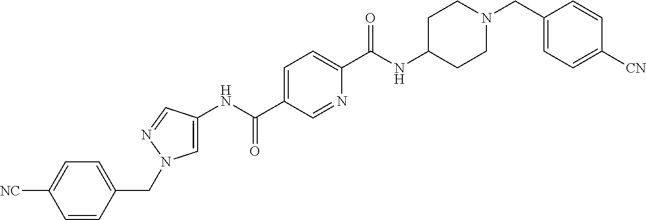 [SMILES: N#Cc1ccc(CN2CCC(NC(=O)c3ccc(C(=O)Nc4cnn(Cc5ccc(C#N)cc5)c4)cn3)CC2)cc1] |
| 44 | (6-(1-(4-fluorobenzyl)-1H-pyrazol-4- ylamino)pyridin-3-yl)(4-(4- fluorobenzyl)piperazin-1-yl)methanone | 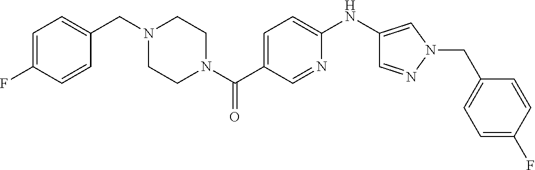 [SMILES: O=C(c1ccc(Nc2cnn(Cc3ccc(F)cc3)c2)nc1)N1CCN(Cc2ccc(F)cc2)CC1] |
| 45 | N-(1-(4-cyanobenzyl)piperidin-4-yl)-5-(1- (4-fluorobenzyl)-1H-pyrazol-4- ylamino)picolinamide | 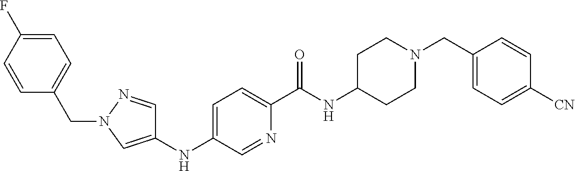 [SMILES: N#Cc1ccc(CN2CCC(NC(=O)c3ccc(Nc4cnn(Cc5ccc(F)cc5)c4)cn3)CC2)cc1] |
| 46 | (6-(1-(4-cyanobenzyl)piperidine-4- carboxamido)pyridin-3-yl)(4-(4- fluorobenzyl)piperazin-1-yl)methanone | 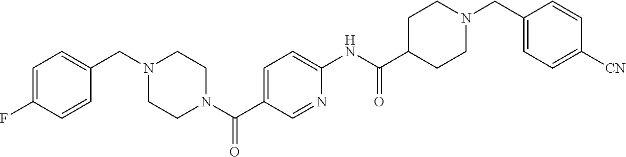 [SMILES: N#Cc1ccc(CN2CCC(C(=O)Nc3ccc(C(=O)N4CCN(Cc5ccc(F)cc5)CC4)cn3)CC2)cc1] |
| 47 | N-(4-(4-cyanobenzylcarbamoyl)phenyl)-5- (4-(4-fluorobenzyl)piperazine-1- carbonyl)picolinamide | 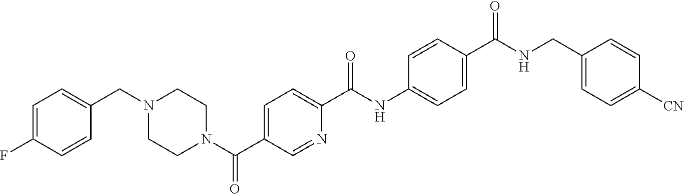 [SMILES: N#Cc1ccc(CNC(=O)c2ccc(NC(=O)c3ccc(C(=O)N4CCN(Cc5ccc(F)cc5)CC4)cn3)cc2)cc1] |
| 48 | (6-(4-(4- cyanobenzylcarbamoyl)phenylamino) pyridin-3-yl)(4-(4-fluorobenzyl)piperazin-1- yl)methanone | 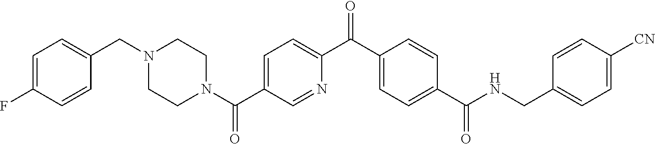 [SMILES: N#Cc1ccc(CNC(=O)c2ccc(C(=O)c3ccc(C(=O)N4CCN(Cc5ccc(F)cc5)CC4)cn3)cc2)cc1] |
| 49 | N-(1-(3,5-difluorobenzyl)piperidin-4-yl)-5- (4-(4-fluorobenzyl)piperazine-1- carbonyl)picolinamide | 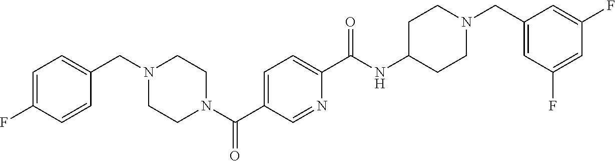 [SMILES: O=C(NC1CCN(Cc2cc(F)cc(F)c2)CC1)c1ccc(C(=O)N2CCN(Cc3ccc(F)cc3)CC2)cn1] |
| 50 | 5-(4-(4-fluorobenzyl)piperazine-1- carbonyl)-N-(1-(4-fluoro-3- methylbenzyl)piperidin-4-yl)picolinamide | 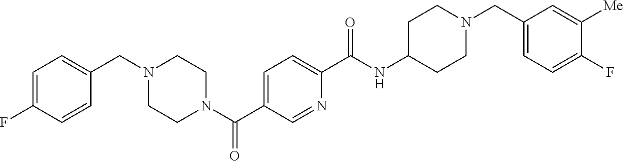 [SMILES: Cc1cc(CN2CCC(NC(=O)c3ccc(C(=O)N4CCN(Cc5ccc(F)cc5)CC4)cn3)CC2)ccc1F] |
| 51 | N-(1-(4-chlorobenzyl)piperidin-4-yl)-5-(4- (4-fluorobenzyl)piperazine-1- carbonyl)picolinamide | 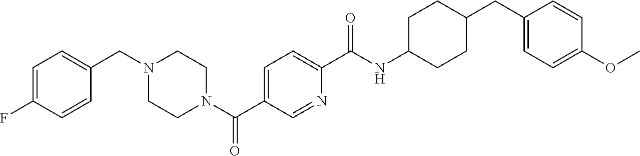 [SMILES: COc1ccc(CC2CCC(NC(=O)c3ccc(C(=O)N4CCN(Cc5ccc(F)cc5)CC4)cn3)CC2)cc1] |
| 52 | N-(1-(4-chlorobenzyl)piperidin-4-yl)-5-(4- (4-fluorobenzyppiperazine-1- carbonyl)picolinamide | 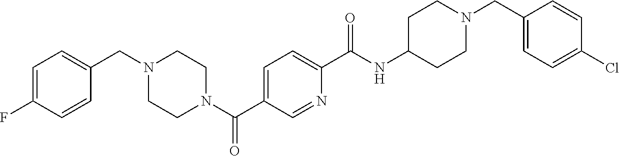 [SMILES: O=C(NC1CCN(Cc2ccc(Cl)cc2)CC1)c1ccc(C(=O)N2CCN(Cc3ccc(F)cc3)CC2)cn1] |
| 53 | 5-(4-(4-fluorobenzyl)piperazine-1- carbonyl)-N-(4-(4- methylphenoxy)phenyl)picolinamide | 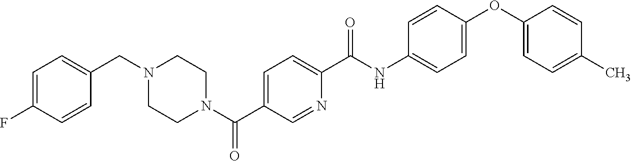 [SMILES: Cc1ccc(Oc2ccc(NC(=O)c3ccc(C(=O)N4CCN(Cc5ccc(F)cc5)CC4)cn3)cc2)cc1] |
| 54 | 5-(4-(4-fluorobenzyl)piperazine-1- carbonyl)-N-(4-(4- methoxyphenoxy)phenyl)picolinamide | 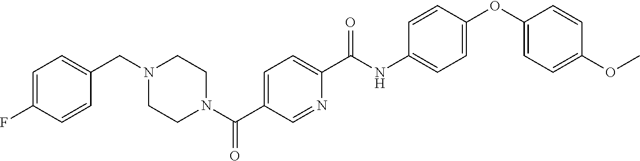 [SMILES: COc1ccc(Oc2ccc(NC(=O)c3ccc(C(=O)N4CCN(Cc5ccc(F)cc5)CC4)cn3)cc2)cc1] |
| 55 | 5-(4-(4-fluorobenzyl)piperazine-1- carbonyl)-N-(4-(3- fluorophenoxy)phenyl)picolinamide | 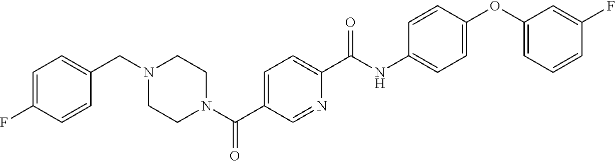 [SMILES: O=C(Nc1ccc(Oc2cccc(F)c2)cc1)c1ccc(C(=O)N2CCN(Cc3ccc(F)cc3)CC2)cn1] |
| 56 | N-(4-(3-cyanophenoxy)phenyl)-5- (4-(4-fluorobenzyl)piperazine-1- carbonyl)picolinamide | 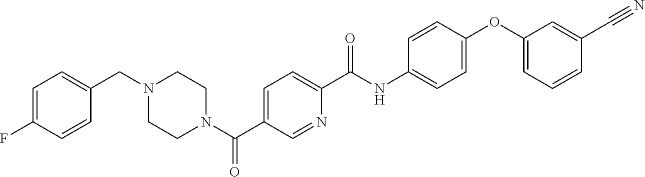 [SMILES: N#Cc1cccc(Oc2ccc(NC(=O)c3ccc(C(=O)N4CCN(Cc5ccc(F)cc5)CC4)cn3)cc2)c1] |
| 57 | 5-(4-(4-fluorobenzyl)piperazine-1- carbonyl)-N-(4-(3- methoxyphenoxy)phenyl)picolinamide | 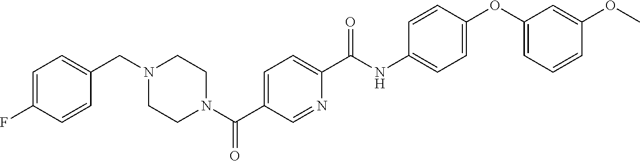 [SMILES: COc1cccc(Oc2ccc(NC(=O)c3ccc(C(=O)N4CCN(Cc5ccc(F)cc5)CC4)cn3)cc2)c1] |
| 58 | 5-(4-(4-fluorobenzyl)piperazine-1- carbonyl)-N-(4-(3- methylphenoxy)phenyl)picolinamide | 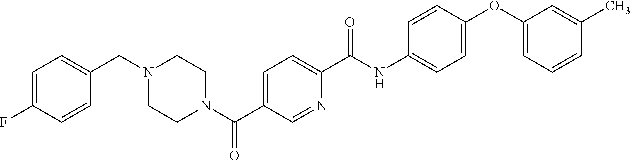 [SMILES: Cc1cccc(Oc2ccc(NC(=O)c3ccc(C(=O)N4CCN(Cc5ccc(F)cc5)CC4)cn3)cc2)c1] |
| 59 | N-(4-(4-cyanophenoxy)phenyl)-5-(4-(4- fluorobenzyl)piperazine-1- carbonyl)picolinamide | 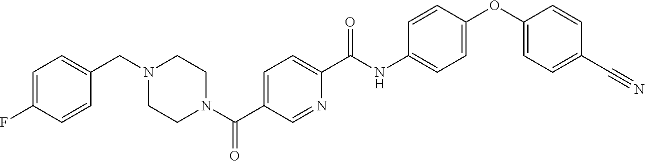 [SMILES: N#Cc1ccc(Oc2ccc(NC(=O)c3ccc(C(=O)N4CCN(Cc5ccc(F)cc5)CC4)cn3)cc2)cc1] |
| 60 | 5-(4-(4-fluorobenzyl)piperazine-1- carbonyl)-N-(4-(4- fluorophenoxy)phenyl)picolinamide | 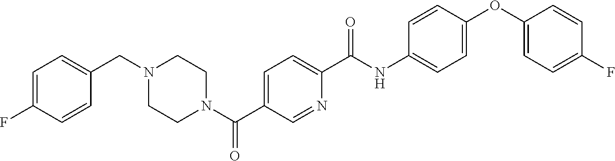 [SMILES: O=C(Nc1ccc(Oc2ccc(F)cc2)cc1)c1ccc(C(=O)N2CCN(Cc3ccc(F)cc3)CC2)cn1] |
| 61 | 5-(4-(4-fluorobenzyl)piperazine-1- carbonyl)-N-(4-(pyridine-3- yl)phenyl)picolinamide | 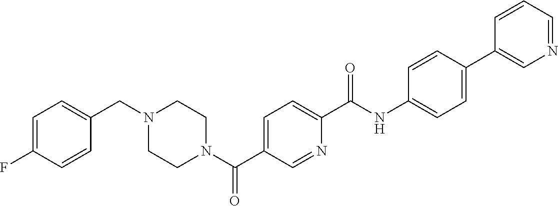 [SMILES: O=C(Nc1ccc(-c2cccnc2)cc1)c1ccc(C(=O)N2CCN(Cc3ccc(F)cc3)CC2)cn1] |
| 62 | 5-(4-(4-fluorobenzyl)piperazine-1- carbonyl)-N-(4-(thiophen-3- yl)phenyl)picolinamide | 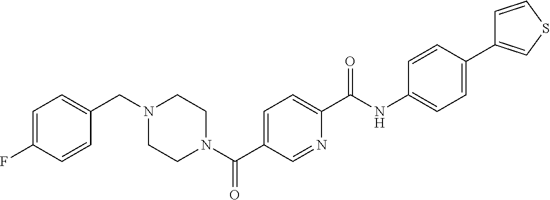 [SMILES: O=C(Nc1ccc(-c2ccsc2)cc1)c1ccc(C(=O)N2CCN(Cc3ccc(F)cc3)CC2)cn1] |
| 63 | 5-(4-(4-fluorobenzyl)piperazine-1- carbonyl)-(6-(4-cyanophenoxy)pyridin-3- yl)picolinamide | 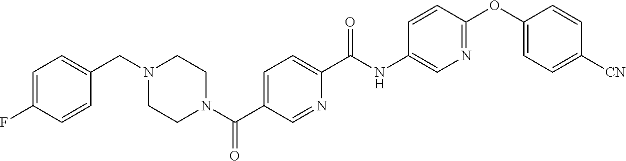 [SMILES: N#Cc1ccc(Oc2ccc(NC(=O)c3ccc(C(=O)N4CCN(Cc5ccc(F)cc5)CC4)cn3)cn2)cc1] |
| 64 | 5-(4-(4-fluorobenzyl)piperazine-1- carbonyl)-(6-(3-cyanophenoxy)pyridin-3- yl)picolinamide | 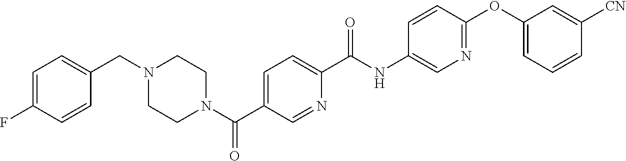 [SMILES: N#Cc1cccc(Oc2ccc(NC(=O)c3ccc(C(=O)N4CCN(Cc5ccc(F)cc5)CC4)cn3)cn2)c1] |
| 65 | 5-(4-(4-fluorobenzyl)piperazine-1- carbonyl)-N-(6-(4-fluorophenoxy) pyridin-3- yl)picolinamide | 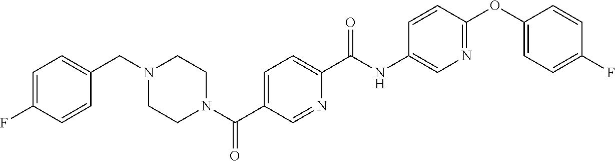 [SMILES: O=C(Nc1ccc(Oc2ccc(F)cc2)nc1)c1ccc(C(=O)N2CCN(Cc3ccc(F)cc3)CC2)cn1] |
| 66 | 5-(4-(4-cyano-2- methoxyphenoxy)piperidine-1- carbonyl)-N- (1-(4-cyanobenzyl)piperidin-4- yl)picolinamide | 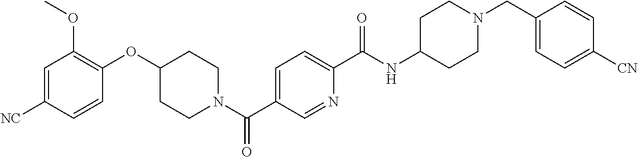 [SMILES: COc1cc(C#N)ccc1OC1CCN(C(=O)c2ccc(C(=O)NC3CCN(Cc4ccc(C#N)cc4)CC3)nc2)CC1] |
| 67 | 5-(4-(4-fluoro-4-fluorobenzoyl) piperidine-1-carbonyl)-N-(6-(4- fluorophenoxy)pyridin- 3-yl)picolinamide | 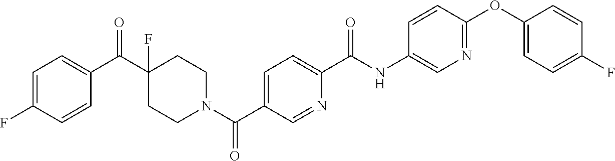 [SMILES: O=C(Nc1ccc(Oc2ccc(F)cc2)nc1)c1ccc(C(=O)N2CCC(F)(C(=O)c3ccc(F)cc3)CC2)cn1] |
| 68 | N-(1-(4-cyanobenzyl)piperidin-4-yl)-5-(4- (4-fluoro-4-fluorobenzoyl)piperidine-1- carbonyl)picolinamide | 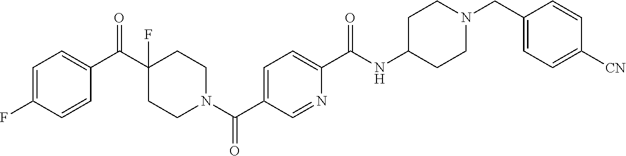 [SMILES: N#Cc1ccc(CN2CCC(NC(=O)c3ccc(C(=O)N4CCC(F)(C(=O)c5ccc(F)cc5)CC4)cn3)CC2)cc1] |
| 69 | 5-(4-(4-methoxybenzoyl)piperidine-1- carbonyl)-N-(6-(4-fluorophenoxy)pyridin-3- yl)picolinamide | 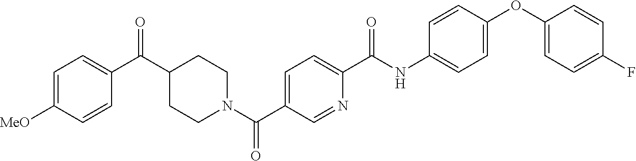 [SMILES: COc1ccc(C(=O)C2CCN(C(=O)c3ccc(C(=O)Nc4ccc(Oc5ccc(F)cc5)cc4)nc3)CC2)cc1] |
| 70 | 5-(4-(4-methoxyphenoxy)piperidine-1- carbonyl)-N-(6-(4-fluorophenoxy)pyridin-3- yl)picolinamide | 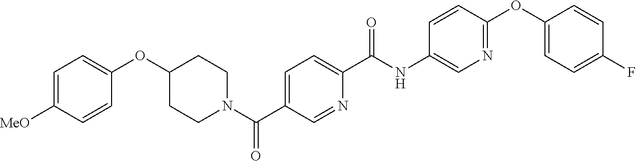 [SMILES: COc1ccc(OC2CCN(C(=O)c3ccc(C(=O)Nc4ccc(Oc5ccc(F)cc5)nc4)nc3)CC2)cc1] |
| 71 | trans-N-(4-(4-cyanophenoxy)cyclohexyl)-5- (4-(4-fluorobenzyl)piperazine-1- carbonyl)picolinamide | 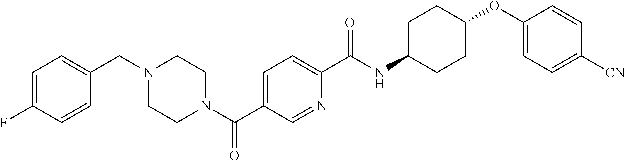 [SMILES: N#Cc1ccc(O[C@H]2CC[C@H](NC(=O)c3ccc(C(=O)N4CCN(Cc5ccc(F)cc5)CC4)cn3)CC2)cc1] |
| 72 | 5-(4-benzylpiperazine-1-carbonyl)-N-(1- benzylpiperidin-4-yl)picolinamide | 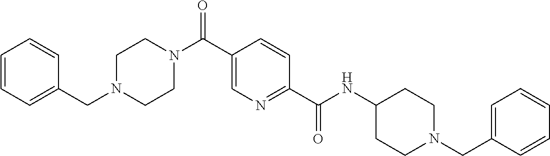 [SMILES: O=C(NC1CCN(Cc2ccccc2)CC1)c1ccc(C(=O)N2CCN(Cc3ccccc3)CC2)cn1] |
| 73 | pyridine-2,5-diylbis((4-benzylpiperazin-1- yl)methanone) | 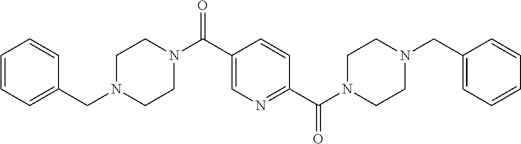 [SMILES: O=C(c1ccc(C(=O)N2CCN(Cc3ccccc3)CC2)nc1)N1CCN(Cc2ccccc2)CC1] |
| 74 | 6-(4-benzylpiperazine-1-carbonyl)-N-(1- benzylpiperidin-4-yl)nicotinamide | 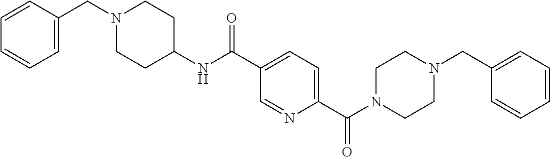 [SMILES: O=C(NC1CCN(Cc2ccccc2)CC1)c1ccc(C(=O)N2CCN(Cc3ccccc3)CC2)nc1] |
| 75 | 5,5′-(piperazine-1,4- diylbis(oxomethylene))bis(N-(1-(4- cyanobenzyl)piperidin-4-yl) picolinamide) | 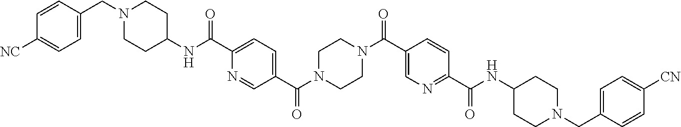 [SMILES: N#Cc1ccc(CN2CCC(NC(=O)c3ccc(C(=O)N4CCN(C(=O)c5ccc(C(=O)NC6CCN(Cc7ccc(C#N)cc7)CC6)nc5)CC4)cn3)CC2)cc1] |
| 76 | N-(1-(4-cyanobenzyl)piperidin-4-yl)- 5-(4-(4-fluorobenzoyl)piperazine-1- carbonyl)picolinamide | 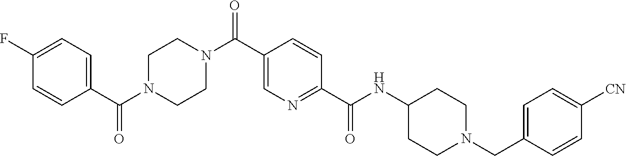 [SMILES: N#Cc1ccc(CN2CCC(NC(=O)c3ccc(C(=O)N4CCN(C(=O)c5ccc(F)cc5)CC4)cn3)CC2)cc1] |
| 77 | N-(1-(4-cyanobenzyl)piperidin-4-yl)-5-(4- (4-methoxybenzoyl)piperazine-1- carbonyl)picolinamide | 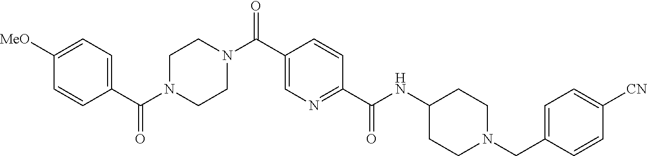 [SMILES: COc1ccc(C(=O)N2CCN(C(=O)c3ccc(C(=O)NC4CCN(Cc5ccc(C#N)cc5)CC4)nc3)CC2)cc1] |
| 78 | N-(1-(4-cyanobenzyl)piperidin-4-yl)-5-(4- (4-fluorophenylsulfonyl)piperazine-1- carbonyl)picolinamide | 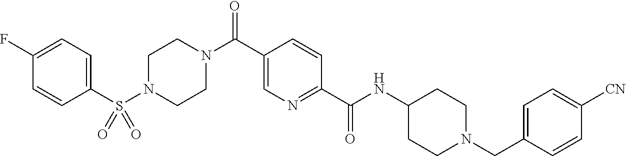 [SMILES: N#Cc1ccc(CN2CCC(NC(=O)c3ccc(C(=O)N4CCN(S(=O)(=O)c5ccc(F)cc5)CC4)cn3)CC2)cc1] |
| 79 | N-(1-(4-cyanobenzyl)piperidin-4-yl)-5-(4- (4-methoxyphenylsulfonyl)piperazine-1- carbonyl)picolinamide | 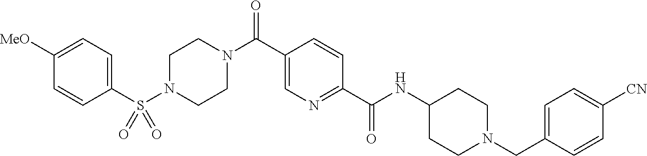 [SMILES: COc1ccc(S(=O)(=O)N2CCN(C(=O)c3ccc(C(=O)NC4CCN(Cc5ccc(C#N)cc5)CC4)nc3)CC2)cc1] |
| 80 | 5-(4-benzoylpiperazine-1-carbonyl)-N-(1- (4-cyanobenzyl)piperidin-4-yl)picolinamide | 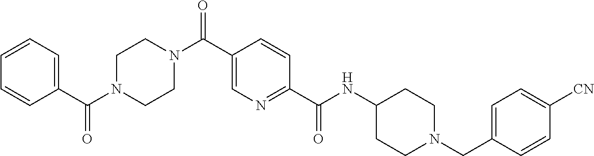 [SMILES: N#Cc1ccc(CN2CCC(NC(=O)c3ccc(C(=O)N4CCN(C(=O)c5ccccc5)CC4)cn3)CC2)cc1] |
| 81 | N-(1-(4-cyanobenzyl)piperidin-4-yl)-5-(4- pivaloylpiperazine-1-carbonyl)picolinamide | 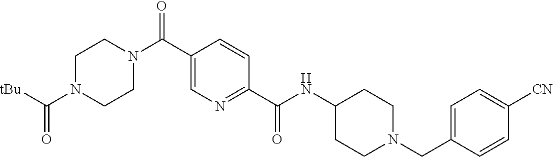 [SMILES: CC(C)(C)C(=O)N1CCN(C(=O)c2ccc(C(=O)NC3CCN(Cc4ccc(C#N)cc4)CC3)nc2)CC1] |
| 82 | N-(1-(4-cyanobenzyl)piperidin-4-yl)-5-(4- (phenylsulfonyl)piperazine-1- carbonyl)picolinamide | 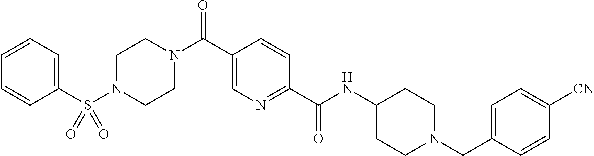 [SMILES: N#Cc1ccc(CN2CCC(NC(=O)c3ccc(C(=O)N4CCN(S(=O)(=O)c5ccccc5)CC4)cn3)CC2)cc1] |
| 83 | N-(1-(4-cyanobenzyl)piperidin-4-yl)-5-(4- (tetrahydro-2H-pyran-4-yl)piperazine-1- carbonyl)picolinamide | 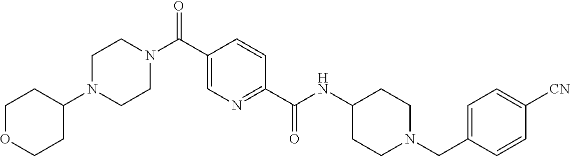 [SMILES: N#Cc1ccc(CN2CCC(NC(=O)c3ccc(C(=O)N4CCN(C5CCOCC5)CC4)cn3)CC2)cc1] |
| 84 | N-(1-(4-cyanobenzyl)piperidin-4-yl)-5-(4- isopropylpiperazine-1- carbonyl)picolinamide | 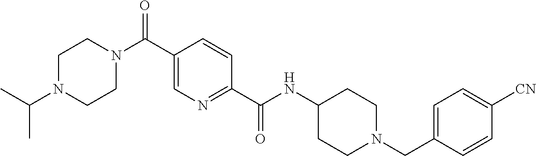 [SMILES: CC(C)N1CCN(C(=O)c2ccc(C(=O)NC3CCN(Cc4ccc(C#N)cc4)CC3)nc2)CC1] |
| 85 | N-(1-benzylpiperidin-4-yl)-5-(4-((5- methylisoxazol-3-yl)methyl)piperazine-1- carbonyl)picolinamide | 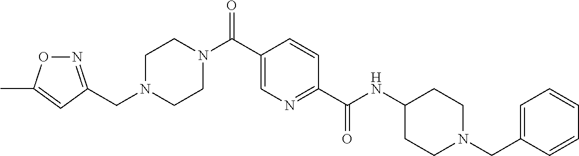 [SMILES: Cc1cc(CN2CCN(C(=O)c3ccc(C(=O)NC4CCN(Cc5ccccc5)CC4)nc3)CC2)no1] |
| 86 | N2,N6-bis(1-(4-cyanobenzyl)piperidin-4- yl)pyridine-2,6-dicarboxamide | 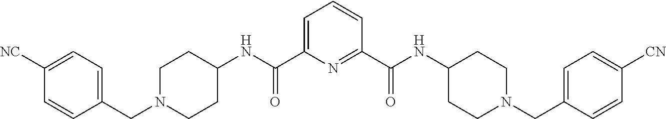 [SMILES: N#Cc1ccc(CN2CCC(NC(=O)c3cccc(C(=O)NC4CCN(Cc5ccc(C#N)cc5)CC4)n3)CC2)cc1] |
| 87 | N2,N6-bis(1-(4-fluorobenzyl)piperidin-4- yl)pyridine-2,6-dicarboxamide | 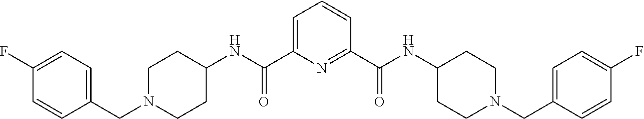 [SMILES: O=C(NC1CCN(Cc2ccc(F)cc2)CC1)c1cccc(C(=O)NC2CCN(Cc3ccc(F)cc3)CC2)n1] |
| 88 | (4-(4-fluorobenzyl)piperazin-1-yl)(6-(4- phenethylpiperazine-1-carbonyl)pyridin-3- yl)methanone | 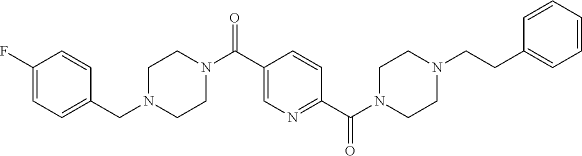 [SMILES: O=C(c1ccc(C(=O)N2CCN(CCc3ccccc3)CC2)nc1)N1CCN(Cc2ccc(F)cc2)CC1] |
| 89 | N-(1-(4-cyanobenzyl)piperidin-4-yl)-5-(4- (cyclohexanecarbonyl)piperazine-1- carbonyl)picolinamide | 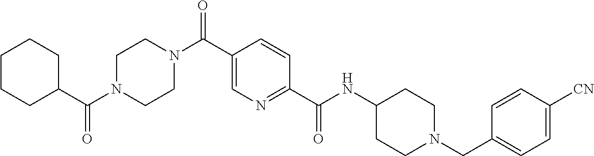 [SMILES: N#Cc1ccc(CN2CCC(NC(=O)c3ccc(C(=O)N4CCN(C(=O)C5CCCCC5)CC4)cn3)CC2)cc1] |
| 90 | (4-phenethylpiperazin-1-yl)(5-(4- phenylpiperazine-1-carbonyl)pyridin-2- yl)methanone | 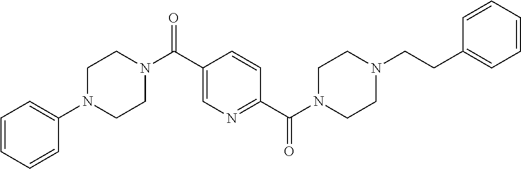 [SMILES: O=C(c1ccc(C(=O)N2CCN(CCc3ccccc3)CC2)nc1)N1CCN(c2ccccc2)CC1] |
| 91 | (4-isopropylpiperazin-1-yl)(6-(4- phenethylpiperazine-1-carbonyl)pyridin-3- yl)methanone | 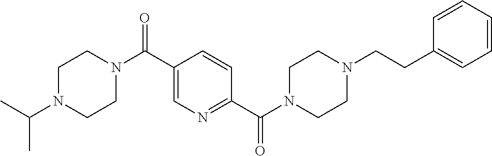 [SMILES: CC(C)N1CCN(C(=O)c2ccc(C(=O)N3CCN(CCc4ccccc4)CC3)nc2)CC1] |
| 92 | pyridine-2,5-diylbis((4-phenethylpiperazin- 1-yl)methanone) | 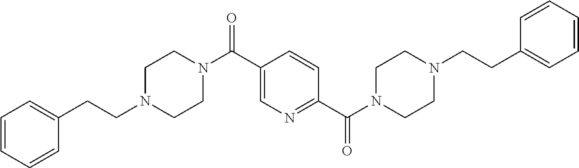 [SMILES: O=C(c1ccc(C(=O)N2CCN(CCc3ccccc3)CC2)nc1)N1CCN(CCc2ccccc2)CC1] |
| 93 | (4-(4-fluorobenzyl)piperazin-1-yl)(6-(4- phenethylpiperazine-1-carbonyl)pyridin-2- yl)methanone | 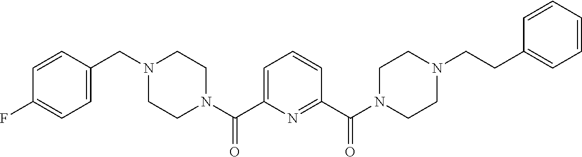 [SMILES: O=C(c1cccc(C(=O)N2CCN(Cc3ccc(F)cc3)CC2)n1)N1CCN(CCc2ccccc2)CC1] |
| 94 | (4-(4-fluorobenzyl)piperazin-1-yl)(6-(4- phenylpiperazine-1-carbonyl)pyridin-2- yl)methanone | 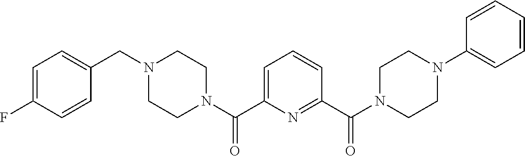 [SMILES: O=C(c1cccc(C(=O)N2CCN(c3ccccc3)CC2)n1)N1CCN(Cc2ccc(F)cc2)CC1] |
| 95 | N-(1-(4-cyanobenzyl)piperidin-4-yl)-5-(4- (cyclohexylmethyl)piperazine-1- carbonyl)picolinamide | 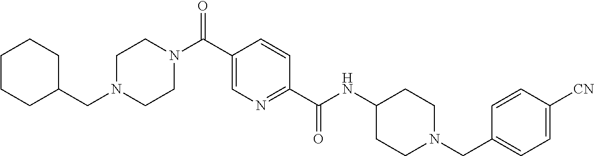 [SMILES: N#Cc1ccc(CN2CCC(NC(=O)c3ccc(C(=O)N4CCN(CC5CCCCC5)CC4)cn3)CC2)cc1] |
| 96 | N-(1-benzylpiperidin-4-yl)-5-(4-(pyridin-4- yl)piperazine-1-carbonyl)picolinamide | 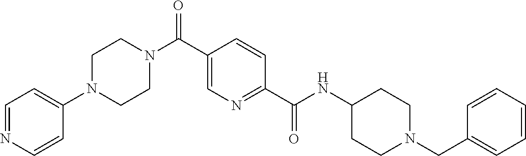 [SMILES: O=C(NC1CCN(Cc2ccccc2)CC1)c1ccc(C(=O)N2CCN(c3ccncc3)CC2)cn1] |
| 97 | N-(1-benzylpiperidin-4-yl)-5-(4- phenylpiperazine-1-carbonyl)picolinamide | 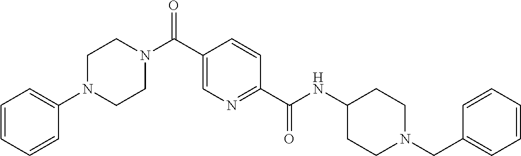 [SMILES: O=C(NC1CCN(Cc2ccccc2)CC1)c1ccc(C(=O)N2CCN(c3ccccc3)CC2)cn1] |
| 98 | ethyl 2-(4-(5-(4-(4-fluorobenzyl)piperazine- 1-carbonyl)picolinamido)piperidin-1-yl)-2- phenylacetate | 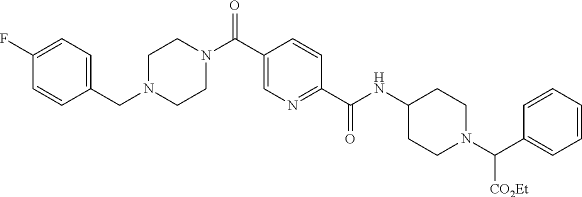 [SMILES: CCOC(=O)C(c1ccccc1)N1CCC(NC(=O)c2ccc(C(=O)N3CCN(Cc4ccc(F)cc4)CC3)cn2)CC1] |
| 99 | N-(4-(4-cyanobenzyl)cyclohexyl)-6-(4-(4- fluorobenzyl)piperazine-1- carbonyl)picolinamide | 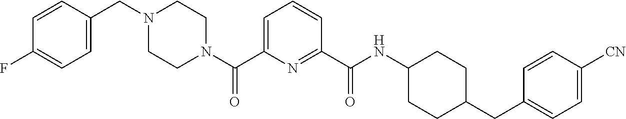 [SMILES: N#Cc1ccc(CC2CCC(NC(=O)c3cccc(C(=O)N4CCN(Cc5ccc(F)cc5)CC4)n3)CC2)cc1] |
| 100 | cis-1-(4-cyanobenzyl)-3-fluoropiperidin-4- yl)-5-(4-(4-fluorobenzyl)piperazine-1- carbonyl)picolinamide | 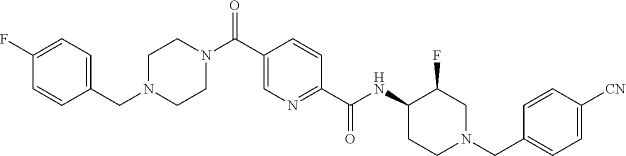 [SMILES: N#Cc1ccc(CN2CC[C@@H](NC(=O)c3ccc(C(=O)N4CCN(Cc5ccc(F)cc5)CC4)cn3)[C@@H](F)C2)cc1] |
| 101 | N-(1-(4-cyanobenzyl)piperidin-4-yl)-5-(4- (4-methoxybenzyl)piperazine-1- carbonyl)picolinamide | 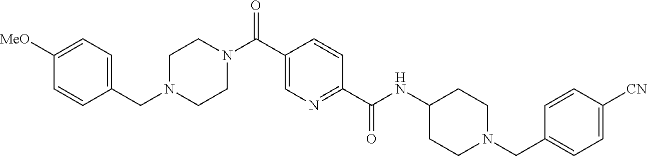 [SMILES: COc1ccc(CN2CCN(C(=O)c3ccc(C(=O)NC4CCN(Cc5ccc(C#N)cc5)CC4)nc3)CC2)cc1] |
| 102 | 5-(4-(4-fluorobenzyl)piperazine-1- carbonyl)-N-(cis-3-fluoropiperidin-4- yl)picolinamide | 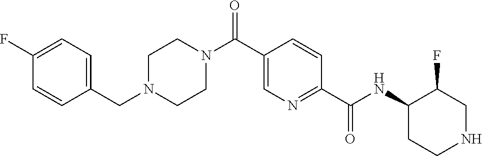 [SMILES: O=C(N[C@@H]1CCNC[C@@H]1F)c1ccc(C(=O)N2CCN(Cc3ccc(F)cc3)CC2)cn1] |
| 103 | N-(1-(4-cyanobenzyl)piperidin-4-yl)-5-(4- (pyridin-4-ylmethyl)piperazine-1- carbonyl)picolinamide | 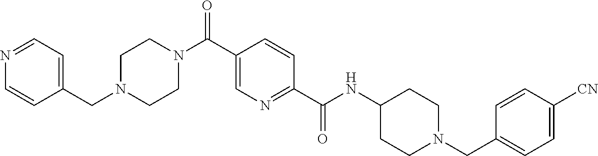 [SMILES: N#Cc1ccc(CN2CCC(NC(=O)c3ccc(C(=O)N4CCN(Cc5ccncc5)CC4)cn3)CC2)cc1] |
| 104 | N-(cis-3-fluoro-1-(pyridin-4- ylmethyl)piperidin-4-yl)-5-(4-(4- fluorobenzyl)piperazine-1- carbonyl)picolinamide | 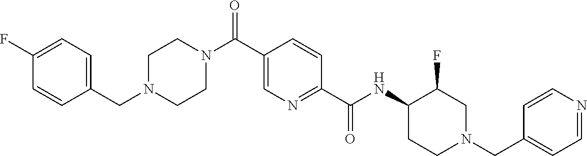 [SMILES: O=C(N[C@@H]1CCN(Cc2ccncc2)C[C@@H]1F)c1ccc(C(=O)N2CCN(Cc3ccc(F)cc3)CC2)cn1] |
| 105 | N2-(1-benzylpiperidin-4-yl)-N5-(biphenyl- 4-yl)pyridine-2,5-dicarboxamide | 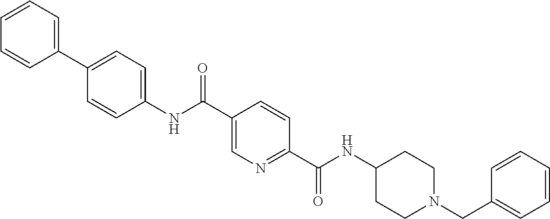 [SMILES: O=C(Nc1ccc(-c2ccccc2)cc1)c1ccc(C(=O)NC2CCN(Cc3ccccc3)CC2)nc1] |
| 106 | N2-(1-benzylpiperidin-4-yl)-N5-(biphenyl- 3-yl)pyridine-2,5-dicarboxamide | 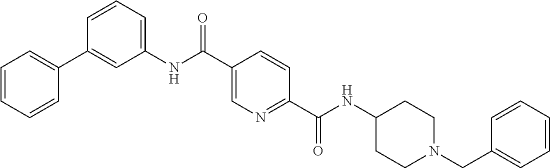 [SMILES: O=C(Nc1cccc(-c2ccccc2)c1)c1ccc(C(=O)NC2CCN(Cc3ccccc3)CC2)nc1] |
| 107 | 5-(4-(4-fluorobenzyl)piperazine-1- carbonyl)-N-phenylpicolinamide | 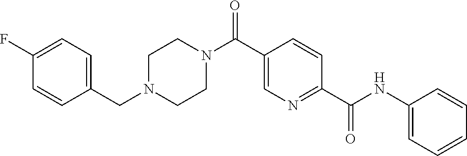 [SMILES: O=C(Nc1ccccc1)c1ccc(C(=O)N2CCN(Cc3ccc(F)cc3)CC2)cn1] |
| 108 | 5-(4-benzylphenylamino)-N-(1-(4- cyanobenzyl)piperidin-4-yl)picolinamide | 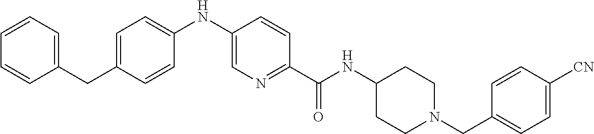 [SMILES: N#Cc1ccc(CN2CCC(NC(=O)c3ccc(Nc4ccc(Cc5ccccc5)cc4)cn3)CC2)cc1] |
| 109 | 5-(biphenyl-4-ylamino)-N-(1-(4- cyanobenzyl)piperidin-4-yl)picolinamide | 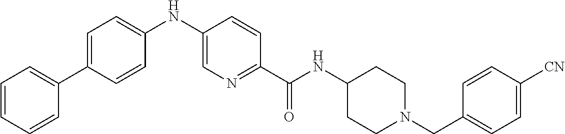 [SMILES: N#Cc1ccc(CN2CCC(NC(=O)c3ccc(Nc4ccc(-c5ccccc5)cc4)cn3)CC2)cc1] |
| 110 | 5-(4-benzylpiperazin-1-yl)-N-(1-(4- cyanobenzyl)piperidin-4-yl)picolinamide | 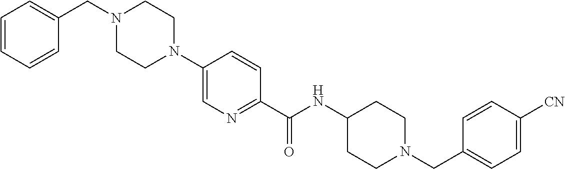 [SMILES: N#Cc1ccc(CN2CCC(NC(=O)c3ccc(N4CCN(Cc5ccccc5)CC4)cn3)CC2)cc1] |
| 111 | N-(1-(2-(4-cyanophenyl)propan-2- yl)piperidin-4-yl)-5-(4-(4- fluorobenzyl)piperazine-1- carbonyl)picolinamide | 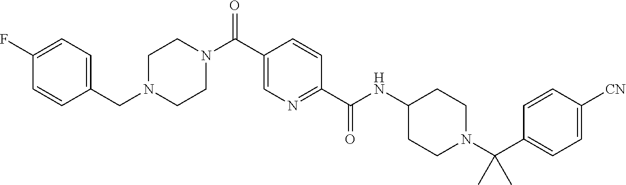 [SMILES: CC(C)(c1ccc(C#N)cc1)N1CCC(NC(=O)c2ccc(C(=O)N3CCN(Cc4ccc(F)cc4)CC3)cn2)CC1] |
| 112 | N-(1-benzylpiperidin-4-yl)-5-(3- phenoxyphenylamino)picolinamide | 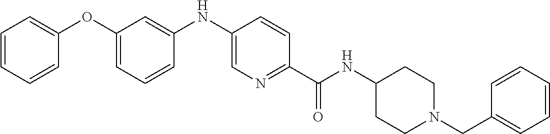 [SMILES: O=C(NC1CCN(Cc2ccccc2)CC1)c1ccc(Nc2cccc(Oc3ccccc3)c2)cn1] |
| 113 | N-(1-benzylpiperidin-4-yl)-5-(4- phenoxyphenylamino)picolinamide | 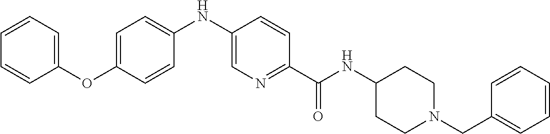 [SMILES: O=C(NC1CCN(Cc2ccccc2)CC1)c1ccc(Nc2ccc(Oc3ccccc3)cc2)cn1] |
| 114 | N-(1-benzylpiperidin-4-yl)-5-(biphenyl-3- ylamino)picolinamide | 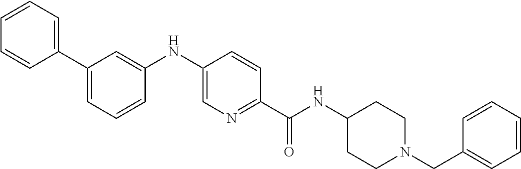 [SMILES: O=C(NC1CCN(Cc2ccccc2)CC1)c1ccc(Nc2cccc(-c3ccccc3)c2)cn1] |
| 115 | N-benzyl-5-(4-(4-fluorobenzyl)piperazine- 1-carbonyl)picolinamide | 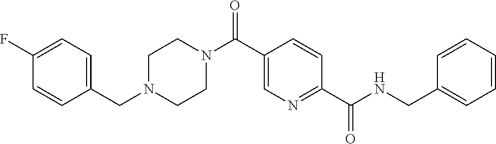 [SMILES: O=C(NCc1ccccc1)c1ccc(C(=O)N2CCN(Cc3ccc(F)cc3)CC2)cn1] |
| 116 | N-benzyl-5-(4-benzylpiperazine-1- carbonyl)picolinamide | 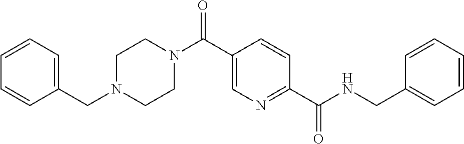 [SMILES: O=C(NCc1ccccc1)c1ccc(C(=O)N2CCN(Cc3ccccc3)CC2)cn1] |
| 117 | 5-(4-(4-fluorobenzyl)piperazine-1- carbonyl)-N-(1-(4- methoxybenzyl)piperidin-4-yl)picolinamide | 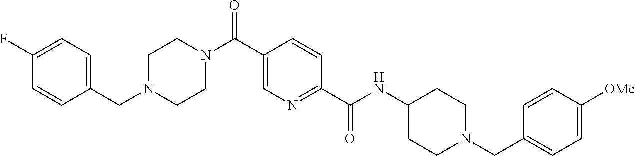 [SMILES: COc1ccc(CN2CCC(NC(=O)c3ccc(C(=O)N4CCN(Cc5ccc(F)cc5)CC4)cn3)CC2)cc1] |
| 118 | (R)-N-(1-(4-cyanobenzyl)pyrrolidin-3-yl)-5- (4-(4-fluorobenzyl)piperazine-1- carbonyl)picolinamide | 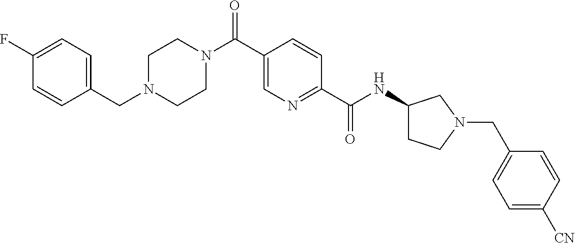 [SMILES: N#Cc1ccc(CN2CC[C@@H](NC(=O)c3ccc(C(=O)N4CCN(Cc5ccc(F)cc5)CC4)cn3)C2)cc1] |
| 119 | N-(1-benzylpiperidin-4-yl)-5-(4′- cyanobiphenyl-4-ylamino)picolinamide | 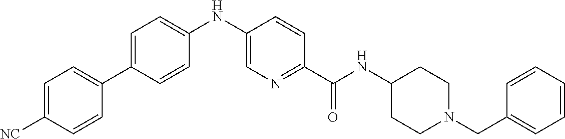 [SMILES: N#Cc1ccc(-c2ccc(Nc3ccc(C(=O)NC4CCN(Cc5ccccc5)CC4)nc3)cc2)cc1] |
| 120 | N-(1-benzylpiperidin-4-yl)-5-(4′- methoxybiphenyl-4-ylamino)picolinamide | 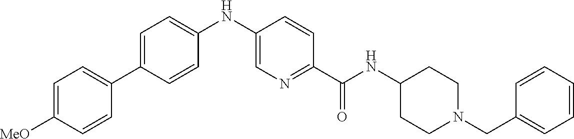 [SMILES: COc1ccc(-c2ccc(Nc3ccc(C(=O)NC4CCN(Cc5ccccc5)CC4)nc3)cc2)cc1] |
| 121 | 5-(1-benzyl-1H-1,2,3-triazol-4-yl)-N-(1-(4- cyanobenzyl)piperidin-4-yl)picolinamide | 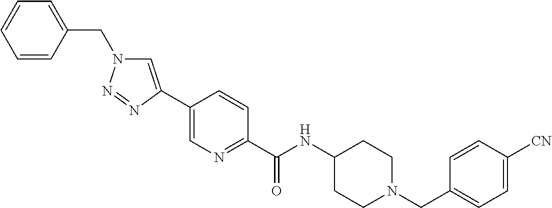 [SMILES: N#Cc1ccc(CN2CCC(NC(=O)c3ccc(-c4cn(Cc5ccccc5)nn4)cn3)CC2)cc1] |
| 122 | 5-(1-benzyl-1H-1,2,3-triazol-4-yl)-N-(1-(4- cyanobenzyl)piperidin-4-yl)picolinamide | 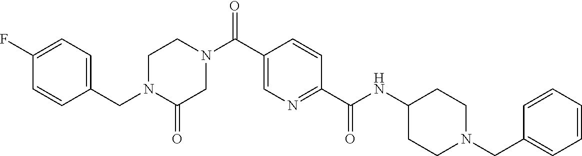 [SMILES: O=C(NC1CCN(Cc2ccccc2)CC1)c1ccc(C(=O)N2CCN(Cc3ccc(F)cc3)C(=O)C2)cn1] |
| 123 | (S)-N-(1-(4-cyanobenzyl)pyrrolidin-3-yl)-5- (4-(4-fluorobenzyl)piperazine-1- carbonyl)picolinamide | 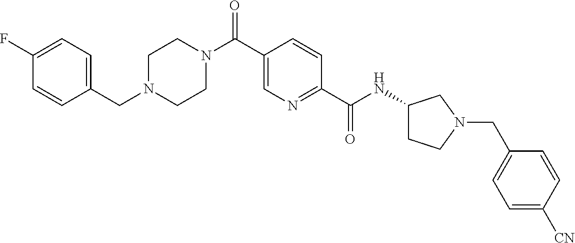 [SMILES: N#Cc1ccc(CN2CC[C@H](NC(=O)c3ccc(C(=O)N4CCN(Cc5ccc(F)cc5)CC4)cn3)C2)cc1] |
| 124 | 5-(4-benzylpiperidine-1-carbonyl)-N-(1-(4- cyanobenzyl)piperidin-4-yl)picolinamide | 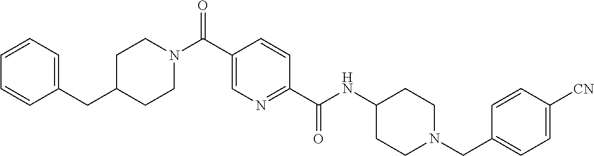 [SMILES: N#Cc1ccc(CN2CCC(NC(=O)c3ccc(C(=O)N4CCC(Cc5ccccc5)CC4)cn3)CC2)cc1] |
| 125 | N-(1-(4-cyanobenzyl)piperidin-4-yl)-5-(4- (4-fluorobenzyl)-3,3-dimethylpiperazine-1- carbonyl)picolinamide | 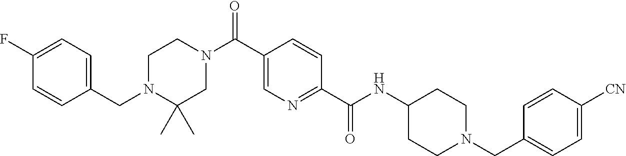 [SMILES: CC1(C)CN(C(=O)c2ccc(C(=O)NC3CCN(Cc4ccc(C#N)cc4)CC3)nc2)CCN1Cc1ccc(F)cc1] |
| 126 | N-(1-(4-cyanobenzyl)piperidin-4-yl)-5-(1- phenylpiperidin-4-ylamino)picolinamide | 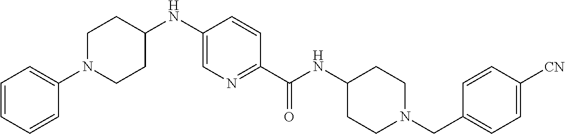 [SMILES: N#Cc1ccc(CN2CCC(NC(=O)c3ccc(NC4CCN(c5ccccc5)CC4)cn3)CC2)cc1] |
| 127 | N-(cis-1-(4-chlorobenzyl)-3- fluoropiperidin-4-yl)-5-(4-(4- fluorobenzyl)piperazine-1- carbonyl)picolinamide | 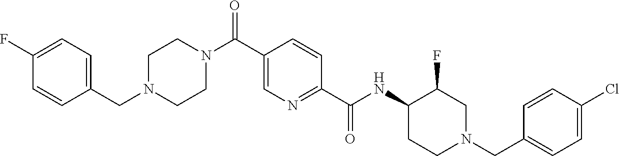 [SMILES: O=C(N[C@@H]1CCN(Cc2ccc(Cl)cc2)C[C@@H]1F)c1ccc(C(=O)N2CCN(Cc3ccc(F)cc3)CC2)cn1] |
| 128 | N-(1-(4-cyanobenzyl)piperidin-4-yl)-5-(4- (4-cyanobenzyl)piperidine-1- carbonyl)picolinamide | 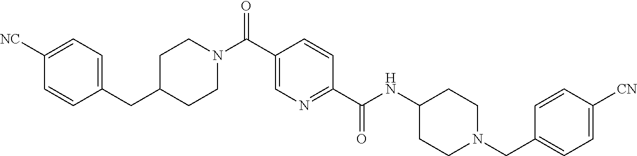 [SMILES: N#Cc1ccc(CC2CCN(C(=O)c3ccc(C(=O)NC4CCN(Cc5ccc(C#N)cc5)CC4)nc3)CC2)cc1] |
| 129 | N-(1-(4-cyanobenzyl)piperidin-4-yl)-5-(4- (4-methoxybenzoyl)piperidine-1- carbonyl)picolinamide | 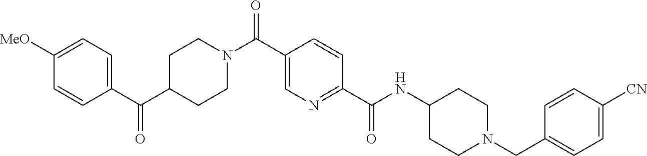 [SMILES: COc1ccc(C(=O)C2CCN(C(=O)c3ccc(C(=O)NC4CCN(Cc5ccc(C#N)cc5)CC4)nc3)CC2)cc1] |
| 130 | N-(1-(4-cyanobenzyl)piperidin-4-yl)-5-(4- (4-fluorobenzyl)piperidine-1- carbonyl)picolinamide | 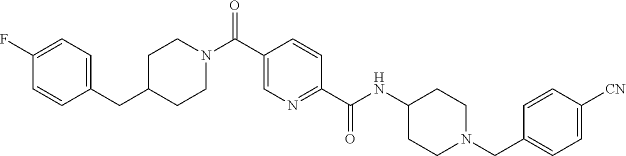 [SMILES: N#Cc1ccc(CN2CCC(NC(=O)c3ccc(C(=O)N4CCC(Cc5ccc(F)cc5)CC4)cn3)CC2)cc1] |
| 131 | N-(1-(4-cyanobenzyl)piperidin-4-yl)-5-(4- (4-methoxybenzyl)piperidine-1- carbonyl)picolinamide | 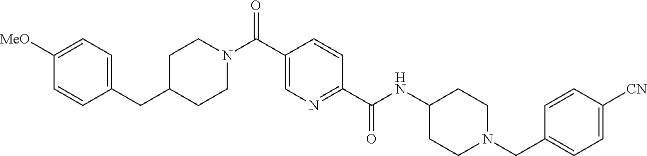 [SMILES: COc1ccc(CC2CCN(C(=O)c3ccc(C(=O)NC4CCN(Cc5ccc(C#N)cc5)CC4)nc3)CC2)cc1] |
| 132 | N-(2-(4-cyanobenzyl)-1,2,3,4- tetrahydroisoquinolin-7-yl)-5-(4-(4- fluorobenzyl)piperazine-1- carbonyl)picolinamide | 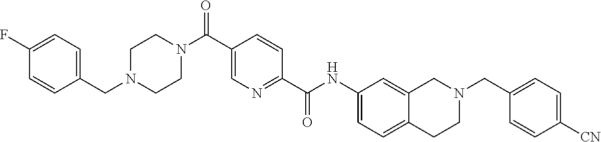 [SMILES: N#Cc1ccc(CN2CCc3ccc(NC(=O)c4ccc(C(=O)N5CCN(Cc6ccc(F)cc6)CC5)cn4)cc3C2)cc1] |
| 133 | N-(1-(4-cyanobenzyl)piperidin-4-yl)-5-(4- (2-phenylpropan-2-yl)piperazine-1- carbonyl)picolinamide | 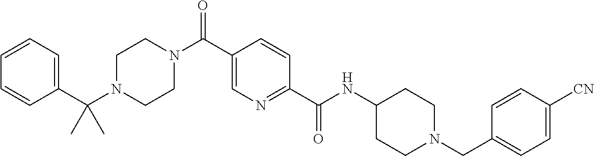 [SMILES: CC(C)(c1ccccc1)N1CCN(C(=O)c2ccc(C(=O)NC3CCN(Cc4ccc(C#N)cc4)CC3)nc2)CC1] |
| 134 | 5-(4-(4-chlorophenyl)piperidine-1- carbonyl)-N-(1-(4-cyanobenzyl)piperidin- 4-yl)picolinamide | 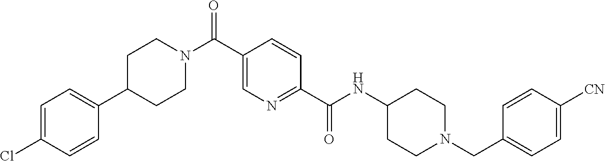 [SMILES: N#Cc1ccc(CN2CCC(NC(=O)c3ccc(C(=O)N4CCC(c5ccc(Cl)cc5)CC4)cn3)CC2)cc1] |
| 135 | N-(1-(4-cyanobenzyl)piperidin-4-yl)-5-(4- (4-cyanophenoxy)piperidine-1- carbonyl)picolinamide | 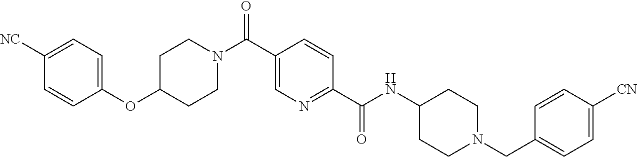 [SMILES: N#Cc1ccc(CN2CCC(NC(=O)c3ccc(C(=O)N4CCC(Oc5ccc(C#N)cc5)CC4)cn3)CC2)cc1] |
| 136 | N-(1-(4-cyanobenzyl)piperidin-4-yl)-5-(4- (4-fluorobenzoyl)piperidine-1- carbonyl)picolinamide | 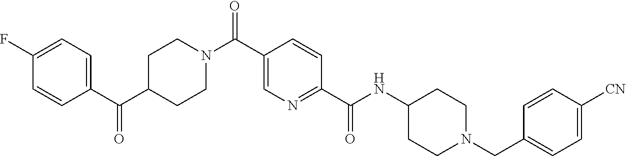 [SMILES: N#Cc1ccc(CN2CCC(NC(=O)c3ccc(C(=O)N4CCC(C(=O)c5ccc(F)cc5)CC4)cn3)CC2)cc1] |
| 137 | N-(1-(4-cyanobenzyl)piperidin-4-yl)-5-(3- (4-cyanophenoxy)piperidine-1- carbonyl)picolinamide | 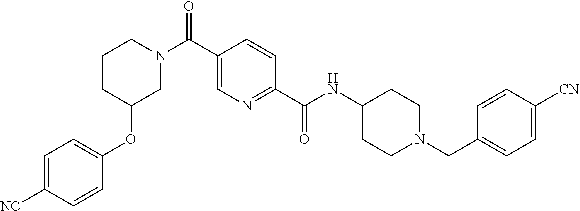 [SMILES: N#Cc1ccc(CN2CCC(NC(=O)c3ccc(C(=O)N4CCCC(Oc5ccc(C#N)cc5)C4)cn3)CC2)cc1] |
| 138 | N-(1-(4-cyanobenzyl)piperidin-4-yl)-5-(4- (fluoro(4-fluorophenyl)methyl)piperidine- 1-carbonyl)picolinamide | 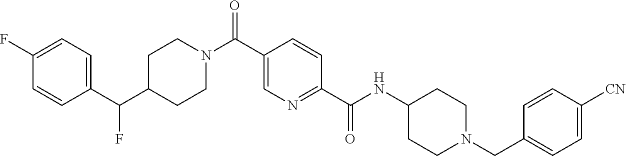 [SMILES: N#Cc1ccc(CN2CCC(NC(=O)c3ccc(C(=O)N4CCC(C(F)c5ccc(F)cc5)CC4)cn3)CC2)cc1] |
| 139 | 5-(1-(4-chlorophenyl)piperidin-4-ylamino)- N-(1-(4-cyanobenzyl)piperidin-4- yl)picolinamide | 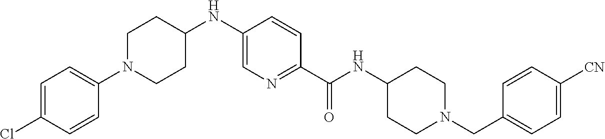 [SMILES: N#Cc1ccc(CN2CCC(NC(=O)c3ccc(NC4CCN(c5ccc(Cl)cc5)CC4)cn3)CC2)cc1] |
| 140 | N-(1-(4-cyanobenzyl)piperidin-4-yl)-5-(4- (3,5-difluorobenzyl)piperazine-1- carbonyl)picolinamide | 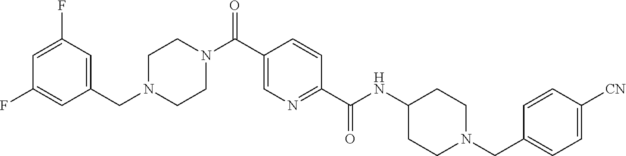 [SMILES: N#Cc1ccc(CN2CCC(NC(=O)c3ccc(C(=O)N4CCN(Cc5cc(F)cc(F)c5)CC4)cn3)CC2)cc1] |
| 141 | 5-(4-(4-carbamoylbenzyl)piperidine-1- carbonyl)-N-(1-(4-cyanobenzyl)piperidin-4- yl)picolinamide | 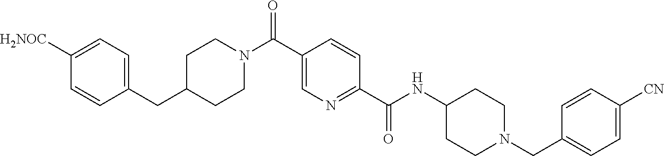 [SMILES: N#Cc1ccc(CN2CCC(NC(=O)c3ccc(C(=O)N4CCC(Cc5ccc(C(N)=O)cc5)CC4)cn3)CC2)cc1] |
| 142 | N-(1-(4-cyanobenzyl)piperidin-4-yl)-5- (4-((4-fluorophenyl)(hydroxy)methyl) piperidine-1-carbonyl)picolinamide | 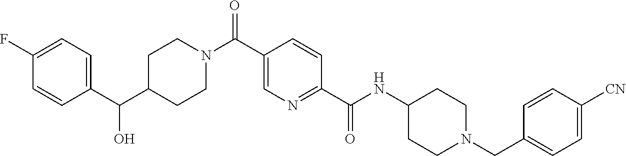 [SMILES: N#Cc1ccc(CN2CCC(NC(=O)c3ccc(C(=O)N4CCC(C(O)c5ccc(F)cc5)CC4)cn3)CC2)cc1] |
| 143 | N-(1-(4-cyanobenzyl)piperidin-4-yl)-5-(4- (4-methoxyphenoxy)piperidine-1- carbonyl)picolinamide | 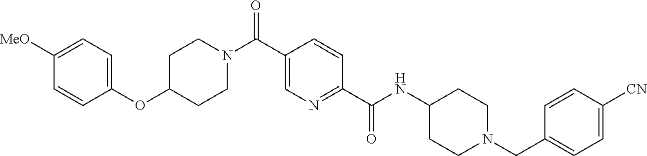 [SMILES: COc1ccc(OC2CCN(C(=O)c3ccc(C(=O)NC4CCN(Cc5ccc(C#N)cc5)CC4)nc3)CC2)cc1] |
| 144 | N2-(2-(4-cyanobenzyl)-1,2,3,4- tetrahydroisoquinolin-7-yl)-N5-(4- fluorobenzyl)pyridine-2,5-dicarboxamide | 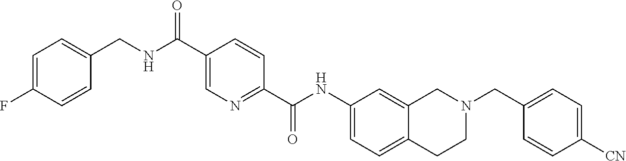 [SMILES: N#Cc1ccc(CN2CCc3ccc(NC(=O)c4ccc(C(=O)NCc5ccc(F)cc5)cn4)cc3C2)cc1] |
| 145 | N-(1-(4-cyanobenzyl)piperidin-4-yl)-5-(4- (4-methylbenzyl)piperidine-1- carbonyl)picolinamide | 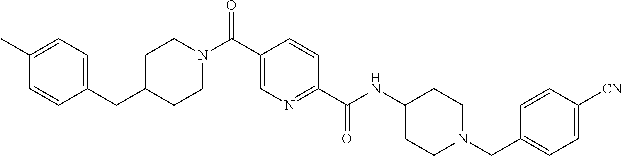 [SMILES: Cc1ccc(CC2CCN(C(=O)c3ccc(C(=O)NC4CCN(Cc5ccc(C#N)cc5)CC4)nc3)CC2)cc1] |
| 146 | N-(1-(4-cyanobenzyl)piperidin-4-yl)-5-(4- (3-fluoro-4-methoxybenzyl)piperidine-1- carbonyl)picolinamide | 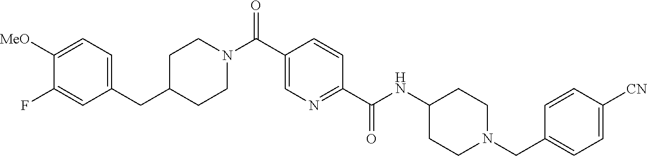 [SMILES: COc1ccc(CC2CCN(C(=O)c3ccc(C(=O)NC4CCN(Cc5ccc(C#N)cc5)CC4)nc3)CC2)cc1F] |
| 147 | N-(1-(4-cyanobenzyl)piperidin-4-yl)-5-(4- (3-methoxybenzyl)piperidine-1- carbonyl)picolinamide | 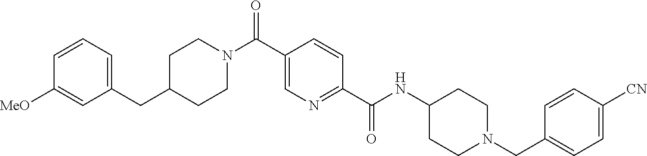 [SMILES: COc1cccc(CC2CCN(C(=O)c3ccc(C(=O)NC4CCN(Cc5ccc(C#N)cc5)CC4)nc3)CC2)c1] |
| 148 | N-(1-(4-cyanobenzyl)piperidin-4-yl)-5-(4- (4-fluorophenoxy)piperidine-1- carbonyl)picolinamide | 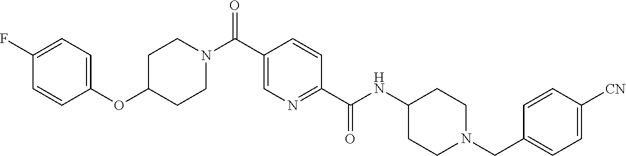 [SMILES: N#Cc1ccc(CN2CCC(NC(=O)c3ccc(C(=O)N4CCC(Oc5ccc(F)cc5)CC4)cn3)CC2)cc1] |
| 149 | N2-(1-(4-cyanobenzyl)piperidin-4-yl)-N5- (2-(4-fluorophenoxy)ethyl)pyridine-2,5- dicarboxamide | 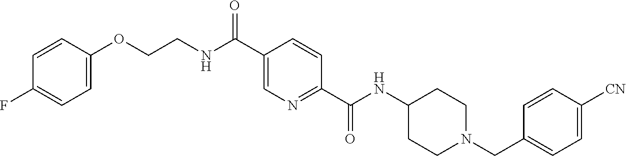 [SMILES: N#Cc1ccc(CN2CCC(NC(=O)c3ccc(C(=O)NCCOc4ccc(F)cc4)cn3)CC2)cc1] |
| 150 | N-(cis-4-(4-cyanophenoxy)cyclohexyl)-5- (4-(4-fluorobenzyl)piperazine-1- carbonyl)picolinamide | 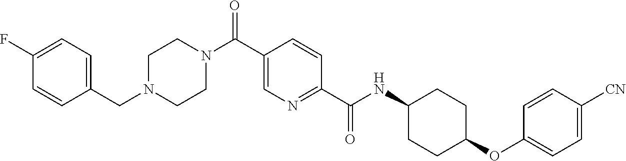 [SMILES: N#Cc1ccc(O[C@H]2CC[C@@H](NC(=O)c3ccc(C(=O)N4CCN(Cc5ccc(F)cc5)CC4)cn3)CC2)cc1] |
| 151 | N-(trans-4-(4-cyanophenoxy)cyclohexyl)-5- (4-(4-fluorobenzoyl)piperazine-1- carbonyl)picolinamide | 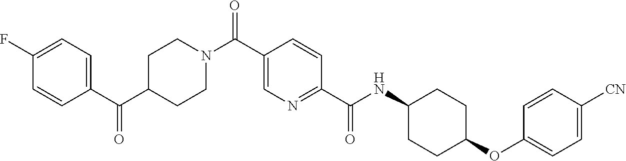 [SMILES: N#Cc1ccc(O[C@H]2CC[C@@H](NC(=O)c3ccc(C(=O)N4CCC(C(=O)c5ccc(F)cc5)CC4)cn3)CC2)cc1] |
| 152 | N-(1-(4-cyanobenzyl)piperidin-4-yl)-5-(3- (4-fluorobenzyl)piperidine-1- carbonyl)picolinamide | 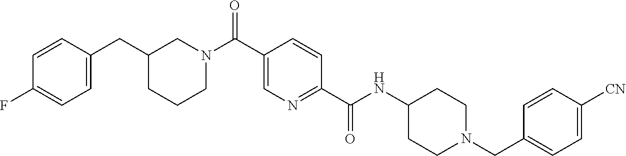 [SMILES: N#Cc1ccc(CN2CCC(NC(=O)c3ccc(C(=O)N4CCCC(Cc5ccc(F)cc5)C4)cn3)CC2)cc1] |
| 153 | N-(1-(4-cyanobenzyl)piperidin-4-yl)-5-(2- (4-fluorobenzyl)piperidine-1- carbonyl)picolinamide | 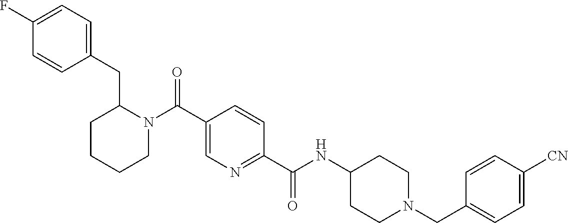 [SMILES: N#Cc1ccc(CN2CCC(NC(=O)c3ccc(C(=O)N4CCCCC4Cc4ccc(F)cc4)cn3)CC2)cc1] |
| 154 | 5-(4-(4-chlorobenzoyl)piperidine-1- carbonyl)-N-(1-(4-cyanobenzyl)piperidin-4- yl)picolinamide | 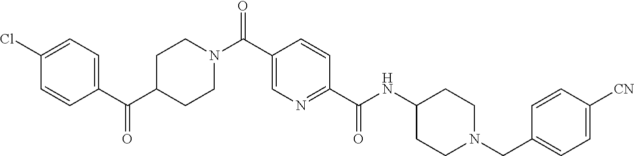 [SMILES: N#Cc1ccc(CN2CCC(NC(=O)c3ccc(C(=O)N4CCC(C(=O)c5ccc(Cl)cc5)CC4)cn3)CC2)cc1] |
| 155 | N-(1-(4-cyanobenzyl)piperidin-4-yl)-5-(4- (3-cyanophenoxy)piperidine-1- carbonyl)picolinamide | 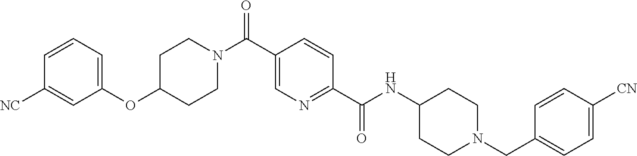 [SMILES: N#Cc1ccc(CN2CCC(NC(=O)c3ccc(C(=O)N4CCC(Oc5cccc(C#N)c5)CC4)cn3)CC2)cc1] |
| 156 | 5-(4-(3-chloro-4-cyanophenoxy)piperidine- 1-carbonyl)-N-(1-(4-cyanobenzyl)piperidin- 4-yl)picolinamide | 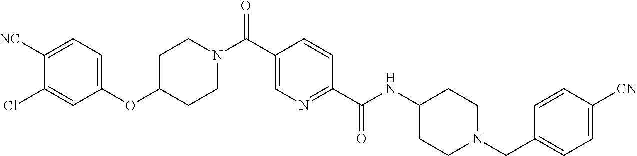 [SMILES: N#Cc1ccc(CN2CCC(NC(=O)c3ccc(C(=O)N4CCC(Oc5ccc(C#N)c(Cl)c5)CC4)cn3)CC2)cc1] |
| 157 | N-(1-(4-cyanobenzyl)piperidin-4-yl)-5-(4- (4-(trifluoromethyl)phenoxy)piperidine-1- carbonyl)picolinamide | 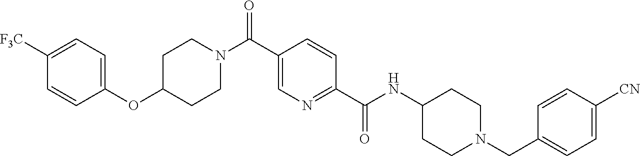 [SMILES: N#Cc1ccc(CN2CCC(NC(=O)c3ccc(C(=O)N4CCC(Oc5ccc(C(F)(F)F)cc5)CC4)cn3)CC2)cc1] |
| 158 | N-(1-(4-cyanobenzyl)piperidin-4-yl)-5-(4- (3,4-difluorophenoxy)piperidine-1- carbonyl)picolinamide | 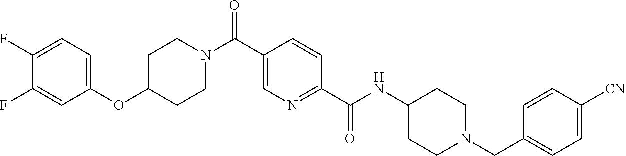 [SMILES: N#Cc1ccc(CN2CCC(NC(=O)c3ccc(C(=O)N4CCC(Oc5ccc(F)c(F)c5)CC4)cn3)CC2)cc1] |
| 159 | N-(1-(4-cyanobenzyl)piperidin-4-yl)-3- (5,20-dioxo-24-((3aS,4S,6aR)-2- oxohexahydro-1H-thieno[3,4-d]imidazol-4- yl)-7,10,13,16-tetraoxa-4,19-diazatetracos- 1-ynyl)-5-(4-(4-fluorobenzyl)piperazine-1- carbonyl)picolinamide | 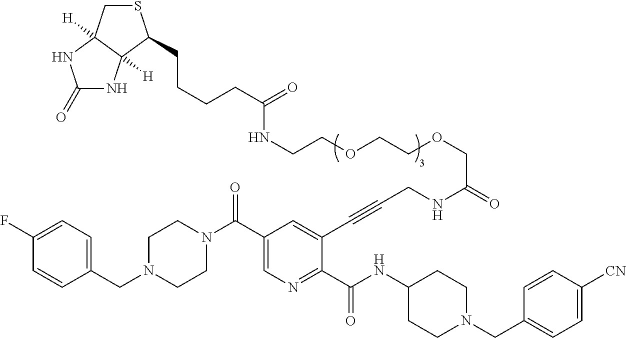 |
| 160 | 5-(4-(4-fluorobenzoyl)piperidine-1- carbonyl)-N-(1-(4- methoxybenzyl)piperidin-4-yl)picolinamide | 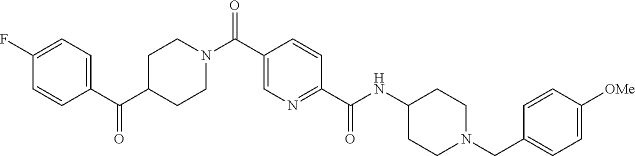 [SMILES: COc1ccc(CN2CCC(NC(=O)c3ccc(C(=O)N4CCC(C(=O)c5ccc(F)cc5)CC4)cn3)CC2)cc1] |
| 161 | 5-(4-(4-fluorophenoxy)piperidine-1- carbonyl)-N-(1-(4- methoxybenzyl)piperidin-4-yl)picolinamide | 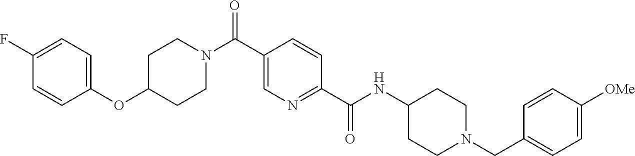 [SMILES: COc1ccc(CN2CCC(NC(=O)c3ccc(C(=O)N4CCC(Oc5ccc(F)cc5)CC4)cn3)CC2)cc1] |
| 162 | 5-(4-(4-cyanophenoxy)piperidine-1- carbonyl)-N-(1-(4- methoxybenzyl)piperidin-4-yl)picolinamide | 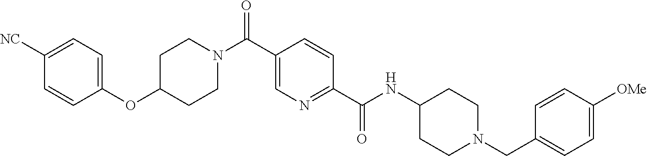 [SMILES: COc1ccc(CN2CCC(NC(=O)c3ccc(C(=O)N4CCC(Oc5ccc(C#N)cc5)CC4)cn3)CC2)cc1] |
| 163 | 5-(4-(4-methoxybenzoyl)piperidine-1- carbonyl)-N-(1-(4- methoxybenzyl)piperidin-4-yl)picolinamide | 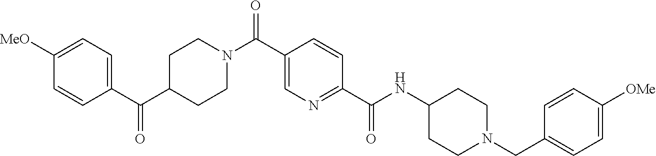 [SMILES: COc1ccc(CN2CCC(NC(=O)c3ccc(C(=O)N4CCC(C(=O)c5ccc(OC)cc5)CC4)cn3)CC2)cc1] |
| 164 | tert-butyl 3-(2-(1-(4-cyanobenzyl)piperidin- 4-ylcarbamoyl)-5-(4-(4- fluorobenzyl)piperazine-1-carbonyl)pyridin- 3-yl)prop-2-ynylcarbamate | 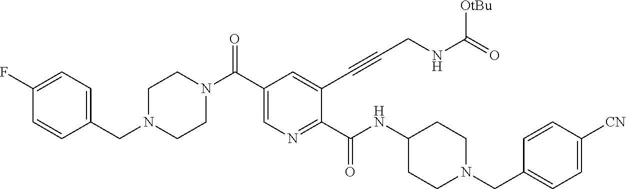 [SMILES: CC(C)(C)OC(=O)NCC#Cc1cc(C(=O)N2CCN(Cc3ccc(F)cc3)CC2)cnc1C(=O)NC1CCN(Cc2ccc(C#N)cc2)CC1] |
| 165 | N-(1-(4-cyanobenzyl)piperidin-4-yl)-5-(4- (4-cyanophenoxy)piperidin-1- yl)picolinamide | 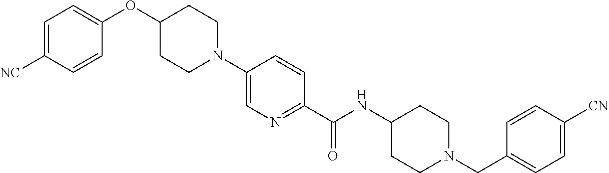 [SMILES: N#Cc1ccc(CN2CCC(NC(=O)c3ccc(N4CCC(Oc5ccc(C#N)cc5)CC4)cn3)CC2)cc1] |
| 166 | N2-(1-(4-cyanobenzyl)piperidin-4-yl)-N5- (1-(4-cyanophenyl)piperidin-4-yl)pyridine- 2,5-dicarboxamide | 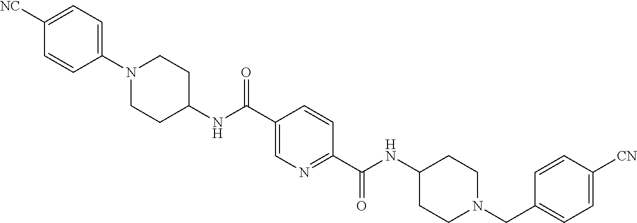 [SMILES: N#Cc1ccc(CN2CCC(NC(=O)c3ccc(C(=O)NC4CCN(c5ccc(C#N)cc5)CC4)cn3)CC2)cc1] |
| 167 | N-((cis)-4-(4-cyanophenoxy)cyclohexyl)-5- (4-(4-fluorophenoxy)piperidine-1- carbonyl)picolinamide | 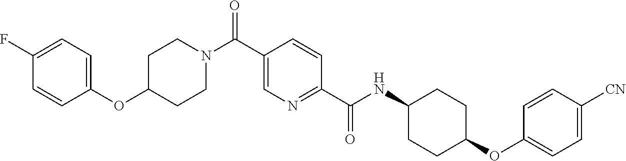 [SMILES: N#Cc1ccc(O[C@H]2CC[C@@H](NC(=O)c3ccc(C(=O)N4CCC(Oc5ccc(F)cc5)CC4)cn3)CC2)cc1] |
| 168 | N-((trans)-4-(4-cyanophenoxy)cyclohexyl)- 5-(4-(4-fluorobenzoyl)piperidine-1- carbonyl)picolinamide | 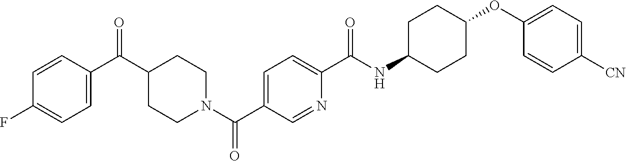 [SMILES: N#Cc1ccc(O[C@H]2CC[C@H](NC(=O)c3ccc(C(=O)N4CCC(C(=O)c5ccc(F)cc5)CC4)cn3)CC2)cc1] |
| 169 | N-((trans)-4-(4-cyanophenoxy)cyclohexyl)- 5-(4-(4-fluorophenoxy)piperidine-1- carbonyl)picolinamide | 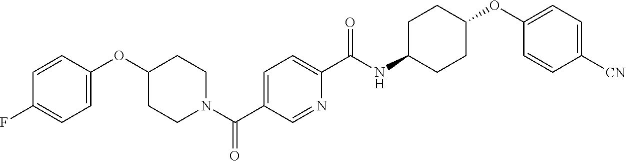 [SMILES: N#Cc1ccc(O[C@H]2CC[C@H](NC(=O)c3ccc(C(=O)N4CCC(Oc5ccc(F)cc5)CC4)cn3)CC2)cc1] |
| 170 | N-(5-(4-(4-fluorobenzyl)piperazine-1- carbonyl)pyridin-2-yl)biphenyl-4- carboxamide | 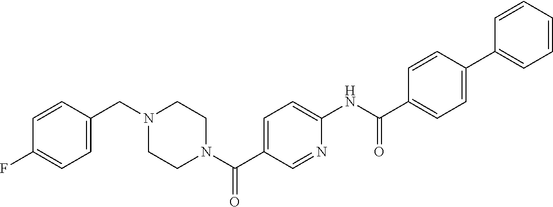 [SMILES: O=C(Nc1ccc(C(=O)N2CCN(Cc3ccc(F)cc3)CC2)cn1)c1ccc(-c2ccccc2)cc1] |
| 171 | N-((cis)-4-(4-cyanophenoxy)cyclohexyl)-5- (4-(4-methoxybenzoyl)piperidine-1- carbonyl)picolinamide | 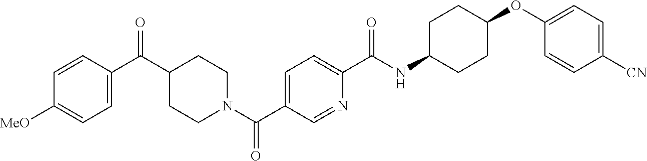 [SMILES: COc1ccc(C(=O)C2CCN(C(=O)c3ccc(C(=O)N[C@H]4CC[C@@H](Oc5ccc(C#N)cc5)CC4)nc3)CC2)cc1] |
| 172 | N-((trans)-4-(4-cyanophenoxy)cyclohexyl)- 5-(4-(4-methoxyphenoxy)piperidine-1- carbonyl)picolinamide | 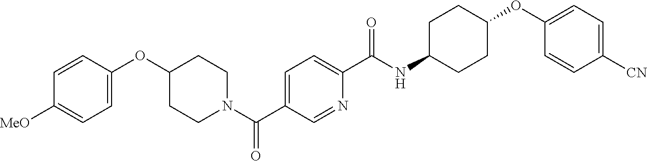 [SMILES: COc1ccc(OC2CCN(C(=O)c3ccc(C(=O)N[C@H]4CC[C@H](Oc5ccc(C#N)cc5)CC4)nc3)CC2)cc1] |
| 173 | 1-(4-cyanobenzyl)-N-(5-(4-(4- fluorophenoxy)piperidine-1- carbonyl)pyridin-2-yl)piperidine-4- carboxamide | 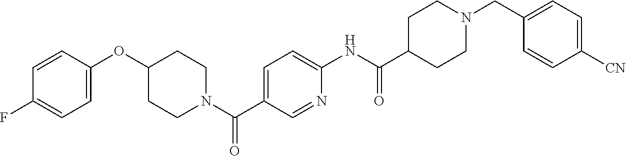 [SMILES: N#Cc1ccc(CN2CCC(C(=O)Nc3ccc(C(=O)N4CCC(Oc5ccc(F)cc5)CC4)cn3)CC2)cc1] |
| 174 | N-((cis)-4-(4-cyanophenoxy)cyclohexyl)-5- (4-(4-methoxyphenoxy)piperidine-1- carbonyl)picolinamide | 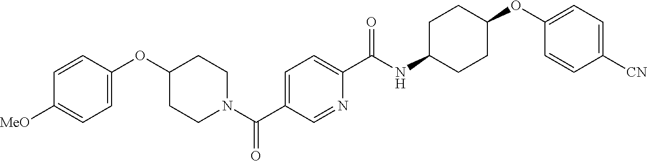 [SMILES: COc1ccc(OC2CCN(C(=O)c3ccc(C(=O)N[C@H]4CC[C@@H](Oc5ccc(C#N)cc5)CC4)nc3)CC2)cc1] |
| 175 | 1-(4-cyanobenzyl)-N-(5-(4-(4- fluorobenzoyl)piperidine-1- carbonyl)pyridin-2-yl)piperidine-4- carboxamide | 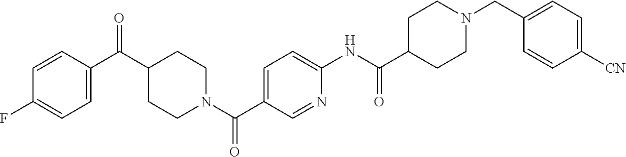 [SMILES: N#Cc1ccc(CN2CCC(C(=O)Nc3ccc(C(=O)N4CCC(C(=O)c5ccc(F)cc5)CC4)cn3)CC2)cc1] |
| 176 | N-((cis)-4-(4-cyanophenoxy)cyclohexyl)-5- ((S)-3-(4-fluorophenoxy)pyrrolidine-1- carbonyl)picolinamide | 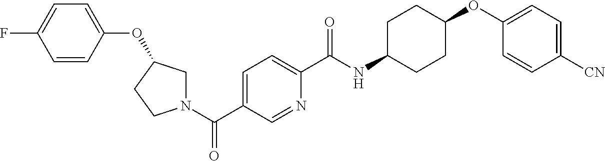 [SMILES: N#Cc1ccc(O[C@H]2CC[C@@H](NC(=O)c3ccc(C(=O)N4CC[C@H](Oc5ccc(F)cc5)C4)cn3)CC2)cc1] |
| 177 | N-(5-(4-(4-fluorobenzyl)piperazine-1- carbonyl)pyridin-2-yl)-6-(4- fluorophenoxy)nicotinamide | 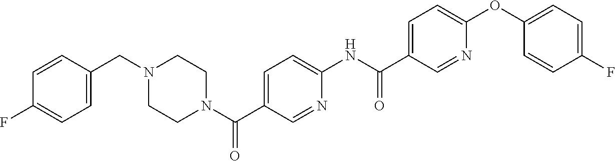 [SMILES: O=C(Nc1ccc(C(=O)N2CCN(Cc3ccc(F)cc3)CC2)cn1)c1ccc(Oc2ccc(F)cc2)nc1] |
| 178 | N-(1-(4-fluorobenzyl)piperidin-4-yl)-5-(4- (4-methoxybenzoyl)piperidine-1- carbonyl)picolinamide | 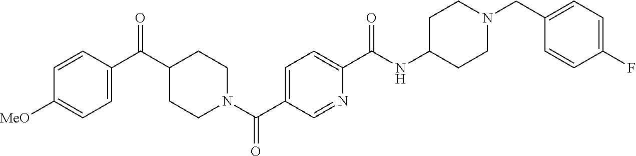 [SMILES: COc1ccc(C(=O)C2CCN(C(=O)c3ccc(C(=O)NC4CCN(Cc5ccc(F)cc5)CC4)nc3)CC2)cc1] |
| 179 | N-(1-(4-fluorobenzyl)piperidin-4-yl)-5-(4- (4-(trifluoromethyl)phenoxy)piperidine-1- carbonyl)picolinamide | 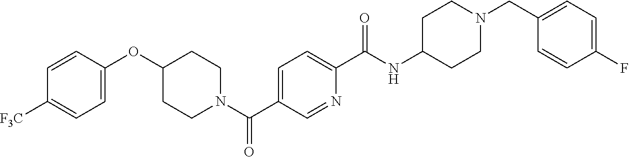 [SMILES: O=C(NC1CCN(Cc2ccc(F)cc2)CC1)c1ccc(C(=O)N2CCC(Oc3ccc(C(F)(F)F)cc3)CC2)cn1] |
| 180 | N-(1-(4-fluorobenzyl)piperidin-4-yl)-5-(4- (4-fluorophenoxy)piperidine-1- carbonyl)picolinamide | 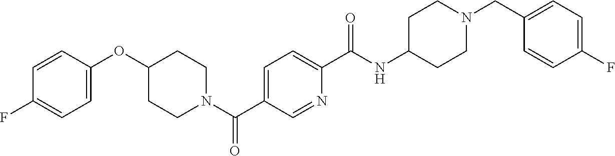 [SMILES: O=C(NC1CCN(Cc2ccc(F)cc2)CC1)c1ccc(C(=O)N2CCC(Oc3ccc(F)cc3)CC2)cn1] |
| 181 | 5-(4-(4-fluorobenzoyl)piperidine-1- carbonyl)-N-(1-(4-fluorobenzyl)piperidin-4- yl)picolinamide | 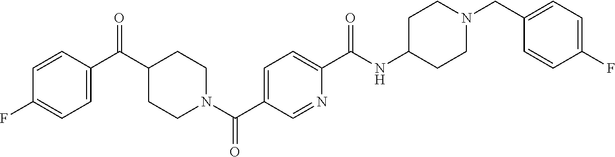 [SMILES: O=C(NC1CCN(Cc2ccc(F)cc2)CC1)c1ccc(C(=O)N2CCC(C(=O)c3ccc(F)cc3)CC2)cn1] |
| 182 | (S)-N-(1-(4-fluorobenzyl)piperidin-4-yl)-5- (3-(4-fluorophenoxy)pyrrolidine-1- carbonyl)picolinamide | 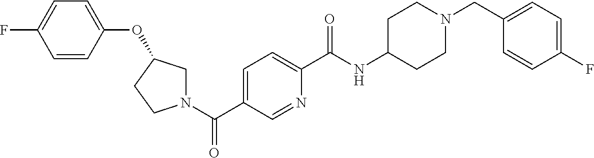 [SMILES: O=C(NC1CCN(Cc2ccc(F)cc2)CC1)c1ccc(C(=O)N2CC[C@H](Oc3ccc(F)cc3)C2)cn1] |
| 183 | N-(1-(4-fluorobenzyl)piperidin-4-yl)-5-(4- (4-methoxyphenoxy)piperidine-1- carbonyl)picolinamide | 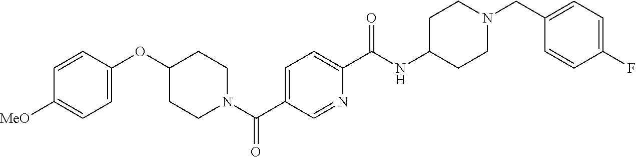 [SMILES: COc1ccc(OC2CCN(C(=O)c3ccc(C(=O)NC4CCN(Cc5ccc(F)cc5)CC4)nc3)CC2)cc1] |
| 184 | 5-(4-(4-methoxybenzoyl)piperidine-1- carbonyl)-N-((cis)-4-(4- methoxyphenoxy)cyclohexyl)picolinamide | 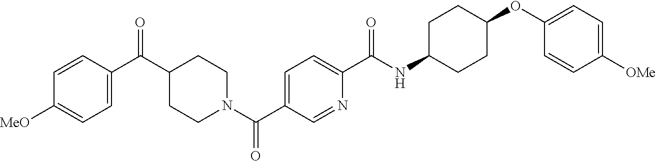 [SMILES: COc1ccc(O[C@H]2CC[C@@H](NC(=O)c3ccc(C(=O)N4CCC(C(=O)c5ccc(OC)cc5)CC4)cn3)CC2)cc1] |
| 185 | N-((cis)-4-(4-methoxyphenoxy)cyclohexyl)- 5-(4-(4- (trifluoromethyl)phenoxy)piperidine-1- carbonyl)picolinamide | 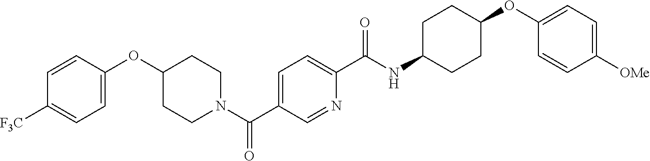 [SMILES: COc1ccc(O[C@H]2CC[C@@H](NC(=O)c3ccc(C(=O)N4CCC(Oc5ccc(C(F)(F)F)cc5)CC4)cn3)CC2)cc1] |
| 186 | N-((cis)-4-(4-methoxyphenoxy)cyclohexyl)- 5-(4-(4-methoxyphenoxy)piperidine-1- carbonyl)picolinamide | 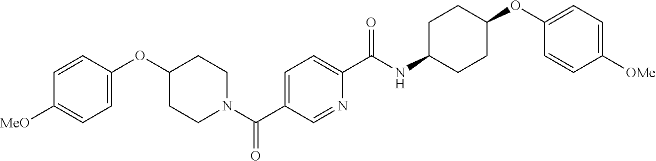 [SMILES: COc1ccc(OC2CCN(C(=O)c3ccc(C(=O)N[C@H]4CC[C@@H](Oc5ccc(OC)cc5)CC4)nc3)CC2)cc1] |
| 187 | (4-(4-fluorobenzyl)piperazin-1-yl)(6-(4-(4- (trifluoromethyl)phenoxy)piperidin-1- yl)pyridin-3-yl)methanone | 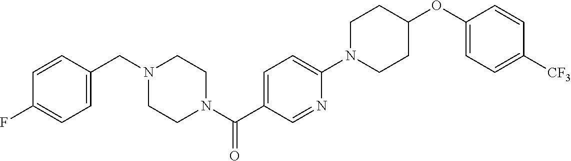 [SMILES: O=C(c1ccc(N2CCC(Oc3ccc(C(F)(F)F)cc3)CC2)nc1)N1CCN(Cc2ccc(F)cc2)CC1] |
| 188 | 4-(1-(5-(4-(4-fluorobenzyl)piperazine-1- carbonyl)pyridin-2-yl)piperidin-4- yloxy)benzonitrile | 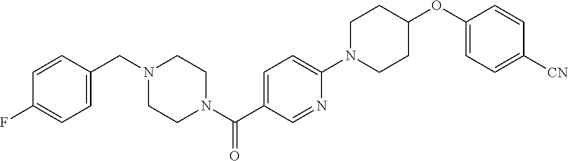 [SMILES: N#Cc1ccc(OC2CCN(c3ccc(C(=O)N4CCN(Cc5ccc(F)cc5)CC4)cn3)CC2)cc1] |
| 189 | (4-(4-fluorobenzyl)piperazin-1-yl)(6-(4-(4- methoxybenzoyl)piperidin-1-yl)pyridin-3- yl)methanone | 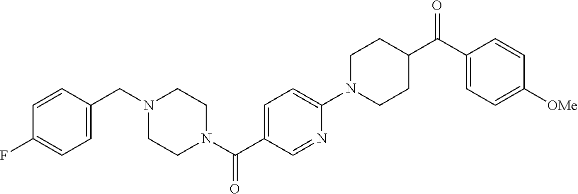 [SMILES: COc1ccc(C(=O)C2CCN(c3ccc(C(=O)N4CCN(Cc5ccc(F)cc5)CC4)cn3)CC2)cc1] |
| 190 | N-((cis)-4-(4-cyanophenoxy)cyclohexyl)-5- (4-(4-(trifluoromethyl)phenoxy)piperidine- 1-carbonyl)picolinamide | 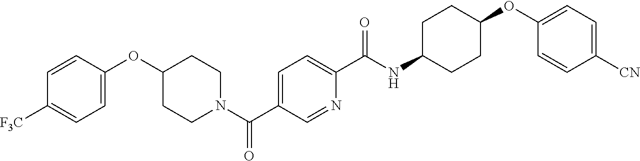 [SMILES: N#Cc1ccc(O[C@H]2CC[C@@H](NC(=O)c3ccc(C(=O)N4CCC(Oc5ccc(C(F)(F)F)cc5)CC4)cn3)CC2)cc1] |
| 191 | 5-(4-(4-methoxyphenoxy)piperidine-1- carbonyl)-N-((cis)-4-(4- (trifluoromethyl)phenoxy)cyclohexyl) picolinamide | 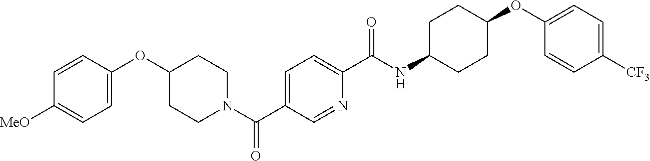 [SMILES: COc1ccc(OC2CCN(C(=O)c3ccc(C(=O)N[C@H]4CC[C@@H](Oc5ccc(C(F)(F)F)cc5)CC4)nc3)CC2)cc1] |
| 192 | 5-(4-(4-methoxybenzoyl)piperidine-1- carbonyl)-N-((cis)-4-(4- (trifluoromethyl)phenoxy)cyclohexyl) picolinamide | 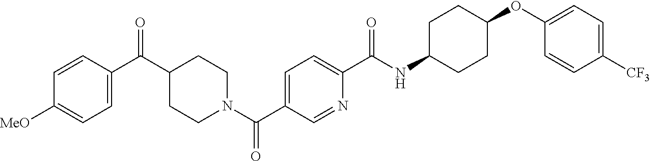 [SMILES: COc1ccc(C(=O)C2CCN(C(=O)c3ccc(C(=O)N[C@H]4CC[C@@H](Oc5ccc(C(F)(F)F)cc5)CC4)nc3)CC2)cc1] |
| 193 | 5-(4-(4-cyanophenoxy)piperidine-1- carbonyl)-N-((cis)-4-(4- (trifluoromethyl)phenoxy)cyclohexyl) picolinamide | 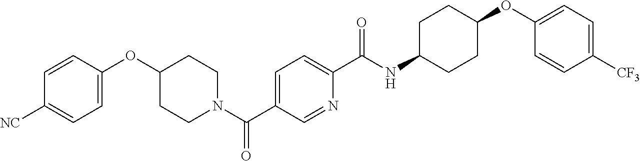 [SMILES: N#Cc1ccc(OC2CCN(C(=O)c3ccc(C(=O)N[C@H]4CC[C@@H](Oc5ccc(C(F)(F)F)cc5)CC4)nc3)CC2)cc1] |
| 194 | 5-(4-(4-methoxybenzoyl)piperidine-1- carbonyl)-N-(1-(4-(pyrrolidin-1- yl)benzyl)piperidin-4-yl)picolinamide | 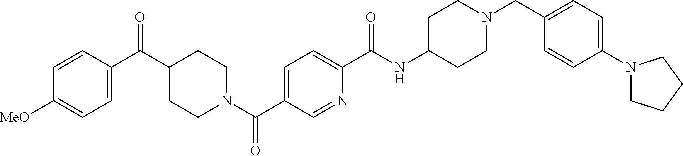 [SMILES: COc1ccc(C(=O)C2CCN(C(=O)c3ccc(C(=O)NC4CCN(Cc5ccc(N6CCCC6)cc5)CC4)nc3)CC2)cc1] |
| 195 | 5-(4-(4-methoxyphenoxy)piperidine-1- carbonyl)-N-(1-(4-(pyrrolidin-1- yl)benzyl)piperidin-4-yl)picolinamide | 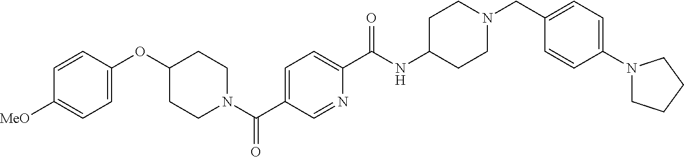 [SMILES: COc1ccc(OC2CCN(C(=O)c3ccc(C(=O)NC4CCN(Cc5ccc(N6CCCC6)cc5)CC4)nc3)CC2)cc1] |
| 196 | 5-(4-(4-fluorophenoxy)piperidine-1- carbonyl)-N-(1-(4-(pyrrolidin-1- yl)benzyl)piperidin-4-yl)picolinamide | 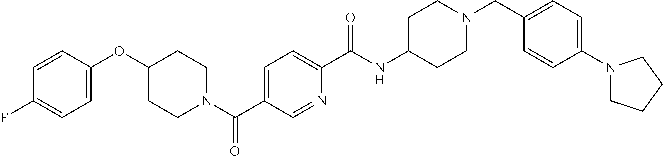 [SMILES: O=C(NC1CCN(Cc2ccc(N3CCCC3)cc2)CC1)c1ccc(C(=O)N2CCC(Oc3ccc(F)cc3)CC2)cn1] |
| 197 | 5-(4-(4-cyanophenoxy)piperidine-1- carbonyl)-N-(1-(4-(pyrrolidin-1- yl)benzyl)piperidin-4-yl)picolinamide | 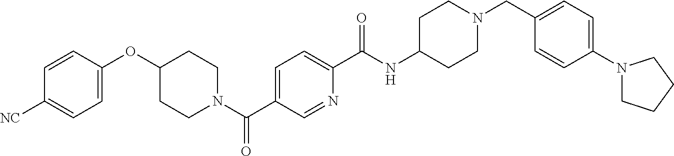 [SMILES: N#Cc1ccc(OC2CCN(C(=O)c3ccc(C(=O)NC4CCN(Cc5ccc(N6CCCC6)cc5)CC4)nc3)CC2)cc1] |
| 198 | N-((cis)-4-(4-cyano-3- fluorophenoxy)cyclohexyl)-5-(4-(4- methoxybenzoyl)piperidine-1- carbonyl)picolinamide | 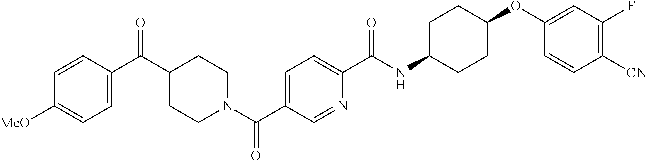 [SMILES: COc1ccc(C(=O)C2CCN(C(=O)c3ccc(C(=O)N[C@H]4CC[C@@H](Oc5ccc(C#N)c(F)c5)CC4)nc3)CC2)cc1] |
| 199 | N-((cis)-4-(4-cyano-3- fluorophenoxy)cyclohexyl)-5-(4-(4- (trifluoromethyl)phenoxy)piperidine-1- carbonyl)picolinamide | 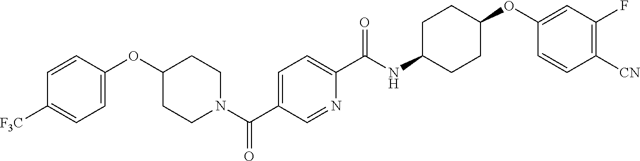 [SMILES: N#Cc1ccc(O[C@H]2CC[C@@H](NC(=O)c3ccc(C(=O)N4CCC(Oc5ccc(C(F)(F)F)cc5)CC4)cn3)CC2)cc1F] |
| 200 | 5-(4-(4-cyanophenoxy)piperidine-1- carbonyl)-N-(1-(4-fluorobenzyl)piperidin-4- yl)picolinamide | 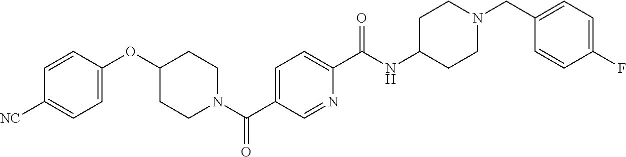 [SMILES: N#Cc1ccc(OC2CCN(C(=O)c3ccc(C(=O)NC4CCN(Cc5ccc(F)cc5)CC4)nc3)CC2)cc1] |
| 201 | N-(1-(4-carbamoylbenzyl)piperidin-4-yl)-5- (4-(4-fluorobenzyl)piperazine-1- carbonyl)picolinamide | 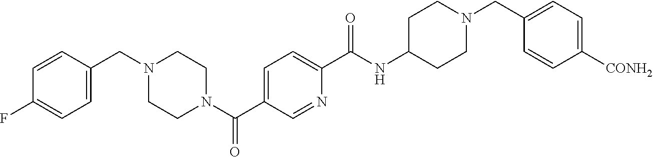 [SMILES: NC(=O)c1ccc(CN2CCC(NC(=O)c3ccc(C(=O)N4CCN(Cc5ccc(F)cc5)CC4)cn3)CC2)cc1] |
| 202 | N-(1-(4-methoxybenzyl)piperidin-4-yl)-5- (4-(4-(methylsulfonyl)phenoxy)piperidine- 1-carbonyl)picolinamide | 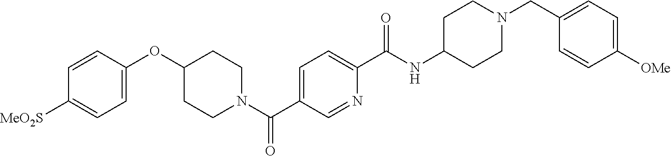 [SMILES: COc1ccc(CN2CCC(NC(=O)c3ccc(C(=O)N4CCC(Oc5ccc(S(C)(=O)=O)cc5)CC4)cn3)CC2)cc1] |
| 203 | N-(1-(3,5-difluorobenzyl)piperidin-4-yl)-5- (4-(4-(methylsulfonyl)phenoxy)piperidine- 1-carbonyl)picolinamide | 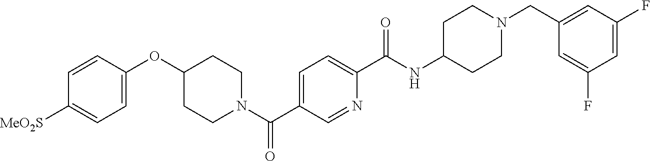 [SMILES: CS(=O)(=O)c1ccc(OC2CCN(C(=O)c3ccc(C(=O)NC4CCN(Cc5cc(F)cc(F)c5)CC4)nc3)CC2)cc1] |
| 204 | N-(6-(4-fluorophenoxy)pyridin-3-yl)-5-(4- (4-methoxybenzoyl)piperidine-1- carbonyl)pyrazine-2-carboxamide | 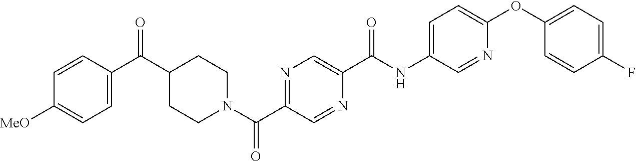 [SMILES: COc1ccc(C(=O)C2CCN(C(=O)c3cnc(C(=O)Nc4ccc(Oc5ccc(F)cc5)nc4)cn3)CC2)cc1] |
| 205 | N-(6-(4-fluorophenoxy)pyridin-3-yl)-5-(4- (4-(trifluoromethyl)phenoxy)piperidine-1- carbonyl)pyrazine-2-carboxamide | 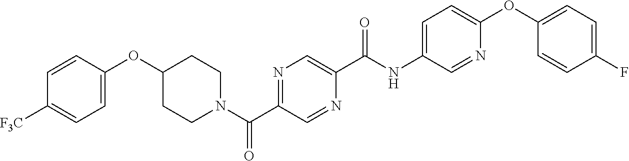 [SMILES: O=C(Nc1ccc(Oc2ccc(F)cc2)nc1)c1cnc(C(=O)N2CCC(Oc3ccc(C(F)(F)F)cc3)CC2)cn1] |
| 206 | 5-(4-(2,4-difluorobenzoyl)piperidine-1- carbonyl)-N-(6-(4-fluorophenoxy)pyridin-3- yl)pyrazine-2-carboxamide | 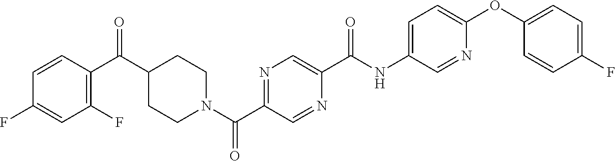 [SMILES: O=C(Nc1ccc(Oc2ccc(F)cc2)nc1)c1cnc(C(=O)N2CCC(C(=O)c3ccc(F)cc3F)CC2)cn1] |
| 207 | N-(6-(4-fluorophenoxy)pyridin-3-yl)-5-(4- (4-(methylsulfonyl)phenoxy)piperidine-1- carbonyl)pyrazine-2-carboxamide | 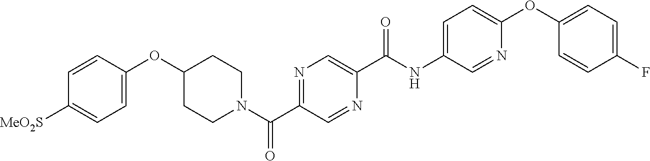 [SMILES: CS(=O)(=O)c1ccc(OC2CCN(C(=O)c3cnc(C(=O)Nc4ccc(Oc5ccc(F)cc5)nc4)cn3)CC2)cc1] |
| 208 | 5-(4-(4- (methylsulfonyl)phenoxy)piperidine-1- carbonyl)-N-(1-(4-(pyrrolidin-1- yl)benzyl)piperidin-4-yl)picolinamide | 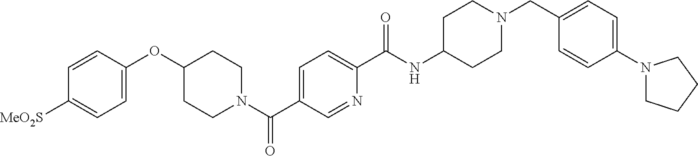 [SMILES: CS(=O)(=O)c1ccc(OC2CCN(C(=O)c3ccc(C(=O)NC4CCN(Cc5ccc(N6CCCC6)cc5)CC4)nc3)CC2)cc1] |
| 209 | N-(1-(4-cyanobenzyl)piperidin-4-yl)-5-(3- (4-cyanophenoxy)azetidine-1- carbonyl)picolinamide | 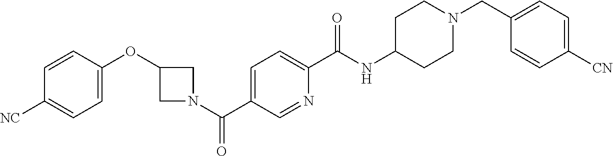 [SMILES: N#Cc1ccc(CN2CCC(NC(=O)c3ccc(C(=O)N4CC(Oc5ccc(C#N)cc5)C4)cn3)CC2)cc1] |
| 210 | 5-(3-(4-cyanophenoxy)azetidine-1- carbonyl)-N-(6-(4-fluorophenoxy)pyridin-3- yl)picolinamide | 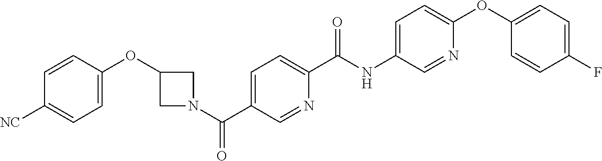 [SMILES: N#Cc1ccc(OC2CN(C(=O)c3ccc(C(=O)Nc4ccc(Oc5ccc(F)cc5)nc4)nc3)C2)cc1] |
| 211 | N-(1-(4-cyanobenzyl)piperidin-4-yl)-5-(4- (4-methoxybenzoyl)piperidine-1- carbonyl)pyrazine-2-carboxamide | 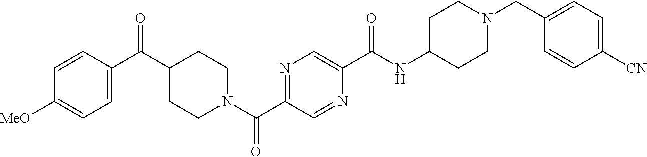 [SMILES: COc1ccc(C(=O)C2CCN(C(=O)c3cnc(C(=O)NC4CCN(Cc5ccc(C#N)cc5)CC4)cn3)CC2)cc1] |
| 212 | N-(1-(4-cyanobenzyl)piperidin-4-yl)-5-(4- (4-(trifluoromethyl)phenoxy)piperidine-1- carbonyl)pyrazine-2-carboxamide | 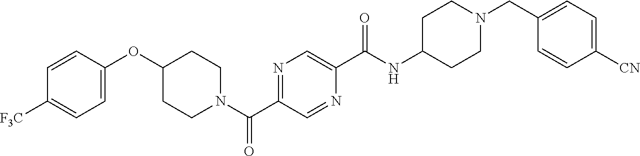 [SMILES: N#Cc1ccc(CN2CCC(NC(=O)c3cnc(C(=O)N4CCC(Oc5ccc(C(F)(F)F)cc5)CC4)cn3)CC2)cc1] |
| 213 | 6-(4-(2,4-difluorobenzoyl)piperidine-1- carbonyl)-N-(1-(4- (methylsulfonyl)benzyl)piperidin-4- yl)nicotinamide | 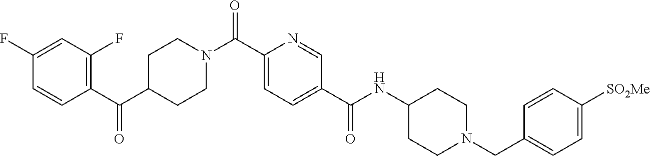 [SMILES: CS(=O)(=O)c1ccc(CN2CCC(NC(=O)c3ccc(C(=O)N4CCC(C(=O)c5ccc(F)cc5F)CC4)nc3)CC2)cc1] |
| 214 | 6-(4-(4-cyanophenoxy)piperidine-1- carbonyl)-N-(1-(4- (methylsulfonyl)benzyl)piperidin-4- yl)nicotinamide | 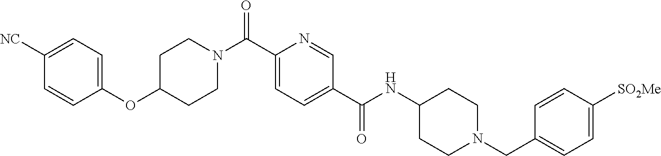 [SMILES: CS(=O)(=O)c1ccc(CN2CCC(NC(=O)c3ccc(C(=O)N4CCC(Oc5ccc(C#N)cc5)CC4)nc3)CC2)cc1] |
| 215 | 6-(4-(4-methoxybenzoyl)piperidine-1- carbonyl)-N-(1-(4- (methylsulfonyl)benzyl)piperidin-4- yl)nicotinamide | 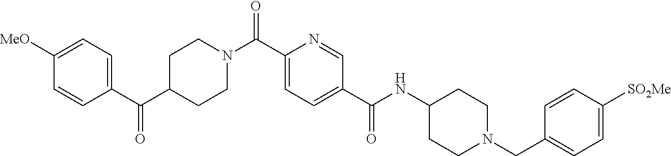 [SMILES: COc1ccc(C(=O)C2CCN(C(=O)c3ccc(C(=O)NC4CCN(Cc5ccc(S(C)(=O)=O)cc5)CC4)cn3)CC2)cc1] |
| 216 | 6-(4-(2,4-difluorobenzoyl)piperidine-1- carbonyl)-N-(1-(4- (methylsulfonamido)benzyl)piperidin-4- yl)nicotinamide | 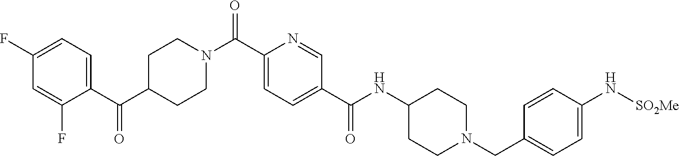 [SMILES: CS(=O)(=O)Nc1ccc(CN2CCC(NC(=O)c3ccc(C(=O)N4CCC(C(=O)c5ccc(F)cc5F)CC4)nc3)CC2)cc1] |
| 217 | 6-(4-(4-cyanophenoxy)piperidine-1- carbonyl)-N-(1-(4- (methylsulfonamido)benzyl)piperidin-4- yl)nicotinamide | 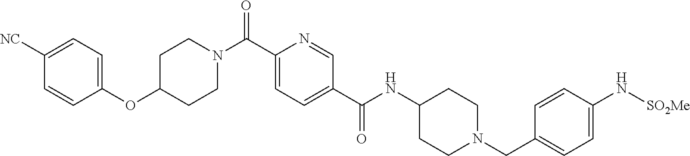 [SMILES: CS(=O)(=O)Nc1ccc(CN2CCC(NC(=O)c3ccc(C(=O)N4CCC(Oc5ccc(C#N)cc5)CC4)nc3)CC2)cc1] |
| 218 | N-(1-(4-cyanobenzyl)piperidin-4-yl)-5-(4- (4-(methylsulfonyl)phenoxy)piperidine-1- carbonyl)pyrazine-2-carboxamide | 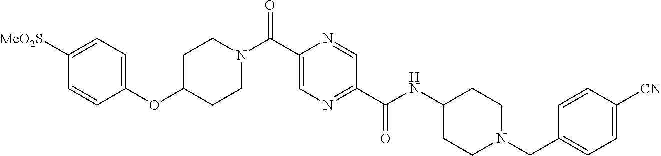 [SMILES: CS(=O)(=O)c1ccc(OC2CCN(C(=O)c3cnc(C(=O)NC4CCN(Cc5ccc(C#N)cc5)CC4)cn3)CC2)cc1] |
| 219 | N-(1-(4-cyanobenzyl)piperidin-4-yl)-5-(4- (4-(pyrrolidin-1-yl)benzoyl)piperidine-1- carbonyl)pyrazine-2-carboxamide | 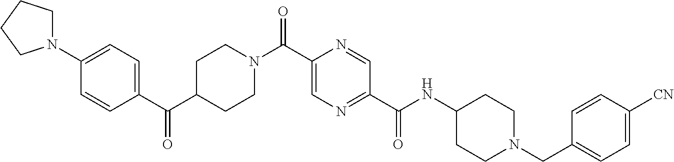 [SMILES: N#Cc1ccc(CN2CCC(NC(=O)c3cnc(C(=O)N4CCC(C(=O)c5ccc(N6CCCC6)cc5)CC4)cn3)CC2)cc1] |
| 220 | N-(1-(4-cyanobenzyl)piperidin-4-yl)-5-(4- (4-(4-methylpiperazin-1- yl)benzoyl)piperidine-1-carbonyl)pyrazine- 2-carboxamide | 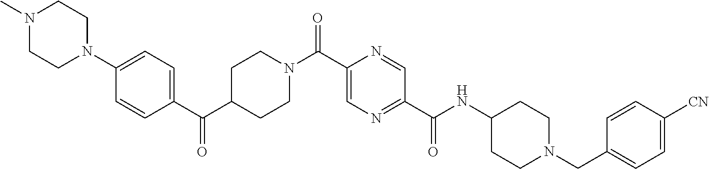 [SMILES: CN1CCN(c2ccc(C(=O)C3CCN(C(=O)c4cnc(C(=O)NC5CCN(Cc6ccc(C#N)cc6)CC5)cn4)CC3)cc2)CC1] |
| 221 | N-(1-(4-cyanobenzyl)piperidin-4-yl)-6-(4- (4-(methylsulfonyl)benzoyl)piperidine-1- carbonyl)nicotinamide | 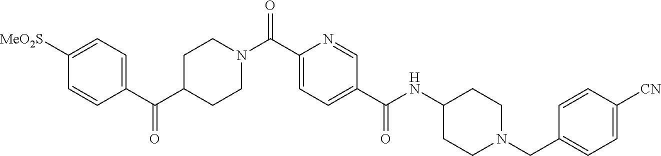 [SMILES: CS(=O)(=O)c1ccc(C(=O)C2CCN(C(=O)c3ccc(C(=O)NC4CCN(Cc5ccc(C#N)cc5)CC4)cn3)CC2)cc1] |
| 222 | N-(1-(4-fluorobenzyl)piperidin-4-yl)-6-(4- (4-(methylsulfonyl)benzoyl)piperidine-1- carbonyl)nicotinamide | 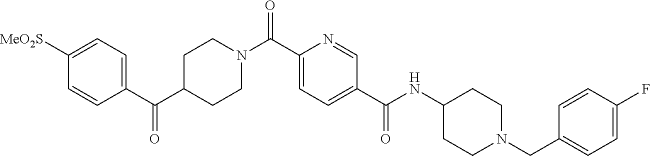 [SMILES: CS(=O)(=O)c1ccc(C(=O)C2CCN(C(=O)c3ccc(C(=O)NC4CCN(Cc5ccc(F)cc5)CC4)cn3)CC2)cc1] |
| 223 | N-(1-(4-methoxybenzyl)piperidin-4-yl)-6- (4-(4-(methylsulfonyl)benzoyl)piperidine-1- carbonyl)nicotinamide | 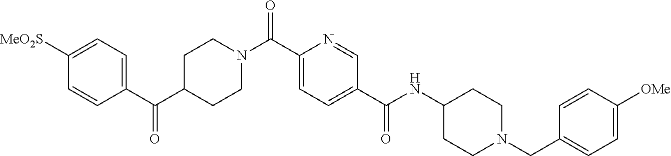 [SMILES: COc1ccc(CN2CCC(NC(=O)c3ccc(C(=O)N4CCC(C(=O)c5ccc(S(C)(=O)=O)cc5)CC4)nc3)CC2)cc1] |
| 224 | N-(6-(4-fluorophenoxy)pyridin-3-yl)-6-(4- (4-(methylsulfonyl)benzoyl)piperidine-1- carbonyl)nicotinamide | 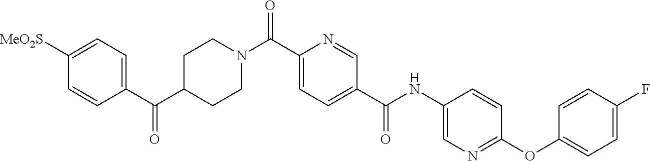 [SMILES: CS(=O)(=O)c1ccc(C(=O)C2CCN(C(=O)c3ccc(C(=O)Nc4ccc(Oc5ccc(F)cc5)nc4)cn3)CC2)cc1] |
| 225 | N-(1-(4-fluorobenzyl)piperidin-4-yl)-5-(4- (4-(methylsulfonyl)benzoyl)piperidine-1- carbonyl)picolinamide | 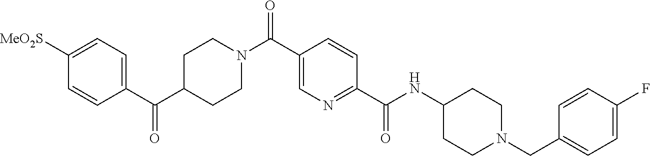 [SMILES: CS(=O)(=O)c1ccc(C(=O)C2CCN(C(=O)c3ccc(C(=O)NC4CCN(Cc5ccc(F)cc5)CC4)nc3)CC2)cc1] |
| 226 | N-(1-(4-fluorobenzyl)piperidin-4-yl)-5-(4- (4-(methylsulfonyl)phenoxy)piperidine-1- carbonyl)picolinamide | 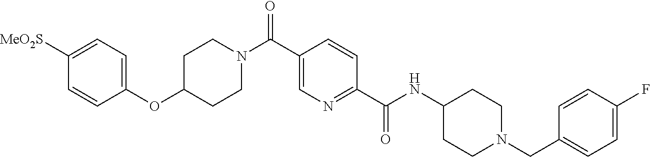 [SMILES: CS(=O)(=O)c1ccc(OC2CCN(C(=O)c3ccc(C(=O)NC4CCN(Cc5ccc(F)cc5)CC4)nc3)CC2)cc1] |
| 227 | N-(1-(4-cyanobenzyl)piperidin-4-yl)-5-(4- (4-(methylsulfonyl)phenoxy)piperidine-1- carbonyl)picolinamide | 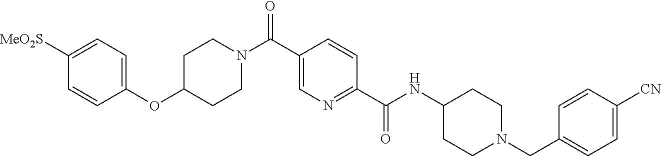 [SMILES: CS(=O)(=O)c1ccc(OC2CCN(C(=O)c3ccc(C(=O)NC4CCN(Cc5ccc(C#N)cc5)CC4)nc3)CC2)cc1] |
| 228 | N-(1-(4-cyanobenzyl)piperidin-4-yl)-5-(4- (4-(methylsulfonyl)benzoyl)piperidine-1- carbonyl)picolinamide | 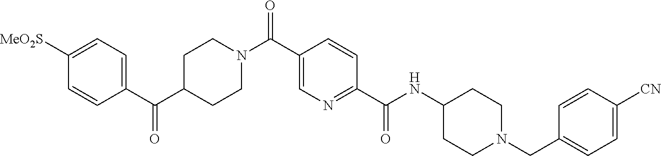 [SMILES: CS(=O)(=O)c1ccc(C(=O)C2CCN(C(=O)c3ccc(C(=O)NC4CCN(Cc5ccc(C#N)cc5)CC4)nc3)CC2)cc1] |
| 229 | 6-(4-(4-(methylsulfonyl)benzoyl)piperidine- 1-carbonyl)-N-(1-(4-(pyrrolidin-1- yl)benzyl)piperidin-4-yl)nicotinamide | 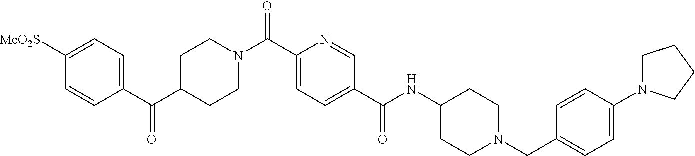 [SMILES: CS(=O)(=O)c1ccc(C(=O)C2CCN(C(=O)c3ccc(C(=O)NC4CCN(Cc5ccc(N6CCCC6)cc5)CC4)cn3)CC2)cc1] |
| 230 | 6-(4-(4-(methylsulfonyl)phenoxy)piperidine-1- carbonyl)-N-(1-(4-(pyrrolidin-1- yl)benzyl)piperidin-4-yl)nicotinamide | 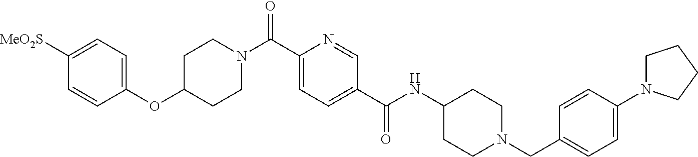 [SMILES: CS(=O)(=O)c1ccc(OC2CCN(C(=O)c3ccc(C(=O)NC4CCN(Cc5ccc(N6CCCC6)cc5)CC4)cn3)CC2)cc1] |
| 231 | N-(6-(4-fluorophenoxy)pyridin-3-yl)-5-(4- (4-(methylsulfonyl)phenoxy)piperidine-1- carbonyl)picolinamide | 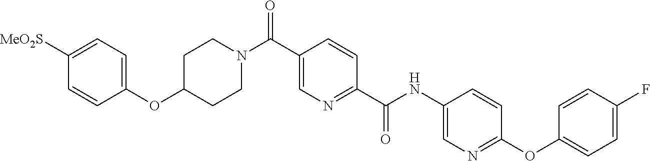 [SMILES: CS(=O)(=O)c1ccc(OC2CCN(C(=O)c3ccc(C(=O)Nc4ccc(Oc5ccc(F)cc5)nc4)nc3)CC2)cc1] |
| 232 | N-(6-(4-fluorophenoxy)pyridin-3-yl)-5-(4- (4-(methylsulfonyl)benzoyl)piperidine-1- carbonyl)picolinamide | 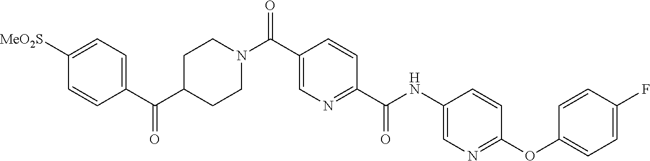 [SMILES: CS(=O)(=O)c1ccc(C(=O)C2CCN(C(=O)c3ccc(C(=O)Nc4ccc(Oc5ccc(F)cc5)nc4)nc3)CC2)cc1] |
| 233 | 5-(4-(4-(methylsulfonyl)benzoyl)piperidine- 1-carbonyl)-N-(1-(4-(pyrro lidin-1- yl)benzyl)piperidin-4-yl)picolinamide | 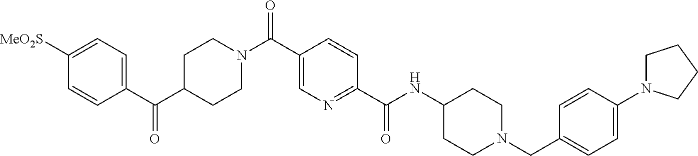 [SMILES: CS(=O)(=O)c1ccc(C(=O)C2CCN(C(=O)c3ccc(C(=O)NC4CCN(Cc5ccc(N6CCCC6)cc5)CC4)nc3)CC2)cc1] |
| 234 | N-(1-(4-methoxybenzyl)piperidin-4-yl)-5- (4-(4-(methylsulfonyl)benzoyl)piperidine-1- carbonyl)picolinamide | 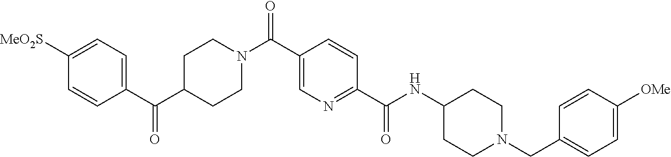 [SMILES: COc1ccc(CN2CCC(NC(=O)c3ccc(C(=O)N4CCC(C(=O)c5ccc(S(C)(=O)=O)cc5)CC4)cn3)CC2)cc1] |
| 235 | N-(6-(4-fluorophenoxy)pyridin-3-yl)-6-(4- (4-(methylsulfonyl)phenoxy)piperidine-1- carbonyl)nicotinamide | 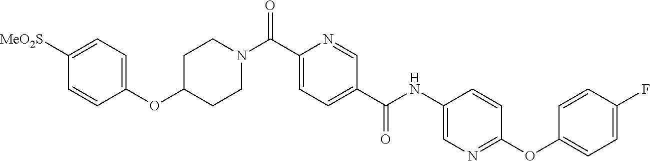 [SMILES: CS(=O)(=O)c1ccc(OC2CCN(C(=O)c3ccc(C(=O)Nc4ccc(Oc5ccc(F)cc5)nc4)cn3)CC2)cc1] |
| 236 | N-(1-(3,5-difluorobenzyl)piperidin-4-yl)-6- (4-(4-(methylsulfonyl)phenoxy)piperidine- 1-carbonyl)nicotinamide | 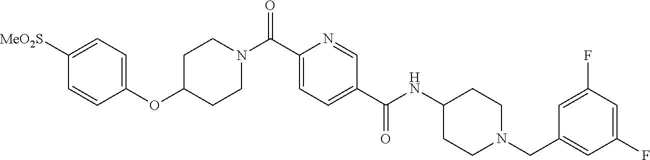 [SMILES: CS(=O)(=O)c1ccc(OC2CCN(C(=O)c3ccc(C(=O)NC4CCN(Cc5cc(F)cc(F)c5)CC4)cn3)CC2)cc1] |
| 237 | N-(1-(4-methoxybenzyl)piperidin-4-yl)-6- (4-(4-(methylsulfonyl)phenoxy)piperidine- 1-carbonyl)nicotinamide | 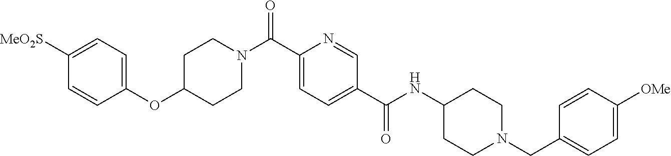 [SMILES: COc1ccc(CN2CCC(NC(=O)c3ccc(C(=O)N4CCC(Oc5ccc(S(C)(=O)=O)cc5)CC4)nc3)CC2)cc1] |
| 238 | N-(1-(3-methoxybenzyl)piperidin-4-yl)-6- (4-(4-(methylsulfonyl)phenoxy)piperidine- 1-carbonyl)nicotinamide | 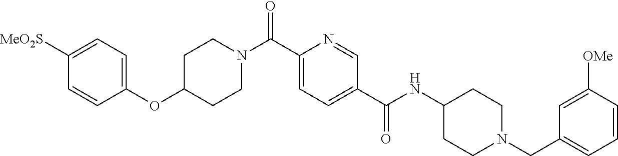 [SMILES: COc1cccc(CN2CCC(NC(=O)c3ccc(C(=O)N4CCC(Oc5ccc(S(C)(=O)=O)cc5)CC4)nc3)CC2)c1] |
| 239 | 6-(4-(4-(methylsulfonyl)phenoxy) piperidine-1-carbonyl)-N-(1-(3- (trifluoromethoxy)benzyl)piperidin-4- yl)nicotinamide | 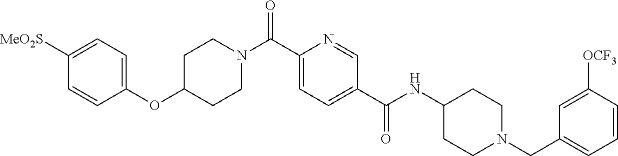 [SMILES: CS(=O)(=O)c1ccc(OC2CCN(C(=O)c3ccc(C(=O)NC4CCN(Cc5cccc(OC(F)(F)F)c5)CC4)cn3)CC2)cc1] |
| 240 | 6-(4-(4-azidobenzoyl)piperidine-1- carbonyl)-N-(1-(4-cyanobenzyl)piperidin-4- yl)nicotinamide | 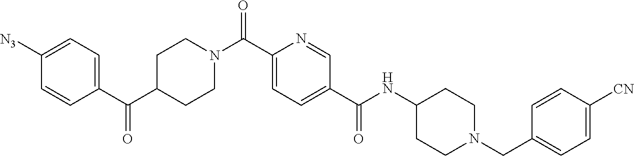 [SMILES: N#Cc1ccc(CN2CCC(NC(=O)c3ccc(C(=O)N4CCC(C(=O)c5ccc(N=[N+]=[N-])cc5)CC4)nc3)CC2)cc1] |
| 241 | N-(1-(3-methoxybenzyl)piperidin-4-yl)-5- (4-(4-(methylsulfonyl)phenoxy)piperidine- 1-carbonyl)picolinamide | 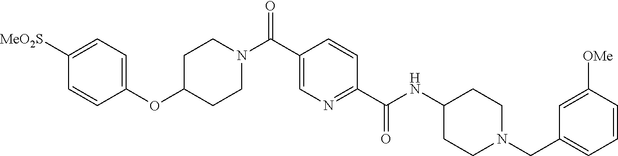 [SMILES: COc1cccc(CN2CCC(NC(=O)c3ccc(C(=O)N4CCC(Oc5ccc(S(C)(=O)=O)cc5)CC4)cn3)CC2)c1] |
| 242 | 5-(4-(4-(methylsulfonyl)phenoxy) piperidine-1- carbonyl)-N-(1-(3-(trifluoromethoxy) benzyl)piperidin-4- yl)picolinamide | 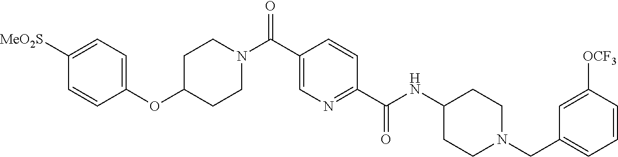 [SMILES: CS(=O)(=O)c1ccc(OC2CCN(C(=O)c3ccc(C(=O)NC4CCN(Cc5cccc(OC(F)(F)F)c5)CC4)nc3)CC2)cc1] |
| 243 | N-(1-(4-cyanobenzyl)piperidin-4-yl)- 6-(4-(4-(4-methylpiperazin-1-yl) benzoyl)piperidine-1- carbonyl)nicotinamide | 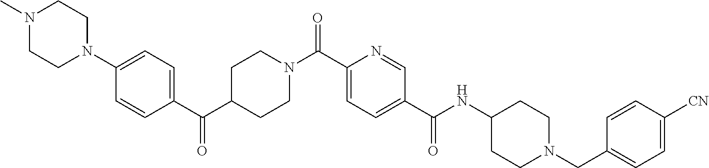 [SMILES: CN1CCN(c2ccc(C(=O)C3CCN(C(=O)c4ccc(C(=O)NC5CCN(Cc6ccc(C#N)cc6)CC5)cn4)CC3)cc2)CC1] |
| 244 | 6-(4-(4-(4-methylpiperazin-1- yl)benzoyl)piperidine-1- carbonyl)-N-(1-(4- (trifluoromethoxy)benzyl) piperidin-4-yl)nicotinamide | 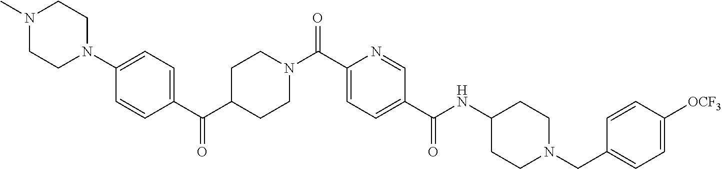 [SMILES: CN1CCN(c2ccc(C(=O)C3CCN(C(=O)c4ccc(C(=O)NC5CCN(Cc6ccc(OC(F)(F)F)cc6)CC5)cn4)CC3)cc2)CC1] |
| 245 | N-(6-(4-fluorophenoxy)pyridin-3-yl)- 6-(4-(4-(4-methylpiperazin-1-yl) benzoyl)piperidine-1- carbonyl)nicotinamide | 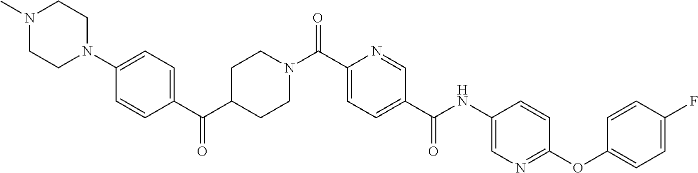 [SMILES: CN1CCN(c2ccc(C(=O)C3CCN(C(=O)c4ccc(C(=O)Nc5ccc(Oc6ccc(F)cc6)nc5)cn4)CC3)cc2)CC1] |
| 246 | N-(1-(4-cyanobenzyl)piperidin-4- yl)-6-(4-(4-(cyclopropylsulfonyl) phenoxy)piperidine-1- carbonyl)nicotinamide | 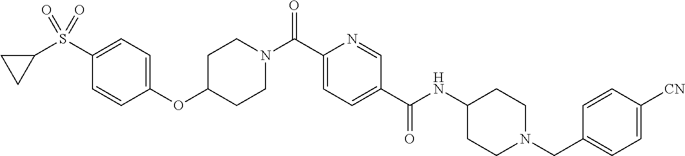 [SMILES: N#Cc1ccc(CN2CCC(NC(=O)c3ccc(C(=O)N4CCC(Oc5ccc(S(=O)(=O)C6CC6)cc5)CC4)nc3)CC2)cc1] |
| 247 | 6-(4-(4-(cyclopropylsulfonyl) phenoxy)piperidine-1- carbonyl)-N-(6-(4-fluorophenoxy) pyridin-3-yl)nicotinamide | 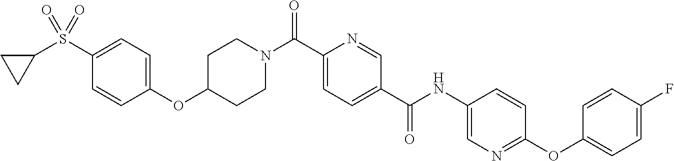 [SMILES: O=C(Nc1ccc(Oc2ccc(F)cc2)nc1)c1ccc(C(=O)N2CCC(Oc3ccc(S(=O)(=O)C4CC4)cc3)CC2)nc1] |
| 248 | 6-(4-(4-(cyclopropylsulfonyl) phenoxy)piperidine-1- carbonyl)-N-(1-(4-(pyrrolidin-1- yl)benzyl)piperidin-4-yl)nicotinamide | 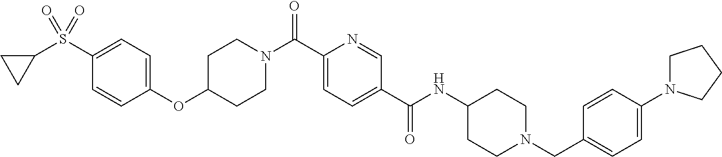 [SMILES: O=C(NC1CCN(Cc2ccc(N3CCCC3)cc2)CC1)c1ccc(C(=O)N2CCC(Oc3ccc(S(=O)(=O)C4CC4)cc3)CC2)nc1] |
| 249 | 6-(4-(4-(cyclopropylsulfonyl) phenoxy)piperidine-1- carbonyl)-N-(1-(4-(trifluoromethoxy) benzyl)piperidin-4- yl)nicotinamide | 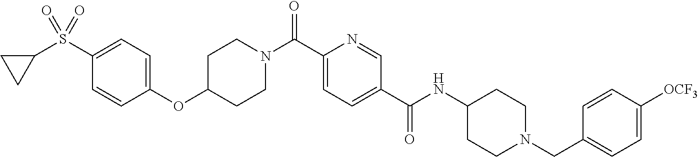 [SMILES: O=C(NC1CCN(Cc2ccc(OC(F)(F)F)cc2)CC1)c1ccc(C(=O)N2CCC(Oc3ccc(S(=O)(=O)C4CC4)cc3)CC2)nc1] |
| 250 | N-(1-(4-cyanobenzyl)piperidin-4-yl)-5-(4- (4-(methylsulfonyl)phenyl)piperazine-1- carbonyl)picolinamide | 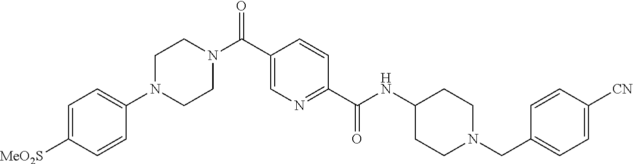 [SMILES: CS(=O)(=O)c1ccc(N2CCN(C(=O)c3ccc(C(=O)NC4CCN(Cc5ccc(C#N)cc5)CC4)nc3)CC2)cc1] |
| 251 | N-(1-(4-cyanobenzyl)piperidin-4-yl)-5-(4- (4-(isopropylsulfonyl)phenyl)piperazine-1- carbonyl)picolinamide | 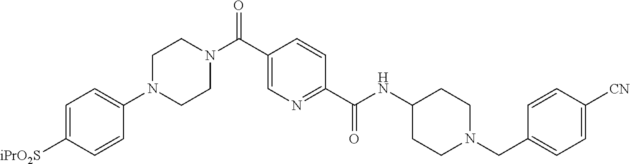 |
| 252 | N-((trans)-1-(4-cyanobenzyl)-3- fluoropiperidin-4-yl)-5-(4-(4- (methylsulfonyl)benzoyl)piperidine-1- carbonyl)picolinamide | 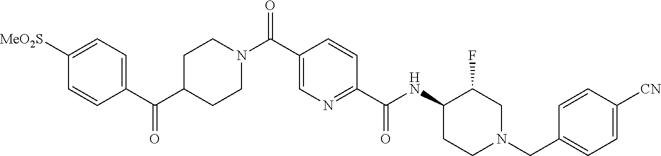 [SMILES: CS(=O)(=O)c1ccc(C(=O)C2CCN(C(=O)c3ccc(C(=O)N[C@@H]4CCN(Cc5ccc(C#N)cc5)C[C@H]4F)nc3)CC2)cc1] |
| 253 | N-((trans)-3-fluoro-1-(4- (trifluoromethoxy)benzyl)piperidin-4-yl)-5- (4-(4-(methylsulfonyl)benzoyl)piperidine-1- carbonyl)picolinamide | 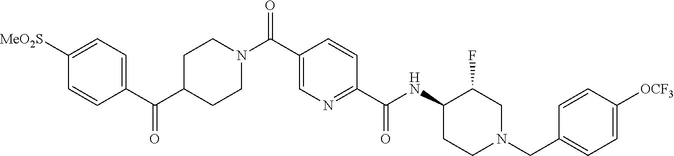 [SMILES: CS(=O)(=O)c1ccc(C(=O)C2CCN(C(=O)c3ccc(C(=O)N[C@@H]4CCN(Cc5ccc(OC(F)(F)F)cc5)C[C@H]4F)nc3)CC2)cc1] |
| 254 | N-((trans)-1-(4-cyanobenzyl)-3- fluoropiperidin-4-yl)-5-(4-(4- (methylsulfonyl)phenoxy)piperidine-1- carbonyl)picolinamide | 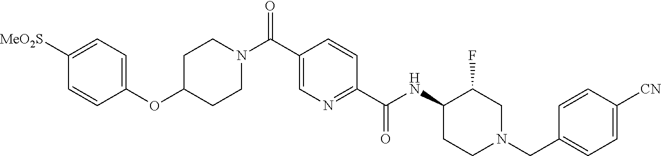 [SMILES: CS(=O)(=O)c1ccc(OC2CCN(C(=O)c3ccc(C(=O)N[C@@H]4CCN(Cc5ccc(C#N)cc5)C[C@H]4F)nc3)CC2)cc1] |
| 255 | N-((trans)-3-fluoro-1-(4- (trifluoromethoxy)benzyl)piperidin-4-yl)-5- (4-(4-(methylsulfonyl)phenoxy)piperidine- 1-carbonyl)picolinamide | 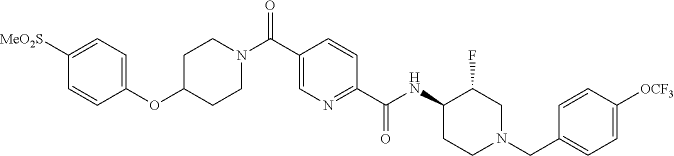 [SMILES: CS(=O)(=O)c1ccc(OC2CCN(C(=O)c3ccc(C(=O)N[C@@H]4CCN(Cc5ccc(OC(F)(F)F)cc5)C[C@H]4F)nc3)CC2)cc1] |
| 256 | N-(1-(4-cyanobenzyl)piperidin-4-yl)-5-(4- (4-(cyclopropylsulfonyl)phenyl)piperazine- 1-carbonyl)picolinamide | 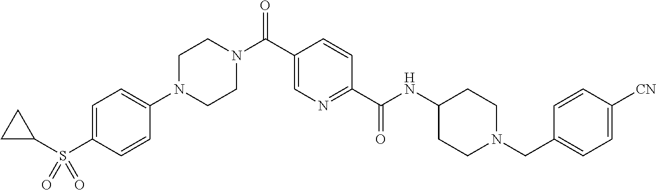 [SMILES: N#Cc1ccc(CN2CCC(NC(=O)c3ccc(C(=O)N4CCN(c5ccc(S(=O)(=O)C6CC6)cc5)CC4)cn3)CC2)cc1] |
| 257 | N-(1-(4-cyanobenzyl)piperidin-4-yl)-5-(4- (4-(trifluoromethylsulfonyl)phenyl) piperazine-1-carbonyl)picolinamide | 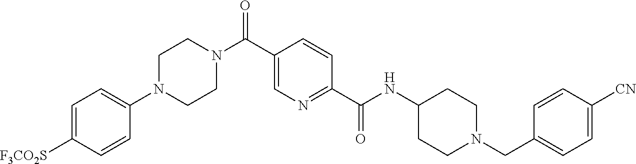 |
| 258 | N-(1-(4-cyanobenzyl)piperidin-4-yl)-5-(4- (4-(cyclopropanecarbonyl)phenyl)piperazine- 1-carbonyl)picolinamide | 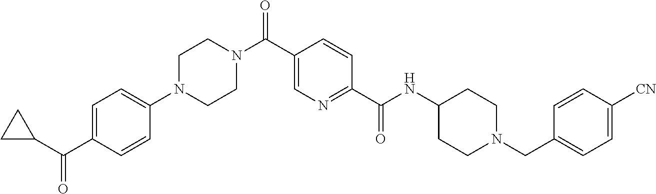 [SMILES: N#Cc1ccc(CN2CCC(NC(=O)c3ccc(C(=O)N4CCN(c5ccc(C(=O)C6CC6)cc5)CC4)cn3)CC2)cc1] |
| 259 | N-(6-(4-acetylphenoxy)pyridin-3-yl)-5-(4- (4-(methylsulfonyl)phenoxy)piperidine-1- carbonyl)picolinamide | 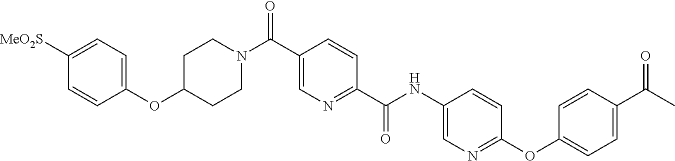 [SMILES: CC(=O)c1ccc(Oc2ccc(NC(=O)c3ccc(C(=O)N4CCC(Oc5ccc(S(C)(=O)=O)cc5)CC4)cn3)cn2)cc1] |
| 260 | N-(1-(4-cyanobenzyl)piperidin-4-yl)-5-(4- (4-(ethylsulfonyl)benzoyl)piperidine-1- carbonyl)picolinamide | 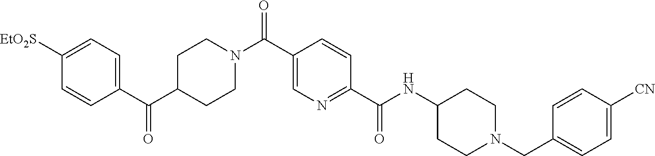 |
| 261 | N-(6-(4-fluorophenylsulfonyl)pyridin-3-yl)- 5-(4-(4-(methylsulfonyl)phenoxy)piperidine-1- carbonyl)picolinamide | 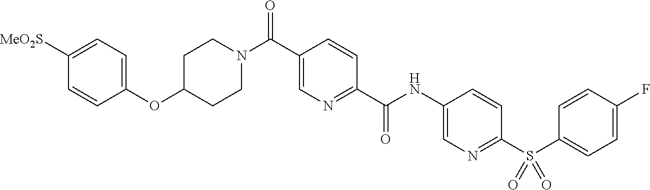 [SMILES: CS(=O)(=O)c1ccc(OC2CCN(C(=O)c3ccc(C(=O)Nc4ccc(S(=O)(=O)c5ccc(F)cc5)nc4)nc3)CC2)cc1] |
| 262 | N-(1-(4-cyanobenzyl)piperidin-4-yl)-5-(4- (4-fluorophenylsulfonyl)piperidine-1- carbonyl)picolinamide | 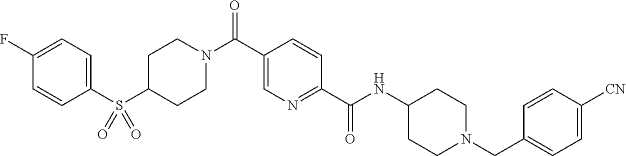 [SMILES: N#Cc1ccc(CN2CCC(NC(=O)c3ccc(C(=O)N4CCC(S(=O)(=O)c5ccc(F)cc5)CC4)cn3)CC2)cc1] |
| 263 | N-(1-(4-cyanobenzyl)piperidin-4-yl)-5-(4- (4-(2,2,2-trifluoroacetyl)phenyl)piperazine- 1-carbonyl)picolinamide | 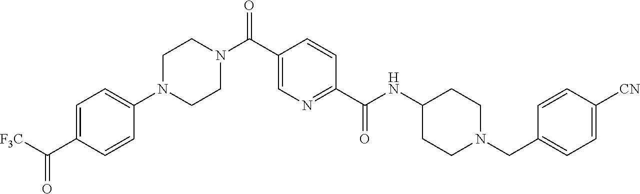 [SMILES: N#Cc1ccc(CN2CCC(NC(=O)c3ccc(C(=O)N4CCN(c5ccc(C(=O)C(F)(F)F)cc5)CC4)cn3)CC2)cc1] |
| 264 | N2,N5-bis(1-benzylpiperidin-4-yl)pyridine- 2,5-dicarboxamide | 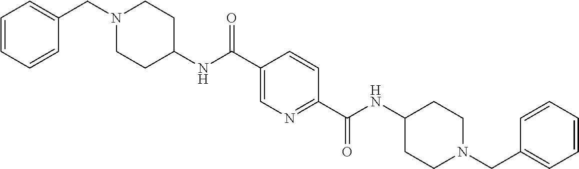 [SMILES: O=C(NC1CCN(Cc2ccccc2)CC1)c1ccc(C(=O)NC2CCN(Cc3ccccc3)CC2)nc1] |
| 265 | N-(1-(4-cyanobenzyl)piperidin-4-yl)-5-(3- (4-cyanophenoxy)piperidin-1- yl)picolinamide | 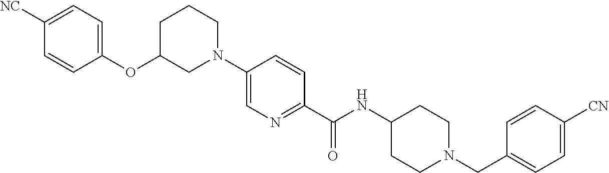 [SMILES: N#Cc1ccc(CN2CCC(NC(=O)c3ccc(N4CCCC(Oc5ccc(C#N)cc5)C4)cn3)CC2)cc1] |
| 266 | 5-(4-(4-chlorobenzoyl)piperidin-1-yl)-N-(1- (4-cyanobenzyl)piperidin-4-yl)picolinamide | 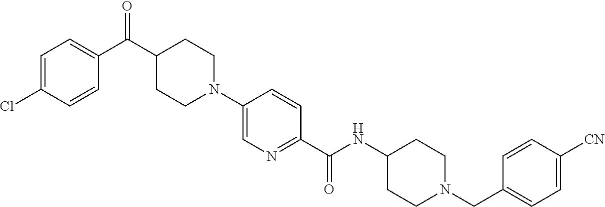 [SMILES: N#Cc1ccc(CN2CCC(NC(=O)c3ccc(N4CCC(C(=O)c5ccc(Cl)cc5)CC4)cn3)CC2)cc1] |
| 267 | N-(1-(4-cyanobenzyl)piperidin-4-yl)-5-(1- (4-cyanophenyl)piperidin-4- ylamino)picolinamide | 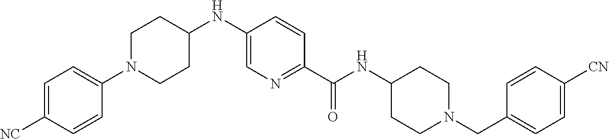 [SMILES: N#Cc1ccc(CN2CCC(NC(=O)c3ccc(NC4CCN(c5ccc(C#N)cc5)CC4)cn3)CC2)cc1] |
| 268 | N-(1-(4-cyanobenzyl)piperidin-4-yl)-5-(4- (2-(4-fluorophenyl)propan-2-yl)piperazine- 1-carbonyl)picolinamide | 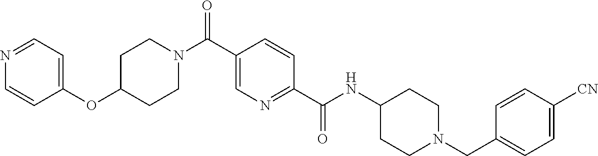 [SMILES: N#Cc1ccc(CN2CCC(NC(=O)c3ccc(C(=O)N4CCC(Oc5ccncc5)CC4)cn3)CC2)cc1] |
| 269 | N-(1-(4-cyanobenzyl)piperidin-4-yl)-5-(4- (pyridin-4-yloxy)piperidine-1- carbonyl)picolinamide | 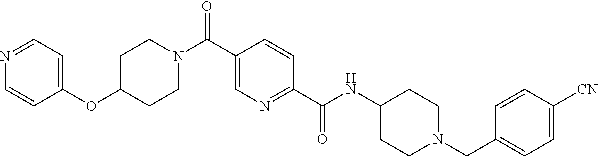 [SMILES: N#Cc1ccc(CN2CCC(NC(=O)c3ccc(C(=O)N4CCC(Oc5ccncc5)CC4)cn3)CC2)cc1] |
| 270 | (S)-N-(1-(4-cyanobenzyl)piperidin-4-yl)-5- (3-(4-fluorophenoxy)pyrrolidine-1- carbonyl)picolinamide | 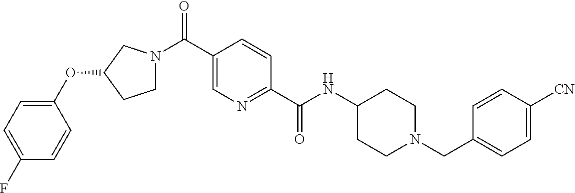 |
| 271 | N-(1-(4-cyanobenzyl)piperidin-4-yl)-5-(4- (2,4-difluorobenzoyl)piperidine-1- carbonyl)picolinamide | 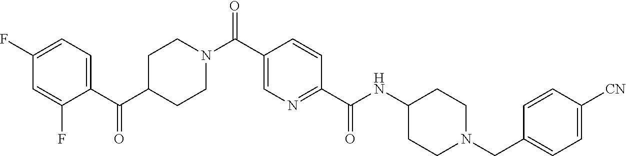 [SMILES: N#Cc1ccc(CN2CCC(NC(=O)c3ccc(C(=O)N4CCC(C(=O)c5ccc(F)cc5F)CC4)cn3)CC2)cc1] |
| 272 | 5-(4-(4-fluorobenzoyl)piperidine-1- carbonyl)-N-(6-(4-fluorophenoxy)pyridin-3- yl)picolinamide | 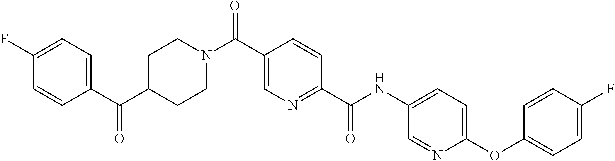 [SMILES: O=C(Nc1ccc(Oc2ccc(F)cc2)nc1)c1ccc(C(=O)N2CCC(C(=O)c3ccc(F)cc3)CC2)cn1] |
| 273 | 5-(4-(4-fluorophenoxy)piperidine-1- carbonyl)-N-(6-(4-fluorophenoxy)pyridin-3- yl)picolinamide | 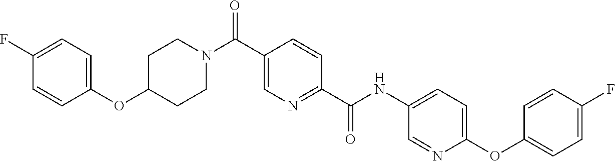 [SMILES: O=C(Nc1ccc(Oc2ccc(F)cc2)nc1)c1ccc(C(=O)N2CCC(Oc3ccc(F)cc3)CC2)cn1] |
| 274 | N-(1-(4-cyanobenzyl)piperidin-4-yl)-5-(3- (4-methoxyphenoxy)piperidine-1- carbonyl)picolinamide | 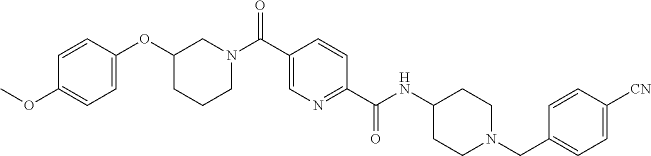 [SMILES: COc1ccc(OC2CCCN(C(=O)c3ccc(C(=O)NC4CCN(Cc5ccc(C#N)cc5)CC4)nc3)C2)cc1] |
| 275 | N-(1-(4-cyanobenzyl)piperidin-4-yl)-5-(1- (4-methoxyphenyl)piperidin-4- ylamino)picolinamide | 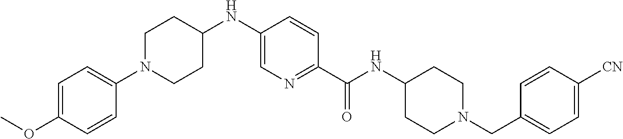 [SMILES: COc1ccc(N2CCC(Nc3ccc(C(=O)NC4CCN(Cc5ccc(C#N)cc5)CC4)nc3)CC2)cc1] |
| 276 | N-(1-(4-cyanobenzyl)piperidin-4-yl)-5-(1- (4-fluorophenyl)piperidin-4- ylamino)picolinamide | 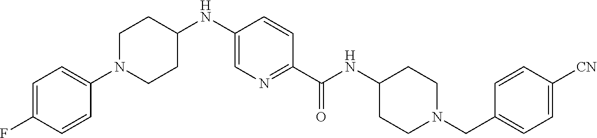 [SMILES: N#Cc1ccc(CN2CCC(NC(=O)c3ccc(NC4CCN(c5ccc(F)cc5)CC4)cn3)CC2)cc1] |
| 277 | N-(1-(4-cyanobenzyl)piperidin-4-yl)-5-(3- (3-methoxyphenoxy)piperidine-1- carbonyl)picolinamide | 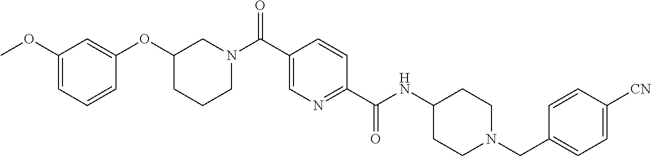 [SMILES: COc1cccc(OC2CCCN(C(=O)c3ccc(C(=O)NC4CCN(Cc5ccc(C#N)cc5)CC4)nc3)C2)c1] |
| 278 | (R)-N-(1-(4-cyanobenzyl)piperidin-4-yl)-5- (3-(4-fluorophenoxy)pyrrolidine-1- carbonyl)picolinamide | 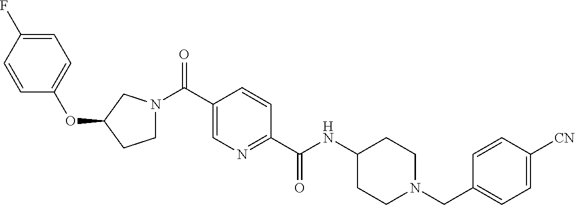 [SMILES: N#Cc1ccc(CN2CCC(NC(=O)c3ccc(C(=O)N4CC[C@@H](Oc5ccc(F)cc5)C4)cn3)CC2)cc1] |
| 279 | N-(1-(4-cyanobenzyl)piperidin-4-yl)-5- ((trans)-4-(4-cyanophenoxy)-3- fluoropiperidine-1-carbonyl)picolinamide | 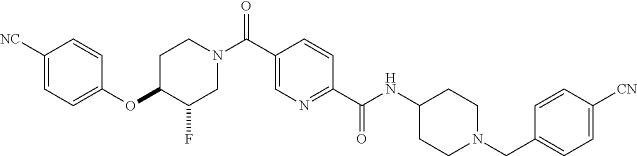 |
| 280 | N-(1-(4-cyanobenzyl)piperidin-4-yl)-5- ((1R,3r,5S)-3-(4-cyanophenoxy)-8- azabicyclo[3.2.1]octane-8- carbonyl)picolinamide | 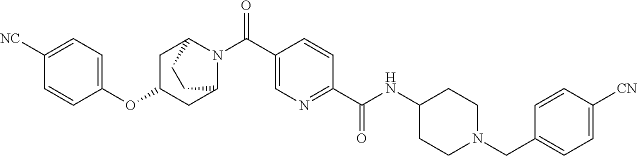 |
| 281 | N-(1-(4-cyanobenzyl)piperidin-4-yl)-5-(4- (3,4-difluorobenzoyl)piperidine-1- carbonyl)picolinamide | 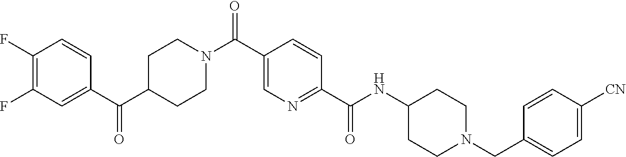 [SMILES: N#Cc1ccc(CN2CCC(NC(=O)c3ccc(C(=O)N4CCC(C(=O)c5ccc(F)c(F)c5)CC4)cn3)CC2)cc1] |
| 282 | N-(1-(4-cyanobenzyl)piperidin-4-yl)-5-(4- (2,4-difluorophenoxy)piperidine-1- carbonyl)picolinamide | 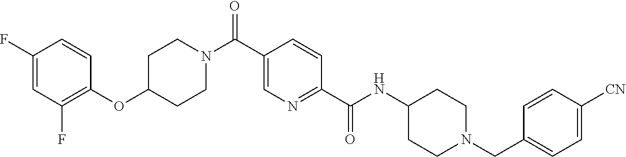 [SMILES: N#Cc1ccc(CN2CCC(NC(=O)c3ccc(C(=O)N4CCC(Oc5ccc(F)cc5F)CC4)cn3)CC2)cc1] |
| 283 | N-(1-(4-cyanobenzyl)piperidin-4-yl)-5-(4- (pyridin-3-yloxy)piperidine-1- carbonyl)picolinamide | 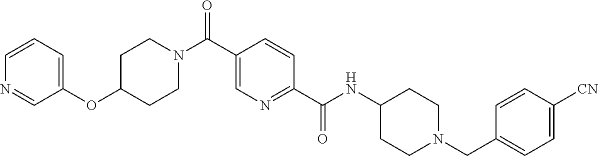 [SMILES: N#Cc1ccc(CN2CCC(NC(=O)c3ccc(C(=O)N4CCC(Oc5cccnc5)CC4)cn3)CC2)cc1] |
| 284 | ethyl 4-(1-(6-(1-(4-cyanobenzyl)piperidin-4- ylcarbamoyl)nicotinoyl)piperidin-4- yloxy)benzoate | 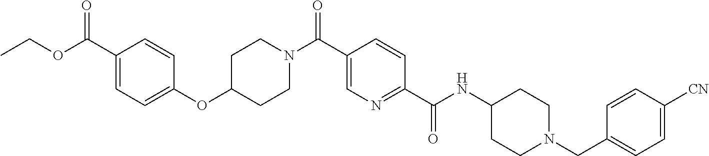 [SMILES: CCOC(=O)c1ccc(OC2CCN(C(=O)c3ccc(C(=O)NC4CCN(Cc5ccc(C#N)cc5)CC4)nc3)CC2)cc1] |
| 285 | 5-(4-(4-cyanobenzyl)piperazine-1- carbonyl)-N-(1-(4- methoxybenzyl)piperidin-4-yl)picolinamide | 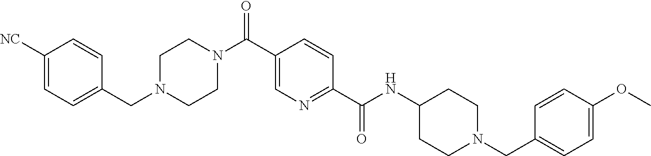 [SMILES: COc1ccc(CN2CCC(NC(=O)c3ccc(C(=O)N4CCN(Cc5ccc(C#N)cc5)CC4)cn3)CC2)cc1] |
| 286 | 5-(4-(4-cyano-2- methoxyphenoxy)piperidin-1-yl)-N-(1-(4- cyanobenzyl)piperidin-4-yl)picolinamide | 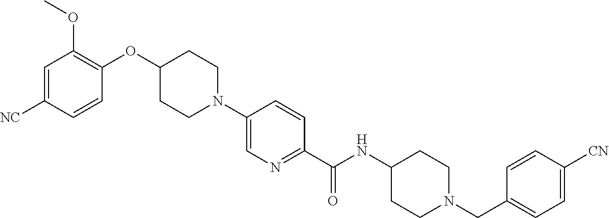 [SMILES: COc1cc(C#N)ccc1OC1CCN(c2ccc(C(=O)NC3CCN(Cc4ccc(C#N)cc4)CC3)nc2)CC1] |
| 287 | N-(1-(3,5-difluorobenzyl)piperidin-4-yl)-5- (4-(4-methoxybenzoyl)piperidine-1- carbonyl)picolinamide | 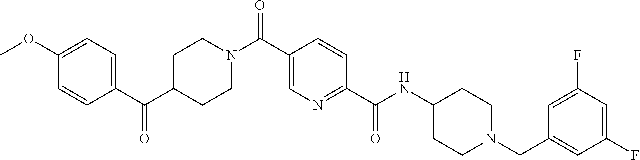 [SMILES: COc1ccc(C(=O)C2CCN(C(=O)c3ccc(C(=O)NC4CCN(Cc5cc(F)cc(F)c5)CC4)nc3)CC2)cc1] |
| 288 | N-(1-(3,5-difluorobenzyl)piperidin-4-yl)-5- (4-(4-fluorobenzoyl)piperidine-1- carbonyl)picolinamide | 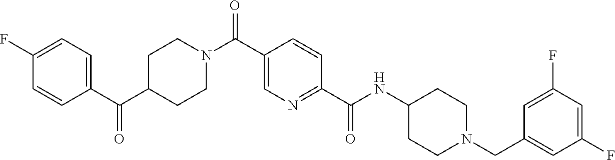 [SMILES: O=C(NC1CCN(Cc2cc(F)cc(F)c2)CC1)c1ccc(C(=O)N2CCC(C(=O)c3ccc(F)cc3)CC2)cn1] |
| 289 | 5-(4-(4-cyanophenoxy)piperidine-1- carbonyl)-N-(1-(3,5- difluorobenzyl)piperidin-4-yl)picolinamide | 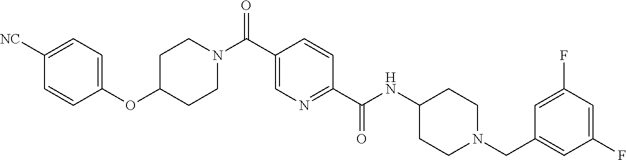 [SMILES: N#Cc1ccc(OC2CCN(C(=O)c3ccc(C(=O)NC4CCN(Cc5cc(F)cc(F)c5)CC4)nc3)CC2)cc1] |
| 290 | tert-butyl 3-(2-(1-(4-cyanobenzyl)piperidin- 4-ylcarbamoyl)-5-(4-(4- fluorobenzyl)piperazine-1-carbonyl)pyridin- 3-yl)propylcarbamate | 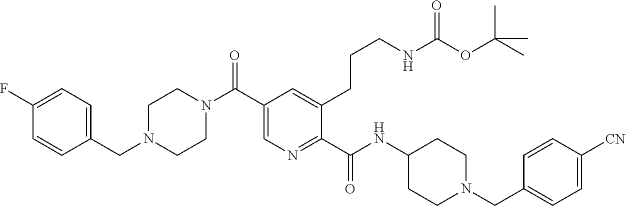 [SMILES: CC(C)(C)OC(=O)NCCCc1cc(C(=O)N2CCN(Cc3ccc(F)cc3)CC2)cnc1C(=O)NC1CCN(Cc2ccc(C#N)cc2)CC1] |
| 291 | N-(1-(4-cyanobenzyl)piperidin-4-yl)-3- (5,21-dioxo-25-((3aS,4S,6aR)-2- oxohexahydro-1H-thieno[3,4-d]imidazol-4- yl)-8,11,14,17-tetraoxa-4,20- diazapentacosyl)-5-(4-(4- fluorobenzyl)piperazine-1- carbonyl)picolinamide | 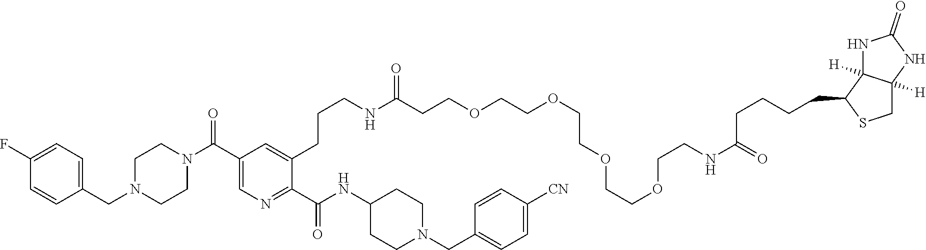 |
| 292 | N-(1-(3,5-difluorobenzyl)piperidin-4-yl)-5- ((S)-3-(4-fluorophenoxy)pyrrolidine-1- carbonyl)picolinamide | 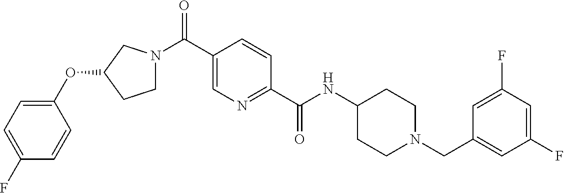 |
| 293 | N-(1-(3,5-difluorobenzyl)piperidin-4-yl)-5- (4-(p-tolyloxy)piperidine-1- carbonyl)picolinamide | 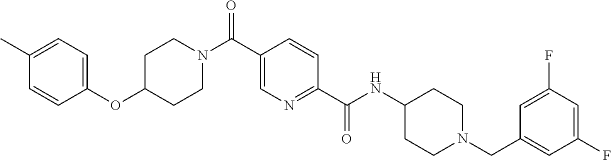 [SMILES: Cc1ccc(OC2CCN(C(=O)c3ccc(C(=O)NC4CCN(Cc5cc(F)cc(F)c5)CC4)nc3)CC2)cc1] |
| 294 | N-(1-(3,5-difluorobenzyl)piperidin-4-yl)-5- (4-(4-(trifluoromethyl)phenoxy)piperidine- 1-carbonyl)picolinamide | 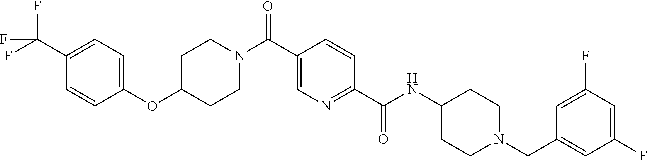 [SMILES: O=C(NC1CCN(Cc2cc(F)cc(F)c2)CC1)c1ccc(C(=O)N2CCC(Oc3ccc(C(F)(F)F)cc3)CC2)cn1] |
| 295 | N-(1-(3,5-difluorobenzyl)piperidin-4-yl)-5- (4-(4-fluorophenoxy)piperidine-1- carbonyl)picolinamide | 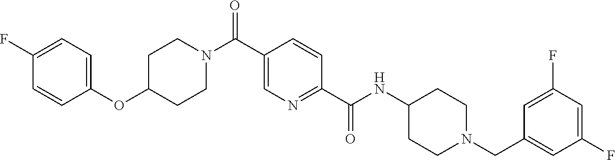 [SMILES: O=C(NC1CCN(Cc2cc(F)cc(F)c2)CC1)c1ccc(C(=O)N2CCC(Oc3ccc(F)cc3)CC2)cn1] |
| 296 | N-(1-(3,5-difluorobenzyl)piperidin-4-yl)-5- (4-(4-methoxyphenoxy)piperidine-1- carbonyl)picolinamide | 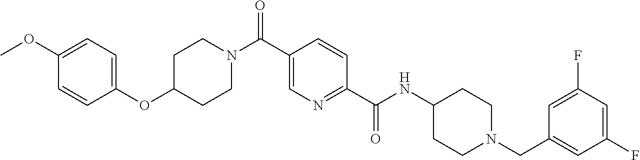 [SMILES: COc1ccc(OC2CCN(C(=O)c3ccc(C(=O)NC4CCN(Cc5cc(F)cc(F)c5)CC4)nc3)CC2)cc1] |
| 297 | N-(1-(3,5-difluorobenzyl)piperidin-4-yl)-5- (4-(3,4-difluorophenoxy)piperidine-1- carbonyl)picolinamide | 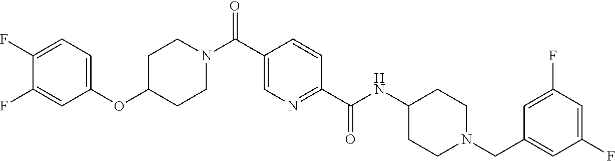 [SMILES: O=C(NC1CCN(Cc2cc(F)cc(F)c2)CC1)c1ccc(C(=O)N2CCC(Oc3ccc(F)c(F)c3)CC2)cn1] |
| 298 | 5-(4-(3,4-difluorobenzoyl)piperidine-1- carbonyl)-N-(1-(3,5- difluorobenzyl)piperidin-4-yl)picolinamide | 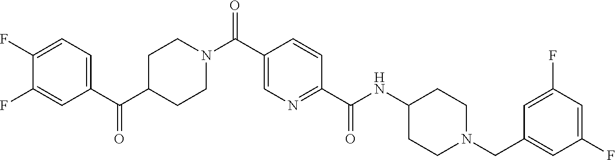 [SMILES: O=C(NC1CCN(Cc2cc(F)cc(F)c2)CC1)c1ccc(C(=O)N2CCC(C(=O)c3ccc(F)c(F)c3)CC2)cn1] |
| 299 | N-((cis)-4-(3,5- difluorophenoxy)cyclohexyl)-5-(4-(4- fluorophenoxy)piperidine-1- carbonyl)picolinamide | 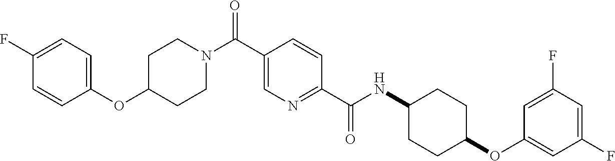 |
| 300 | N-((cis)-4-(3,5- difluorophenoxy)cyclohexyl)-5-(4-(4- methoxybenzoyl)piperidine-1- carbonyl)picolinamide | 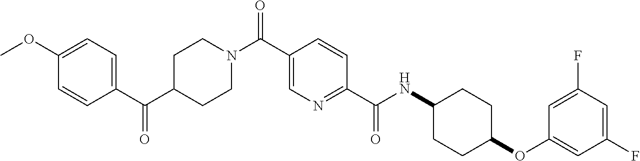 |
| 301 | N-(1-(4-cyanobenzyl)piperidin-4-yl)-5-(4- (4-(trifluoromethyl)phenoxy)piperidin-1- yl)picolinamide | 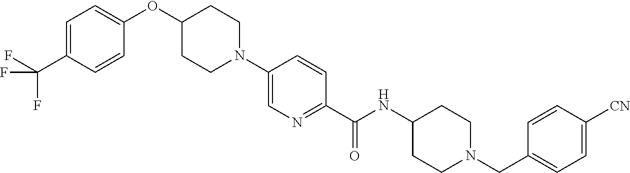 [SMILES: N#Cc1ccc(CN2CCC(NC(=O)c3ccc(N4CCC(Oc5ccc(C(F)(F)F)cc5)CC4)cn3)CC2)cc1] |
| 302 | N-(1-(4-cyanobenzyl)piperidin-4-yl)-5-(4- (4-methoxybenzoyl)piperidin-1- yl)picolinamide | 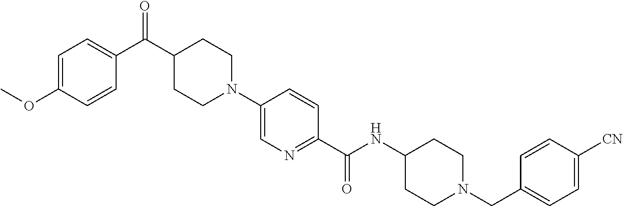 [SMILES: COc1ccc(C(=O)C2CCN(c3ccc(C(=O)NC4CCN(Cc5ccc(C#N)cc5)CC4)nc3)CC2)cc1] |
| 303 | 5-(4-(4-cyanophenoxy)piperidine-1- carbonyl)-N-((cis)-4-(4- fluorophenoxy)cyclohexyl)picolinamide | 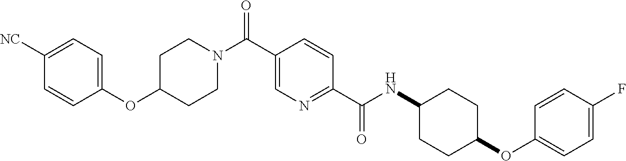 |
| 304 | 5-(4-(4-fluorobenzoyl)piperidine-1- carbonyl)-N-((cis)-4-(4- fluorophenoxy)cyclohexyl)picolinamide | 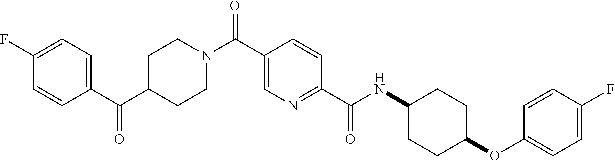 |
| 305 | N-(2-(4-fluorophenoxy)ethyl)-5-(4-(4- methoxybenzoyl)piperidine-1- carbonyl)picolinamide | 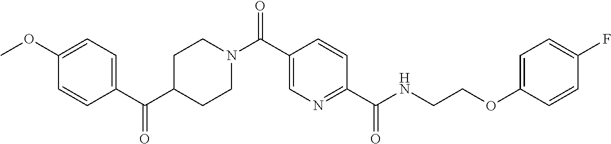 [SMILES: COc1ccc(C(=O)C2CCN(C(=O)c3ccc(C(=O)NCCOc4ccc(F)cc4)nc3)CC2)cc1] |
| 306 | 5-(4-(4-cyanophenoxy)piperidine-1- carbonyl)-N-(2-(4- fluorophenoxy)ethyl)picolinamide | 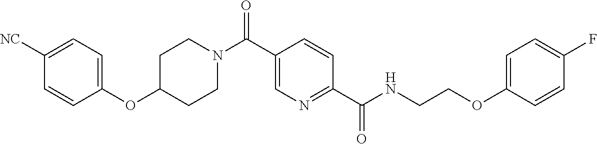 [SMILES: N#Cc1ccc(OC2CCN(C(=O)c3ccc(C(=O)NCCOc4ccc(F)cc4)nc3)CC2)cc1] |
| 307 | N-(1-(4-cyanobenzyl)piperidin-4-yl)-5-(3- (4-fluorobenzyloxy)azetidine-1- carbonyl)picolinamide | 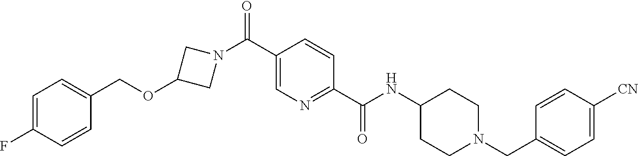 [SMILES: N#Cc1ccc(CN2CCC(NC(=O)c3ccc(C(=O)N4CC(OCc5ccc(F)cc5)C4)cn3)CC2)cc1] |
| 308 | N-(1-(3,5-difluorobenzyl)piperidin-4-yl)-5- (3-(4-fluorobenzyloxy)azetidine-1- carbonyl)picolinamide | 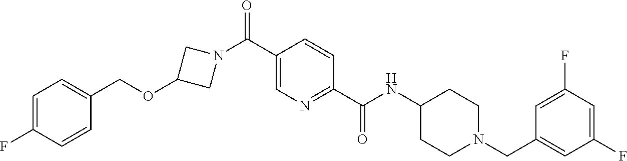 [SMILES: O=C(NC1CCN(Cc2cc(F)cc(F)c2)CC1)c1ccc(C(=O)N2CC(OCc3ccc(F)cc3)C2)cn1] |
| 309 | N-(1-(4-cyanobenzyl)piperidin-4-yl)-6-(4- (4-methoxybenzoyl)piperidine-1- carbonyl)nicotinamide | 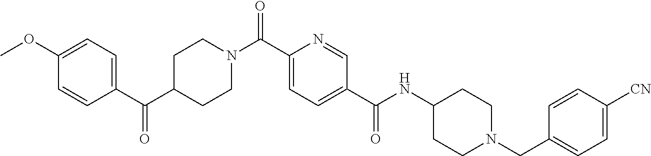 [SMILES: COc1ccc(C(=O)C2CCN(C(=O)c3ccc(C(=O)NC4CCN(Cc5ccc(C#N)cc5)CC4)cn3)CC2)cc1] |
| 310 | N-(1-(3,5-difluorobenzyl)piperidin-4-yl)-6- (4-(4-methoxybenzoyl)piperidine-1- carbonyl)nicotinamide | 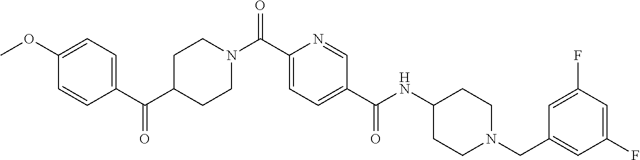 [SMILES: COc1ccc(C(=O)C2CCN(C(=O)c3ccc(C(=O)NC4CCN(Cc5cc(F)cc(F)c5)CC4)cn3)CC2)cc1] |
| 311 | N-((cis)-4-(4-fluorophenoxy)cyclohexyl)-5- (4-(4-methoxybenzoyl)piperidine-1- carbonyl)picolinamide | 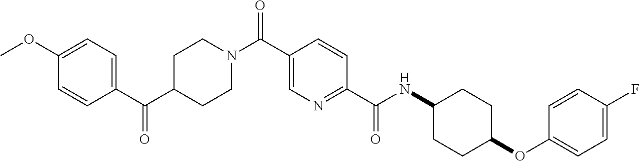 |
| 312 | N-((cis)-4-(4-fluorophenoxy)cyclohexyl)-5- (4-(4-fluorophenoxy)piperidine-1- carbonyl)picolinamide | 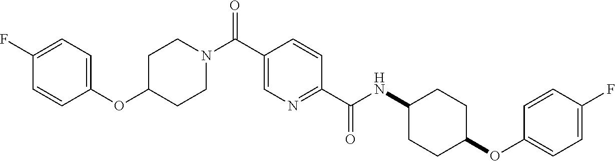 |
| 313 | 5-(3-(4-cyanophenoxy)azetidine-1- carbonyl)-N-(1-(3,5- difluorobenzyl)piperidin-4-yl)picolinamide | 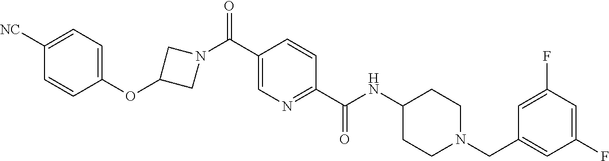 [SMILES: N#Cc1ccc(OC2CN(C(=O)c3ccc(C(=O)NC4CCN(Cc5cc(F)cc(F)c5)CC4)nc3)C2)cc1] |
| 314 | 5-(3-(4-cyanophenyl)-5,6,7,8-tetrahydro- [1,2,4]triazolo[4,3-a]pyrazine-7-carbonyl)- N-(1-(3,5-difluorobenzyl)piperidin-4- yl)picolinamide | 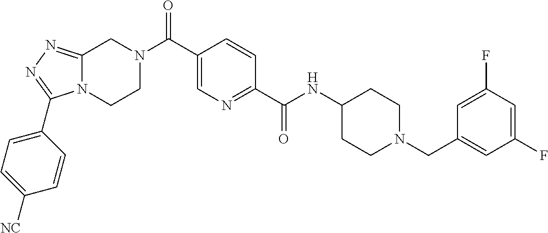 [SMILES: N#Cc1ccc(-c2nnc3n2CCN(C(=O)c2ccc(C(=O)NC4CCN(Cc5cc(F)cc(F)c5)CC4)nc2)C3)cc1] |
| 315 | N-((1s,4s)-4-(4-cyanophenoxy)cyclohexyl)- 6-(4-(4-methoxybenzoyl)piperidine-1- carbonyl)nicotinamide | 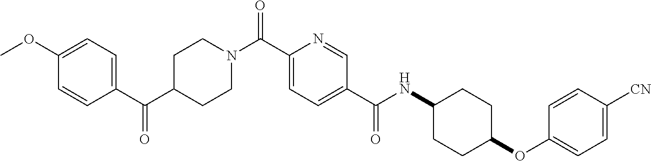 |
| 316 | N-((cis)-4-(4-fluorophenoxy)cyclohexyl)-6- (4-(4-methoxybenzoyl)piperidine-1- carbonyl)nicotinamide | 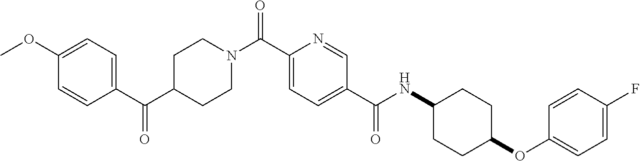 |
| 317 | N-(1-(4-fluorobenzyl)piperidin-4-yl)-6-(4- (4-methoxybenzoyl)piperidine-1- carbonyl)nicotinamide | 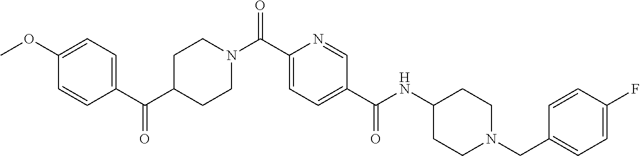 [SMILES: COc1ccc(C(=O)C2CCN(C(=O)c3ccc(C(=O)NC4CCN(Cc5ccc(F)cc5)CC4)cn3)CC2)cc1] |
| 318 | 6-(4-(4-methoxybenzoyl)piperidine-1- carbonyl)-N-(1-(4- methoxybenzyl)piperidin-4-yl) nicotinamide | 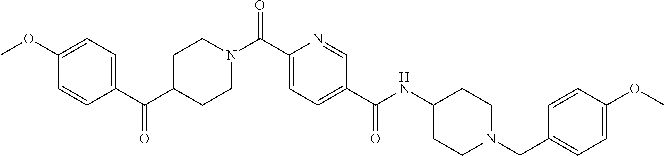 |
| 319 | 6-(4-(4-cyanophenoxy)piperidine-1- carbonyl)-N-(1-(4- methoxybenzyl)piperidin-4-yl) nicotinamide | 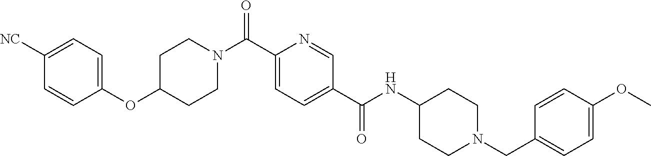 [SMILES: COc1ccc(CN2CCC(NC(=O)c3ccc(C(=O)N4CCC(Oc5ccc(C#N)cc5)CC4)nc3)CC2)cc1] |
| 320 | 6-(4-(4-cyanophenoxy)piperidine-1- carbonyl)-N-(1-(4-fluorobenzyl) piperidin-4-yl)nicotinamide | 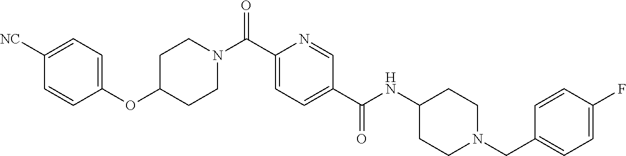 [SMILES: N#Cc1ccc(OC2CCN(C(=O)c3ccc(C(=O)NC4CCN(Cc5ccc(F)cc5)CC4)cn3)CC2)cc1] |
| 321 | N-((cis)-4-(4-cyanophenoxy)cyclohexyl)-6- (4-(4-cyanophenoxy)piperidine-1- carbonyl)nicotinamide | 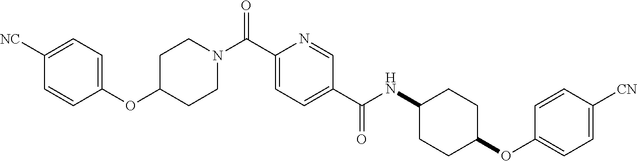 |
| 322 | 6-(4-(4-cyanophenoxy)piperidine-1- carbonyl)-N-(1-(3,5- difluorobenzyl)piperidin-4-yl)nicotinamide | 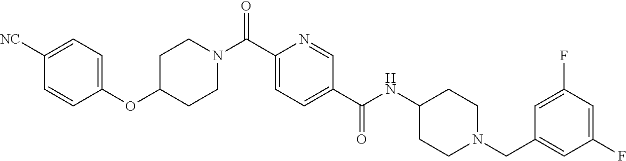 [SMILES: N#Cc1ccc(OC2CCN(C(=O)c3ccc(C(=O)NC4CCN(Cc5cc(F)cc(F)c5)CC4)cn3)CC2)cc1] |
| 323 | N-(1-(4-cyanobenzyl)piperidin-4-yl)-6-(4- (4-cyanophenoxy)piperidine-1- carbonyl)nicotinamide | 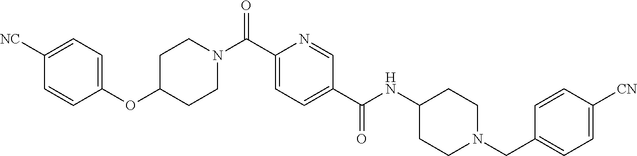 [SMILES: N#Cc1ccc(CN2CCC(NC(=O)c3ccc(C(=O)N4CCC(Oc5ccc(C#N)cc5)CC4)nc3)CC2)cc1] |
| 324 | 6-(4-(4-cyanophenoxy)piperidine-1- carbonyl)-N-((cis)-4-(4- fluorophenoxy)cyclohexyl)nicotinamide | 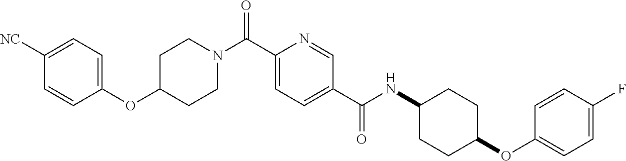 |
| 325 | N-(6-(4-fluorophenoxy)pyridin-3-yl)-6-(4- (4-methoxybenzoyl)piperidine-1- carbonyl)nicotinamide | 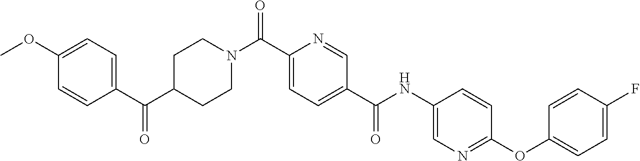 [SMILES: COc1ccc(C(=O)C2CCN(C(=O)c3ccc(C(=O)Nc4ccc(Oc5ccc(F)cc5)nc4)cn3)CC2)cc1] |
| 326 | 6-(4-(4-cyanophenoxy)piperidine-1- carbonyl)-N-(6-(4-fluorophenoxy) pyridin-3-yl)nicotinamide | 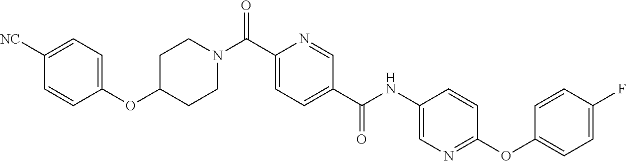 [SMILES: N#Cc1ccc(OC2CCN(C(=O)c3ccc(C(=O)Nc4ccc(Oc5ccc(F)cc5)nc4)cn3)CC2)cc1] |
| 327 | 6-(4-(4-fluorobenzyl)piperazine-1- carbonyl)-N-(1-(4- methoxybenzyl)piperidin-4-yl) nicotinamide | 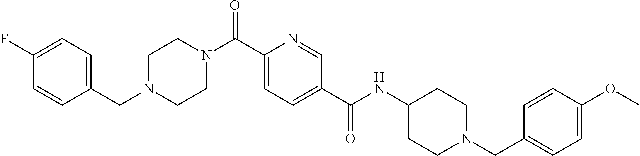 [SMILES: COc1ccc(CN2CCC(NC(=O)c3ccc(C(=O)N4CCN(Cc5ccc(F)cc5)CC4)nc3)CC2)cc1] |
| 328 | 6-(4-(4-fluorobenzyl)piperazine-1- carbonyl)-N-(1-(4-fluorobenzyl) piperidin-4-yl)nicotinamide | 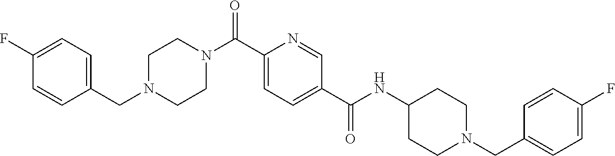 [SMILES: O=C(NC1CCN(Cc2ccc(F)cc2)CC1)c1ccc(C(=O)N2CCN(Cc3ccc(F)cc3)CC2)nc1] |
| 329 | 5-(4-(3,4-difluorobenzoyl)piperidine-1- carbonyl)-N-(1-(4- methoxybenzyl)piperidin-4-yl) picolinamide | 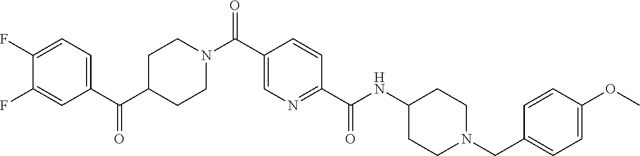 [SMILES: COc1ccc(CN2CCC(NC(=O)c3ccc(C(=O)N4CCC(C(=O)c5ccc(F)c(F)c5)CC4)cn3)CC2)cc1] |
| 330 | 5-(4-(3,4-difluorobenzoyl)piperidine-1- carbonyl)-N-(6-(4-fluorophenoxy) pyridin-3-yl)picolinamide | 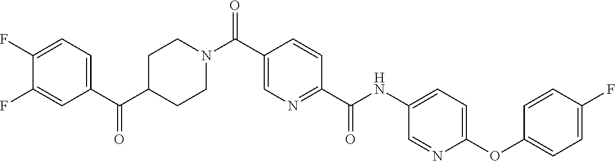 [SMILES: O=C(Nc1ccc(Oc2ccc(F)cc2)nc1)c1ccc(C(=O)N2CCC(C(=O)c3ccc(F)c(F)c3)CC2)cn1] |
| 331 | 5-(4-(2,4-difluorobenzoyl)piperidine-1- carbonyl)-N-(1-(4- methoxybenzyl)piperidin-4-yl) picolinamide | 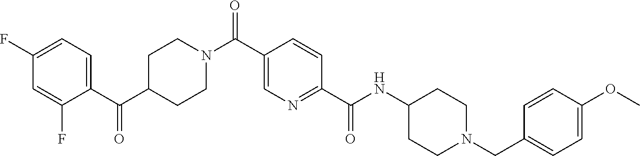 [SMILES: COc1ccc(CN2CCC(NC(=O)c3ccc(C(=O)N4CCC(C(=O)c5ccc(F)cc5F)CC4)cn3)CC2)cc1] |
| 332 | N-((cis)-4-(4-cyanophenoxy) cyclohexyl)-6- (4-(4-fluorobenzyl)piperazine-1- carbonyl)nicotinamide | 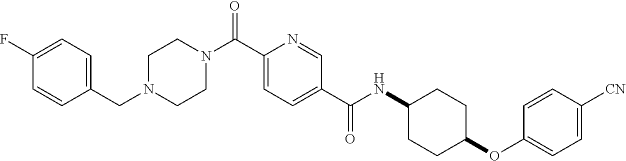 |
| 333 | tert-butyl 4-(6-(4-(4- cyanophenoxy)piperidine-1- carbonyl)nicotinamido) piperidine-1-carboxylate | 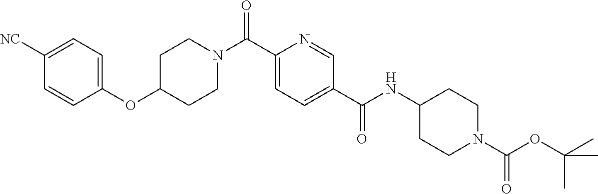 [SMILES: CC(C)(C)OC(=O)N1CCC(NC(=O)c2ccc(C(=O)N3CCC(Oc4ccc(C#N)cc4)CC3)nc2)CC1] |
| 334 | 6-(4-(4-fluorobenzyl)piperazine-1- carbonyl)-N-(6-(4-fluorophenoxy) pyridin-3- yl)nicotinamide | 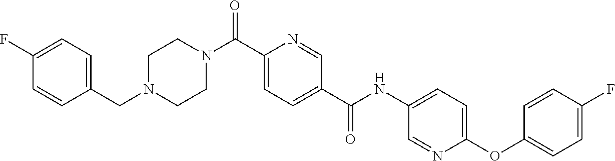 [SMILES: O=C(Nc1ccc(Oc2ccc(F)cc2)nc1)c1ccc(C(=O)N2CCN(Cc3ccc(F)cc3)CC2)nc1] |
| 335 | 6-(4-(4-cyanophenoxy)piperidine-1- carbonyl)-N-(piperidin-4-yl) nicotinamide | 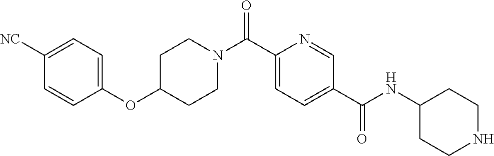 [SMILES: N#Cc1ccc(OC2CCN(C(=O)c3ccc(C(=O)NC4CCNCC4)cn3)CC2)cc1] |
| 336 | 6-(4-(4-cyanophenoxy)piperidine-1- carbonyl)-N-(1-(4-(pyrrolidin-1- yl)benzyl)piperidin-4-yl)nicotinamide | 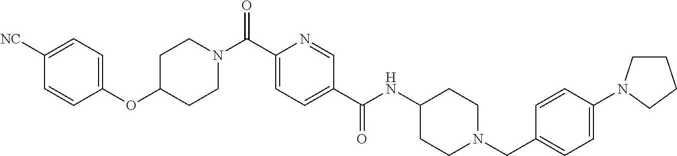 [SMILES: N#Cc1ccc(OC2CCN(C(=O)c3ccc(C(=O)NC4CCN(Cc5ccc(N6CCCC6)cc5)CC4)cn3)CC2)cc1] |
| 337 | 6-(4-(4-cyanophenoxy)piperidine-1- carbonyl)-N-(1-(4-morpholinobenzyl) piperidin-4-yl)nicotinamide | 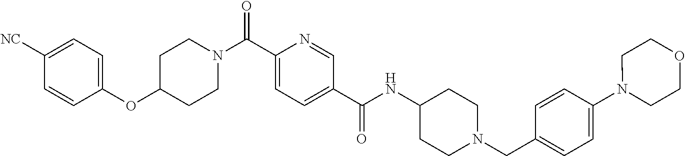 [SMILES: N#Cc1ccc(OC2CCN(C(=O)c3ccc(C(=O)NC4CCN(Cc5ccc(N6CCOCC6)cc5)CC4)cn3)CC2)cc1] |
| 338 | 6-(4-(4-cyanophenoxy)piperidine-1- carbonyl)-N-(1-(4- (trifluoromethoxy)benzyl)piperidin-4- yl)nicotinamide | 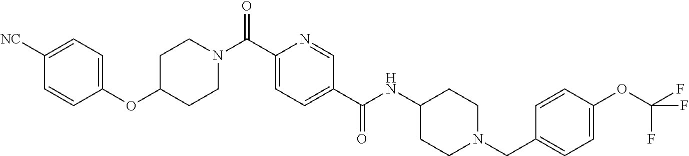 [SMILES: N#Cc1ccc(OC2CCN(C(=O)c3ccc(C(=O)NC4CCN(Cc5ccc(OC(F)(F)F)cc5)CC4)cn3)CC2)cc1] |
| 339 | N-(1-(4-cyanobenzyl)piperidin-4-yl)- 5-(4-(4-(trifluoromethyl)phenyl) piperazine-1- carbonyl)picolinamide | 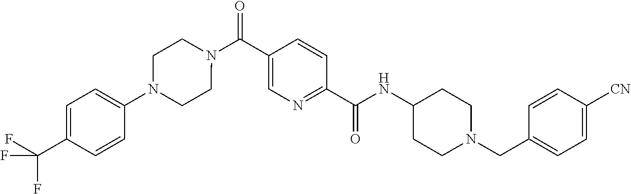 [SMILES: N#Cc1ccc(CN2CCC(NC(=O)c3ccc(C(=O)N4CCN(c5ccc(C(F)(F)F)cc5)CC4)cn3)CC2)cc1] |
| 340 | N-(1-(4-cyanobenzyl)piperidin-4- yl)-5-(4- (4-cyanophenyl)piperazine-1- carbonyl)picolinamide | 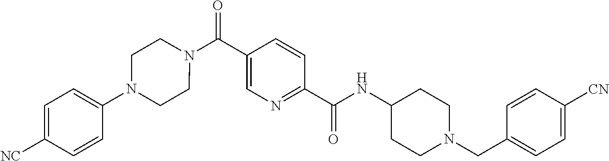 [SMILES: N#Cc1ccc(CN2CCC(NC(=O)c3ccc(C(=O)N4CCN(c5ccc(C#N)cc5)CC4)cn3)CC2)cc1] |
| 341 | N-(1-(4-cyanobenzyl)piperidin-4- yl)-5-(4- (4-fluorophenyl)piperazine-1- carbonyl)picolinamide | 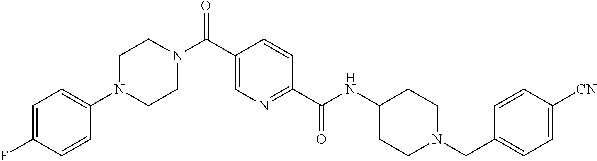 [SMILES: N#Cc1ccc(CN2CCC(NC(=O)c3ccc(C(=O)N4CCN(c5ccc(F)cc5)CC4)cn3)CC2)cc1] |
| 342 | 5-(4-(2,4-difluorobenzoyl)piperidine- 1-carbonyl)-N-(6-(4-fluorophenoxy) pyridin-3- yl)picolinamide | 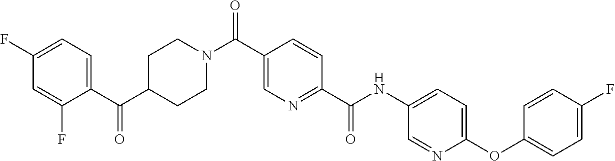 [SMILES: O=C(Nc1ccc(Oc2ccc(F)cc2)nc1)c1ccc(C(=O)N2CCC(C(=O)c3ccc(F)cc3F)CC2)cn1] |
| 343 | 6-(4-(2,4-difluorophenoxy)piperidine-1- carbonyl)-N-(6-(4-fluorophenoxy)pyridin-3- yl)nicotinamide | 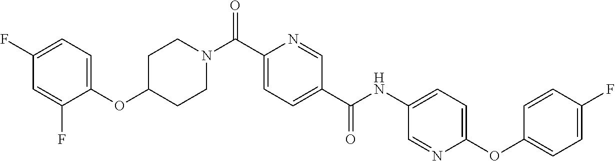 [SMILES: O=C(Nc1ccc(Oc2ccc(F)cc2)nc1)c1ccc(C(=O)N2CCC(Oc3ccc(F)cc3F)CC2)nc1] |
| 344 | 6-(4-(2,4-difluorophenoxy)piperidine-1- carbonyl)-N-(1-(4- methoxybenzyl)piperidin-4-yl)nicotinamide | 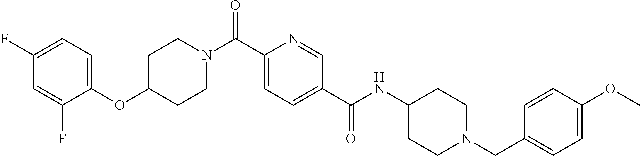 [SMILES: COc1ccc(CN2CCC(NC(=O)c3ccc(C(=O)N4CCC(Oc5ccc(F)cc5F)CC4)nc3)CC2)cc1] |
| 345 | N-(1-(4-cyanobenzyl)piperidin-4-yl)-6-(4- (2,4-difluorophenoxy)piperidine-1- carbonyl)nicotinamide | 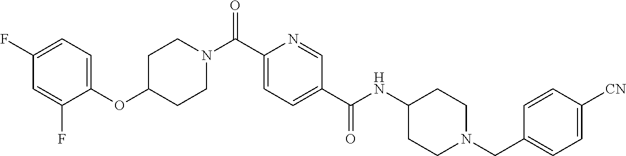 [SMILES: N#Cc1ccc(CN2CCC(NC(=O)c3ccc(C(=O)N4CCC(Oc5ccc(F)cc5F)CC4)nc3)CC2)cc1] |
| 346 | N-(1-(4-cyanobenzyl)piperidin-4-yl)-6-(4- (2,4-difluorobenzoyl)piperidine-1- carbonyl)nicotinamide | 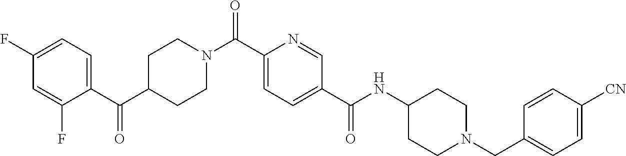 [SMILES: N#Cc1ccc(CN2CCC(NC(=O)c3ccc(C(=O)N4CCC(C(=O)c5ccc(F)cc5F)CC4)nc3)CC2)cc1] |
| 347 | 6-(4-(2,4-difluorobenzoyl)piperidine-1- carbonyl)-N-(1-(4- methoxybenzyl)piperidin-4-yl)nicotinamide | 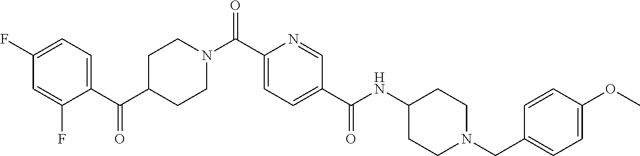 [SMILES: COc1ccc(CN2CCC(NC(=O)c3ccc(C(=O)N4CCC(C(=O)c5ccc(F)cc5F)CC4)nc3)CC2)cc1] |
| 348 | 6-(4-(2,4-difluorobenzoyl)piperidine-1- carbonyl)-N-(6-(4-fluorophenoxy)pyridin-3- yl)nicotinamide | 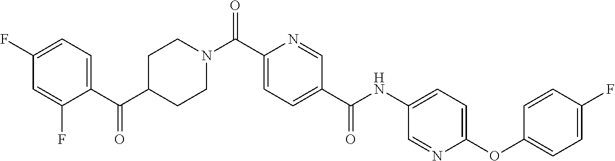 [SMILES: O=C(Nc1ccc(Oc2ccc(F)cc2)nc1)c1ccc(C(=O)N2CCC(C(=O)c3ccc(F)cc3F)CC2)nc1] |
| 349 | N-((trans)-1-(4-cyanobenzyl)-3- fluoropiperidin-4-yl)-6-(4-(4- methoxybenzoyl)piperidine-1- carbonyl)nicotinamide | 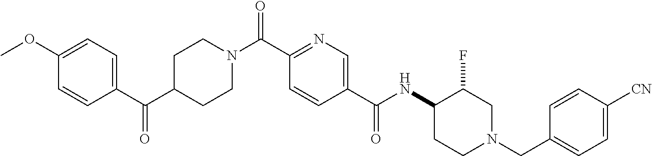 |
| 350 | N-((trans)-3-fluoro-1-(4- (trifluoromethoxy)benzyl)piperidin-4-yl)- 6-(4-(4-methoxybenzoyl)piperidine-1- carbonyl)nicotinamide | 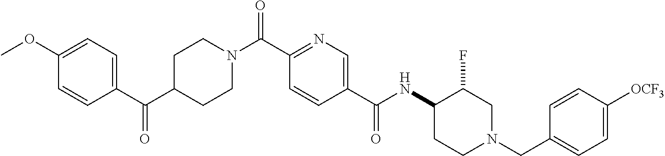 |
| 351 | N-(1-(4-cyanobenzyl)piperidin-4-yl)-6-(4- (4-cyanophenoxy)piperidin-1-yl) pyridazine- 3-carboxamide | 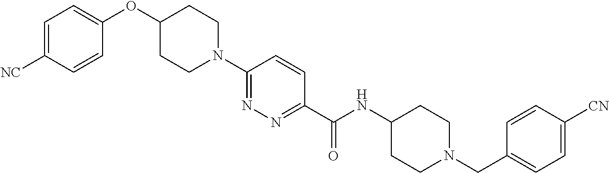 [SMILES: N#Cc1ccc(CN2CCC(NC(=O)c3ccc(N4CCC(Oc5ccc(C#N)cc5)CC4)nn3)CC2)cc1] |
| 352 | N-((trans)-3-fluoro-1-(4-(pyrrolidin-1- yl)benzyl)piperidin-4-yl)-6-(4-(4- methoxybenzoyl)piperidine-1- carbonyl)nicotinamide | 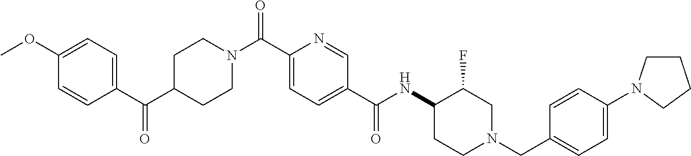 |
| 353 | N-((trans)-3-fluoro-1-(4- isopropoxybenzyl)piperidin-4-yl)-6-(4-(4- methoxybenzoyl)piperidine-1- carbonyl)nicotinamide | 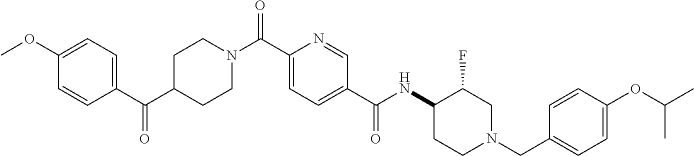 |
| 354 | N-((trans)-1-(4-cyano-3-fluorobenzyl)-3- fluoropiperidin-4-yl)-6-(4-(4- methoxybenzoyl)piperidine-1- carbonyl)nicotinamide | 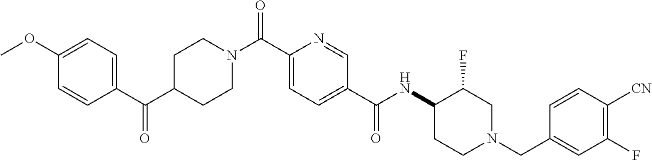 |
| 355 | 6-(4-(4-cyanophenoxy)piperidine-1- carbonyl)-N-(1-(oxazol-4- ylmethyl)piperidin-4-yl)nicotinamide | 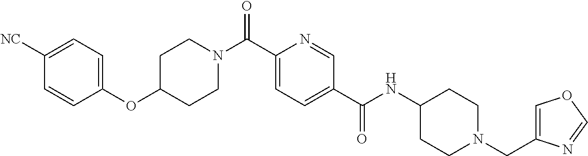 [SMILES: N#Cc1ccc(OC2CCN(C(=O)c3ccc(C(=O)NC4CCN(Cc5cocn5)CC4)cn3)CC2)cc1] |
| 356 | 6-(4-(4-cyanophenoxy)piperidine-1- carbonyl)-N-(1-(thiazol-2- ylmethyl)piperidin-4-yl)nicotinamide | 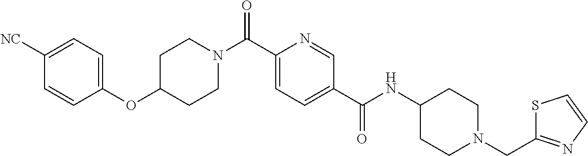 [SMILES: N#Cc1ccc(OC2CCN(C(=O)c3ccc(C(=O)NC4CCN(Cc5nccs5)CC4)cn3)CC2)cc1] |
| 357 | N-(1-(4-cyanobenzyl)piperidin-4-yl)-5-(4- (4-(dimethylcarbamoyl)phenoxy)piperidine- 1-carbonyl)picolinamide | 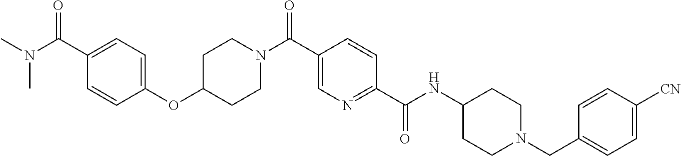 [SMILES: CN(C)C(=O)c1ccc(OC2CCN(C(=O)c3ccc(C(=O)NC4CCN(Cc5ccc(C#N)cc5)CC4)nc3)CC2)cc1] |
| 358 | 5-(4-(4-acetylphenoxy)piperidine-1- carbonyl)-N-(1-(4-cyanobenzyl)piperidin-4- yl)picolinamide | 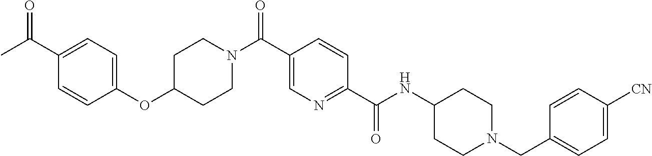 [SMILES: CC(=O)c1ccc(OC2CCN(C(=O)c3ccc(C(=O)NC4CCN(Cc5ccc(C#N)cc5)CC4)nc3)CC2)cc1] |
| 359 | 5-(4-(4-acetylphenoxy)piperidine-1- carbonyl)-N-(6-(4-fluorophenoxy)pyridin-3- yl)picolinamide | 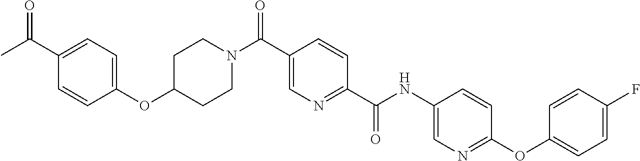 [SMILES: CC(=O)c1ccc(OC2CCN(C(=O)c3ccc(C(=O)Nc4ccc(Oc5ccc(F)cc5)nc4)nc3)CC2)cc1] |
| 360 | 5-(4-(4-(dimethylcarbamoyl) phenoxy)piperidine-1- carbonyl)-N-(6-(4-fluorophenoxy)pyridin- 3-yl)picolinamide | 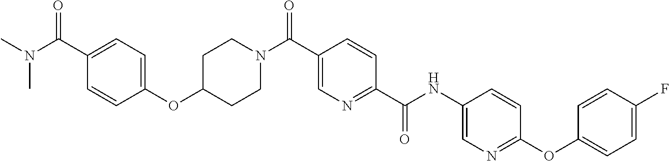 [SMILES: CN(C)C(=O)c1ccc(OC2CCN(C(=O)c3ccc(C(=O)Nc4ccc(Oc5ccc(F)cc5)nc4)nc3)CC2)cc1] |
| 361 | N-(1-(4-cyanobenzyl)piperidin-4-yl)-6-(4- (4-(trifluoromethyl)phenoxy)piperidin-1- yl)pyridazine-3-carboxamide | 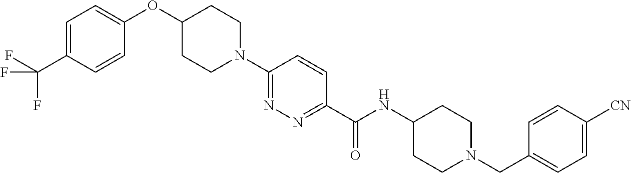 [SMILES: N#Cc1ccc(CN2CCC(NC(=O)c3ccc(N4CCC(Oc5ccc(C(F)(F)F)cc5)CC4)nn3)CC2)cc1] |
| 362 | N-(1-(4-cyanobenzyl)piperidin-4-yl)-6-(4- (4-methoxybenzoyl)piperidin-1- yl)pyridazine-3-carboxamide | 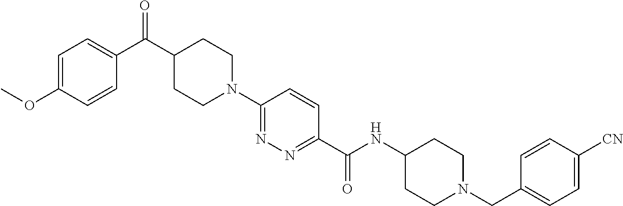 [SMILES: COc1ccc(C(=O)C2CCN(c3ccc(C(=O)NC4CCN(Cc5ccc(C#N)cc5)CC4)nn3)CC2)cc1] |
| 363 | N-(1-(4-cyanobenzyl)piperidin-4-yl)-6-(4- (4-nitrophenoxy)piperidine-1- carbonyl)nicotinamide | 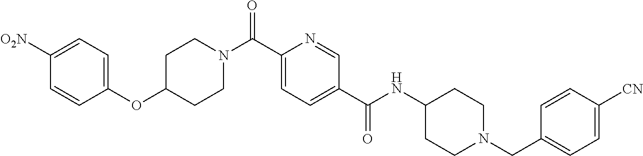 [SMILES: N#Cc1ccc(CN2CCC(NC(=O)c3ccc(C(=O)N4CCC(Oc5ccc([N+](=O)[O-])cc5)CC4)nc3)CC2)cc1] |
| 364 | 6-(4-(4-aminophenoxy)piperidine-1- carbonyl)-N-(1-(4-cyanobenzyl)piperidin-4- yl)nicotinamide | 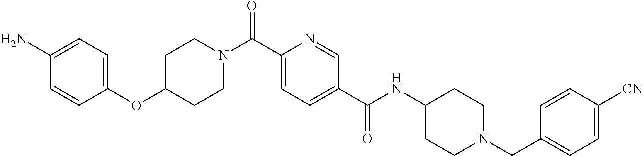 [SMILES: N#Cc1ccc(CN2CCC(NC(=O)c3ccc(C(=O)N4CCC(Oc5ccc(N)cc5)CC4)nc3)CC2)cc1] |
| 365 | N-(1-(4-cyanobenzyl)piperidin-4-yl)-5-(4- (4-(pyrrolidin-1-yl)benzoyl)piperidine-1- carbonyl)picolinamide | 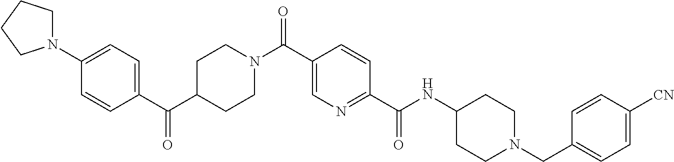 [SMILES: N#Cc1ccc(CN2CCC(NC(=O)c3ccc(C(=O)N4CCC(C(=O)c5ccc(N6CCCC6)cc5)CC4)cn3)CC2)cc1] |
| 366 | 6-(4-(4-acetamidophenoxy)piperidine-1- carbonyl)-N-(1-(4-cyanobenzyl)piperidin-4- yl)nicotinamide | 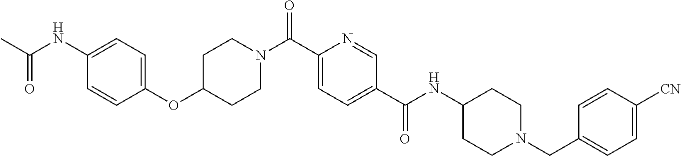 [SMILES: CC(=O)Nc1ccc(OC2CCN(C(=O)c3ccc(C(=O)NC4CCN(Cc5ccc(C#N)cc5)CC4)cn3)CC2)cc1] |
| 367 | N-(1-(4-cyanobenzyl)piperidin-4-yl)-6-(4- (4-(methylsulfonamido)phenoxy)piperidine- 1-carbonyl)nicotinamide | 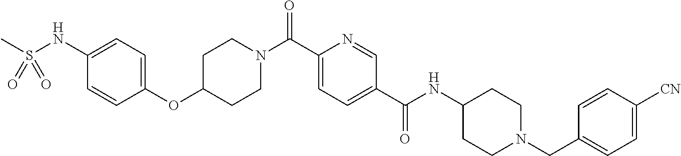 [SMILES: CS(=O)(=O)Nc1ccc(OC2CCN(C(=O)c3ccc(C(=O)NC4CCN(Cc5ccc(C#N)cc5)CC4)cn3)CC2)cc1] |
| 368 | N-(6-(4-fluorophenoxy)pyridin-3-yl)-5-(4- (4-(pyrrolidin-1-yl)benzoyl)piperidine-1- carbonyl)picolinamide | 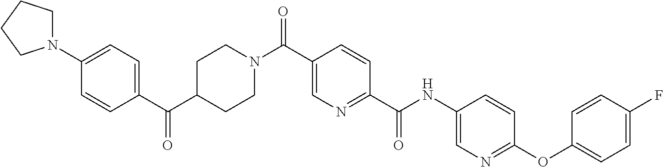 [SMILES: O=C(Nc1ccc(Oc2ccc(F)cc2)nc1)c1ccc(C(=O)N2CCC(C(=O)c3ccc(N4CCCC4)cc3)CC2)cn1] |
| 369 | 5-(4-(4-cyanobenzoyl)piperidine-1- carbonyl)-N-(1-(4-cyanobenzyl)piperidin-4- yl)picolinamide | 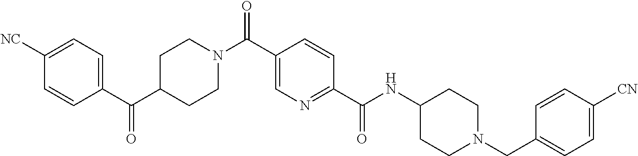 [SMILES: N#Cc1ccc(CN2CCC(NC(=O)c3ccc(C(=O)N4CCC(C(=O)c5ccc(C#N)cc5)CC4)cn3)CC2)cc1] |
| 370 | N-(1-(4-cyanobenzyl)piperidin-4-yl)-6-(4- (4-(dimethylamino)phenoxy)piperidine-1- carbonyl)nicotinamide | 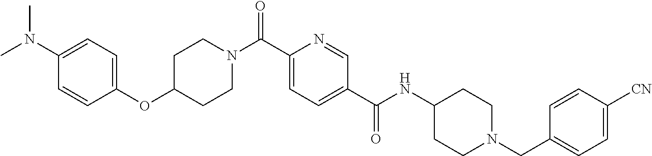 [SMILES: CN(C)c1ccc(OC2CCN(C(=O)c3ccc(C(=O)NC4CCN(Cc5ccc(C#N)cc5)CC4)cn3)CC2)cc1] |
| 371 | N-(1-(4-cyanobenzyl)piperidin-4-yl)-6-(4- (4-(17-oxo-20-((3aS,4S,6aR)-2- oxohexahydro-1H-thieno[3,4-d]imidazol-4- yl)-4,7,10,13-tetraoxa-16- azaicosanamido)phenoxy)piperidine-1- carbonyl)nicotinamide | 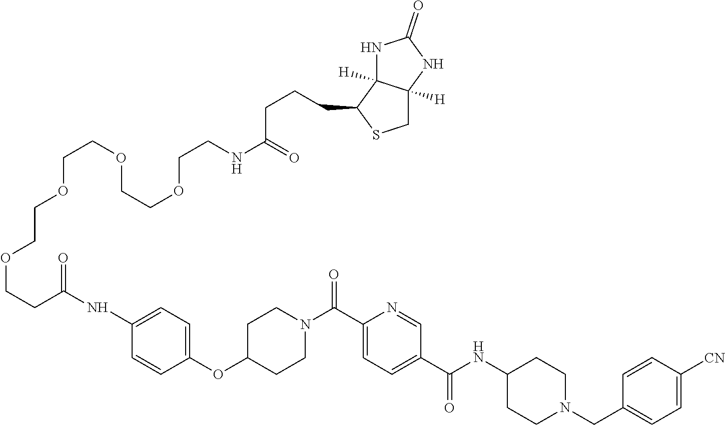 [SMILES: N#Cc1ccc(CN2CCC(NC(=O)c3ccc(C(=O)N4CCC(Oc5ccc(NC(=O)CCOCCOCCOCCOCCNC(=O)CCC[C@@H]6SC[C@@H]7NC(=O)N[C@@H]76)cc5)CC4)nc3)CC2)cc1] |
| 372 | N-(1-(4-cyanobenzyl)piperidin-4-yl)-5-(4- (4-(methylthio)benzoyl)piperidine-1- carbonyl)picolinamide | 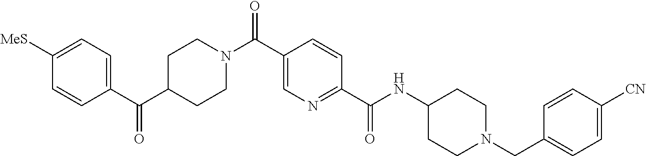 [SMILES: CSc1ccc(C(=O)C2CCN(C(=O)c3ccc(C(=O)NC4CCN(Cc5ccc(C#N)cc5)CC4)nc3)CC2)cc1] |
| 373 | 6-(4-(4-methoxybenzoyl)piperidine-1- carbonyl)-N-(1-(4-nitrobenzyl)piperidin-4- yl)nicotinamide | 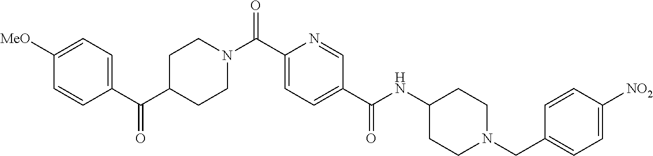 [SMILES: COc1ccc(C(=O)C2CCN(C(=O)c3ccc(C(=O)NC4CCN(Cc5ccc([N+](=O)[O-])cc5)CC4)cn3)CC2)cc1] |
| 374 | 1-(4-cyanobenzyl)-4-(5-(4-(4- (methylsulfinyl)benzoyl)piperidine-1- carbonyl)picolinamido)piperidine 1-oxide | 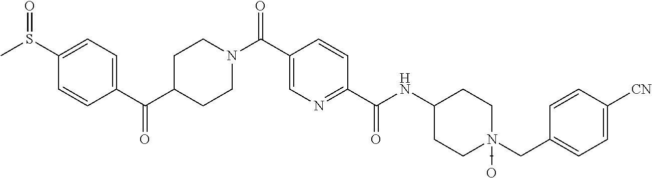 |
| 375 | 5-(4-(4-(1H-pyrazol-1- yl)benzoyl)piperidine-1-carbonyl)-N-(1-(4- cyanobenzyl)piperidin-4-yl)picolinamide | 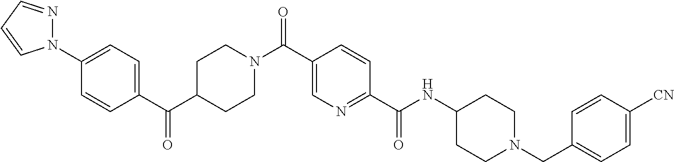 [SMILES: N#Cc1ccc(CN2CCC(NC(=O)c3ccc(C(=O)N4CCC(C(=O)c5ccc(-n6cccn6)cc5)CC4)cn3)CC2)cc1] |
| 376 | N-(1-(4-cyanobenzyl)piperidin-4-yl)-5-(4- (4-morpholinobenzoyl)piperidine-1- carbonyl)picolinamide | 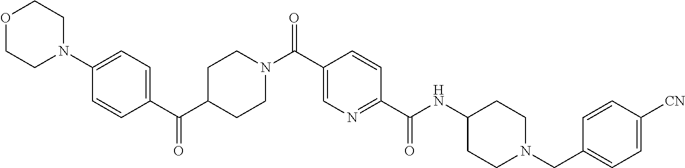 [SMILES: N#Cc1ccc(CN2CCC(NC(=O)c3ccc(C(=O)N4CCC(C(=O)c5ccc(N6CCOCC6)cc5)CC4)cn3)CC2)cc1] |
| 377 | N-(1-(4-cyanobenzyl)piperidin-4-yl)-6-(4- (4-(pyrrolidin-1-yl)benzoyl)piperidine-1- carbonyl)nicotinamide | 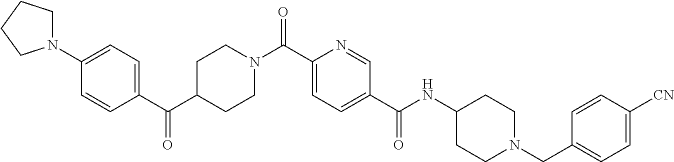 [SMILES: N#Cc1ccc(CN2CCC(NC(=O)c3ccc(C(=O)N4CCC(C(=O)c5ccc(N6CCCC6)cc5)CC4)nc3)CC2)cc1] |
| 378 | N-(1-(4-cyanobenzyl)piperidin-4-yl)-6-(4- (4-methoxy-2-nitrophenoxy)piperidine-1- carbonyl)nicotinamide | 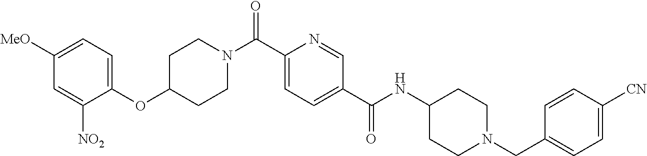 [SMILES: COc1ccc(OC2CCN(C(=O)c3ccc(C(=O)NC4CCN(Cc5ccc(C#N)cc5)CC4)cn3)CC2)c([N+](=O)[O-])c1] |
| 379 | N-(6-(4-fluorophenoxy)pyridin-3-yl)-5-(4- (4-morpholinobenzoyl)piperidine-1- carbonyl)picolinamide | 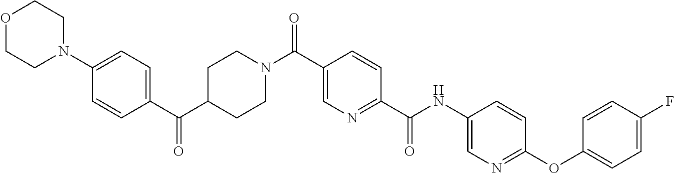 [SMILES: O=C(Nc1ccc(Oc2ccc(F)cc2)nc1)c1ccc(C(=O)N2CCC(C(=O)c3ccc(N4CCOCC4)cc3)CC2)cn1] |
| 380 | N-(1-(4-cyanobenzyl)piperidin-4-yl)-5-(4- (4-(4-methylpiperazin-1- yl)benzoyl)piperidine-1- carbonyl)picolinamide | 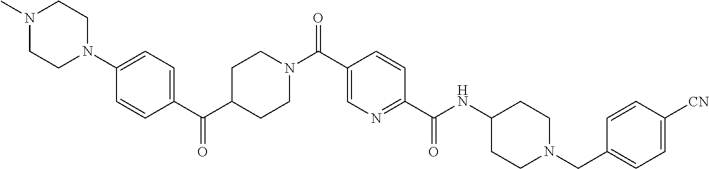 [SMILES: CN1CCN(c2ccc(C(=O)C3CCN(C(=O)c4ccc(C(=O)NC5CCN(Cc6ccc(C#N)cc6)CC5)nc4)CC3)cc2)CC1] |
| 381 | N-(1-(4-fluorobenzyl)piperidin-4-yl)-6-(4- (4-(pyrrolidin-1-yl)benzoyl)piperidine-1- carbonyl)nicotinamide | 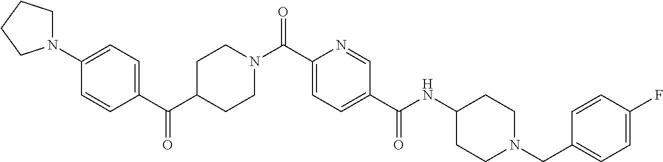 [SMILES: O=C(NC1CCN(Cc2ccc(F)cc2)CC1)c1ccc(C(=O)N2CCC(C(=O)c3ccc(N4CCCC4)cc3)CC2)nc1] |
| 382 | N-(6-(4-fluorophenoxy)pyridin-3-yl)-6-(4- (4-(pyrrolidin-1-yl)benzoyl)piperidine-1- carbonyl)nicotinamide | 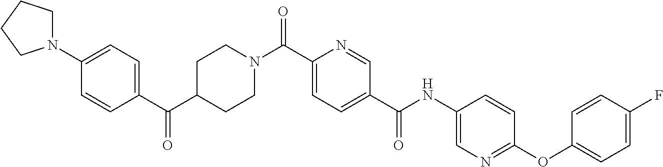 [SMILES: O=C(Nc1ccc(Oc2ccc(F)cc2)nc1)c1ccc(C(=O)N2CCC(C(=O)c3ccc(N4CCCC4)cc3)CC2)nc1] |
| 383 | 6-(4-(2-acetamido-4- methoxyphenoxy)piperidine-1- carbonyl)-N- (1-(4-cyanobenzyl)piperidin-4- yl)nicotinamide | 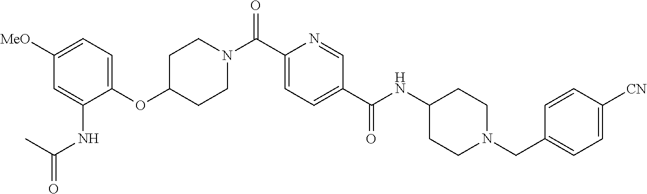 [SMILES: COc1ccc(OC2CCN(C(=O)c3ccc(C(=O)NC4CCN(Cc5ccc(C#N)cc5)CC4)cn3)CC2)c(NC(C)=O)c1] |
| 384 | 6-(4-(2-amino-4- methoxyphenoxy)piperidine-1- carbonyl)-N- (1-(4-cyanobenzyl)piperidin-4- yl)nicotinamide | 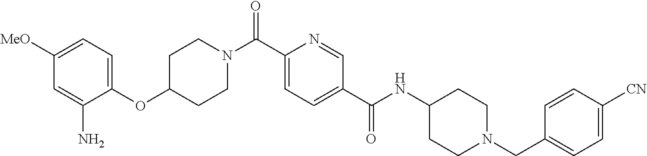 [SMILES: COc1ccc(OC2CCN(C(=O)c3ccc(C(=O)NC4CCN(Cc5ccc(C#N)cc5)CC4)cn3)CC2)c(N)c1] |
| 385 | N-(1-(4-cyanobenzyl)piperidin-4-yl)- 6-(4-(2-(dimethylamino)-4- methoxyphenoxy)piperidine-1- carbonyl)nicotinamide | 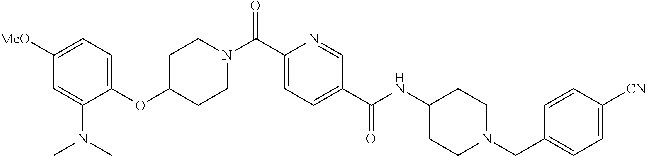 [SMILES: COc1ccc(OC2CCN(C(=O)c3ccc(C(=O)NC4CCN(Cc5ccc(C#N)cc5)CC4)cn3)CC2)c(N(C)C)c1] |
| 386 | N3,N6-bis(1-(4-cyanobenzyl)piperidin- 4-yl)pyridazine-3,6-dicarboxamide | 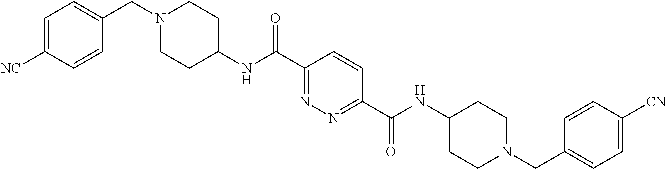 [SMILES: N#Cc1ccc(CN2CCC(NC(=O)c3ccc(C(=O)NC4CCN(Cc5ccc(C#N)cc5)CC4)nn3)CC2)cc1] |
| 387 | N-(1-(4-cyanobenzyl)piperidin-4-yl)-6-(4- (4-methoxybenzoyl)piperidine-1- carbonyl)pyridazine-3-carboxamide | 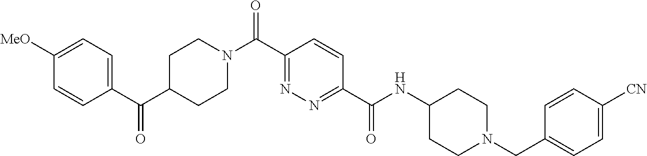 [SMILES: COc1ccc(C(=O)C2CCN(C(=O)c3ccc(C(=O)NC4CCN(Cc5ccc(C#N)cc5)CC4)nn3)CC2)cc1] |
| 388 | N-(1-(4-cyanobenzyl)piperidin-4-yl)-6-(4- (4-cyanophenoxy)piperidine-1- carbonyl)pyridazine-3-carboxamide | 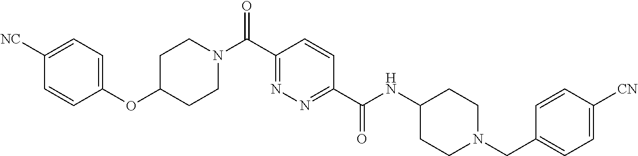 [SMILES: N#Cc1ccc(CN2CCC(NC(=O)c3ccc(C(=O)N4CCC(Oc5ccc(C#N)cc5)CC4)nn3)CC2)cc1] |
| 389 | N-(1-(4-cyanobenzyl)piperidin-4-yl)-6-(4- (4-methoxy-2-(methylsulfonamido)phenoxy) piperidine-1-carbonyl)nicotinamide | 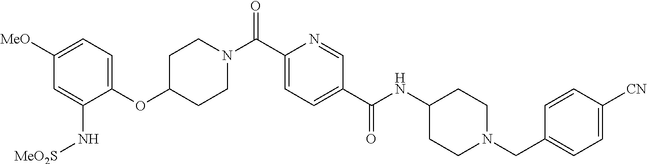 [SMILES: COc1ccc(OC2CCN(C(=O)c3ccc(C(=O)NC4CCN(Cc5ccc(C#N)cc5)CC4)cn3)CC2)c(NS(C)(=O)=O)c1] |
| 390 | 6-(4-(4-acetylphenoxy)piperidine-1- carbonyl)-N-(1-(4-cyanobenzyl)piperidin-4- yl)nicotinamide | 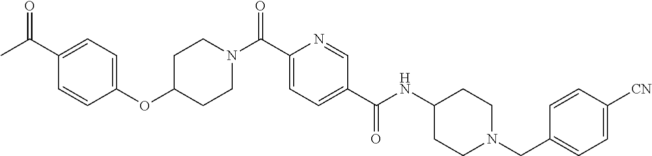 [SMILES: CC(=O)c1ccc(OC2CCN(C(=O)c3ccc(C(=O)NC4CCN(Cc5ccc(C#N)cc5)CC4)cn3)CC2)cc1] |
| 391 | 6-(4-(4-acetylphenoxy)piperidine-1- carbonyl)-N-(1-(4-fluorobenzyl)piperidin-4- yl)nicotinamide | 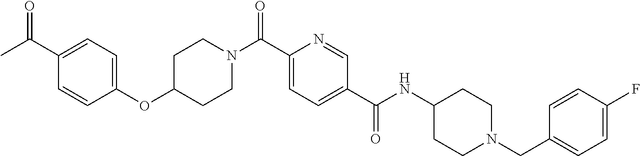 [SMILES: CC(=O)c1ccc(OC2CCN(C(=O)c3ccc(C(=O)NC4CCN(Cc5ccc(F)cc5)CC4)cn3)CC2)cc1] |
| 392 | 6-(4-(4-(1H-pyrazol-1- yl)benzoyl)piperidine-1-carbonyl)-N-(1-(4- fluorobenzyl)piperidin-4-yl)nicotinamide | 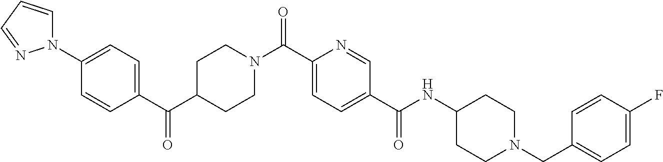 [SMILES: O=C(NC1CCN(Cc2ccc(F)cc2)CC1)c1ccc(C(=O)N2CCC(C(=O)c3ccc(-n4cccn4)cc3)CC2)nc1] |
| 393 | 6-(4-(4-(1H-pyrazol-1- yl)benzoyl)piperidine-1-carbonyl)-N-(1-(4- cyanobenzyl)piperidin-4-yl)nicotinamide | 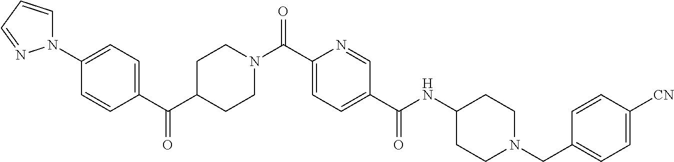 [SMILES: N#Cc1ccc(CN2CCC(NC(=O)c3ccc(C(=O)N4CCC(C(=O)c5ccc(-n6cccn6)cc5)CC4)nc3)CC2)cc1] |
| 394 | N-(1-(4-cyanobenzyl)piperidin-4-yl)- 6-(4-(4-methoxy-2-(17-oxo-21- ((3aS,4S,6aR)-2-oxohexahydro-1H- thieno[3,4-d]imidazol-4- yl)-4,7,10,13-tetraoxa-16- azahenicosanamido)phenoxy) piperidine-1-carbonyl)nicotinamide | 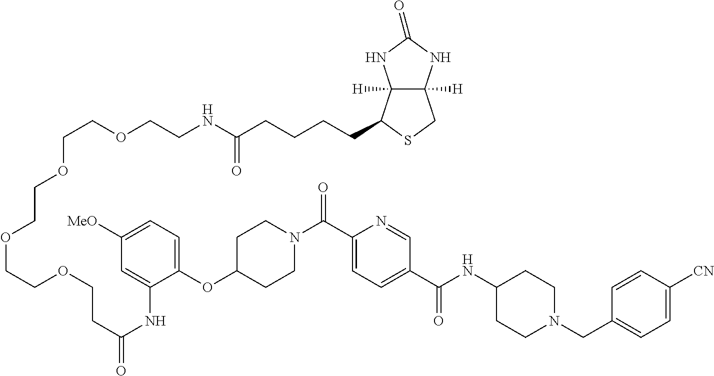 [SMILES: COc1ccc(OC2CCN(C(=O)c3ccc(C(=O)NC4CCN(Cc5ccc(C#N)cc5)CC4)cn3)CC2)c(NC(=O)CCOCCOCCOCCOCCNC(=O)CCCC[C@@H]2SC[C@@H]3NC(=O)N[C@@H]32)c1] |
| 395 | 6-(4-(4-acetylphenoxy)piperidine-1- carbonyl)-N-(6-(4-fluorophenoxy)pyridin- 3-yl)nicotinamide | 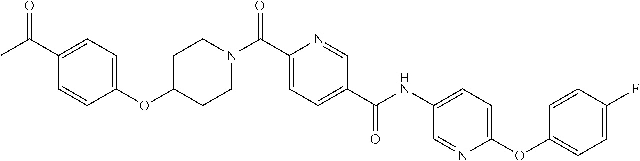 [SMILES: CC(=O)c1ccc(OC2CCN(C(=O)c3ccc(C(=O)Nc4ccc(Oc5ccc(F)cc5)nc4)cn3)CC2)cc1] |
| 396 | N-(1-(4-fluorobenzyl)piperidin-4-yl)-6-(4- (4-(methylsulfonyl)phenoxy)piperidine-1- carbonyl)nicotinamide | 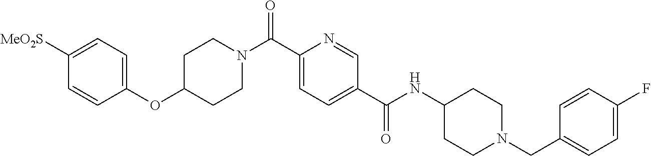 [SMILES: CS(=O)(=O)c1ccc(OC2CCN(C(=O)c3ccc(C(=O)NC4CCN(Cc5ccc(F)cc5)CC4)cn3)CC2)cc1] |
| 397 | N-(1-(4-cyanobenzyl)piperidin-4-yl)-6-(4- (4-(methylsulfonyl)phenoxy)piperidine-1- carbonyl)nicotinamide | 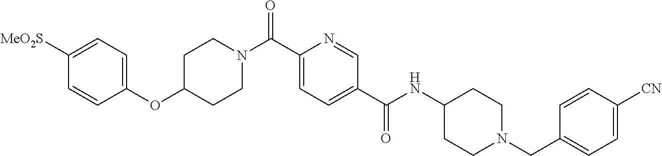 [SMILES: CS(=O)(=O)c1ccc(OC2CCN(C(=O)c3ccc(C(=O)NC4CCN(Cc5ccc(C#N)cc5)CC4)cn3)CC2)cc1] |
| 398 | N-(4-(4-cyanophenoxy)cyclohexyl)-6-(4-(4- (methylsulfonyl)phenoxy)piperidine-1- carbonyl)nicotinamide | 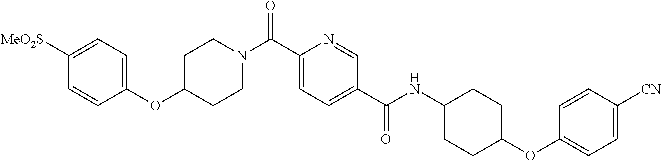 [SMILES: CS(=O)(=O)c1ccc(OC2CCN(C(=O)c3ccc(C(=O)NC4CCC(Oc5ccc(C#N)cc5)CC4)cn3)CC2)cc1] |
| 399 | N-(1-(4-cyanobenzyl)piperidin-4-yl)-6-(4- (4-(methylsulfonyl)phenoxy)piperidine-1- carbonyl)pyridazine-3-carboxamide | 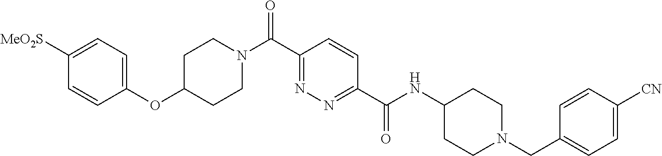 [SMILES: CS(=O)(=O)c1ccc(OC2CCN(C(=O)c3ccc(C(=O)NC4CCN(Cc5ccc(C#N)cc5)CC4)nn3)CC2)cc1] |
| 400 | N-(1-(4-aminobenzyl)piperidin-4-yl)-6-(4- (4-methoxybenzoyl)piperidine-1- carbonyl)nicotinamide | 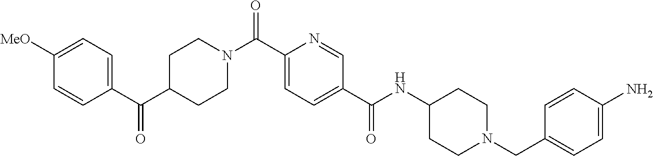 [SMILES: COc1ccc(C(=O)C2CCN(C(=O)c3ccc(C(=O)NC4CCN(Cc5ccc(N)cc5)CC4)cn3)CC2)cc1] |
| 401 | N-(1-(4-acetamidobenzyl)piperidin-4-yl)-6- (4-(4-methoxybenzoyl)piperidine-1- carbonyl)nicotinamide | 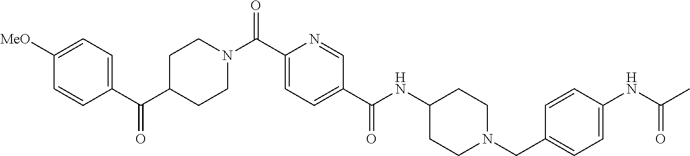 [SMILES: COc1ccc(C(=O)C2CCN(C(=O)c3ccc(C(=O)NC4CCN(Cc5ccc(NC(C)=O)cc5)CC4)cn3)CC2)cc1] |
| 402 | 6-(4-(4-acetylphenoxy)piperidine-1- carbonyl)-N-(4-(4- cyanophenoxy)cyclohexyl)nicotinamide | 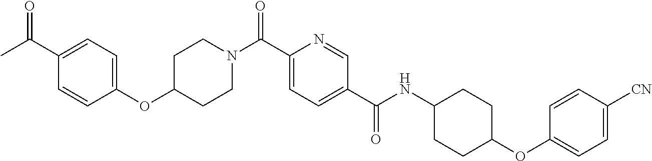 [SMILES: CC(=O)c1ccc(OC2CCN(C(=O)c3ccc(C(=O)NC4CCC(Oc5ccc(C#N)cc5)CC4)cn3)CC2)cc1] |
| 403 | 5-(4-(4-methoxybenzoyl)piperidine-1- carbonyl)-N-(1-(4-(14-oxo-18- ((3aS,4S,6aR)-2-oxohexahydro-1H- thieno[3,4-d]imidazol-4-yl)- 4,7,10-trioxa-13-azaoctadecanamido) benzyl)piperidin-4-yl)picolinamide | 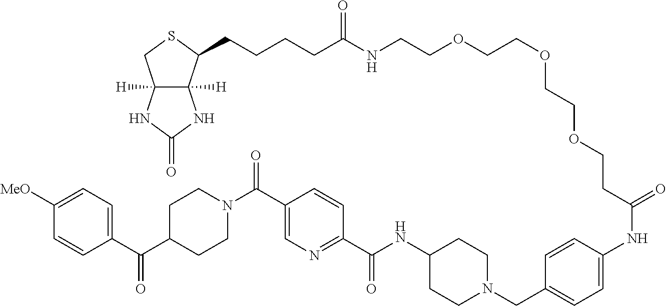 [SMILES: COc1ccc(C(=O)C2CCN(C(=O)c3ccc(C(=O)NC4CCN(Cc5ccc(NC(=O)CCOCCOCCOCCNC(=O)CCCC[C@@H]6SC[C@@H]7NC(=O)N[C@@H]76)cc5)CC4)nc3)CC2)cc1] |
| 404 | 6-(4-(4-fluorobenzyl)piperazine-1- carbonyl)-N-(1-(4-fluorophenyl)piperidin- 4-yl)nicotinamide | 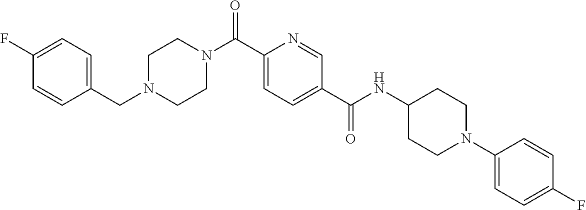 [SMILES: O=C(NC1CCN(c2ccc(F)cc2)CC1)c1ccc(C(=O)N2CCN(Cc3ccc(F)cc3)CC2)nc1] |
| 405 | 6-(4-(4-fluorobenzyl)piperazine-1- carbonyl)-N-(1-(4- methoxyphenyl)piperidin-4-yl) nicotinamide | 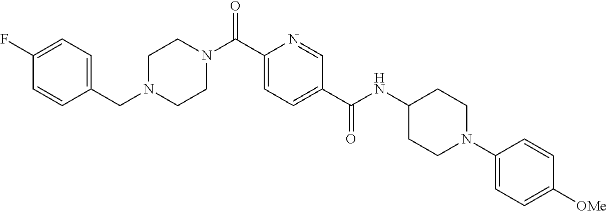 [SMILES: COc1ccc(N2CCC(NC(=O)c3ccc(C(=O)N4CCN(Cc5ccc(F)cc5)CC4)nc3)CC2)cc1] |
| 406 | 6-(4-(4-acetamidophenoxy)piperidine-1- carbonyl)-N-(1-(4-fluorobenzyl)piperidin- 4-yl)nicotinamide | 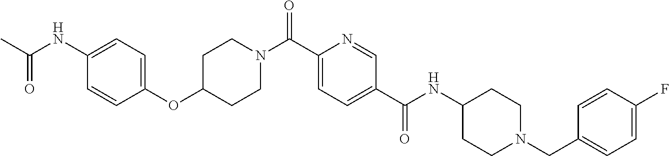 [SMILES: CC(=O)Nc1ccc(OC2CCN(C(=O)c3ccc(C(=O)NC4CCN(Cc5ccc(F)cc5)CC4)cn3)CC2)cc1] |
| 407 | 6-(4-(4-acetamidophenoxy)piperidine-1- carbonyl)-N-(1-(4- methoxybenzyl)piperidin-4-yl) nicotinamide | 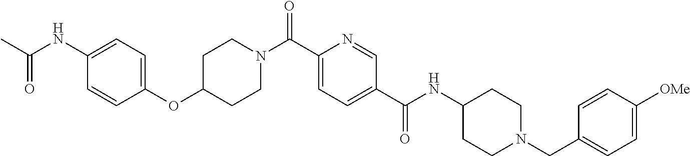 [SMILES: COc1ccc(CN2CCC(NC(=O)c3ccc(C(=O)N4CCC(Oc5ccc(NC(C)=O)cc5)CC4)nc3)CC2)cc1] |
| 408 | 5-(4-(4-acetamidophenoxy)piperidine-1- carbonyl)-N-(6-(4-fluorophenoxy)pyridin- 3-yl)picolinamide | 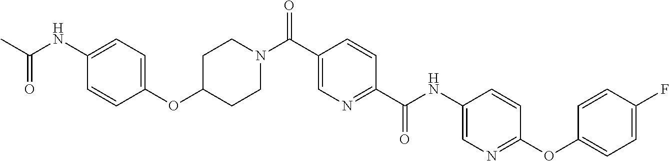 [SMILES: CC(=O)Nc1ccc(OC2CCN(C(=O)c3ccc(C(=O)Nc4ccc(Oc5ccc(F)cc5)nc4)nc3)CC2)cc1] |
| 409 | N-(1-(4-cyanobenzyl)piperidin-4-yl)-6-(4- (4-(cyclopropanecarboxamido)phenoxy) piperidine-1-carbonyl)nicotinamide | 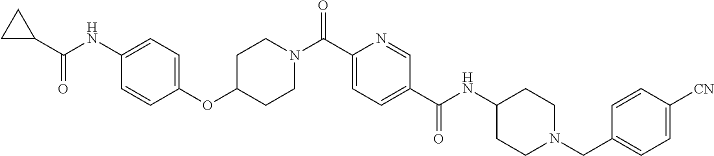 [SMILES: N#Cc1ccc(CN2CCC(NC(=O)c3ccc(C(=O)N4CCC(Oc5ccc(NC(=O)C6CC6)cc5)CC4)nc3)CC2)cc1] |
| 410 | N-(1-(4-cyanobenzyl)piperidin-4-yl)-5-(4- (4-(trifluoromethylthio)phenoxy)piperidine- 1-carbonyl)picolinamide | 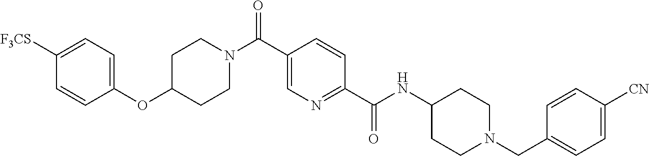 |
| 411 | 6-(4-(3-acetamidophenoxy)piperidine-1- carbonyl)-N-(1-(4-cyanobenzyl)piperidin-4- yl)nicotinamide | 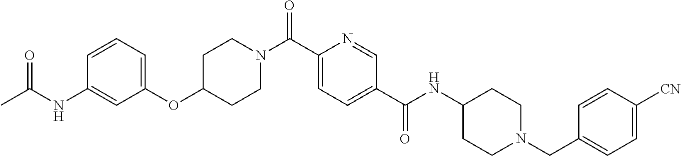 [SMILES: CC(=O)Nc1cccc(OC2CCN(C(=O)c3ccc(C(=O)NC4CCN(Cc5ccc(C#N)cc5)CC4)cn3)CC2)c1] |
| 412 | 6-(4-(3-acetamidophenoxy)piperidine-1- carbonyl)-N-(1-(4- methoxybenzyl)piperidin-4-yl)nicotinamide | 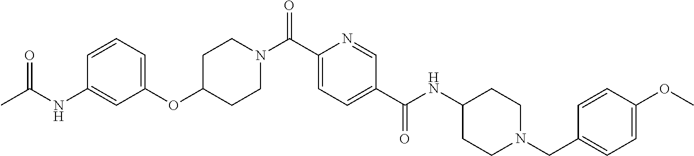 [SMILES: COc1ccc(CN2CCC(NC(=O)c3ccc(C(=O)N4CCC(Oc5cccc(NC(C)=O)c5)CC4)nc3)CC2)cc1] |
| 413 | 6-(4-(3-acetamidophenoxy)piperidine-1- carbonyl)-N-(1-(4-fluorobenzyl)piperidin-4- yl)nicotinamide | 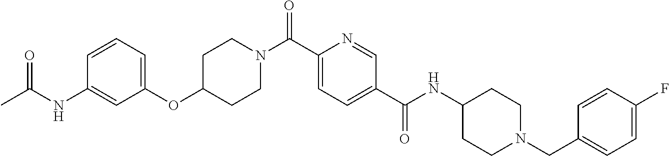 [SMILES: CC(=O)Nc1cccc(OC2CCN(C(=O)c3ccc(C(=O)NC4CCN(Cc5ccc(F)cc5)CC4)cn3)CC2)c1] |
| 414 | 6-(4-(3-acetamidophenoxy)piperidine-1- carbonyl)-N-(6-(4-fluorophenoxy)pyridin-3- yl)nicotinamide | 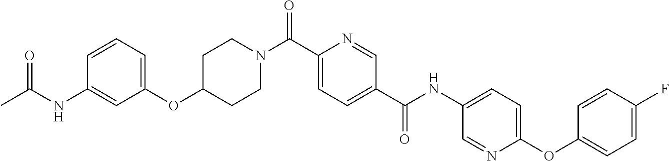 [SMILES: CC(=O)Nc1cccc(OC2CCN(C(=O)c3ccc(C(=O)Nc4ccc(Oc5ccc(F)cc5)nc4)cn3)CC2)c1] |
| 415 | N-(1-(4-cyanobenzyl)piperidin-4-yl)-5-(4- (4-(trifluoromethylsulfonyl) phenoxy)piperidine- 1-carbonyl)picolinamide | 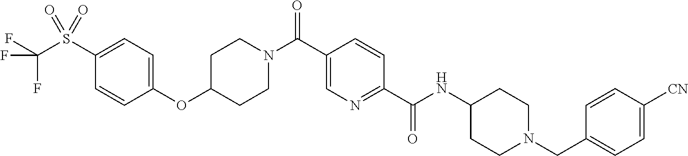 [SMILES: N#Cc1ccc(CN2CCC(NC(=O)c3ccc(C(=O)N4CCC(Oc5ccc(S(=O)(=O)C(F)(F)F)cc5)CC4)cn3)CC2)cc1] |
| 416 | tert-butyl 3-(5-(1-(4-cyanobenzyl)piperidin- 4-ylcarbamoyl)-2-(4-(4- methoxybenzoyl)piperidine-1- carbonyl)pyridin-3-yl)propylcarbamate | 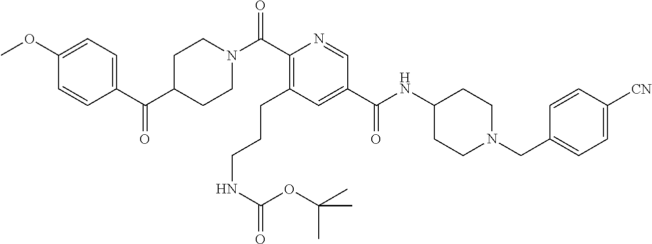 [SMILES: COc1ccc(C(=O)C2CCN(C(=O)c3ncc(C(=O)NC4CCN(Cc5ccc(C#N)cc5)CC4)cc3CCCNC(=O)OC(C)(C)C)CC2)cc1] |
| 417 | N-(1-(4-cyanophenyl)piperidin-4-yl)-6-(4- (4-fluorobenzyl)piperazine-1- carbonyl)nicotinamide | 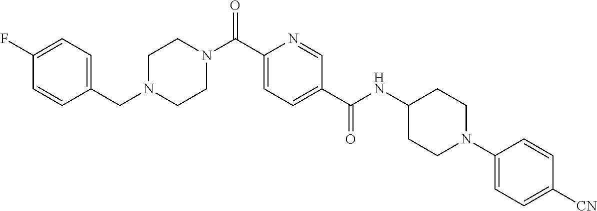 [SMILES: N#Cc1ccc(N2CCC(NC(=O)c3ccc(C(=O)N4CCN(Cc5ccc(F)cc5)CC4)nc3)CC2)cc1] |
| 418 | 6-(4-(4-cyanophenoxy)piperidine-1- carbonyl)-N-(1-(4-cyanophenyl)piperidin-4- yl)nicotinamide | 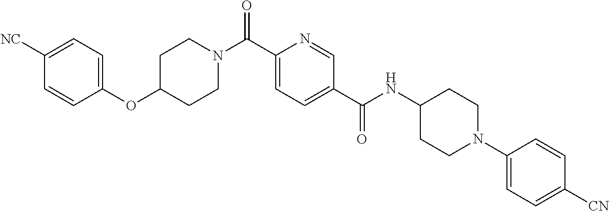 [SMILES: N#Cc1ccc(OC2CCN(C(=O)c3ccc(C(=O)NC4CCN(c5ccc(C#N)cc5)CC4)cn3)CC2)cc1] |
| 419 | N-(1-(4-cyanobenzyl)piperidin-4-yl)-5-(4- (thiophene-2-carbonyl)piperidine-1- carbonyl)picolinamide | 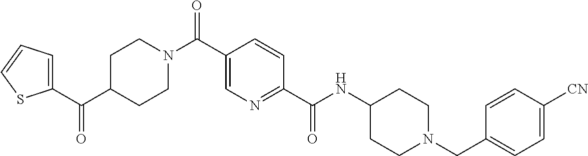 [SMILES: N#Cc1ccc(CN2CCC(NC(=O)c3ccc(C(=O)N4CCC(C(=O)c5cccs5)CC4)cn3)CC2)cc1] |
| 420 | 6-(4-(4-cyanophenoxy)piperidine-1- carbonyl)-N-(1-(4- (methylsulfonyl)phenyl)piperidin-4- yl)nicotinamide | 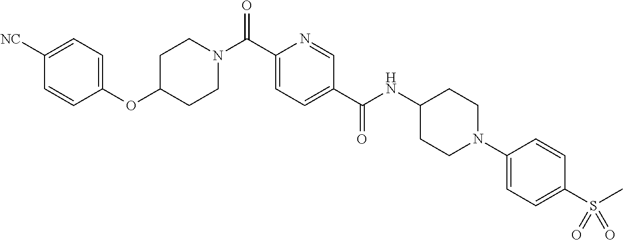 [SMILES: CS(=O)(=O)c1ccc(N2CCC(NC(=O)c3ccc(C(=O)N4CCC(Oc5ccc(C#N)cc5)CC4)nc3)CC2)cc1] |
| 421 | 6-(4-(4-fluorobenzyl)piperazine-1- carbonyl)-N-(1-(4- (methylsulfonyl)phenyl)piperidin-4- yl)nicotinamide | 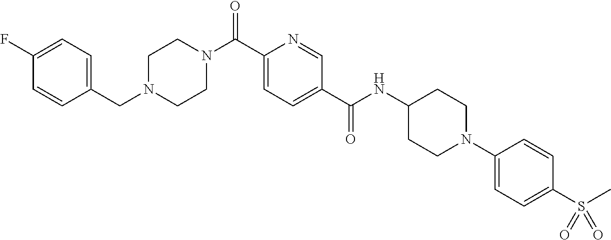 [SMILES: CS(=O)(=O)c1ccc(N2CCC(NC(=O)c3ccc(C(=O)N4CCN(Cc5ccc(F)cc5)CC4)nc3)CC2)cc1] |
| 422 | 6-(4-(4-cyanophenoxy)piperidine-1- carbonyl)-N-(1-(4- fluorophenyl)piperidin-4- yl)nicotinamide | 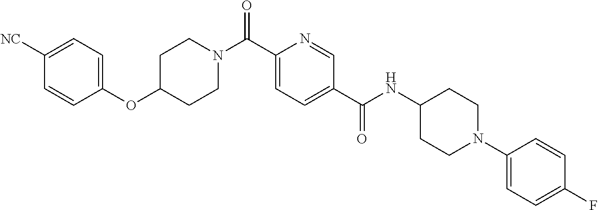 [SMILES: N#Cc1ccc(OC2CCN(C(=O)c3ccc(C(=O)NC4CCN(c5ccc(F)cc5)CC4)cn3)CC2)cc1] |
| 423 | 6-(4-(4-cyanophenoxy)piperidine-1- carbonyl)-N-(1-(4- methoxyphenyl)piperidin-4- yl)nicotinamide | 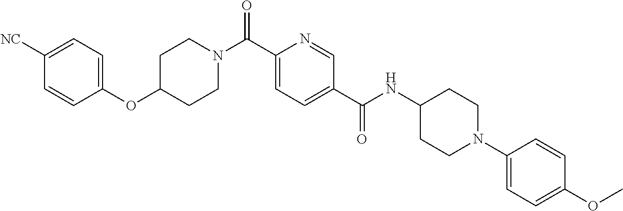 [SMILES: COc1ccc(N2CCC(NC(=O)c3ccc(C(=O)N4CCC(Oc5ccc(C#N)cc5)CC4)nc3)CC2)cc1] |
| 424 | 6-(4-(4-methoxybenzoyl)piperidine-1- carbonyl)-N-(1-(3- (trifluoromethoxy)benzyl)piperidin-4- yl)nicotinamide | 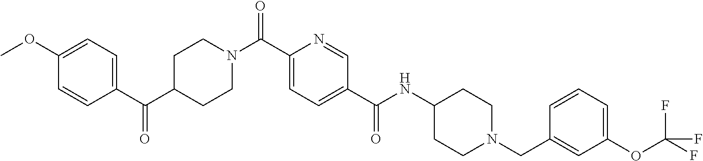 [SMILES: COc1ccc(C(=O)C2CCN(C(=O)c3ccc(C(=O)NC4CCN(Cc5cccc(OC(F)(F)F)c5)CC4)cn3)CC2)cc1] |
| 425 | 6-(4-(4-methoxybenzoyl)piperidine-1- carbonyl)-N-(1-(3- methoxybenzyl)piperidin-4-yl) nicotinamide | 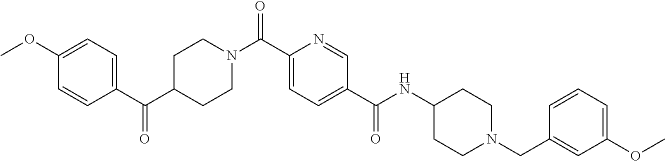 |
| 426 | N-((3S,4R)-3-fluoro-1- ((5-methylisoxazol- 3-yl)methyl)piperidin-4-yl)-6-(4-(4- methoxybenzoyl)piperidine-1- carbonyl)nicotinamide | 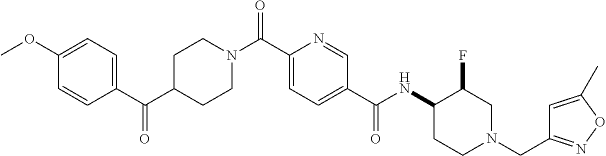 |
| 427 | N-((3S,4R)-3-fluoro-1-((2-methylthiazol-4- yl)methyl)piperidin-4-yl)-6-(4-(4- methoxybenzoyl)piperidine-1- carbonyl)nicotinamide | 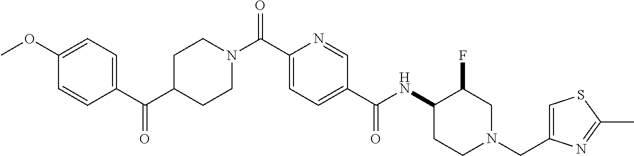 |
| 428 | 6-(4-(4-acetamidophenoxy)piperidine-1- carbonyl)-N-(1-(3- (trifluoromethoxy)benzyl)piperidin-4- yl)nicotinamide | 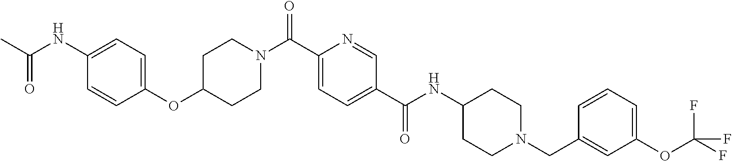 [SMILES: CC(=O)Nc1ccc(OC2CCN(C(=O)c3ccc(C(=O)NC4CCN(Cc5cccc(OC(F)(F)F)c5)CC4)cn3)CC2)cc1] |
| 429 | 6-(4-(3-acetamidophenoxy)piperidine-1- carbonyl)-N-(1-(3- (trifluoromethoxy)benzyl)piperidin-4- yl)nicotinamide | 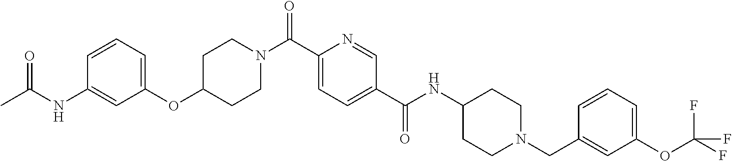 [SMILES: CC(=O)Nc1cccc(OC2CCN(C(=O)c3ccc(C(=O)NC4CCN(Cc5cccc(OC(F)(F)F)c5)CC4)cn3)CC2)c1] |
| 430 | 6-(4-(4-methoxybenzoyl)piperidine-1- carbonyl)-N-(1-(4- (trifluoromethoxy)benzyl)piperidin-4- yl)nicotinamide | 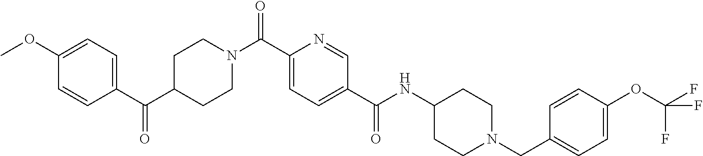 [SMILES: COc1ccc(C(=O)C2CCN(C(=O)c3ccc(C(=O)NC4CCN(Cc5ccc(OC(F)(F)F)cc5)CC4)cn3)CC2)cc1] |
| 431 | N-(1-(4-cyanobenzyl)piperidin-4-yl)-6-(4- (3-(cyclopropanecarboxamido)phenoxy) piperidine-1-carbonyl)nicotinamide | 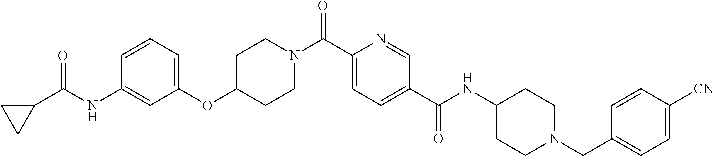 [SMILES: N#Cc1ccc(CN2CCC(NC(=O)c3ccc(C(=O)N4CCC(Oc5cccc(NC(=O)C6CC6)c5)CC4)nc3)CC2)cc1] |
| 432 | 6-(4-(3-(cyclopropanecarboxamido)phenoxy) piperidine-1-carbonyl)-N-(1-(4- fluorobenzyl)piperidin-4-yl)nicotinamide | 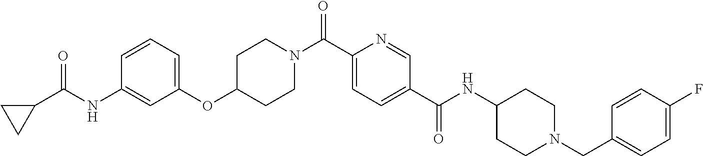 [SMILES: O=C(NC1CCN(Cc2ccc(F)cc2)CC1)c1ccc(C(=O)N2CCC(Oc3cccc(NC(=O)C4CC4)c3)CC2)nc1] |
| 433 | 6-(4-(3-(cyclopropanecarboxamido)phenoxy) piperidine-1-carbonyl)-N-(6-(4- fluorophenoxy)pyridin-3-yl)nicotinamide | 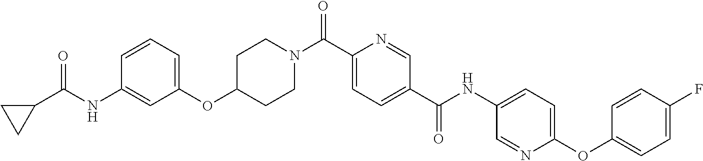 [SMILES: O=C(Nc1ccc(Oc2ccc(F)cc2)nc1)c1ccc(C(=O)N2CCC(Oc3cccc(NC(=O)C4CC4)c3)CC2)nc1] |
| 434 | N-((cis)-4-(4-cyanophenoxy)cyclohexyl)-6- (4-(3-(cyclopropanecarboxamido)phenoxy) piperidine-1-carbonyl)nicotinamide | 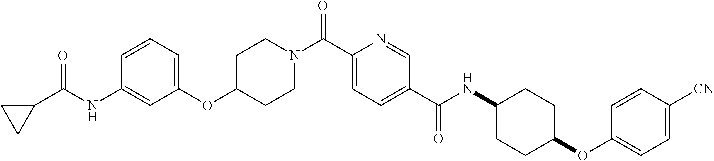 |
| 435 | 6-(4-(3-(cyclopropanecarboxamido)phenoxy) piperidine-1-carbonyl)-N-(1-(4- methoxybenzyl)piperidin-4-yl)nicotinamide | 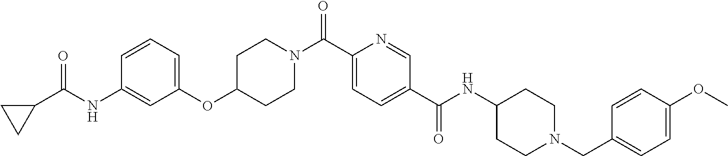 [SMILES: COc1ccc(CN2CCC(NC(=O)c3ccc(C(=O)N4CCC(Oc5cccc(NC(=O)C6CC6)c5)CC4)nc3)CC2)cc1] |
| 436 | N-(1-(4-cyanobenzyl)piperidin-4-yl)-6-(4- (4-(trifluoromethylthio)phenoxy)piperidine- 1-carbonyl)pyridazine-3-carboxamide | 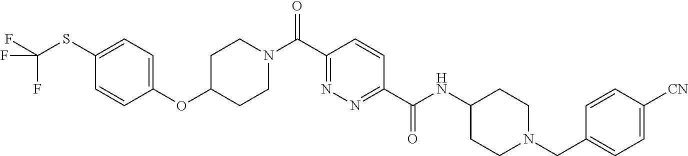 [SMILES: N#Cc1ccc(CN2CCC(NC(=O)c3ccc(C(=O)N4CCC(Oc5ccc(SC(F)(F)F)cc5)CC4)nn3)CC2)cc1] |
| 437 | 6-(4-(4-acetylphenoxy)piperidine-1- carbonyl)-N-(1-(4-cyanobenzyl)piperidin-4- yl)pyridazine-3-carboxamide | 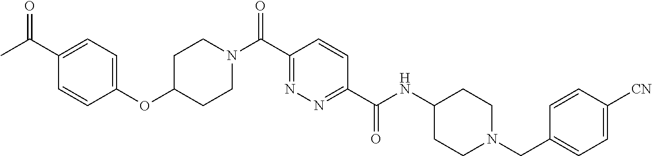 [SMILES: CC(=O)c1ccc(OC2CCN(C(=O)c3ccc(C(=O)NC4CCN(Cc5ccc(C#N)cc5)CC4)nn3)CC2)cc1] |
| 438 | 6-(4-(3-(cyclopropanecarboxamido)phenoxy) piperidine-1-carbonyl)-N-(1-(4- (trifluoromethoxy)benzyl)piperidin-4- yl)nicotinamide | 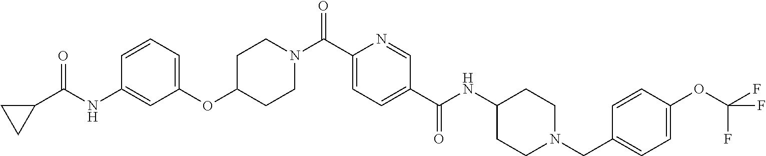 [SMILES: O=C(NC1CCN(Cc2ccc(OC(F)(F)F)cc2)CC1)c1ccc(C(=O)N2CCC(Oc3cccc(NC(=O)C4CC4)c3)CC2)nc1] |
| 439 | N-(1-(4-methoxybenzyl)piperidin-4-yl)-6- (4-(4-(pyrrolidin-1-yl)benzoyl)piperidine-1- carbonyl)nicotinamide | 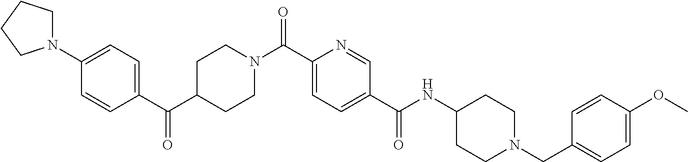 [SMILES: COc1ccc(CN2CCC(NC(=O)c3ccc(C(=O)N4CCC(C(=O)c5ccc(N6CCCC6)cc5)CC4)nc3)CC2)cc1] |
| 440 | 6-(4-(4-(pyrrolidin-1-yl)benzoyl)piperidine- 1-carbonyl)-N-(1-(4- (trifluoromethoxy)benzyl)piperidin-4- yl)nicotinamide | 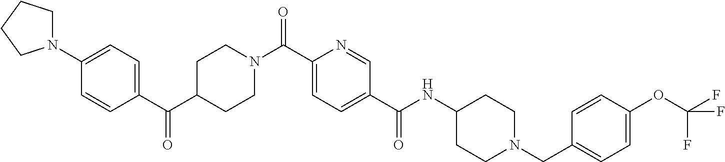 [SMILES: O=C(NC1CCN(Cc2ccc(OC(F)(F)F)cc2)CC1)c1ccc(C(=O)N2CCC(C(=O)c3ccc(N4CCCC4)cc3)CC2)nc1] |
| 441 | 6-(4-(4-(pyrrolidin-1-yl)benzoyl)piperidine- 1-carbonyl)-N-(1-(3- (trifluoromethoxy)benzyl)piperidin-4- yl)nicotinamide | 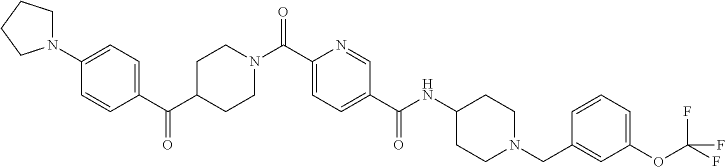 [SMILES: O=C(NC1CCN(Cc2cccc(OC(F)(F)F)c2)CC1)c1ccc(C(=O)N2CCC(C(=O)c3ccc(N4CCCC4)cc3)CC2)nc1] |
| 442 | N-((cis)-4-(4-cyanophenoxy)cyclohexyl)-6- (4-(4-(pyrrolidin-1-yl)benzoyl)piperidine-1- carbonyl)nicotinamide | 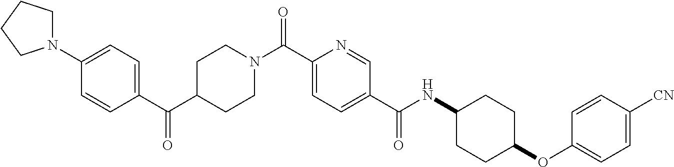 |
| 443 | N-(1-(3-fluoro-4-methoxybenzyl)piperidin- 4-yl)-6-(4-(4-(pyrrolidin-1-yl)benzoyl) piperidine-1- carbonyl)nicotinamide | 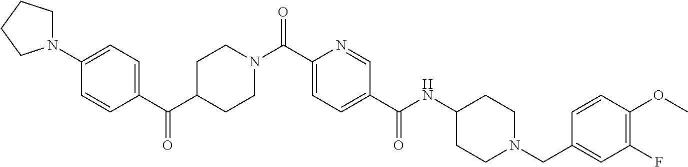 [SMILES: COc1ccc(CN2CCC(NC(=O)c3ccc(C(=O)N4CCC(C(=O)c5ccc(N6CCCC6)cc5)CC4)nc3)CC2)cc1F] |
| 444 | 6-(4-(4-(pyrrolidin-1-yl)benzoyl)piperidine- 1-carbonyl)-N-(1-(4-(pyrrolidin-1- yl)benzyl)piperidin-4-yl)nicotinamide | 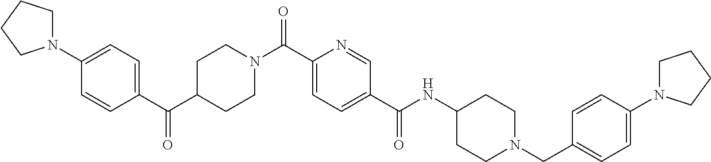 [SMILES: O=C(NC1CCN(Cc2ccc(N3CCCC3)cc2)CC1)c1ccc(C(=O)N2CCC(C(=O)c3ccc(N4CCCC4)cc3)CC2)nc1] |
| 445 | 6-(4-(4-methoxybenzoyl)piperidine-1- carbonyl)-N-(piperidin-4-yl)nicotinamide | 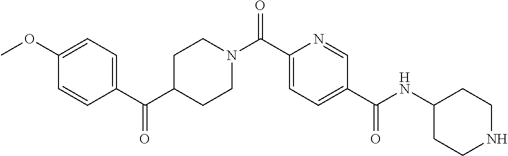 [SMILES: COc1ccc(C(=O)C2CCN(C(=O)c3ccc(C(=O)NC4CCNCC4)cn3)CC2)cc1] |
| 446 | N-(1-(4-isopropoxybenzyl)piperidin-4-yl)- 6-(4-(4-(pyrrolidin-1-yl)benzoyl)piperidine- 1-carbonyl)nicotinamide | 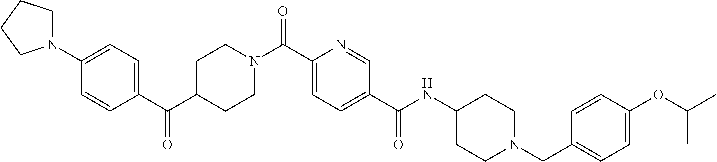 [SMILES: CC(C)Oc1ccc(CN2CCC(NC(=O)c3ccc(C(=O)N4CCC(C(=O)c5ccc(N6CCCC6)cc5)CC4)nc3)CC2)cc1] |
| 447 | N-(1-(4-cyano-3-fluorobenzyl)piperidin-4- yl)-6-(4-(4-(pyrrolidin-1-yl)benzoyl) piperidine-1- carbonyl)nicotinamide | 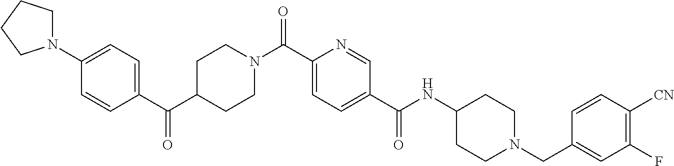 [SMILES: N#Cc1ccc(CN2CCC(NC(=O)c3ccc(C(=O)N4CCC(C(=O)c5ccc(N6CCCC6)cc5)CC4)nc3)CC2)cc1F] |
| 448 | N-(1-(4-cyanobenzyl)piperidin-4-yl)-6-(4- (4-(cyclopropanesulfonamido)phenoxy) piperidine-1-carbonyl)nicotinamide | 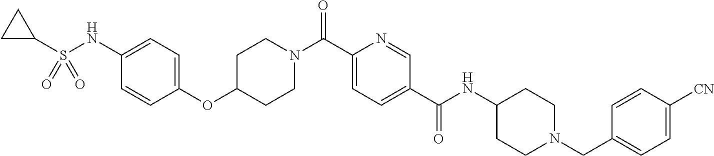 [SMILES: N#Cc1ccc(CN2CCC(NC(=O)c3ccc(C(=O)N4CCC(Oc5ccc(NS(=O)(=O)C6CC6)cc5)CC4)nc3)CC2)cc1] |
| 449 | 6-(4-(4-(cyclopropanesulfonamido) phenoxy)piperidine-1-carbonyl)-N-(6-(4- fluorophenoxy)pyridin-3-yl)nicotinamide | 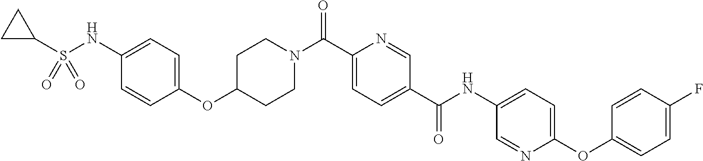 [SMILES: O=C(Nc1ccc(Oc2ccc(F)cc2)nc1)c1ccc(C(=O)N2CCC(Oc3ccc(NS(=O)(=O)C4CC4)cc3)CC2)nc1] |
| 450 | N-(1-(4-cyanobenzyl)piperidin-4-yl)-6-(4- (4-(trifluoromethylsulfonyl)phenoxy) piperidine-1-carbonyl)nicotinamide | 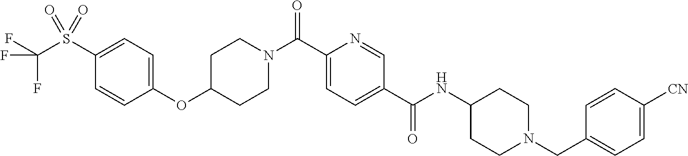 [SMILES: N#Cc1ccc(CN2CCC(NC(=O)c3ccc(C(=O)N4CCC(Oc5ccc(S(=O)(=O)C(F)(F)F)cc5)CC4)nc3)CC2)cc1] |
| 451 | N-((trans)-1-(4-cyanobenzyl)-3- fluoropiperidin-4-yl)-6-(4-(4- (trifluoromethylsulfonyl)phenoxy) piperidine-1-carbonyl)nicotinamide | 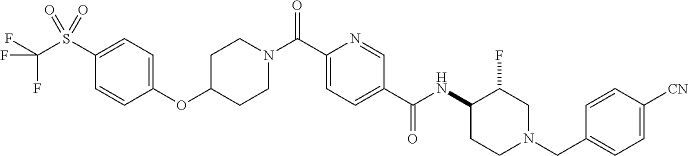 |
| 452 | N-((3R,4R)-1-(4-cyanobenzyl)-3- fluoropiperidin-4-yl)-6-(4-(4- methoxybenzoyl)piperidine-1- carbonyl)nicotinamide | 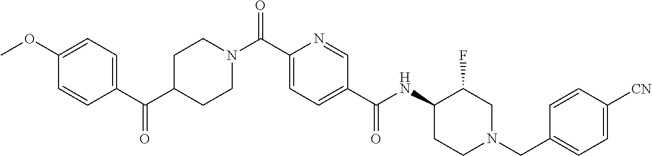 [SMILES: COc1ccc(C(=O)C2CCN(C(=O)c3ccc(C(=O)N[C@@H]4CCN(Cc5ccc(C#N)cc5)C[C@H]4F)cn3)CC2)cc1] |
| 453 | N-((3S,4S)-1-(4-cyanobenzyl)-3- fluoropiperidin-4-yl)-6-(4-(4- methoxybenzoyl)piperidine-1- carbonyl)nicotinamide | 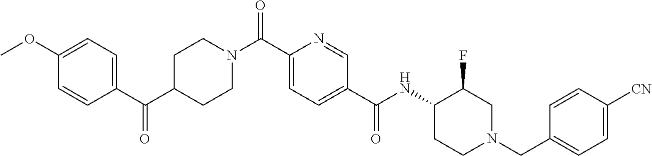 [SMILES: COc1ccc(C(=O)C2CCN(C(=O)c3ccc(C(=O)N[C@H]4CCN(Cc5ccc(C#N)cc5)C[C@@H]4F)cn3)CC2)cc1] |
| 454 | N-((cis)-1-(4-cyanobenzyl)-3- fluoropiperidin-4-yl)-6-(4-(4- methoxybenzoyl)piperidine-1- carbonyl)nicotinamide | 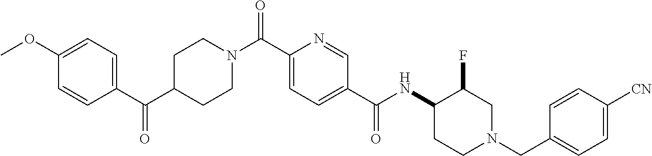 |
| 455 | 6-(4-(4-(cyclopropanecarbonyl) phenoxy)piperidine-1-carbonyl)-N-(1- (4-(trifluoromethoxy)benzyl)piperidin- 4-yl)nicotinamide | 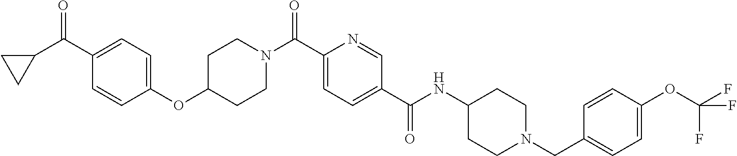 [SMILES: O=C(NC1CCN(Cc2ccc(OC(F)(F)F)cc2)CC1)c1ccc(C(=O)N2CCC(Oc3ccc(C(=O)C4CC4)cc3)CC2)nc1] |
| 456 | N-(1-(4-cyanobenzyl)piperidin-4-yl)-6-(4- (4-(cyclopropanecarbonyl)phenoxy) piperidine- 1-carbonyl)nicotinamide | 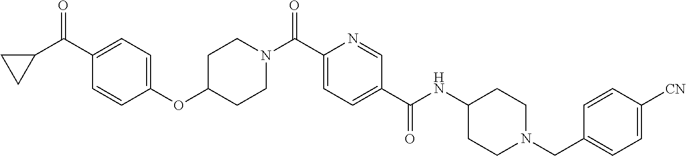 [SMILES: N#Cc1ccc(CN2CCC(NC(=O)c3ccc(C(=O)N4CCC(Oc5ccc(C(=O)C6CC6)cc5)CC4)nc3)CC2)cc1] |
| 457 | 6-(4-(4-(cyclopropanecarbonyl)phenoxy) piperidine-1-carbonyl)-N-(6-(4-fluorophenoxy) pyridin-3-yl)nicotinamide | 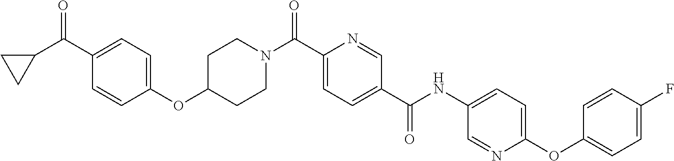 [SMILES: O=C(Nc1ccc(Oc2ccc(F)cc2)nc1)c1ccc(C(=O)N2CCC(Oc3ccc(C(=O)C4CC4)cc3)CC2)nc1] |
| 458 | 6-(4-(4-(cyclopropanecarbonyl) phenoxy)piperidine- 1-carbonyl)-N-(1-(4-methoxybenzyl) piperidin-4-yl)nicotinamide | 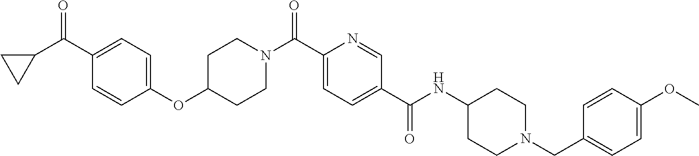 [SMILES: COc1ccc(CN2CCC(NC(=O)c3ccc(C(=O)N4CCC(Oc5ccc(C(=O)C6CC6)cc5)CC4)nc3)CC2)cc1] |
| 459 | N-(6-(4-cyanophenoxy)pyridin-3-yl)-6-(4- (4-(methylsulfonyl)phenoxy)piperidine-1- carbonyl)nicotinamide | 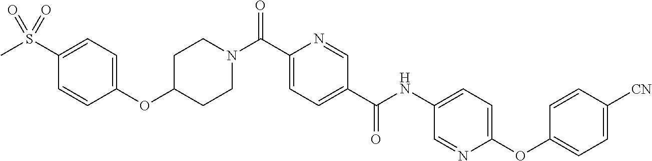 [SMILES: CS(=O)(=O)c1ccc(OC2CCN(C(=O)c3ccc(C(=O)Nc4ccc(Oc5ccc(C#N)cc5)nc4)cn3)CC2)cc1] |
| 460 | N-(6-(4-cyanophenoxy)pyridin-3-yl)-6-(4- (4-methoxybenzoyl)piperidine-1- carbonyl)nicotinamide | 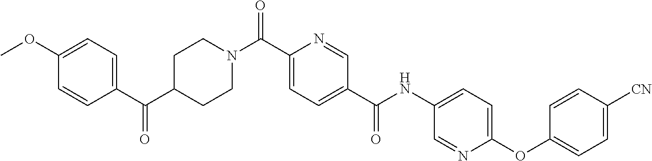 [SMILES: COc1ccc(C(=O)C2CCN(C(=O)c3ccc(C(=O)Nc4ccc(Oc5ccc(C#N)cc5)nc4)cn3)CC2)cc1] |
| 461 | N-((cis)-3-fluoro-1-(4- (trifluoromethoxy)benzyl)piperidin-4-yl)-6- (4-(4-methoxybenzoyl)piperidine-1- carbonyl)nicotinamide | 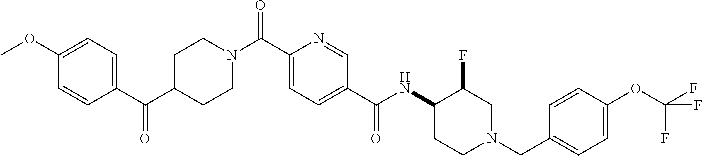 |
| 462 | N-(6-(4-acetylphenoxy)pyridin-3-yl)-6-(4- (4-methoxybenzoyl)piperidine-1- carbonyl)nicotinamide | 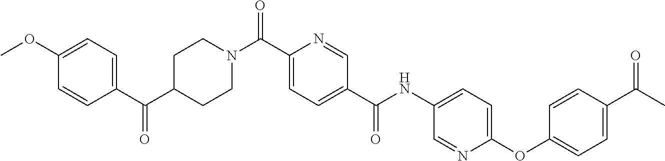 [SMILES: COc1ccc(C(=O)C2CCN(C(=O)c3ccc(C(=O)Nc4ccc(Oc5ccc(C(C)=O)cc5)nc4)cn3)CC2)cc1] |
| 463 | N-(6-(4-cyanophenoxy)pyridin-3-yl)-6-(4- (2,4-difluorobenzoyl)piperidine-1- carbonyl)nicotinamide | 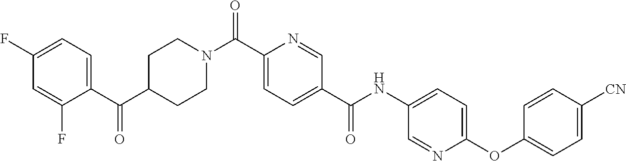 [SMILES: N#Cc1ccc(Oc2ccc(NC(=O)c3ccc(C(=O)N4CCC(C(=O)c5ccc(F)cc5F)CC4)nc3)cn2)cc1] |
| 464 | N-(6-(4-acetylphenoxy)pyridin-3-yl)-6-(4- (2,4-difluorobenzoyl)piperidine-1- carbonyl)nicotinamide | 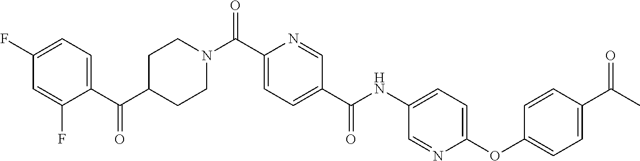 [SMILES: CC(=O)c1ccc(Oc2ccc(NC(=O)c3ccc(C(=O)N4CCC(C(=O)c5ccc(F)cc5F)CC4)nc3)cn2)cc1] |
| 465 | 6-(4-(4-methoxybenzoyl)piperidine-1- carbonyl)-N-(6-(4- (methylsulfonyl)phenoxy)pyridin-3- yl)nicotinamide | 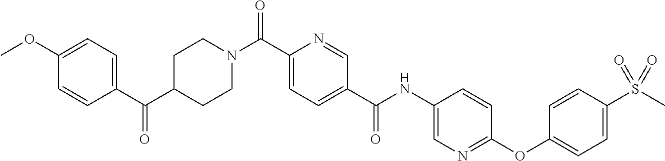 |
| 466 | 6-(4-(2,4-difluorobenzoyl)piperidine-1- carbonyl)-N-(6-(4- (methylsulfonyl)phenoxy)pyridin-3- yl)nicotinamide | 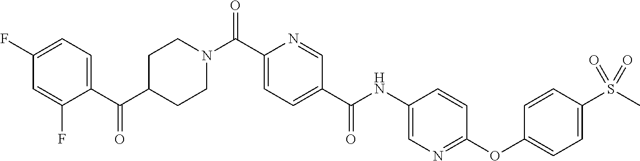 [SMILES: CS(=O)(=O)c1ccc(Oc2ccc(NC(=O)c3ccc(C(=O)N4CCC(C(=O)c5ccc(F)cc5F)CC4)nc3)cn2)cc1] |
| 467 | N-(6-(4-fluorophenylsulfonyl)pyridin-3-yl)- 6-(4-(4-methoxybenzoyl)piperidine-1- carbonyl)nicotinamide | 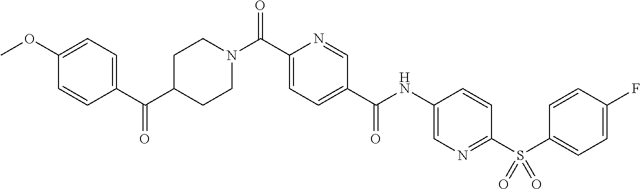 [SMILES: COc1ccc(C(=O)C2CCN(C(=O)c3ccc(C(=O)Nc4ccc(S(=O)(=O)c5ccc(F)cc5)nc4)cn3)CC2)cc1] |
| 468 | N-(5-(4-cyanophenoxy)pyridin-2-yl)-6-(4- (4-methoxybenzoyl)piperidine-1- carbonyl)nicotinamide | 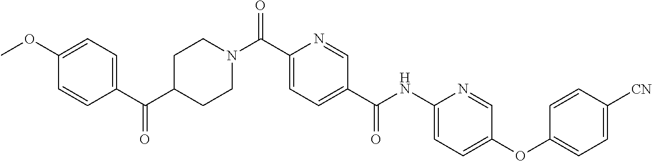 [SMILES: COc1ccc(C(=O)C2CCN(C(=O)c3ccc(C(=O)Nc4ccc(Oc5ccc(C#N)cc5)cn4)cn3)CC2)cc1] |
| 469 | N-(5-(4-cyanophenoxy)pyridin-2-yl)-6-(4- (2,4-difluorobenzoyl)piperidine-1- carbonyl)nicotinamide | 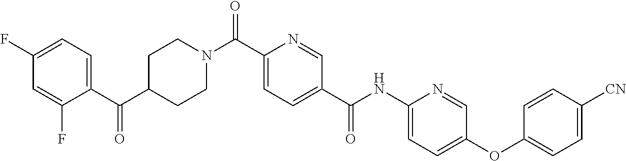 [SMILES: N#Cc1ccc(Oc2ccc(NC(=O)c3ccc(C(=O)N4CCC(C(=O)c5ccc(F)cc5F)CC4)nc3)nc2)cc1] |
| 470 | 6-(4-(4-fluorophenylsulfonyl)piperidine-1- carbonyl)-N-(1-(4- (trifluoromethoxy)benzyl)piperidin-4- yl)nicotinamide | 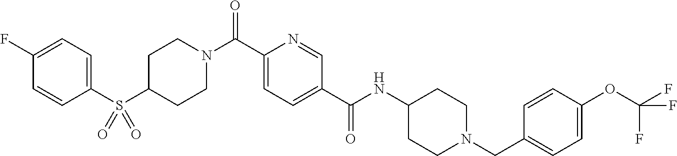 [SMILES: O=C(NC1CCN(Cc2ccc(OC(F)(F)F)cc2)CC1)c1ccc(C(=O)N2CCC(S(=O)(=O)c3ccc(F)cc3)CC2)nc1] |
| 471 | N-(1-(4-cyanobenzyl)piperidin-4-yl)-6-(4- (4-fluorophenylsulfonyl)piperidine-1- carbonyl)nicotinamide | 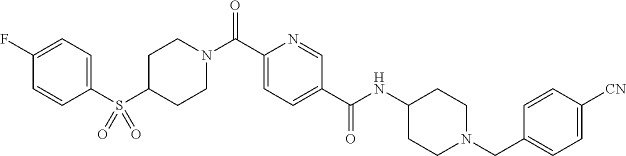 [SMILES: N#Cc1ccc(CN2CCC(NC(=O)c3ccc(C(=O)N4CCC(S(=O)(=O)c5ccc(F)cc5)CC4)nc3)CC2)cc1] |
| 472 | N-(6-(4-cyanophenoxy)pyridin-3-yl)-6-(4- (4-fluorophenylsulfonyl)piperidine-1- carbonyl)nicotinamide | 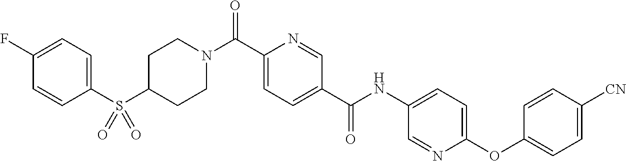 [SMILES: N#Cc1ccc(Oc2ccc(NC(=O)c3ccc(C(=O)N4CCC(S(=O)(=O)c5ccc(F)cc5)CC4)nc3)cn2)cc1] |
| 473 | N-(6-(4-acetylphenoxy)pyridin-3-yl)-6-(4- (4-fluorophenylsulfonyl)piperidine-1- carbonyl)nicotinamide | 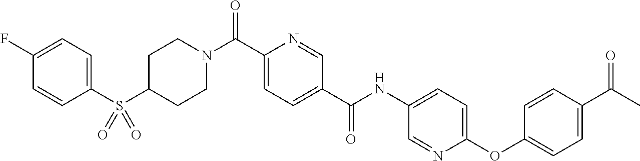 [SMILES: CC(=O)c1ccc(Oc2ccc(NC(=O)c3ccc(C(=O)N4CCC(S(=O)(=O)c5ccc(F)cc5)CC4)nc3)cn2)cc1] |
| 474 | 6-(4-(4-fluorophenylsulfonyl)piperidine-1- carbonyl)-N-(1-(4- methoxybenzyl)piperidin-4-yl)nicotinamide | 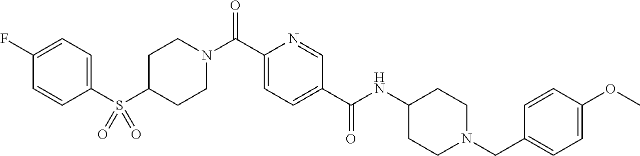 [SMILES: COc1ccc(CN2CCC(NC(=O)c3ccc(C(=O)N4CCC(S(=O)(=O)c5ccc(F)cc5)CC4)nc3)CC2)cc1] |
| 475 | 6-(4-(4-fluorophenylsulfonyl)piperidine-1- carbonyl)-N-(1-(3- methoxybenzyl)piperidin-4-yl)nicotinamide | 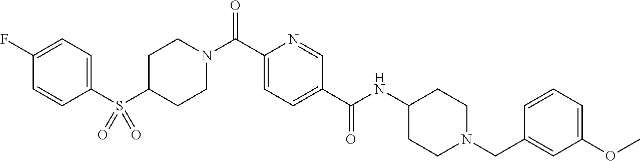 [SMILES: COc1cccc(CN2CCC(NC(=O)c3ccc(C(=O)N4CCC(S(=O)(=O)c5ccc(F)cc5)CC4)nc3)CC2)c1] |
| 476 | N-(6-(4-cyanophenoxy)pyridin-3-yl)-6-(4- (4-fluorobenzyl)piperazine-1- carbonyl)nicotinamide | 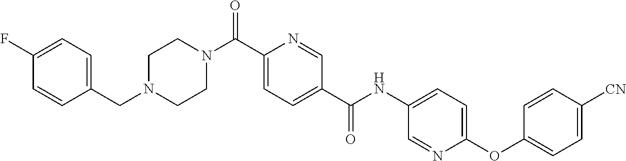 [SMILES: N#Cc1ccc(Oc2ccc(NC(=O)c3ccc(C(=O)N4CCN(Cc5ccc(F)cc5)CC4)nc3)cn2)cc1] |
| 477 | N-(6-(4-acetylphenoxy)pyridin-3-yl)-6-(4- (4-fluorobenzyl)piperazine-1- carbonyl)nicotinamide | 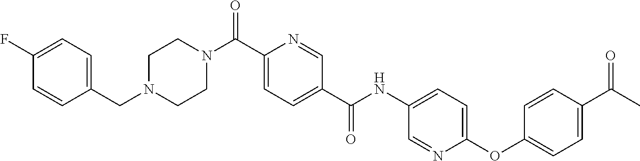 [SMILES: CC(=O)c1ccc(Oc2ccc(NC(=O)c3ccc(C(=O)N4CCN(Cc5ccc(F)cc5)CC4)nc3)cn2)cc1] |
| 478 | N-(6-(4-cyanophenoxy)-2-methylpyridin-3- yl)-6-(4-(4-methoxybenzoyl)piperidine-1- carbonyl)nicotinamide | 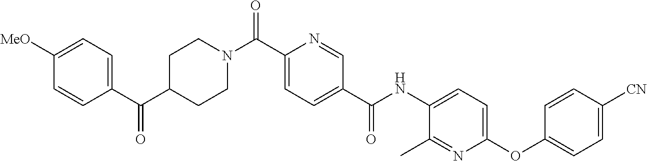 [SMILES: COc1ccc(C(=O)C2CCN(C(=O)c3ccc(C(=O)Nc4ccc(Oc5ccc(C#N)cc5)nc4C)cn3)CC2)cc1] |
| 479 | N-(6-(4-cyanophenoxy)-2-methylpyridin-3- yl)-6-(4-(2,4-difluorobenzoyl)piperidine-1- carbonyl)nicotinamide | 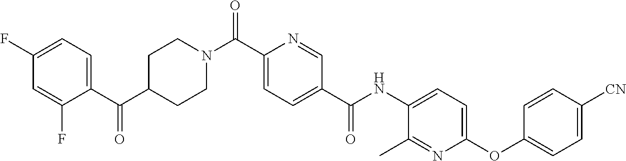 [SMILES: Cc1nc(Oc2ccc(C#N)cc2)ccc1NC(=O)c1ccc(C(=O)N2CCC(C(=O)c3ccc(F)cc3F)CC2)nc1] |
| 480 | N-(6-(4-(dimethylcarbamoyl)phenoxy) pyridin-3-yl)-6-(4-(4-methoxybenzoyl) piperidine-1- carbonyl)nicotinamide | 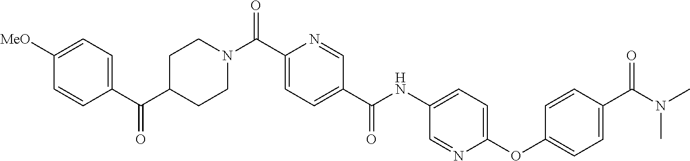 [SMILES: COc1ccc(C(=O)C2CCN(C(=O)c3ccc(C(=O)Nc4ccc(Oc5ccc(C(=O)N(C)C)cc5)nc4)cn3)CC2)cc1] |
| 481 | 6-(4-(2,4-difluorobenzoyl)piperidine-1- carbonyl)-N-(6-(4- (dimethylcarbamoyl)phenoxy)pyridin-3- yl)nicotinamide | 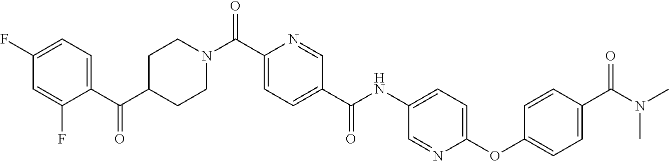 [SMILES: CN(C)C(=O)c1ccc(Oc2ccc(NC(=O)c3ccc(C(=O)N4CCC(C(=O)c5ccc(F)cc5F)CC4)nc3)cn2)cc1] |
| 482 | N-(1-(4-cyanobenzyl)piperidin-4-yl)-6-(4- (4-methoxybenzoyl)piperidine-1-carbonyl)- N-methylnicotinamide | 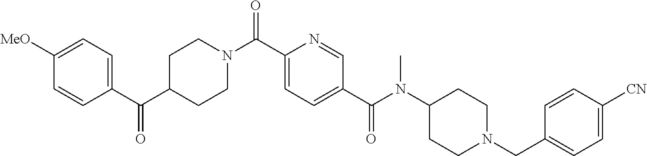 [SMILES: COc1ccc(C(=O)C2CCN(C(=O)c3ccc(C(=O)N(C)C4CCN(Cc5ccc(C#N)cc5)CC4)cn3)CC2)cc1] |
| 483 | 6-(4-(4-methoxybenzoyl)piperidine-1- carbonyl)-N-methyl-N-(1-(4- (trifluoromethoxy)benzyl)piperidin-4- yl)nicotinamide | 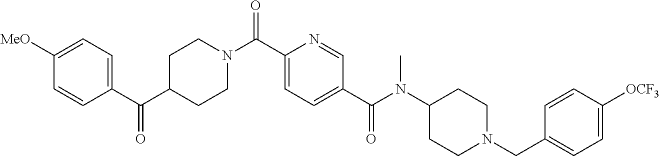 [SMILES: COc1ccc(C(=O)C2CCN(C(=O)c3ccc(C(=O)N(C)C4CCN(Cc5ccc(OC(F)(F)F)cc5)CC4)cn3)CC2)cc1] |
| 484 | 6-(4-(4-methoxybenzoyl)piperidine-1- carbonyl)-N-(1-(4- methoxybenzyl)piperidin-4-yl)-N- methylnicotinamide | 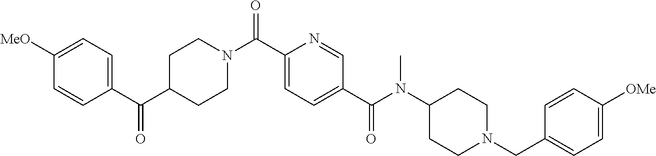 [SMILES: COc1ccc(CN2CCC(N(C)C(=O)c3ccc(C(=O)N4CCC(C(=O)c5ccc(OC)cc5)CC4)nc3)CC2)cc1] |
| 485 | N-(6-(4-acetylphenoxy)pyridin-3-yl)-6-(4- (4-(methylsulfonyl)phenoxy)piperidine-1- carbonyl)nicotinamide | 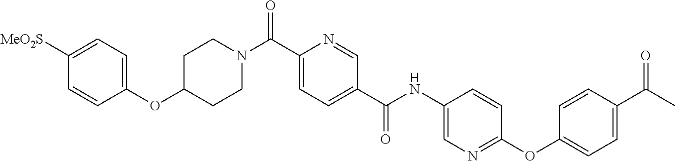 [SMILES: CC(=O)c1ccc(Oc2ccc(NC(=O)c3ccc(C(=O)N4CCC(Oc5ccc(S(C)(=O)=O)cc5)CC4)nc3)cn2)cc1] |
| 486 | N-(6-(4-acetylphenoxy)pyridin-3- yl)-6-(4-(4-(cyclopropylsulfonyl)phenoxy) piperidine-1-carbonyl)nicotinamide | 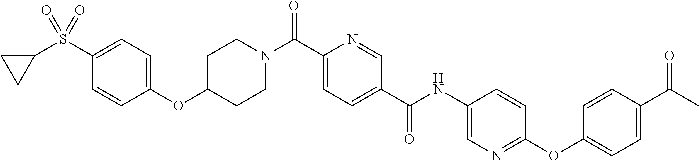 [SMILES: CC(=O)c1ccc(Oc2ccc(NC(=O)c3ccc(C(=O)N4CCC(Oc5ccc(S(=O)(=O)C6CC6)cc5)CC4)nc3)cn2)cc1] |
| 487 | N-(6-(4-acetylphenoxy)pyridin-3-yl)-6-(4- (4-(methylsulfonyl)phenyl)piperazine-1- carbonyl)nicotinamide | 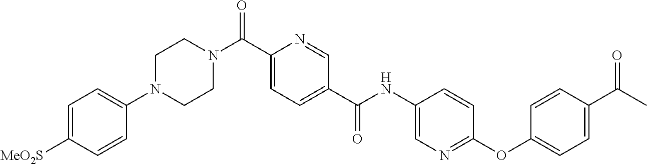 [SMILES: CC(=O)c1ccc(Oc2ccc(NC(=O)c3ccc(C(=O)N4CCN(c5ccc(S(C)(=O)=O)cc5)CC4)nc3)cn2)cc1] |
| 488 | N-(6-(4-acetylphenoxy)pyridin-3-yl)-6-(4- (4-(dimethylcarbamoyl)phenoxy)piperidine- 1-carbonyl)nicotinamide | 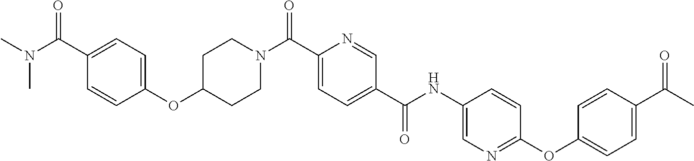 [SMILES: CC(=O)c1ccc(Oc2ccc(NC(=O)c3ccc(C(=O)N4CCC(Oc5ccc(C(=O)N(C)C)cc5)CC4)nc3)cn2)cc1] |
| 489 | N-(6-(4-acetylphenoxy)pyridin-3-yl)-6-(4- (4-(isopropylsulfonyl)phenyl)piperazine-1- carbonyl)nicotinamide | 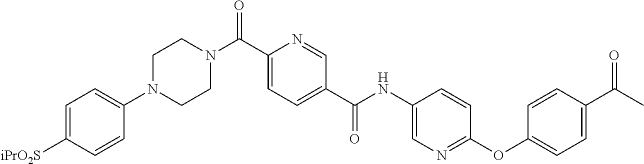 |
| 490 | N-(1-(4-(dimethylcarbamoyl)benzyl) piperidin-4-yl)-6-(4-(4- methoxybenzoyl)piperidine- 1-carbonyl)nicotinamide | 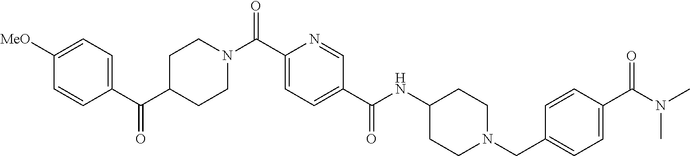 [SMILES: COc1ccc(C(=O)C2CCN(C(=O)c3ccc(C(=O)NC4CCN(Cc5ccc(C(=O)N(C)C)cc5)CC4)cn3)CC2)cc1] |
| 491 | N-(1-(4-cyanobenzyl)piperidin-4-yl)-6-(4- (4-fluorobenzyl)piperazin-1-yl)pyridazine- 3-carboxamide | 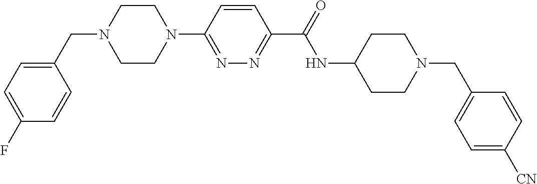 [SMILES: N#Cc1ccc(CN2CCC(NC(=O)c3ccc(N4CCN(Cc5ccc(F)cc5)CC4)nn3)CC2)cc1] |
| 492 | N-(1-(4-cyanobenzyl)piperidin-4-yl)-5-(4- (4-(pentafluorosulfanyl)phenoxy)piperidine- 1-carbonyl)picolinamide | 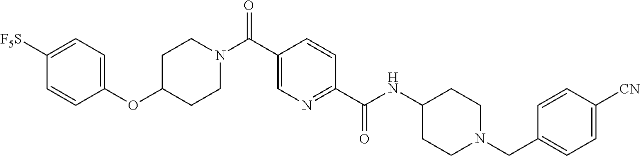 |
| 493 | N-(1-(4-cyanobenzyl)piperidin-4-yl)-6-(4- (4-(pentafluorosulfanyl)phenoxy)piperidine- 1-carbonyl)nicotinamide | 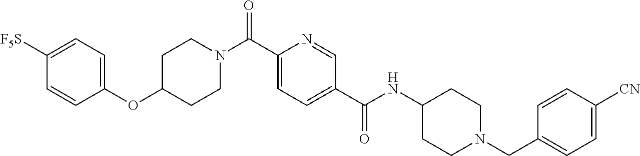 |
| 494 | 6-(4-(4-(pentafluorosulfanyl)phenoxy) piperidine-1-carbonyl)-N-(1-(4- (trifluoromethoxy)benzyl)piperidin-4- yl)nicotinamide | 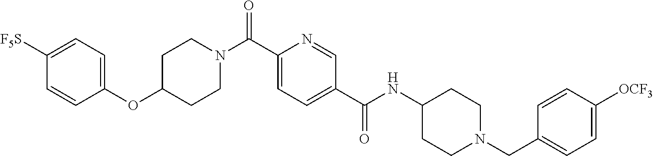 |
| 495 | N-(1-(4-methoxybenzyl)piperidin-4-yl)-6- (4-(4-(pentafluorosulfanyl) phenoxy)piperidine-1- carbonyl)nicotinamide | 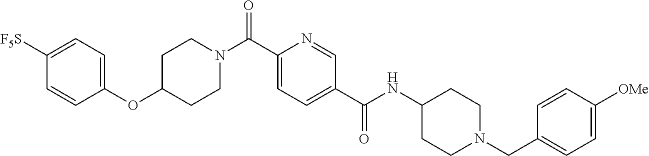 |
| 496 | N-(6-(4-fluorophenoxy)pyridin-3-yl)-6-(4- (4-(pentafluorosulfanyl)phenoxy)piperidine- 1-carbonyl)nicotinamide | 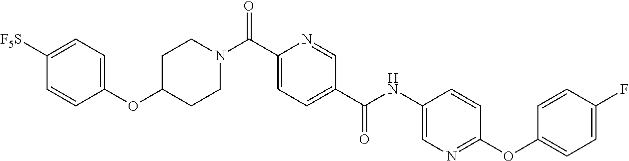 |
| 497 | N-(6-(4-cyanophenoxy)pyridin-3-yl)-6-(4- (4-(pentafluorosulfanyl)phenoxy)piperidine- 1-carbonyl)nicotinamide | 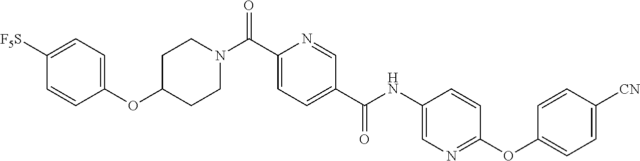 |
| 498 | N-(1-(4-cyanobenzyl)-3,3-difluoropiperidin- 4-yl)-6-(4-(4-methoxybenzoyl)piperidine-1- carbonyl)nicotinamide | 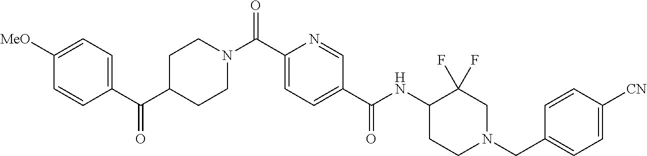 [SMILES: COc1ccc(C(=O)C2CCN(C(=O)c3ccc(C(=O)NC4CCN(Cc5ccc(C#N)cc5)CC4(F)F)cn3)CC2)cc1] |

In other embodiments as described above, the compound is
5-(4-(4-cyanobenzyl)piperazine-1-carbonyl)-N-(1-(4-cyanobenzyl)piperidin-4-yl)picolinamide;
N-(1-(4-cyanobenzyl)piperidin-4-yl)-5-(4-(4-fluorobenzyl)piperazine-1-carbonyl)picolinamide;
N-(1-(4-cyanobenzyl)piperidin-4-yl)-5-(4-(4-(trifluoromethyl)benzyl)piperazine-1-carbon yl)picolinamide
(S)-5-(4-(4-chlorophenyl)piperazine-1-carbonyl)-N-(1-(4-fluorobenzyl)pyrrolidin-3-yl)picolinamide;
(S)-5-(4-(4-chlorophenyl)piperazine-1-carbonyl)-N-(1-(pyridin-4-ylmethyl)pyrrolidin-3-yl) picolinamide;
(S)-5-(4-(4-chlorophenyl)piperazine-1-carbonyl)-N-(1-(4-cyanobenzyl)pyrrolidin-3-yl)picolinamide;
N-(1-(4-chlorobenzyl)pyrrolidin-3-yl)-5-(4-(4-chlorophenyl)piperazine-1-carbonyl)picolinamide; or
5-(4-(4-chlorophenyl)piperazine-1-carbonyl)-N-(1-(4-(trifluoromethyl)benzyl)pyrrolidin-3-yl)picolinamide.
   or a pharmaceutically acceptable salt, prodrug or N-oxide thereof (or a solvate or hydrate thereof).

For simplicity, chemical moieties are defined and referred to throughout primarily as univalent chemical moieties (for example, alkyl, aryl, etc.). Nevertheless, such terms are also used to convey corresponding multivalent moieties under the appropriate structural circumstances clear to those skilled in the art. For example, while an “alkyl” moiety can refer to a monovalent radical (for example CH₃—CH₂—), in some circumstances a bivalent linking moiety can be “alkyl,” in which case those skilled in the art will understand the alkyl to be a divalent radical (for example the C₂ alkylene-CH₂—CH₂— may be described as a C₂ alkyl group), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, for example, an N-oxide or tetrasubstituted ammonium salt. On occasion a moiety may be defined, for example, as (A)ₐ-B—, wherein a is 0 or 1. In such instances, when a is 0 the moiety is B— and when a is 1 the moiety is A-B—.

As used herein, the term “alkyl” includes alkyl, alkenyl and alkynyl groups of a designed number of carbon atoms, desirably from 1 to about 12 carbons (i.e., inclusive of 1 and 12). The term “Cₘ—Cₙ alkyl” means an alkyl group having from m to n carbon atoms (i.e., inclusive of m and n). The term “Cₘ—Cₙ alkyl” means an alkyl group having from m to n carbon atoms. For example, “C₁-C₆ alkyl” is an alkyl group having from one to six carbon atoms. Alkyl and alkyl groups may be straight or branched and depending on context, may be a monovalent radical or a divalent radical (i.e., an alkylene group). In the case of an alkyl or alkyl group having zero carbon atoms (i.e., “C₀ alkyl”), the group is simply a single covalent bond if it is a divalent radical or is a hydrogen atom if it is a monovalent radical. For example, the moiety “—(C₀-C₆ alkyl)-Ar” signifies connection of an optionally substituted aryl through a single bond or an alkylene bridge having from 1 to 6 carbons. Examples of “alkyl” include, for example, methyl, ethyl, propyl, isopropyl, butyl, iso-, sec- and tert-butyl, pentyl, hexyl, heptyl, 3-ethylbutyl, 3-hexenyl and propargyl. If the number of carbon atoms is not specified, the subject “alkyl” or “alkyl” moiety has from 1 to 12 carbons.

The term “haloalkyl” is an alkyl group substituted with one or more halogen atoms, for example F, Cl, Br and I. A more specific term, for example, “fluoroalkyl” is an alkyl group substituted with one or more fluorine atoms. Examples of “fluoroalkyl” include fluoromethyl, difluoromethyl, trifluoromethyl, pentafluoroethyl, hexafluoroisopropyl and the like. In certain embodiments of the compounds disclosed herein, each haloalkyl is a fluoroalkyl.

The term “aryl” represents an aromatic carbocyclic ring system having a single ring (for example, phenyl) which is optionally fused to other aromatic hydrocarbon rings or non-aromatic hydrocarbon rings. “Aryl” includes ring systems having multiple condensed rings and in which at least one is aromatic, (for example, 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, 2,3-dihydrobenzofuranyl and 6,7,8,9-tetrahydro-5H-benzo[α]cycloheptenyl. The aryl groups herein are unsubstituted or, when specified as “optionally substituted”, can unless stated otherwise be substituted in one or more substitutable positions with various groups, as described below.

The term “heteroaryl” refers to an aromatic ring system containing at least one heteroatom selected from nitrogen, oxygen and sulfur in an aromatic ring. The heteroaryl may be fused to one or more cycloalkyl or heterocycloalkyl rings. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as described below.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl may be saturated (i.e., a heterocycloalkyl) or partially unsaturated (i.e., a heterocycloalkenyl). The heterocycloalkyl ring is optionally fused to other heterocycloalkyl rings and/or non-aromatic hydrocarbon rings and/or phenyl rings. In certain embodiments, the heterocycloalkyl groups have from 3 to 7 members in a single ring. In other embodiments, heterocycloalkyl groups have 5 or 6 members in a single ring. Examples of heterocycloalkyl groups include, for example, azabicyclo[2.2.2]octyl (in each case also “quinuclidinyl” or a quinuclidine derivative), azabicyclo[3.2.1]octyl, morpholinyl, thiomorpholinyl, thiomorpholinyl S-oxide, thiomorpholinyl S,S-dioxide, 2-oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S-oxide, tetrahydrothienyl S,S-dioxide and homothiomorpholinyl S-oxide. Especially desirable heterocycloalkyl groups include morpholinyl, 3,4-dihydroisoquinolin-2(1H)-yl, tetrahydropyranyl, piperidinyl, aza-bicyclo[2.2.2]octyl, γ-butyrolactonyl (i.e., an oxo-substituted tetrahydrofuranyl), γ-butryolactamyl (i.e., an oxo-substituted pyrrolidine), pyrrolidinyl, piperazinyl, azepanyl, azetidinyl, thiomorpholinyl, thiomorpholinyl S,S-dioxide, 2-oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as described below.

The term “cycloalkyl” refers to a non-aromatic carbocyclic ring or ring system, which may be saturated (i.e., a cycloalkyl) or partially unsaturated (i.e., a cycloalkenyl). The cycloalkyl ring optionally fused to or otherwise attached (for example, bridged systems) to other cycloalkyl rings. Preferred cycloalkyl groups have from 3 to 7 members in a single ring. More preferred cycloalkyl groups have 5 or 6 members in a single ring. Examples of cycloalkyl groups include, for example, cyclohexyl, cyclopentyl, cyclobutyl, cyclopropyl, tetrahydronaphthyl and bicyclo[2.2.1]heptane. The cycloalkyl groups herein are unsubstituted or, when specified as “optionally substituted”, may be substituted in one or more substitutable positions with various groups.

The term “oxa” means a divalent oxygen radical in a chain, sometimes designated as —O—.

The term “oxo” means a doubly bonded oxygen, sometimes designated as ═O or for example in describing a carbonyl “C(O)” may be used to show an oxo substituted carbon.

The term “electron withdrawing group” means a group that withdraws electron density from the structure to which it is attached than would a similarly-attached hydrogen atom. For example, electron withdrawing groups can be selected from the group consisting of halo, cyano, —(C₁-C₄ fluoroalkyl), —O—(C₁-C₄ fluoroalkyl), —C(O)—(C₀-C₄ alkyl), —C(O)O—(C₀-C₄ alkyl), —C(O)N(C₀-C₄ alkyl)(C₀-C₄ alkyl), —S(O)₂O—(C₀-C₄ alkyl), —SF₅, NO₂ and —C(O)—Hca in which the Hca includes a nitrogen atom to which the —C(O)— is bound, in which no alkyl, fluoroalkyl or heterocycloalkyl is substituted with an aryl, heteroaryl, cycloalkyl or heterocycloalkyl-containing group.

The term “substituted,” when used to modify a specified group or radical, means that one or more hydrogen atoms of the specified group or radical are each, independently of one another, replaced with the same or different substituent groups as defined below.

Substituent groups for substituting for hydrogens on saturated carbon atoms in the specified group or radical are, unless otherwise specified, —R⁶⁰, halo, —O⁻M⁺, ═O, —OR⁷⁰, —SR⁷⁰, —S⁻M⁺, ═S, —NR⁸⁰R⁸⁰, ═NR⁷⁰, ═N—OR⁷⁰, trihalomethyl, —CF₃, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC (O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰R⁷⁰ and —NR⁷⁰C(NR⁷⁰)NR⁸⁰R⁸⁰. Each R⁶⁰ is independently selected from the group consisting of alkyl, heteroalkyl, cycloalkyl, heterocycloalkyl, heterocycloalkylalkyl, cycloalkylalkyl, aryl, arylalkyl, heteroaryl and heteroarylalkyl, each of which is optionally substituted with 1, 2, 3, 4 or 5 groups selected from the group consisting of halo, —O⁻M⁺, ═O, —OR⁷¹, —SR⁷¹, —S⁻M⁺, ═S, —NR⁸¹R⁸¹, ═NR⁷¹, ═N—OR⁷¹, trihalomethyl, —CF₃, —CN, —OCN, —SCN, —NO, —NO₂, ═N₂, —N₃, —SO₂R⁷¹, —SO₂O⁻M⁺, —SO₂OR⁷¹, —OSO₂R⁷¹, —OSO₂O⁻M⁺, —OSO₂OR⁷¹, —P(O)(O⁻)₂(M⁺)₂, —P(O)(OR⁷¹)O⁻M⁻, —P(O)(OR⁷¹)₂, —C(O)R⁷¹, —C(S)R⁷¹, —C(NR⁷¹)R⁷¹, —C(O)O⁻M⁺, —C(O)OR⁷¹, —C(S)OR⁷¹, —C(O)NR⁸¹R⁸¹, —C(NR⁷¹)NR⁸¹R⁸¹, —OC(O)R⁷¹, —OC(S)R⁷¹, —OC(O) O⁻M⁺, —OC(O)OR⁷¹, —OC(S)OR⁷¹, —NR⁷¹C(O)R⁷¹, —NR⁷¹C(S)R⁷¹, —NR⁷¹CO₂⁻M⁺, —NR⁷¹CO₂R⁷¹, —NR⁷¹C(S)OR⁷¹, —NR⁷¹C(O)NR⁸¹R⁸¹, —NR⁷¹C(NR⁷¹)R⁷¹ and —NR⁷¹C(NR⁷¹)NR⁸¹R⁸¹. Each R⁷⁰ is independently hydrogen or R⁶⁰; each R⁸⁰ is independently R⁷⁰ or alternatively, two R^{80'}s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺is a counter ion with a net single positive charge. Each R⁷¹ is independently hydrogen or R⁶¹, in which R⁶¹ is alkyl, heteroalkyl, cycloalkyl, heterocycloalkyl, heterocycloalkylalkyl, cycloalkylalkyl, aryl, arylalkyl, heteroaryl and heteroarylalkyl, each of which is optionally substituted with 1, 2, 3, 4 or 5 groups selected from the group consisting of halo, —O⁻M⁺, ═O, —OR⁷², —SR⁷², —S⁺M⁺, ═S, —NR⁸²R⁸², ═NR⁷², ═N—OR⁷², trihalomethyl, —CF₃, —CN, —OCN, —SCN, —NO, —NO₂, ═N₂, —N₃, —SO₂R⁷¹, —SO₂O⁻M⁺, —SO₂OR⁷², —OSO₂R⁷², OSO₂O⁻M⁺, —OSO₂OR⁷², —P(O)(O⁻)₂(M⁺)₂, —P(O)(OR⁷²)O⁻M⁺, —P(O)(OR⁷²)₂, —C(O)R⁷², —C(S)R⁷², —C(NR⁷²)R⁷², —C(O)O⁻M⁺, —C(O)OR⁷², —C(S)OR⁷², —C(O)NR⁸²R⁸², —C(NR⁷²)NR⁸²R⁸², —OC(O)R⁷², —OC(S)R⁷², —OC(O) O⁻M⁺, —OC(O)OR⁷², —OC(S)OR⁷², —NR⁷²C(O)R⁷², —NR⁷²C(S)R⁷², —NR⁷²CO₂⁻M⁺, —NR⁷²CO₂R⁷², —NR⁷²C(S)OR⁷², —NR⁷²C(O)NR⁸²R⁸², —NR⁷²C(NR⁷²)R⁷² and —NR⁷²C(NR⁷²)NR⁸²R⁸²; and each R⁸¹ is independently R⁷¹ or alternatively, two R⁸¹s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution. Each R⁷² is independently hydrogen, (C₁-C₆ alkyl) or (C₁-C₆ fluoroalkyl); each R⁸² is independently R⁷² or alternatively, two R⁸²s, taken together with the nitrogen atom to which they are bonded, form a 5-, 6- or 7-membered heterocycloalkyl which may optionally include 1, 2, 3 or 4 of the same or different additional heteroatoms selected from the group consisting of O, N and S, of which N may have —H or C₁-C₃ alkyl substitution. Each M⁺may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means for example that one of the counter ions for such divalent alkali earth ions can be an ionized form of a presently disclosed compound and the other a typical counter ion such as chloride, or two ionized presently disclosed molecules can serve as counter ions for such divalent alkali earth ions, or a doubly ionized compound can serve as the counter ion for such divalent alkali earth ions). As specific examples, —NR⁸⁰R⁸⁰ is meant to include —NH₂, —NH-alkyl, N-pyrrolidinyl, N-piperazinyl, 4-methyl-piperazin-1-yl and N-morpholinyl. In certain embodiments, each R⁶⁰ is H or (unsubstituted C₁-C₆ alkyl). In certain embodiments, each R⁷⁰ is H or (unsubstituted C₁-C₆ alkyl). In certain embodiments, each R⁸⁰ is H or (unsubstituted C₁-C₆ alkyl).

Substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰) —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Substituent groups for hydrogens on nitrogen atoms in “substituted” heteroalkyl and heterocycloalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, OC(O)OR⁷⁰, OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺are as previously defined.

In certain embodiments as described above, the substituent groups on carbon atoms can also or alternatively be —SF₅.

In certain embodiments of the compounds disclosed herein, a group that is substituted has 1, 2, 3, or 4 substituents, 1, 2, or 3 substituents, 1 or 2 substituents, or 1 substituent.

In certain embodiments, an “optionally substituted alkyl,” unless otherwise specified, is substituted with halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, “optionally substituted alkyl” is also or alternatively optionally substituted with —N₃ or —SF₅.

In certain embodiments, an “optionally substituted aryl,” unless otherwise specified, is substituted with halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, “optionally substituted aryl” is also or alternatively optionally substituted with —N₃ or —SF₅.

In certain embodiments, an “optionally substituted heteroaryl,” unless otherwise specified, is substituted with halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, “optionally substituted heteroaryl” is also or alternatively optionally substituted with —N₃ or —SF₅.

In certain embodiments, an “optionally substituted cycloalkyl,” unless otherwise specified, is substituted with halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆ alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, “optionally substituted cycloalkyl” is also or alternatively optionally substituted with —N₃ or —SF₅.

In certain embodiments, an “optionally substituted heterocycloalkyl,” unless otherwise specified, is substituted with halogen (e.g., F, Cl), unsubstituted (C₁-C₆ alkoxy) (e.g., methoxy, ethoxy), —(C₁-C₆ haloalkoxy) (e.g., trifluoromethoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³, —(NH)₀₋₁COR³³, heterocycloalkyl optionally substituted with an (unsubstituted C₁-C₆ alkyl) and heteroaryl optionally substituted with an (unsubstituted C₁-C₆alkyl), in which each R³³ is (unsubstituted C₁-C₆ alkyl), (C₁-C₆ haloalkyl(unsubstituted C₃-C₈ cycloalkyl) or (C₃-C₈ heterocycloalkyl) optionally substituted with an (unsubstituted C₁-C₆ alkyl). In certain embodiments, “optionally substituted heterocycloalkyl” is also or alternatively optionally substituted with —N₃ or —SF₅.

The compounds disclosed herein can also be provided as pharmaceutically acceptable salts. The term “pharmaceutically acceptable salts” or “a pharmaceutically acceptable salt thereof” refer to salts prepared from pharmaceutically acceptable non-toxic acids or bases including inorganic acids and bases and organic acids and bases. If the compound is basic, salts may be prepared from pharmaceutically acceptable non-toxic acids. Such salts may be, for example, acid addition salts of at least one of the following acids: benzenesulfonic acid, citric acid, α-glucoheptonic acid, D-gluconic acid, glycolic acid, lactic acid, malic acid, malonic acid, mandelic acid, phosphoric acid, propanoic acid, succinic acid, sulfuric acid, tartaric acid (d, l, or dl), tosic acid (toluenesulfonic acid), valeric acid, palmitic acid, pamoic acid, sebacic acid, stearic acid, lauric acid, acetic acid, adipic acid, carbonic acid, 4-chlorobenzenesulfonic acid, ethanedisulfonic acid, ethylsuccinic acid, fumaric acid, galactaric acid (mucic acid), D-glucuronic acid, 2-oxo-glutaric acid, glycerophosphoric acid, hippuric acid, isethionic acid (ethanolsulfonic acid), lactobionic acid, maleic acid, 1,5-naphthalene-disulfonic acid, 2-naphthalene-sulfonic acid, pivalic acid, terephthalic acid, thiocyanic acid, cholic acid, n-dodecyl sulfate, 3-hydroxy-2-naphthoic acid, 1-hydroxy-2-naphthoic acid, oleic acid, undecylenic acid, ascorbic acid, (+)-camphoric acid, d-camphorsulfonic acid, dichloroacetic acid, ethanesulfonic acid, formic acid, hydriodic acid, hydrobromic acid, hydrochloric acid, methanesulfonic acid, nicotinic acid, nitric acid, orotic acid, oxalic acid, picric acid, L-pyroglutamic acid, saccharine, salicylic acid, gentisic acid, and/or 4-acetamidobenzoic acid.

The compounds described herein can also be provided in prodrug form. “Prodrug” refers to a derivative of an active compound (drug) that requires a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety can proceed spontaneously, such as by way of a hydrolysis reaction, or it can be catalyzed or induced by another agent, such as by an enzyme, by light, by acid, or by a change of or exposure to a physical or environmental parameter, such as a change of temperature. The agent can be endogenous to the conditions of use, such as an enzyme present in the cells to which the prodrug is administered or the acidic conditions of the stomach, or it can be supplied exogenously. A wide variety of progroups, as well as the resultant promoieties, suitable for masking functional groups in the active drugs to yield prodrugs are well-known in the art. For example, a hydroxyl functional group can be masked as a sulfonate, ester or carbonate promoiety, which can be hydrolyzed in vivo to provide the hydroxyl group. An amino functional group can be masked as an amide, carbamate, imine, urea, phosphenyl, phosphoryl or sulfenyl promoiety, which can be hydrolyzed in vivo to provide the amino group. A carboxyl group can be masked as an ester (including silyl esters and thioesters), amide or hydrazide promoiety, which can be hydrolyzed in vivo to provide the carboxyl group. Other specific examples of suitable progroups and their respective promoieties will be apparent to those of skill in the art.

The compounds disclosed herein can also be provided as N-oxides.

The presently disclosed compounds, salts, prodrugs and N-oxides can be provided, for example, in solvate or hydrate form.

The AMPK-activating compounds (e.g., compounds of structural formulae (I)-(LXXXVI)) can be administered, for example, orally, topically, parenterally, by inhalation or spray or rectally in dosage unit formulations containing one or more pharmaceutically acceptable carriers, diluents or excipients. The term parenteral as used herein includes percutaneous, subcutaneous, intravascular (for example, intravenous), intramuscular, or intrathecal injection or infusion techniques and the like.

The AMPK-activating compound can be provided as part of pharmaceutical composition. For example, in one embodiment, a pharmaceutical composition includes a pharmaceutically acceptable carrier, diluent or excipient, and an AMPK-activating compound (e.g., as described above with reference to structural formulae (I)-(LXXXVI)).

In the pharmaceutical compositions disclosed herein, one or more of the AMPK-activating compounds (e.g., of structural formulae (I)-(LXXXVI)) may be present in association with one or more pharmaceutically acceptable carriers, diluents or excipients, and, if desired, other active ingredients. The pharmaceutical compositions containing compounds of structural formulae (I)-(LXXXVI) may be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any suitable method for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by suitable techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use can also be presented as lozenges.

Aqueous suspensions contain the active materials in admixture with excipients suitable for the manufacture of aqueous suspensions. Such excipients can be suspending agents, for example sodium carboxymethylcellulose, methylcellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative, flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils can be employed as a solvent or suspending medium. For this purpose any bland fixed oil can be employed including synthetic mono- or diglycerides. In addition, fatty acids such as oleic acid find use in the preparation of injectables.

AMPK-activating compounds (e.g., compounds of structural formulae (I)-(LXXXVI)) can be formulated into lotions, oils or powders for application to the skin according to certain methods described below.

AMPK-activating compounds (e.g., compounds of structural formulae (I)-(LXXXVI)) can also be administered in the form of suppositories, for example, for rectal administration of the drug. These compositions can be prepared by mixing the compound with a suitable non-irritating excipient that is solid at ordinary temperatures but liquid at the rectal temperature and will therefore melt in the rectum to release the drug. Such materials include cocoa butter and polyethylene glycols.

AMPK-activating compounds (e.g., compounds of structural formulae (I)-(LXXXVI)) can also be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The compounds disclosed herein can be made using procedures familiar to the person of ordinary skill in the art and as described herein. For example, compounds of structural formula (I) can be prepared according to Schemes 1-6, below, or analogous synthetic schemes:

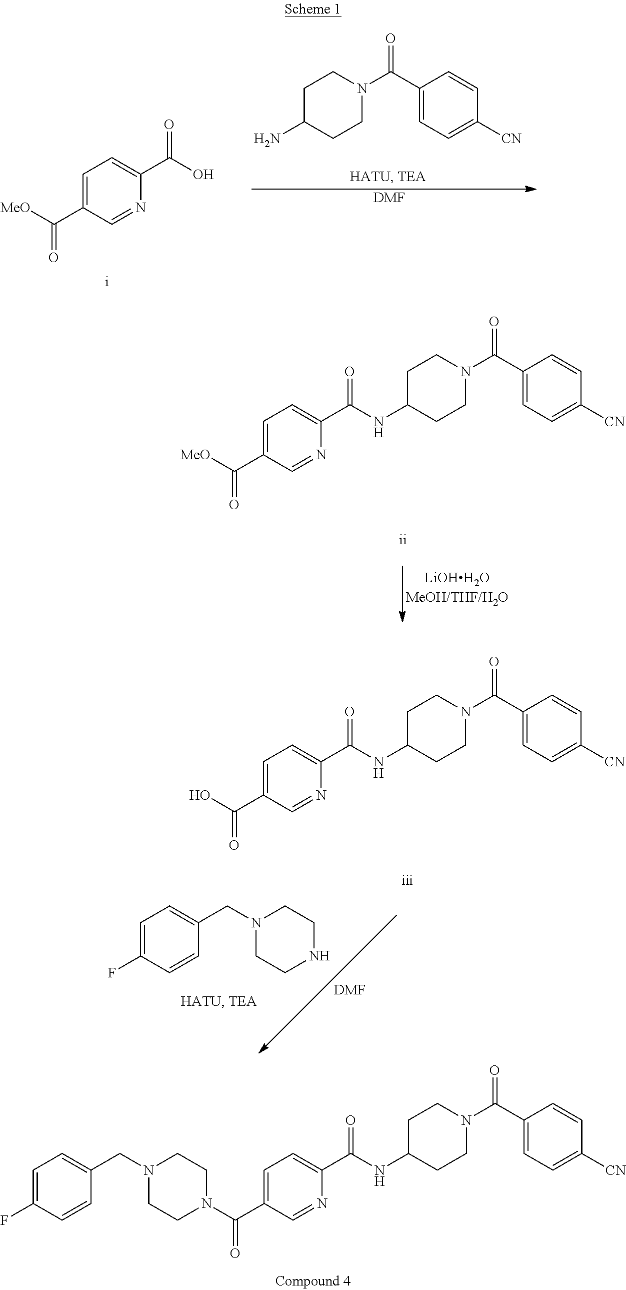

Referring to Scheme 1, a pyridinedicarboxylic acid monomethyl ester (i), for example, is coupled with an amine (here a substituted 1-benzoylpiperidine-4-amine) to form a carboxymethyl-substituted pyridinecarboxamide (ii). The ester is saponified to form the corresponding carboxylic acid (iii), which is then coupled with a suitable amine (in this case, a substituted 1-benzylpiperazine) to form Compound 4 of Table 1.

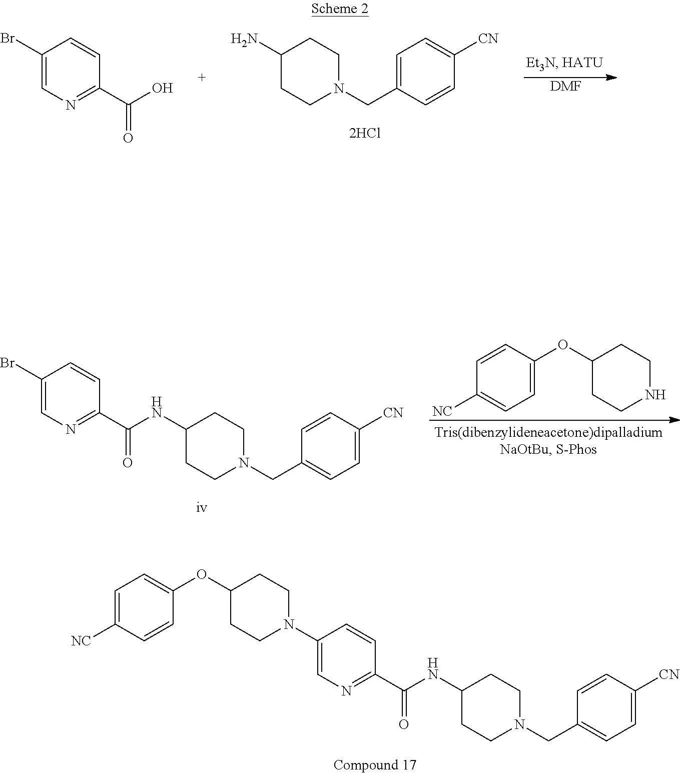

Referring to Scheme 2, a bromopyridinedicarboxylic acid, for example, is coupled with an amine (here a substituted 1-benzylpiperidine-4-amine) to form a bromo-substituted pyridinecarboxamide (iv), which is then coupled with a suitable amine (in this case, a substituted 4-phenoxypiperidine) using a palladium catalyst to form Compound 17 of Table 1.

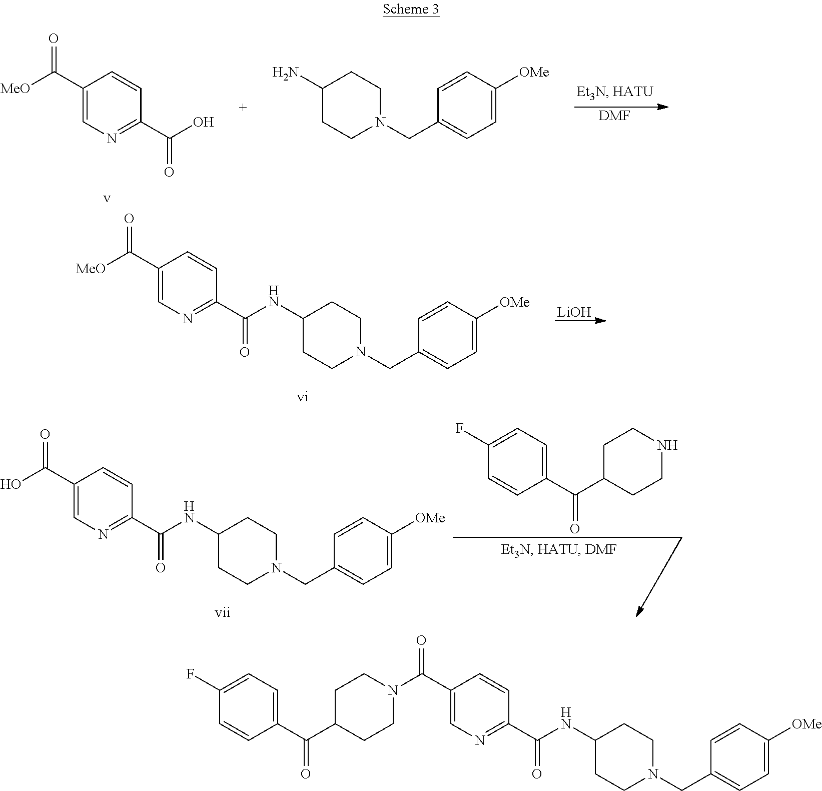

Referring to Scheme 3, a pyridinedicarboxylic acid monomethyl ester (v), for example, is coupled with an amine (here a substituted 1-benzylpiperidine-4-amine) to form a carboxymethyl-substituted pyridinecarboxamide (vi). The ester is saponified to form the corresponding carboxylic acid (vii), which is then coupled with a suitable amine (in this case, a substituted 4-benzoylpiperidine) to form Compound 160 of Table 1.

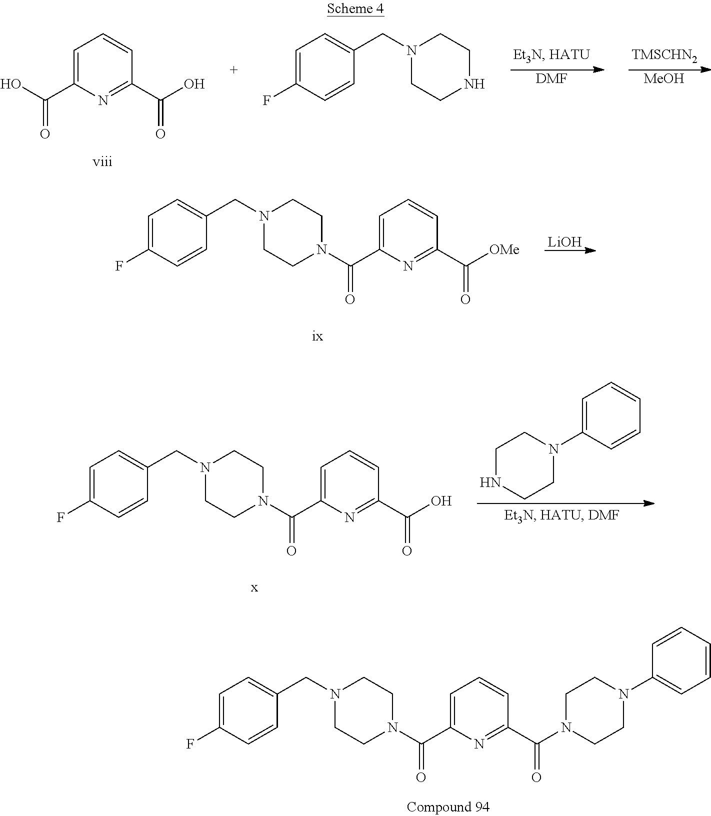

Referring to Scheme 4, a pyridine dicarboxylic acid (viii), for example, is coupled with one equivalent of an amine (here, a substituted 1-benzylepiperizine), then with methanol and trimethylsilyl(diazomethane) to form a carbomethoxy-substituted pyridinecarboxamide (ix), which is saponified to give a carboxylic acid-substituted pyridinecarboxamide (x). An amine (in this case, 1-phenylpiperazine) is coupled with the carboxylic acid-substituted pyridinecarboxamide (x) to form Compound 94 of Table 1.

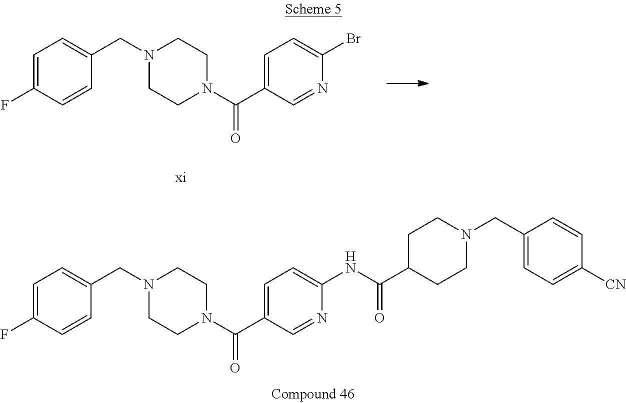 [SMILES: O=C(c1ccc(Br)nc1)N1CCN(Cc2ccc(F)cc2)CC1 ; N#Cc1ccc(CN2CCC(C(=O)Nc3ccc(C(=O)N4CCN(Cc5ccc(F)cc5)CC4)cn3)CC2)cc1]

Referring to Scheme 5, a bromopyridinecarboxamide (xi) is coupled with a substituted 1-benzylpiperidine-4-carboxamide using a palladium catalyst to form Compound 46 of Table 1. Reactions of this general type are described in more detail, for example, in Wrona, Iwona E. et al., Journal of Organic Chemistry (2010), 75(9), 2820-2835.

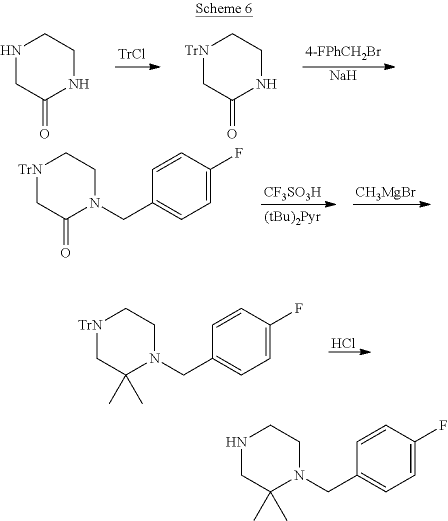

Scheme 6 describes a preparation that can be used to make gem-dimethylpiperazines for use in making compounds analogous to Compound 125 of Table 1. A piperazin-2-one is singly protected with trityl chloride, then coupled with an appropriate bromide (here, a substituted benzyl bromide) to form a 4-protected 1-(substituted benzyl)piperazin-2-one. The oxo is converted to a gem-dimethyl using Grignard chemistry, then the trityl is removed to yield the desired gem-dimethyl piperazine. Details are provided in the Examples below, and in Xiao, K-J.; Luo, J-M.; Ye, K-Y.; Wang, Y.; Huang, P-Q. Angew. Chem. Int. Ed. 2010, 49, 3037-3040.

One of skill in the art can adapt the reaction sequences of Schemes 1-6 to fit the desired target molecule. Of course, in certain situations one of skill in the art will use different reagents to affect one or more of the individual steps or to use protected versions of certain of the substituents. Additionally, one skilled in the art would recognize that compounds of structural formulae (I)-(LXXXVI) can be synthesized using different routes altogether.

Compounds suitable for use in the presently disclosed methods include compounds of Table 1, above. These compounds can be made according to the general scheme described above, for example using the procedures described in International Patent Application Publication no. WO 2012/016217 and in U.S. Patent Application publication no. 2012/0028954, each of which is hereby incorporated herein by reference in its entirety.

The following Examples are intended to further illustrate certain embodiments and are not intended to limit the scope of the disclosure.

### EXAMPLES

The following compounds were made using methods analogous to those of Schemes 1-7; in certain cases, exemplary synthetic procedures and/or characterization data are provided in International Patent Application Publication no. WO 2012/016217 and in U.S. Patent Application publication no. 2012/0028954.

### AMPK Activation Assay

Compounds were assayed for their ability to activate AMPK using an enzyme-linked immunosorbent assay. Reagents and procedures for measuring AMPK activation are well known and kits for AMPK activation assays are commercially available. The EC₅₀ values for AMPK activation for compounds 1-498 are presented in Table 2 below, in which “A” is less than 0.5 μM; “B” is 0.5-1 μM; “C” is 1-5 μM; and “D” is 5-10 μM; and “E” is >10 μM:

TABLE 2

| Cpd No. | AMPK EC₅₀ |
|---|---|
| 1 | A |
| 2 | E |
| 3 | B |
| 4 | B |
| 5 | B |
| 6 | B |
| 7 | A |
| 8 | A |
| 9 | A |
| 10 | A |
| 11 | A |
| 12 | D |
| 13 | C |
| 14 | B |
| 15 | C |
| 16 | A |
| 17 | E |
| 18 | A |
| 19 | F |
| 20 | F |
| 21 | A |
| 22 | A |
| 23 | A |
| 24 | A |
| 25 | A |
| 26 | A |
| 27 | B |
| 28 | B |
| 29 | B |
| 30 | C |
| 31 | A |
| 32 | B |
| 33 | D |
| 34 | C |
| 35 | B |
| 36 | B |
| 37 | D |
| 38 | B |
| 39 | C |
| 40 | C |
| 41 | E |
| 42 | C |
| 43 | C |
| 47 | A |
| 48 | B |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 72 | A |
| 73 | D |
| 74 | A |
| 75 | C |
| 76 | A |
| 77 | A |
| 78 | A |
| 79 | B |
| 80 | C |
| 81 | B |
| 82 | B |
| 83 | E |
| 84 | C |
| 85 | C |
| 86 | C |
| 87 | C |
| 88 | C |
| 89 | C |
| 90 | E |
| 91 | E |
| 92 | E |
| 93 | E |
| 94 | E |
| 95 | A |
| 96 | E |
| 97 | C |
| 98 | C |
| 99 | D |
| 100 | A |
| 101 | A |
| 102 | D |
| 103 | A |
| 104 | A |
| 105 | E |
| 106 | D |
| 107 | D |
| 108 | B |
| 109 | D |
| 110 | C |
| 111 | C |
| 112 | C |
| 113 | C |
| 114 | C |
| 115 | D |
| 116 | C |
| 117 | A |
| 118 | A |
| 119 | C |
| 120 | E |
| 121 | C |
| 122 | A |
| 123 | A |
| 124 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | B |
| 142 | A |
| 143 | A |
| 144 | B |
| 145 | A |
| 146 | A |
| 147 | A |
| 149 | B |
| 150 | A |
| 151 | A |
| 152 | A |
| 153 | C |
| 154 | A |
| 155 | A |
| 156 | A |
| 157 | A |
| 158 | A |
| 159 | C |
| 160 | A |
| 161 | A |
| 162 | A |
| 163 | A |
| 164 | B |
| 165 | A |
| 166 | A |
| 167 | A |
| 168 | A |
| 169 | B |
| 170 | E |
| 171 | A |
| 172 | A |
| 173 | A |
| 174 | A |
| 175 | A |
| 176 | C |
| 177 | C |
| 178 | A |
| 179 | A |
| 180 | A |
| 181 | A |
| 182 | A |
| 183 | A |
| 184 | A |
| 185 | A |
| 186 | A |
| 187 | C |
| 188 | B |
| 189 | C |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | B |
| 202 | A |
| 203 | A |
| 204 | A |
| 205 | A |
| 206 | A |
| 207 | A |
| 208 | A |
| 209 | A |
| 210 | E |
| 211 | A |
| 212 | A |
| 213 | A |
| 214 | E |
| 215 | E |
| 216 | E |
| 217 | E |
| 218 | A |
| 219 | A |
| 220 | A |
| 221 | C |
| 222 | C |
| 223 | C |
| 224 | A |
| 225 | A |
| 226 | A |
| 227 | A |
| 228 | A |
| 229 | C |
| 230 | B |
| 231 | A |
| 232 | A |
| 233 | A |
| 234 | A |
| 235 | A |
| 236 | A |
| 237 | C |
| 238 | C |
| 239 | C |
| 240 | A |
| 241 | A |
| 242 | A |
| 243 | A |
| 244 | D |
| 245 | A |
| 246 | A |
| 247 | A |
| 248 | A |
| 249 | A |
| 250 | A |
| 251 | A |
| 252 | B |
| 253 | B |
| 254 | A |
| 255 | A |
| 256 | A |
| 257 | A |
| 258 | A |
| 259 | A |
| 260 | A |
| 261 | A |
| 262 | A |
| 263 | A |
| 264 | D |
| 265 | C |
| 266 | A |
| 267 | A |
| 268 | A |
| 269 | A |
| 270 | A |
| 271 | A |
| 272 | A |
| 273 | A |
| 274 | E |
| 275 | A |
| 276 | A |
| 277 | A |
| 278 | A |
| 279 | A |
| 280 | A |
| 281 | A |
| 282 | A |
| 283 | A |
| 284 | A |
| 285 | A |
| 286 | A |
| 287 | A |
| 288 | A |
| 289 | A |
| 290 | A |
| 291 | A |
| 292 | A |
| 293 | A |
| 294 | A |
| 295 | A |
| 296 | A |
| 297 | A |
| 298 | A |
| 299 | A |
| 300 | A |
| 301 | A |
| 302 | A |
| 303 | A |
| 304 | A |
| 305 | A |
| 306 | A |
| 307 | B |
| 308 | A |
| 309 | A |
| 310 | A |
| 311 | A |
| 312 | A |
| 313 | A |
| 314 | E |
| 315 | A |
| 316 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | A |
| 330 | B |
| 331 | A |
| 332 | A |
| 333 | A |
| 334 | A |
| 335 | E |
| 336 | A |
| 337 | A |
| 338 | A |
| 339 | A |
| 340 | A |
| 341 | A |
| 342 | A |
| 343 | A |
| 344 | A |
| 345 | A |
| 346 | A |
| 347 | A |
| 348 | A |
| 349 | A |
| 350 | A |
| 351 | A |
| 352 | A |
| 353 | A |
| 354 | A |
| 355 | E |
| 356 | A |
| 357 | A |
| 358 | A |
| 359 | A |
| 360 | A |
| 361 | A |
| 362 | A |
| 363 | A |
| 364 | A |
| 365 | A |
| 366 | A |
| 367 | E |
| 368 | A |
| 369 | A |
| 370 | A |
| 371 | E |
| 372 | A |
| 373 | A |
| 374 | E |
| 375 | A |
| 376 | A |
| 377 | A |
| 378 | A |
| 379 | A |
| 380 | A |
| 381 | A |
| 382 | A |
| 383 | E |
| 384 | B |
| 385 | E |
| 386 | E |
| 387 | A |
| 388 | A |
| 389 | C |
| 390 | A |
| 391 | A |
| 392 | A |
| 393 | A |
| 394 | E |
| 395 | A |
| 396 | C |
| 397 | A |
| 398 | A |
| 399 | A |
| 400 | C |
| 401 | C |
| 402 | A |
| 403 | E |
| 404 | C |
| 405 | A |
| 406 | B |
| 407 | A |
| 408 | A |
| 409 | A |
| 410 | A |
| 411 | C |
| 412 | C |
| 413 | C |
| 414 | C |
| 415 | A |
| 416 | C |
| 417 | A |
| 418 | A |
| 419 | A |
| 420 | C |
| 421 | C |
| 422 | C |
| 423 | A |
| 424 | A |
| 425 | A |
| 426 | A |
| 427 | C |
| 428 | B |
| 429 | B |
| 430 | A |
| 431 | B |
| 432 | B |
| 433 | A |
| 434 | A |
| 435 | A |
| 436 | A |
| 437 | A |
| 438 | A |
| 439 | A |
| 440 | A |
| 441 | A |
| 442 | A |
| 443 | A |
| 444 | A |
| 445 | E |
| 446 | A |
| 447 | A |
| 448 | C |
| 449 | C |
| 450 | A |
| 451 | A |
| 452 | A |
| 453 | A |
| 454 | A |
| 455 | A |
| 456 | A |
| 457 | A |
| 458 | A |
| 459 | A |
| 460 | A |
| 461 | A |
| 462 | A |
| 463 | A |
| 464 | A |
| 465 | A |
| 466 | A |
| 467 | C |
| 468 | A |
| 469 | A |
| 470 | A |
| 471 | C |
| 472 | C |
| 473 | C |
| 474 | C |
| 475 | C |
| 476 | A |
| 477 | A |
| 478 | A |
| 479 | A |
| 480 | A |
| 481 | A |
| 482 | A |
| 483 | A |
| 484 | A |
| 485 | A |
| 486 | A |
| 487 | C |
| 488 | A |
| 489 | A |
| 490 | C |
| 491 | C |
| 492 | A |
| 493 | A |
| 494 | A |
| 495 | A |
| 496 | A |
| 497 | A |
| 498 | A |

### Biological Assays

Modulation of mitochondrial function through inhibiting respiratory complex I activates a key sensor of cellular energy status, the 5′-AMP-activated protein kinase (AMPK). As described in the experiments below, in vivo metabolite profiling of db/db mice treated with an exemplary AMPK activator showed a clear upregulation of fatty acid oxidation and catabolism of branched chain amino acids. Additionally, analyses performed using both ¹³C-palmitate and ¹³C-glucose tracers revealed compound mediated-increases incomplete oxidation of both glucose and palmitate to CO₂ in skeletal muscle, liver, and adipose tissue, indicating that our potent mitochondrial modulator increased mitochondrial function in vivo. Chronic treatment of db/db mice improved glucose tolerance and insulin sensitivity similar to metformin, but with significantly lower doses. Furthermore, treatment of aged DIO mice displaying a clear running deficit pre-treatment improved treadmill endurance and also reduced hepatic steatosis.

### Increase in Palmitate Oxidation in Liver, Skeletal Muscle, and Adipose Tissue.

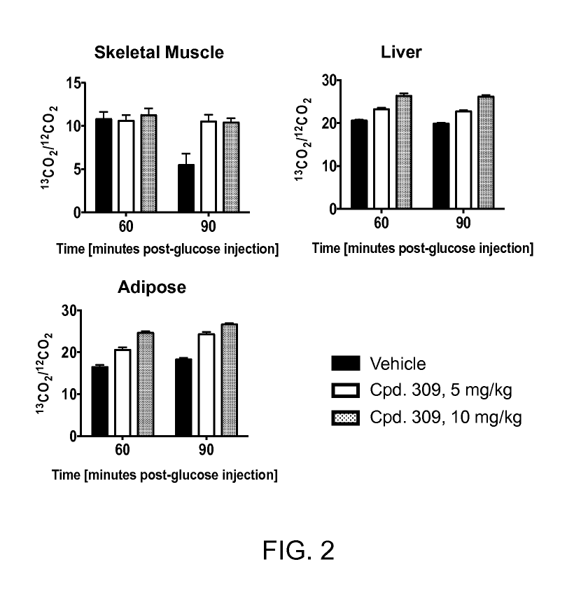

The elevated levels of BHBA detected by the global metabolite profiling are an indirect indicator of an increase in lipid oxidation. In order to determine whether compound 309 had a direct effect on β-oxidation, [U-¹³C]-palmitate was orally administered to non-fasted mice treated with either vehicle or compound 309 for one week followed by measurements of ¹³CO₂ to ¹²CO₂ ratio in various tissues after palmitate feeding. Appearance of ¹³CO₂ occurs via β-oxidation of the labeled palmitate to release ¹³C-acetyl CoA, which must pass through one round of the TCA cycle before the labeled carbon can be given off as ¹³CO₂. An increased β-oxidation rate will result in a corresponding increase in ¹³CO₂ enrichment. In agreement with the elevation in relative BHBA levels detected in liver, adipose, and skeletal muscle tissues, an increase in ¹³CO₂ enrichment is observed for compound 309 treatment across all three tissues at both 60 and 120 minutes after palmitate administration. Ratios of ¹³CO₂ to ¹²CO₂ were higher for all treatment groups, including the vehicle, at 120 minutes compared to 60 minutes after palmitate feeding (FIG. 2). This time-dependent elevation was due to the slow absorption kinetics of the orally administered tracer suspension. In contrast to the liver and adipose tissue, no dose-dependence in ¹³CO₂ enrichment was evident in skeletal muscle. All the data together demonstrate that chronic treatment of db/db mice with compound 309 directly upregulated fatty acid oxidation in liver, skeletal muscle, and adipose tissue.

### Metabolomics Study.

Maledb/db mice (8 weeks) were orally gavaged once daily with either vehicle or 10 mg/kg compound 309. Liver, muscle, adipose tissue, and plasma from treated mice (n=6/treatment group) were collected 30 minutes after dosing both on day 3 of treatment. Frozen tissue and plasma samples were sent to Metabolon for unbiased metabolite analysis [(Durham, N.C.) See, Evans A, DeHaven C, Barrett T, Mitchell M, Milgram E (2009)—Integrated, nontargeted ultrahigh performance liquid chromatography/electrospray ionization tandem mass spectrometry platform for the identification and relative quantification of the small-molecule complement of biological systems. Anal Chem 81: 6656-6667. Lawton K, Berger A, Mitchell M, Milgram K, Evans A, et al. (2008)—Analysis of the adult human plasma metabolome. Pharmacogenomics 9: 383-397. Reitman Z J, Jin G, Karoly E D, Spasojevic I, Yang J, et al. (2011) Profiling the effects of isocitrate dehydrogenase 1 and 2 mutations on the cellular metabolome. PNAS 108: 3270-3265.] Biochemical data were analyzed using Welch's two-sample t-tests.

### ¹³C-Tracer Analyses.

Male db/db mice (8 weeks) were orally gavaged once daily with vehicle, 5 mg/kg compound 309, or 10 mg/kg compound 309. Thirty minutes after dosing at day 8, 0.5 mg/kg of the metabolic tracer was administered, either via intraperitoneal injection for [U-¹³C]-glucose or oral gavage for [U-¹³C]-palmitate, followed by collection of liver, muscle, adipose tissue, and plasma at the timepoints indicated in the figure legends. Frozen samples were sent to SiDMAP, LLC (Los Angeles, Calif.) for isotope tracer analysis [Boros L G, Huang D, Heaney A P (2012) Fructose Drives Glucose via Direct Oxidation and Promotes Palmitate/Oleate Co-Release from Hepg2 Cells: Relevance with the Randle Cycle. Metabolomics 2: 107-115; Huang J, Simcox J, Mitchell T C, Jones D, Cox J, et al. (2013) Iron regulates glucose homeostasis in liver and muscle via AMP-activated protein kinase in mice. FASEB J.]. [U-¹³C]-D-glucose and [U-¹³C]-palmitate were from Sigma-Aldrich. Sample preparation, analysis, and informatics were performed on blinded samples. Statistical analyses were performed using the 2-tailed Student t test.

### Treatment Affects Mitochondrial Function In Vivo.

Metabolite profiling of tissues from db/db mice orally dosed with either vehicle or 10 mg/kg compound 309 was used to characterize in detail the effects of the present AMPK activators on nutrient pathways in a commonly used mouse model of type 2 diabetes. The compound 309 dose was selected based upon the robust AMPK activation observed in both liver and muscle lysates from normal C57BL/6J mice given a single oral administration of 10 mg/kg compound 309. Since AMPK activation results in both acute and chronic changes to metabolic pathways, samples from non-fasted mice treated for three days with compound 309 were analyzed. Samples were collected thirty minutes after compound dosing when compound 309 plasma levels as well as tissue AMPK activation were maximal.

In muscle, fat, and plasma, compound 309-treatment resulted in a significant elevation of 3-hydroxybutyrate (BHBA) (Table 3, below), a ketone body which can be produced from acetyl CoA generated viamitochondrial fatty acid breakdown, and is consistent with the role of AMPK in regulating fatty acid β-oxidation (Hardie D G, Ross F A, Hawley S A (2012) AMP-Activated Protein Kinase: A Target for Drugs both Ancient and Modern. Chem Biol 19: 1222-1236.). In the liver, no difference was apparent in BHBA levels between compound 309-treated animals compared to the vehicle. However, given that the liver is the primary site of ketone body synthesis, the marked increase in the plasma was likely due to increased liver BHBA production, followed by secretion into the plasma. Levels of hydroxybutyrylcarnitine, the carnitine-modified form of BHBA, were similarly increased in all compartments sampled.

TABLE 3
Branched chain amino acid levels in different tisues
following treatment with an exemplary AMPK activator.

| | Ratio (Compound 309/Vehicle) | | | |
|---|---|---|---|---|
| Fatty Acid Oxidation | Liver | Muscle | Adipose | Plasma |
| 3-hydroxybutyrate (BHBA) | 1.15 | 2.2** | 1.68** | 1.93** |
| hydroxybutyrylcarnitine | 1.45** | 1.34* | 1.7** | 2.28** |

*Up, 0.05 < p < 0.1;
**Up, p < 0.05

One novel finding was a significant decrease in intermediates of the branched chain amino acids (BCAA), which are metabolized in the mitochondria into products that feed into the TCA cycle. Marked reductions in intermediates representing the catabolic pathways for all three BCAA were observed across all three major metabolic organs and in plasma with compound 309 treatment (Table 4, below). This cross-compartment decrease mediated by compound 309 appeared specific for the BCAA pathway since relative levels of the other amino acids were not similarly affected (Table 5, below). Also observed was a reduction in the relative levels of butyrylglycine and butyrylcarnitine in liver and plasma. These metabolites can be derived from fatty acid β-oxidation but they can also be produced via an alternate “R” pathway of isoleucine catabolism whose use is limited under normal conditions but can increase when upstream intermediates accumulate during impaired S pathway function. The overall decrease in tissue BCAA in conjunction with the decrease in plasma isoleucine, leucine, and additional BCAA intermediates is consistent with increased BCAA catabolism in the mitochondria and suggests that mitochondrial function is improved by compound 309.

TABLE 4
Branched chain amino acid levels in different tisues
following treatment with an exemplary AMPK activator.

| | Ratio (Compound 309/Vehicle) | | | |
|---|---|---|---|---|
| Branched Chain Amino Acids | Liver | Muscle | Adipose | Plasma |
| 3-methyl-2-oxobutyrate | | | 1 | 1.17 |
| 3-methyl-2-oxovalerate | | 0.78 | 1.31** | 1.06 |
| alpha-hydroxyisocaproate | | | | 0.91 |
| isoleucine | 0.86^{††} | 0.81^{††} | 0.73 | 0.75^{††} |
| leucine | 0.94 | 0.83^{†} | 0.78 | 0.75^{†} |
| N-acetylleucine | 0.94 | | | 0.96 |
| N-acetylisoleucisne | | | | 1.11 |
| tigloylglycine | | | | 0.96 |
| valine | 0.87^{††} | 0.77^{††} | 0.81 | 0.91 |
| 3-hydroxyisobutyrate | | 1.33* | 1.13 | 1.69** |
| 4-methyl-2-oxopentanoate | | 0.96 | 1.23 | 1.05 |
| alpha-hydroxyisovalerate | 0.86 | 0.85 | | 0.91 |
| isovalerylglycine | | | | 0.67^{††} |
| isobutyrylcarnitine | | 0.64^{††} | 0.57^{††} | 0.41^{††} |
| 2-hydroxy-3-methylvalerate | | | | 0.91 |
| 2-methylbutyrlcarnitine (C5) | 0.76 | 0.63^{††} | 0.57^{††} | 0.56^{††} |
| isovalerylcarnitine | 0.54^{††} | 0.58^{††} | 0.58^{††} | 0.45^{††} |
| hydroxylisovaleroyl carnitine | 1 | 0.86^{†} | 0.86 | |
| tiglyl carnitine | | 0.98 | | |
| 3-methylglutaryl carnitine (C6) | 0.57^{††} | | | 0.55^{††} |
| propionylcarnitine | 0.73 | 0.87^{†} | 0.77 | 1.07 |
| butyrylcarnitine | | 0.62 | 0.82 | 0.65^{†} |
| butyrylglycine | 0.41^{††} | 1.07 | | 0.41^{††} |

*Up, 0.05 < p < 0.1;
**Up, p < 0.05;
^{†}Down, 0.05 < p < 0.1;
^{††}Down, p < 0.05

TABLE 5
Unbranched chain amino acid levels in different tisues
following treatment with an exemplary AMPK activator.

Ratio (Compound 309/Vehicle)

| Amino Acids | Liver | Muscle | Adipose | Plasma |
|---|---|---|---|---|
| glycine | 0.96 | 0.96 | 0.81 | 1.03 |
| serine | 1.19 | 0.89 | 0.85 | 0.83^{†} |
| threonine | 0.94 | 0.89 | 0.77^{†} | 0.9 |
| aspartate | 1.59** | 0.82 | 0.72^{††} | |
| asparagine | | | 0.84 | 0.89 |
| alanine | 1.4** | 0.96 | 1 | 1.22 |
| glutamate | 1.61** | 1.15 | 0.89 | 1.22* |
| glutamine | 1.04 | 1.04 | 0.95 | 1.03 |
| histidine | 1.16* | 1 | | 0.99 |
| lysine | 1.11* | 0.96 | 0.74 | 0.91 |
| phenylalanine | 0.96 | 0.91 | 0.82 | 0.87 |
| tyrosine | 1.08 | 0.96 | 0.85 | 1.11 |
| tryptophan | 0.99 | 0.95 | 0.9 | 1.02 |
| cysteine | 0.97 | | 0.62^{†} | 0.94 |
| methionine | 1.03 | 0.78^{†} | 0.87 | 0.88 |
| arginine | 1.67 | 0.71 | 0.86^{††} | 0.95 |
| proline | 0.94 | 0.92 | 0.86 | 0.96 |

*Up, 0.05 < p < 0.1;
**Up, p < 0.05;
^{†}Down, 0.05 < p < 0.1;
^{††}Down, p < 0.05

### Treatment Increases Palmitate Oxidation in Liver, Skeletal Muscle, and Adipose Tissue.

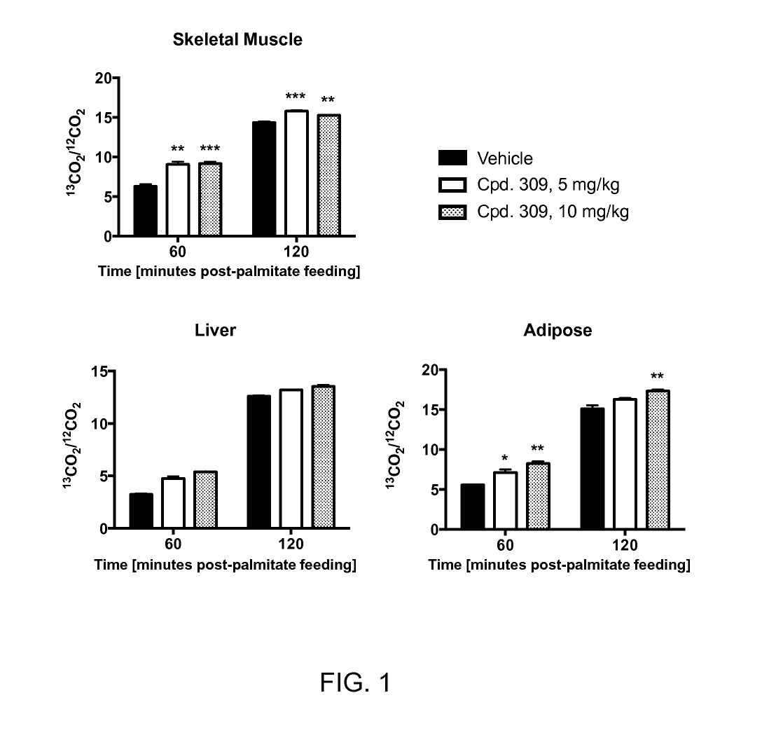

The elevated levels of BHBA detected by the global metabolite profiling are an indirect indicator that compound 309 treatment increases lipid oxidation. In order to determine whether compound 309 had a direct effect on β-oxidation, [U-¹³C]-palmitate was orally administered to non-fasted mice treated with either vehicle or compound 309 for one week followed by measurements of ¹³CO₂ to ¹²CO₂ ratio in various tissues after palmitate feeding. Appearance of ¹³CO₂occurs via β-oxidation of the labeled palmitate to release ¹³C-acetyl CoA, which must pass through one round of the TCA cycle before the labeled carbon can be given off as ¹³CO₂. An increased β-oxidation rate will result in a corresponding increase in ¹³CO₂ enrichment. In agreement with the elevation in relative BHBA levels detected in liver, adipose, and skeletal muscle tissues, an increase in ¹³CO₂ enrichment is observed for compound 309 treatment across all three tissues at both 60 and 120 minutes after palmitate administration. Ratios of ¹³CO₂ to ¹²CO₂ were higher for all treatment groups, including the vehicle, at 120 minutes compared to 60 minutes after palmitate feeding (FIG. 1). This time-dependent elevation was due to the slow absorption kinetics of the orally administered tracer suspension (DeLany J, Windhauser M, Champagne C, Bray G (2000)—Differential oxidation of individual dietary fatty acids in humans. Am J Clin Nutr 72: 905-911.). In contrast to the liver and adipose tissue, no dose-dependence in ¹³CO₂ enrichment was evident in skeletal muscle. All the data together demonstrate that chronic compound 309 treatment of db/db mice directly upregulated fatty acid oxidation in liver, skeletal muscle, and adipose tissue.

### Treatment Increases Glucose Oxidation in Liver, Skeletal Muscle, and Adipose Tissue.

Despite the robust enhancement of glucose uptake and reduction in gluconeogenesis produced by compound 309 treatment in vitro, there were no dramatic effects on glucose metabolic pathways observed in the global metabolite profiling (data not shown).

A more sensitive [U-¹³C]-D-glucose tracer analysis was carried out as follows: Non-fasted db/db mice treated with vehicle or compound 309 for one week were given an intraperitoneal injection of an [U-¹³C]-D-glucose tracer (0.5 mg/kg) followed by measurements of tracer carbon incorporation into various metabolites after glucose injection. In both liver and adipose tissues from compound 309-treated mice, a clear dose-dependent increase in ¹³CO₂ enrichment was observed at both 60 and 90 minutes post-tracer administration (FIG. 2). Skeletal muscle treated with compound 309 displayed a contrasting pattern of ¹³CO₂ enrichment. At 60 minutes, no differences between vehicle and compound treatment were apparent but by 90 minutes, the enrichment in ¹³CO₂ in the vehicle declined by 50% whereas the tracer oxidation was clearly sustained in the compound 309 treated animals. Similar to the palmitate tracer results, no dose-dependence in complete glucose tracer oxidation was observed in skeletal muscle, suggesting that the muscle mitochondrion is fully responsive to compound 309 treatment with the maximal effects reached at the lower dose. Thus, compound 309 increased ¹³CO₂ release in all three tissues, indicating that glucose oxidation via the TCA cycle was increased.

Measurements of skeletal muscle palmitate and myristate following saponification of muscle acylglycerols and acylcarnitines showed significant decreases in the levels of both these fatty acid pools in compound 309-treated mice, and correlates well with the increased palmitate oxidation shown with the ¹³C-palmitate tracer (FIGS. 6 and 4). At both 60 and 90 minutes, the palmitate and myristate pools from compound 309-treated animals were significantly enriched in ¹³C-labeled fatty acids, with the myristate population containing a two-fold higher level of ¹³C-labeled myristate at the 90 minute timepoint in the compound 309-treated mice compared to vehicle. Oxidation of this pool of fatty acids enriched in ¹³C-labeled molecules may contribute to the sustained elevation in ¹³CO₂ release observed in the skeletal muscle at 90 minutes with compound 309 treatment. These ¹³C-labeled fatty acids were muscle-derived (direct muscle data not shown) as no increase in the ¹³C-labeled fraction of palmitate was observed in either the plasma or the liver (data not shown).

What is claimed is:

1. A method of treating cancer, increasing vascular flow, treating a disorder of vascular flow, treating a disorder of glycogen storage, treating pulmonary arterial hypertension, treating vasculitis or treating venous ulcers, in a subject in need thereof, the method comprising administering to the subject a therapeutically-effective amount of an AMPK-activating compound or a pharmaceutically acceptable salt, prodrug or N-oxide thereof, or a solvate or hydrate thereof.

2. The method according to claim 1, wherein the method is a method for treating cancer in a subject in need thereof, the method comprising administering to the subject a therapeutically-effective amount of an AMPK-activating compound or a pharmaceutically acceptable salt, prodrug or N-oxide thereof, or a solvate or hydrate thereof, optionally in combination with one or more of ionizing radiation therapy, a chemotherapeutic agent, p53 gene therapy, chemotherapy and ionizing radiation therapy.

3. The method according to claim 2, wherein the cancer is selected from the group consisting of breast cancer, pancreas cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteosarcoma, chondrosarcoma, Ewing's sarcoma, malignant fibrous histiocytoma, fibrosarcoma, multiple myeloma, reticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, glioblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas.

4. The method according to claim 2, wherein the cancer is one in which wild-type p53 is expressed.

5. The method according to claim 2, wherein the cancer is selected from the group consisting of melanoma, myeloma, endometrial carcinosarcoma, soft tissue sarcoma, hepatocellular carcinoma, lung adenocarcinoma, large lung cell carcinoma and colorectal carcinoma.

6. The method according to claim 1, wherein the method is a method of increasing vascular flow in a subject in need thereof, the method comprising administering to the subject a therapeutically-effective amount of an AMPK-activating compound or a pharmaceutically acceptable salt, prodrug or N-oxide thereof, or a solvate or hydrate thereof.

7. The method according to claim 1, wherein the method is a disorder of vascular flow in a subject in need thereof, the method comprising administering to the subject a therapeutically-effective amount of an AMPK-activating compound or a pharmaceutically acceptable salt, prodrug or N-oxide thereof, or a solvate or hydrate thereof.

8. The method according to claim 7, wherein the disorder of vascular flow is selected from erectile dysfunction, primary or secondary Raynaud's disease, peripheral vascular disease, diabetic angiopathy and peripheral artery disease.

9. The method according to claim 7, wherein the disorder of vascular flow is selected from arteriosclerosis obliterans, Buerger's disease, and progressive systemic sclerosis, systemic erythematosus, vibration syndrome, aneurysm, and vasculitis.

10. The method according to claim 7, wherein the disorder of vascular flow is pulmonary arterial hypertension.

11. The method according to claim 1, wherein the method is a disorder of glycogen storage in a subject in need thereof, the method comprising administering to the subject a therapeutically-effective amount of an AMPK-activating compound or a pharmaceutically acceptable salt, prodrug or N-oxide thereof, or a solvate or hydrate thereof.

12. The method according to claim 11, wherein the disorder of glycogen storage is Pompe disease.

13. The method according to claim 1, wherein the method is a method of treating vasculitis or venous ulcers in a subject in need thereof, the method comprising administering to the subject a therapeutically-effective amount of an AMPK-activating compound or a pharmaceutically acceptable salt, prodrug or N-oxide thereof, or a solvate or hydrate thereof.

14. The method according to claim 1, wherein the AMPK-activating compound is a compound having the structural formula 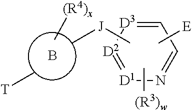
or a pharmaceutically acceptable salt, prodrug, or N-oxide thereof, or a solvate or hydrate thereof, wherein
0 or 1 of D¹, D² and D³ is N, with the others independently being CH or C substituted by one of the w R³;
E is —R², —C(O)NR¹R², —NR¹R² or —NR¹C(O)R², in which R¹ and R² together with the nitrogen to which they are bound form Hca, or R¹ is H, —(C₁-C₄ alkyl), —C(O)—(C₁-C₄ alkyl) or —C(O)O—(C₁-C₄ alkyl), and R² is —C(O)Hca, —(C₀-C₃ alkyl)-Ar, —(C₀-C₃ alkyl)-Het, —(C₀-C₃ alkyl)-Cak or —(C₀-C₃ alkyl)-Hca;
each R³ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
each R⁴ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo, and two R⁴ on different carbons optionally combine to form a —(C₀-C₄ alkylene)- bridge;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene, 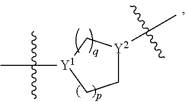
wherein each of Y¹ and Y² is N, C or CH, provided that at least one of Y¹ and Y² is N; p is 0, 1, 2, 3 or 4, q is 1, 2, 3 or 4, and the sum of p and q is 1, 2, 3, 4, 5 or 6, or 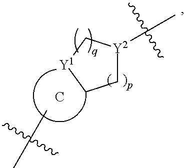
wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6;
T is H, —(C₁-C₆ alkyl), —(C₁-C₆ alkyl)-R²³ in which R²³ is Het or Ar and in which one or more non-adjacent carbons of the alkyl is optionally replaced by —O— or —S—, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰ or 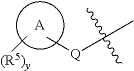
wherein
Q is —O—(C₀-C₃ alkyl)-, —S(O)₂—, -L- or (C₀-C₃ alkyl)-, in which each carbon of the —(C₀-C₃ alkyl)- is optionally and independently substituted with one or two R¹⁶;
the ring system denoted by “A” is heteroaryl, aryl, cycloalkyl or heterocycloalkyl;
each R⁵ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, N₃, —SF₅, —NO₂ and —CN; and
y is 0, 1, 2, 3 or 4;
in which
each L is independently selected
from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR⁹)NR⁹—, —NR⁹SO₂—, —SO₂NR⁹— and —NR⁹SO₂NR⁹—,
each R⁶, R⁷, R⁸ and R¹⁰ is independently selected from H, —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-(C₀-C₆ alkyl), —(C₀-C₆ alkyl)-NR⁹—(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl),
each R⁹ is independently selected from —H, —(C₁-C₄ alkyl), —C(O)—(C₁-C₄ alkyl) and —C(O)O—(C₁-C₄ alkyl),
each Ar is an optionally substituted aryl,
each Het is an optionally substituted heteroaryl,
each Cak is an optionally substituted cycloalkyl,
each Hca is an optionally substituted heterocycloalkyl, and
each alkyl is optionally substituted.

15. The method according to claim 14, wherein the AMPK-activating compound has the structural formula 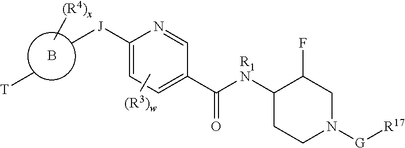
or a pharmaceutically acceptable salt or N-oxide thereof, or a solvate or hydrate thereof, wherein
R¹ is H, —(C₁-C₄ alkyl), —C(O)—(C₁-C₄ alkyl) or —C(O)O—(C₁-C₄ alkyl);
each R³ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
G is —CH₂—, —C(O)—, —S(O)₂—, —CH(CH₃)—, —C(CH₃)₂—, —O—, —C(O)—NH—, —C(O)—NH—CH₂—, —CH₂CH₂—, a single bond, —OCH₂—, CH₂CH₂O—, —CH(COOMe)- or —CH(COOEt)-;
R¹⁷ is aryl or heteroaryl substituted with 1, 2 or 3 substituents independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
each R⁴ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene, 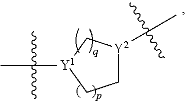
wherein each of Y¹ and Y² is N, C or CH, provided that at least one of Y¹ and Y² is N;
p is 0, 1, 2, 3 or 4, q is 1, 2, 3 or 4, and the sum of p and q is 1, 2, 3, 4, 5 or 6, or 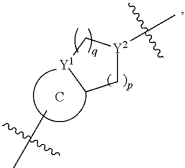
wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6;
T is 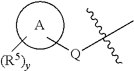
wherein
Q is single bond, —CH₂—, —CH₂O—, —OCH₂CH₂—, —CH₂CH₂—, —O—, —CHF—, —CH(CH₃)—, —C(CH₃)₂—, —CH(OH)—, —CH(COOMe)-, —CH(COOEt)-, —C(O)— or —S(O)₂—;
the ring system denoted by “A” is heteroaryl or aryl;
each R⁵ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —N₃, —SF₅, —NO₂ and —CN; and
y is 0, 1, 2, 3 or 4;
in which
each L is independently selected from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR²)NR⁹—, —NR⁹SO₂—, —SO₂NR⁹— and —NR⁹SO₂NR⁹—,
each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl), and
each R⁹ is independently selected from —H, —(C₁-C₄ alkyl), —C(O)—(C₁-C₄ alkyl) and —C(O)O—(C₁-C₄ alkyl).

16. The method according to claim 15, wherein
G is —CH₂—, —C(O)—, or —S(O)₂—;
R¹⁷ is phenyl or monocyclic heteroaryl substituted with 0, 1 or 2 R³⁰;
each R³ is independently selected from methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂ and —CN;
w is 0 or 1;
J is absent, —C(O)—, —NH—, —NHC(O)— or —C(O)NH—;
the ring system denoted by “B” is 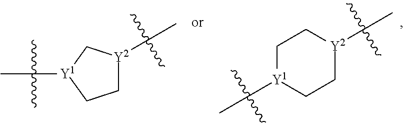
wherein each of Y¹ and Y² is N, C or CH, provided that at least one of Y¹ and Y² is N;
T is 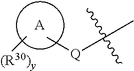
wherein
Q is a single bond, —CH₂—, —O—, —C(O)— or —S(O)₂—;
the ring system denoted by “A” is phenyl or monocyclic heteroaryl; and
y is 0, 1, 2 or 3;
in which
each R³⁰ is independently selected from halogen, unsubstituted (C₁-C₆ alkoxy), —(C₁-C₆ haloalkoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³ and —(NH)₀₋₁COR³³, in which each R³³ is (unsubstituted C₁-C₆ alkyl) or (C₁-C₆ haloalkyl).

16. The method according to claim 15, wherein R¹ is H.

17. The method according to claim 16, wherein w is 0.

18. The method according to claim 1, wherein the AMPK activating compound is N-((trans)-1-(4-cyanobenzyl)-3-fluoropiperidin-4-yl)-6-(4-(4-methoxybenzoyl)piperidine-1-carbonyl)nicotinamide,

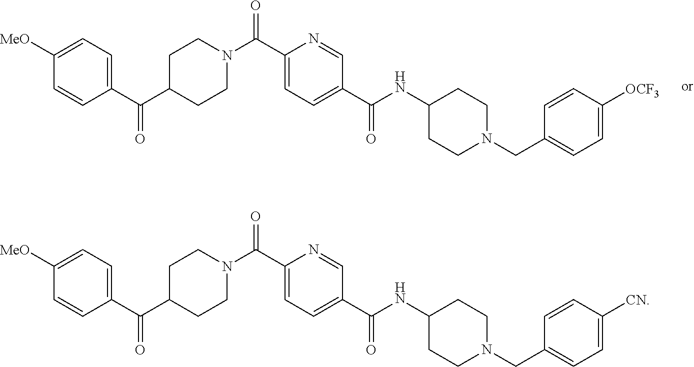 [SMILES: COc1ccc(C(=O)C2CCN(C(=O)c3ccc(C(=O)NC4CCN(Cc5ccc(OC(F)(F)F)cc5)CC4)cn3)CC2)cc1 ; COc1ccc(C(=O)C2CCN(C(=O)c3ccc(C(=O)NC4CCN(Cc5ccc(C#N)cc5)CC4)cn3)CC2)cc1]

19. The method according to claim 1, wherein the AMPK activating compound has an EC₅₀ for AMPK activation of less than about 1 μM.

20. A method of sensitizing a cancer cell to apoptosis, upregulating p53 activity in a cancer cell, inducing a cytotoxic effect in a cancer cell, or down-regulating UHRF1 (Np95) in a cell, the method comprising contacting the cancer cells with an effective amount of an AMPK-activating compound or a pharmaceutically acceptable salt, prodrug or N-oxide thereof, or a solvate or hydrate thereof.

21. The method according to claim 20, wherein the cancer cell is selected from the group consisting of breast cancer, pancreatic cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteosarcoma, chondrosarcoma, Ewing's sarcoma, malignant fibrous histiocytoma, fibrosarcoma, multiple myeloma, reticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, glioblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas.

22. The method according to claim 20, wherein the cancer or cancer cell is one in which wild-type p53 is expressed.

23. The method according to claim 20, wherein the cancer cell is selected from the group consisting of melanoma, myeloma, endometrial carcinosarcoma, soft tissue sarcoma, hepatocellular carcinoma, lung adenocarcinoma, large lung cell carcinoma and colorectal carcinoma.

24. The method according to claim 20, wherein the AMPK-activating compound is a compound having the structural formula 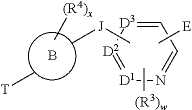
or a pharmaceutically acceptable salt, prodrug, or N-oxide thereof, or a solvate or hydrate thereof, wherein
0 or 1 of D¹, D² and D³ is N, with the others independently being CH or C substituted by one of the w R³;
E is —R², —C(O)NR¹R², —NR¹R² or —NR¹C(O)R², in which R¹ and R² together with the nitrogen to which they are bound form Hca, or R¹ is H, —(C₁-C₄ alkyl), —C(O)—(C₁-C₄ alkyl) or —C(O)O—(C₁-C₄ alkyl), and R² is —C(O)Hca, —(C₀-C₃ alkyl)-Ar, —(C₀-C₃ alkyl)-Het, —(C₀-C₃ alkyl)-Cak or —(C₀-C₃ alkyl)-Hca;
each R³ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
each R⁴ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo, and two R⁴ on different carbons optionally combine to form a —(C₀-C₄ alkylene)- bridge;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene 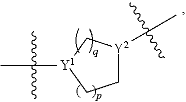
wherein
each of Y¹ and Y² is N, C or CH, provided that at least one of Y¹ and Y² is N; p is 0, 1, 2, 3 or 4, q is 1, 2, 3 or 4, and the sum of p and q is 1, 2, 3, 4, 5 or 6, or 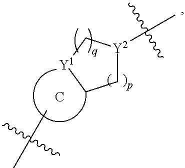
wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6;
T is H, —(C₁-C₆ alkyl), —(C₁-C₆ alkyl)-R²³ in which R²³ is Het or Ar and in which one or more non-adjacent carbons of the alkyl is optionally replaced by —O— or —S—, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰ or 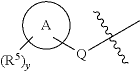
wherein
Q is —O—(C₀-C₃ alkyl)-, —S(O)₂—, -L- or (C₀-C₃ alkyl)-, in which each carbon of the —(C₀-C₃ alkyl)- is optionally and independently substituted with one or two R¹⁶;
the ring system denoted by “A” is heteroaryl, aryl, cycloalkyl or heterocycloalkyl;
each R⁵ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, N₃, —SF₅, —NO₂ and —CN; and
y is 0, 1, 2, 3 or 4;
in which
each L is independently selected
from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR⁹)NR⁹—, —NR⁹SO₂—, —SO₂NR⁹— and —NR⁹SO₂NR⁹—,
each R⁶, R⁷, R⁸ and R¹⁰ is independently selected from H, —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-(C₀-C₆ alkyl), —(C₀-C₆ alkyl)-NR⁹—(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl),
each R⁹ is independently selected from —H, —(C₁-C₄ alkyl), —C(O)—(C₁-C₄ alkyl) and —C(O)O—(C₁-C₄ alkyl),
each Ar is an optionally substituted aryl,
each Het is an optionally substituted heteroaryl,
each Cak is an optionally substituted cycloalkyl,
each Hca is an optionally substituted heterocycloalkyl, and
each alkyl is optionally substituted.

25. The method according to claim 24, wherein the AMPK-activating compound has the structural formula 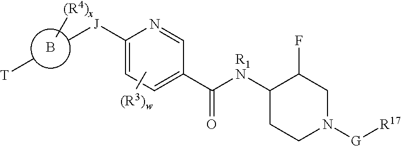
or a pharmaceutically acceptable salt or N-oxide thereof, or a solvate or hydrate thereof, wherein
R¹ is H, —(C₁-C₄ alkyl), —C(O)—(C₁-C₄ alkyl) or —C(O)O—(C₁-C₄ alkyl);
each R³ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
G is —CH₂—, —C(O)—, —S(O)₂—, —CH(CH₃)—, —C(CH₃)₂—, —O—, —C(O)—NH—, —C(O)—NH—CH₂—, —CH₂CH₂—, a single bond, —OCH₂—, CH₂CH₂O—, —CH(COOMe)- or —CH(COOEt)-;
R¹⁷ is aryl or heteroaryl substituted with 1, 2 or 3 substituents independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
each R⁴ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene, 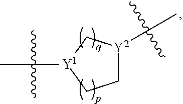
wherein each of Y¹ and Y² is N, C or CH, provided that at least one of Y¹ and Y² is N;
p is 0, 1, 2, 3 or 4, q is 1, 2, 3 or 4, and the sum of p and q is 1, 2, 3, 4, 5 or 6, or 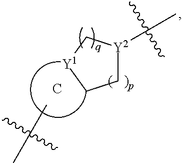
wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6;
T is 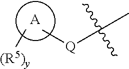
wherein
Q is single bond, —CH₂—, —CH₂O—, —OCH₂CH₂—, —CH₂CH₂—, —O—, —CHF—, —CH(CH₃)—, —C(CH₃)₂—, —CH(OH)—, —CH(COOMe)-, —CH(COOEt)-, —C(O)— or —S(O)₂—;
the ring system denoted by “A” is heteroaryl or aryl;
each R⁵ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —N₃, —SF₅, —NO₂ and —CN; and
y is 0, 1, 2, 3 or 4;
in which
each L is independently selected from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR²)NR⁹—, —NR⁹SO₂—, —SO₂NR⁹— and —NR⁹SO₂NR⁹—,
each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl), and
each R⁹ is independently selected from —H, —(C₁-C₄ alkyl), —C(O)—(C₁-C₄ alkyl) and —C(O)O—(C₁-C₄ alkyl).

26. A method of determining the degree of AMPK activation in a subject comprising:
optionally, administering to the subject an AMPK-activating compound; then
obtaining a sample from the subject; and
measuring the concentration of a biomarker of AMPK activation in the sample from the subject.

27. The method according to claim 26, wherein the AMPK-activating compound is administered to the subject.

28. The method according to claim 26, wherein the concentration of the biomarker of AMPK activation is correlated with a therapeutic dosage for the treatment of an AMPK-linked disorder selected from the group consisting of cancer, disorders of vascular flow, disorder of glycogen storages, increased triglyceride levels, decreased insulin sensitivity, metabolic disorders, diabetes, type I diabetes, type II diabetes, hyperglycemia, hyperinsulinemia, hypertriglyceridemia, atherosclerosis, cardiovascular disease, disorders of decreased or insufficient metabolic efficiency, and increased oxidative stress.

29. The method according to claim 28, further comprising administering to the subject the AMPK-activating compound at at least about the therapeutic dosage.

30. A method according to claim 26, the method comprising
obtaining a first sample from the subject;
measuring the initial concentration of a biomarker of AMPK activation in the first sample from the subject;
after obtaining the first sample from the subject, administering to the subject an AMPK-activating compound;
after administration, obtaining a second sample from the subject; and
measuring the concentration of the biomarker in the second sample from the subject.

31. The method according to claim 30, further comprising correlating the degree of AMPK activation with the concentration of the biomarker of AMPK activation in the first sample and in the second sample.

32. The method according to claim 30, wherein the concentration of the biomarker of AMPK activation in the second sample, optionally together with the concentration of the biomarker of AMPK activation in the first sample, is correlated with a therapeutic dosage for the treatment of an AMPK-linked disorder selected from the group consisting of cancer, disorders of vascular flow, disorder of glycogen storages, increased triglyceride levels, decreased insulin sensitivity, metabolic disorders, diabetes, type I diabetes, type II diabetes, hyperglycemia, hyperinsulinemia, hypertriglyceridemia, atherosclerosis, cardiovascular disease, disorders of decreased or insufficient metabolic efficiency, and increased oxidative stress.

33. The method according to claim 32, further comprising administering to the subject the AMPK-activating compound at at least about the therapeutic dosage.

34. The method according to claim 26, comprising
obtaining a first sample from the subject;
measuring the concentration of a biomarker of AMPK activation in the first sample from the subject; and
selecting a therapeutic dosage of the AMPK-activating compound based on the concentration of the biomarker of AMPK activation in the first sample, the therapeutic dosage being for the treatment of an AMPK-linked disorder selected from the group consisting of cancer, disorders of vascular flow, disorder of glycogen storages, increased triglyceride levels, decreased insulin sensitivity, metabolic disorders, diabetes, type I diabetes, type II diabetes, hyperglycemia, hyperinsulinemia, hypertriglyceridemia, atherosclerosis, cardiovascular disease, disorders of decreased or insufficient metabolic efficiency, and increased oxidative stress.

35. The method according to claim 34, further comprising administering to the subject the AMPK-activating compound at at least about the therapeutic dosage.

36. The method according to claim 26, wherein the method further activates the AMPK pathway in a subject in need thereof, and wherein the method comprises:
obtaining a first sample from the subject;
measuring the concentration of a biomarker of AMPK activation in the first sample from the subject;
after obtaining the first sample, administering to the subject an AMPK-activating compound at a test dosage;
after administration, obtaining a second sample from the subject;
measuring the concentration of the biomarker of AMPK activation in the second sample from the subject;
selecting a therapeutic dosage of the AMPK-activating compound based on the concentration of the biomarker of AMPK activation in the second sample, optionally together with the concentration of the biomarker of AMPK activation in the first sample, the therapeutic dosage being for the treatment of an AMPK-linked disorder selected from the group consisting of cancer, disorders of vascular flow, disorder of glycogen storages, increased triglyceride levels, decreased insulin sensitivity, metabolic disorders, diabetes, type I diabetes, type II diabetes, hyperglycemia, hyperinsulinemia, hypertriglyceridemia, atherosclerosis, cardiovascular disease, disorders of decreased or insufficient metabolic efficiency, and increased oxidative stress; and
administering to the subject the AMPK-activating compound at at least about the therapeutic dosage.

37. The method according to claim 26, wherein the method further activates the AMPK pathway in a subject in need thereof, and wherein the method comprises:
administering to the subject an AMPK-activating compound at a test dosage;
after administration, obtaining a sample from the subject;
measuring the concentration of the biomarker of AMPK activation in the sample from the subject;
selecting a therapeutic dosage of the AMPK-activating compound based on the concentration of the biomarker of AMPK activation in the sample, the therapeutic dosage being for the treatment of an AMPK-linked disorder selected from the group consisting of cancer, disorders of vascular flow, disorder of glycogen storages, increased triglyceride levels, decreased insulin sensitivity, metabolic disorders, diabetes, type I diabetes, type II diabetes, hyperglycemia, hyperinsulinemia, hypertriglyceridemia, atherosclerosis, cardiovascular disease, disorders of decreased or insufficient metabolic efficiency, and increased oxidative stress; and
administering to the subject the AMPK-activating compound at at least about the therapeutic dosage.

38. The method according to claim 26, wherein the biomarker of AMPK activation is a branched chain amino acid, valine, leucine, isoleucine, tyrosine, phenylalanine, an acylcarnitine intermediate, isobutyrlcarnitine, 2-methylbutyrylcarnitine, isovalerylcarnitine, insulin-like growth factor-binding protein-1, a ketone body, 3-hydroxybutyrate, acetone, acetoacetate, a citric acid cycle intermediate, citrate, fumarate, malate, citrulline, a fatty acid, palmitate or myristate.

39. The method according to claim 27, wherein the AMPK-activating compound is administered at a level sufficient to cause the measured concentration of the biomarker post-administration to be within about 40% of a control concentration.

40. The method according to claim 27, wherein the AMPK-activating compound is administered at a level sufficient to cause the measured concentration of the biomarker post-administration to be, for a biomarker whose concentration is positively correlated with AMPK activation, at least about 60% of a control concentration, and for a biomarker whose concentration is negatively correlated with AMPK activation, no greater than about 140% of a control concentration.

41. The method according to claim 27, wherein the AMPK-activating compound is administered at a level sufficient to cause the measured concentration of the biomarker post-administration to change by at least about 10% as compared to the measured concentration pre-administration.

42. The method according to claim 26, wherein the AMPK-activating compound is a compound having the structural formula 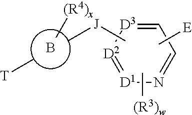
or a pharmaceutically acceptable salt, prodrug, or N-oxide thereof, or a solvate or hydrate thereof, wherein
0 or 1 of D¹, D² and D³ is N, with the others independently being CH or C substituted by one of the w R³;
E is —R², —C(O)NR¹R², —NR¹R² or —NR¹C(O)R², in which R¹ and R² together with the nitrogen to which they are bound form Hca, or R¹ is H, —(C₁-C₄ alkyl), —C(O)—(C₁-C₄ alkyl) or —C(O)O—(C₁-C₄ alkyl), and R² is —C(O)Hca, —(C₀-C₃ alkyl)-Ar, —(C₀-C₃ alkyl)-Het, —(C₀-C₃ alkyl)-Cak or —(C₀-C₃ alkyl)-Hca;
each R³ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
each R⁴ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo, and two R⁴ on different carbons optionally combine to form a —(C₀-C₄ alkylene)- bridge;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene, 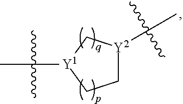
wherein each of Y¹ and Y² is N, C or CH, provided that at least one of Y¹ and Y² is N; p is 0, 1, 2, 3 or 4, q is 1, 2, 3 or 4, and the sum of p and q is 1, 2, 3, 4, 5 or 6, or 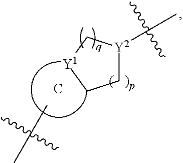
wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6;
T is H, —(C₁-C₆ alkyl), —(C₁-C₆ alkyl)-R²³ in which R²³ is Het or Ar and in which one or more non-adjacent carbons of the alkyl is optionally replaced by —O— or —S—, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰ or 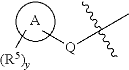
wherein
Q is —O—(C₀-C₃ alkyl)-, —S(O)₂—, -L- or (C₀-C₃ alkyl)-, in which each carbon of the —(C₀-C₃ alkyl)- is optionally and independently substituted with one or two R¹⁶;
the ring system denoted by “A” is heteroaryl, aryl, cycloalkyl or heterocycloalkyl;
each R⁵ is independently selected from —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, N₃, —SF₅, —NO₂ and —CN; and
y is 0, 1, 2, 3 or 4;
in which
each L is independently selected
from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR⁹)NR⁹—, —NR⁹SO₂—, —SO₂NR⁹— and —NR⁹SO₂NR⁹—,
each R⁶, R⁷, R⁸ and R¹⁰ is independently selected from H, —(C₁-C₆ alkyl), —(C₁-C₆ haloalkyl), —(C₀-C₆ alkyl)-Ar, —(C₀-C₆ alkyl)-Het, —(C₀-C₆ alkyl)-Cak, —(C₀-C₆ alkyl)-Hca, —(C₀-C₆ alkyl)-L-(C₀-C₆ alkyl), —(C₀-C₆ alkyl)-NR⁹—(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl),
each R⁹ is independently selected from —H, —(C₁-C₄ alkyl), —C(O)—(C₁-C₄ alkyl) and —C(O)O—(C₁-C₄ alkyl),
each Ar is an optionally substituted aryl,
each Het is an optionally substituted heteroaryl,
each Cak is an optionally substituted cycloalkyl,
each Hca is an optionally substituted heterocycloalkyl, and
each alkyl is optionally substituted.

43. The method according to claim 42, wherein the AMPK-activating compound has the structural formula 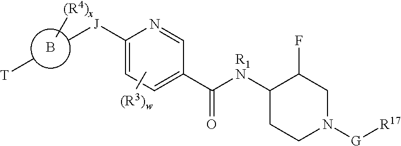
or a pharmaceutically acceptable salt or N-oxide thereof, or a solvate or hydrate thereof, wherein
R¹ is H, —(C₁-C₄ alkyl), —C(O)—(C₁-C₄ alkyl) or —C(O)O—(C₁-C₄ alkyl);
each R³ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
w is 0, 1, 2 or 3;
G is —CH₂—, —C(O)—, —S(O)₂—, —CH(CH₃)—, —C(CH₃)₂—, —O—, —C(O)—NH—, —C(O)—NH—CH₂—, —CH₂CH₂—, a single bond, —OCH₂—, CH₂CH₂O—, —CH(COOMe)- or —CH(COOEt)-;
R¹⁷ is aryl or heteroaryl substituted with 1, 2 or 3 substituents independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN;
each R⁴ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —NO₂ and —CN, and two R⁴ on the same carbon optionally combine to form oxo;
x is 0, 1, 2, 3 or 4;
J is absent, —C(O)—, —NR¹³—, —NR¹³C(O)— or —C(O)NR¹³—, in which R¹³ is selected from —H, —(C₁-C₄ alkyl), —C(O)—(C₁-C₄ alkyl) and —C(O)O—(C₁-C₄ alkyl);
the ring system denoted by “B” is absent, arylene, heteroarylene, 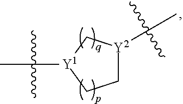
wherein each of Y¹ and Y² is N, C or CH, provided that at least one of Y¹ and Y² is N;
p is 0, 1, 2, 3 or 4, q is 1, 2, 3 or 4, and the sum of p and q is 1, 2, 3, 4, 5 or 6, or 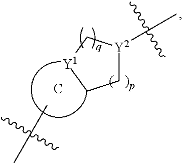
wherein Y¹ is N or C and Y² is N, C or CH, provided that at least one of Y¹ and Y² is N, the ring system denoted by “C” is an arylene or a heteroarylene, p is 0, 1, 2, 3 or 4, q is 1, 2, 3 or 4, and the sum of p and q is 1, 2, 3, 4, 5 or 6;
T is 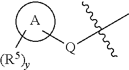
wherein
Q is single bond, —CH₂—, —CH₂O—, —OCH₂CH₂—, —CH₂CH₂—, —O—, —CHF—, —CH(CH₃)—, —C(CH₃)₂—, —CH(OH)—, —CH(COOMe)-, —CH(COOEt)-, —C(O)— or —S(O)₂—;
the ring system denoted by “A” is heteroaryl or aryl;
each R⁵ is independently selected from —(C₁-C₆ alkyl), —(C₁-C₆ haloalkyl), —(C₀-C₆ alkyl)-L-R⁷, —(C₀-C₆ alkyl)-NR⁸R⁹, —(C₀-C₆ alkyl)-OR¹⁰, —(C₀-C₆ alkyl)-C(O)R¹⁰, —(C₀-C₆ alkyl)-S(O)₀₋₂R¹⁰, -halogen, —N₃, —SF₅, —NO₂ and —CN; and
y is 0, 1, 2, 3 or 4;
in which
each L is independently selected from —NR⁹C(O)O—, —OC(O)NR⁹—, —NR⁹C(O)—NR⁹—, —NR⁹C(O)S—, —SC(O)NR⁹—, —NR⁹C(O)—, —C(O)—NR⁹—, —NR⁹C(S)O—, —OC(S)NR⁹—, —NR⁹C(S)—NR⁹—, —NR⁹C(S)S—, —SC(S)NR⁹—, —NR⁹C(S)—, —C(S)NR⁹—, —SC(O)NR⁹—, —NR⁹C(S)—, —S(O)₀₋₂—, —C(O)O, —OC(O)—, —C(S)O—, —OC(S)—, —C(O)S—, —SC(O)—, —C(S)S—, —SC(S)—, —OC(O)O—, —SC(O)O—, —OC(O)S—, —SC(S)O—, —OC(S)S—, —NR⁹C(NR²)NR⁹—, —NR⁹SO₂—, —SO₂NR⁹— and —NR⁹SO₂NR⁹—,
each R⁷, R⁸ and R¹⁰ is independently selected from H, —(C₁-C₆ alkyl), —(C₁-C₆ haloalkyl), —(C₀-C₆ alkyl)-L-(C₀-C₆ alkyl), —(C₀-C₆ alkyl)-NR⁹(C₀-C₆ alkyl), —(C₀-C₆ alkyl)-O—(C₀-C₆ alkyl), —(C₀-C₆ alkyl)-C(O)—(C₀-C₆ alkyl) and —(C₀-C₆ alkyl)-S(O)₀₋₂—(C₀-C₆ alkyl), and
each R⁹ is independently selected from —H, —(C₁-C₄ alkyl), —C(O)—(C₁-C₄ alkyl) and —C(O)O—(C₁-C₄ alkyl).

44. The method according to claim 43, wherein
G is —CH₂—, —C(O)—, or —S(O)₂—;
R¹⁷ is phenyl or monocyclic heteroaryl substituted with 0, 1 or 2 R³⁰;
each R³ is independently selected from methyl, ethyl, n-propyl, isopropyl, trifluoromethyl, pentafluoroethyl, acetyl, —NH₂, —OH, methoxy, ethoxy, trifluoromethoxy, —SO₂Me, -halogen, —NO₂ and —CN;
w is 0 or 1;
J is absent, —C(O)—, —NH—, —NHC(O)— or —C(O)NH—;
the ring system denoted by “B” is 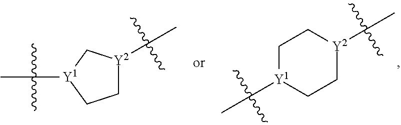
wherein each of Y¹ and Y² is N, C or CH, provided that at least one of Y¹ and Y² is N;
T is 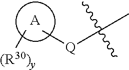
wherein
Q is a single bond, —CH₂—, —O—, —C(O)— or —S(O)₂—;
the ring system denoted by “A” is phenyl or monocyclic heteroaryl; and
y is 0, 1, 2 or 3;
in which
each R³⁹ is independently selected from halogen, unsubstituted (C₁-C₆ alkoxy), —(C₁-C₆ haloalkoxy), —SH, —S(unsubstituted C₁-C₆ alkyl), —S(C₁-C₆ haloalkyl), —OH, —CN, —NO₂, —NH₂, —NH(unsubstituted C₁-C₄ alkyl), —N(unsubstituted C₁-C₄ alkyl)₂, —N₃, —SF₅, —C(O)—NH₂, C(O)NH(unsubstituted C₁-C₄ alkyl), C(O)N(unsubstituted C₁-C₄ alkyl)₂, —C(O)OH, C(O)O(unsubstituted C₁-C₆ alkyl), —(NH)₀₋₁SO₂R³³ and —(NH)₀₋₁COR³³, in which each R³³ is (unsubstituted C₁-C₆ alkyl) or (C₁-C₆ haloalkyl).

45. The method according to claim 44, wherein R¹ is H and w is 0.

46. The method according to claim 42, wherein the AMPK activating compound is N-((trans)-1-(4-cyanobenzyl)-3-fluoropiperidin-4-yl)-6-(4-(4-methoxybenzoyl)piperidine-1-carbonyl)nicotinamide,

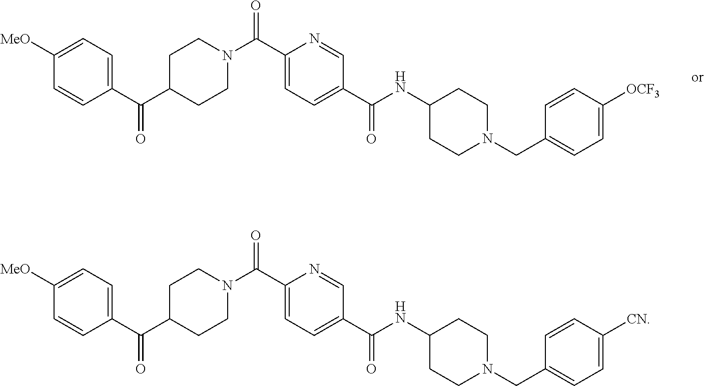 [SMILES: COc1ccc(C(=O)C2CCN(C(=O)c3ccc(C(=O)NC4CCN(Cc5ccc(OC(F)(F)F)cc5)CC4)cn3)CC2)cc1 ; COc1ccc(C(=O)C2CCN(C(=O)c3ccc(C(=O)NC4CCN(Cc5ccc(C#N)cc5)CC4)cn3)CC2)cc1]

47. The method according to claim 42, wherein the AMPK activating compound has an EC₅₀ for AMPK activation of less than about 1 μM.
